(19) United States
(12) Patent Application Publication
(10) Pub. No.: US 2024/0287148 A1
(43) Pub. Date: Aug. 29, 2024

(54) ENGINEERED BIOMOLECULES FOR NUTRIENT REPROGRAMMING

(71) Applicant: CORNELL UNIVERSITY, Ithaca, NY (US)

(72) Inventors: Shu-Bing Qian, Ithaca, NY (US); Robert V. Swanda, Windsor, NY (US)

(21) Appl. No.: 18/578,867

(22) PCT Filed: Jul. 15, 2022
(86) PCT No.: PCT/US2022/073776

§ 371 Date: Jan. 12, 2024

Related U.S. Application Data
(60) Provisional application No. 63/222,339, filed on Jul. 15, 2021.

(51) Int. Cl. C07K 14/47; A61K 38/17; A61K 38/30; A61K 48/00; A61P 35/00; C07K 14/65

(52) U.S. Cl. CPC C07K 14/47; A61K 38/1709; A61K 38/30; A61K 48/0033; A61P 35/00; C07K 14/65; C07K 2319/06; C07K 2319/35

(57) ABSTRACT

Described in several exemplary embodiments herein are engineered biomolecules that can be capable of nutrient reprogramming in a cell. Also described herein are methods of using the engineered biomolecules.

### CROSS-REFERENCE TO RELATED APPLICATIONS

This application claims the benefit of and priority to co-pending U.S. Provisional Patent Application No. 63/222,339, filed on Jul. 15, 2021, entitled “ENGINEERED BIOMOLECULES FOR NUTRIENT REPROGRAMMING,” the contents of which is incorporated by reference herein in its entirety.

### STATEMENT REGARDING FEDERALLY SPONSORED RESEARCH

This invention was made with government support under Grant No. DP1GM142101 awarded by the National Institutes of Health. The government has certain rights in the invention.

### SEQUENCE LISTING

The contents of the electronic sequence listing (CORNL-0700WP_ST26.xml, size is 84,743 bytes and it was created on Jul. 13, 2022) is herein incorporated by reference in its entirety

### TECHNICAL FIELD

The subject matter disclosed herein is generally directed to engineered biomolecules for use in nutrient reprogramming.

### BACKGROUND

The availability of intracellular cysteine and its oxidized form cystine controls oxidative stress response. Upon cyst(e)ine restriction, the accumulation of lipid reactive oxygen species leads to ferroptosis, a non-apoptotic cell death. Inducible ferroptosis has been a focus for the development of cancer therapies. However, the efficacy of cyst(e)ine deprivation within in cells achieved by these potential therapeutics is compromised by the subsequent adaptive response of the cells. As such there exists a need for compositions, methods, and/or techniques to fully realize induced ferroptosis as an intervention and therapy for disease, particularly cancer.

Citation or identification of any document in this application is not an admission that such a document is available as prior art to the present invention.

### SUMMARY

Described in example embodiments herein are engineered biomolecules comprising: (a) a lysosomal targeting moiety; and (b) one or more cysteine-rich motifs, wherein each of the one or more cysteine-rich motifs is coupled to the lysosomal targeting moiety.

In certain example embodiments, the engineered biomolecule is DNA or RNA.

In certain example embodiments, the engineered biomolecule is a polypeptide.

In certain example embodiments, the engineered biomolecule comprises at least two cysteine-rich motifs.

In certain example embodiments, the lysosomal targeting moiety is selected from: IGF2 or M6PR binding domain thereof, any polypeptide set forth in Table 1, a LIMP-2 ligand (e.g., beta-glucocerebrosidase or LIMP-2 binding domain thereof), a sortilin ligand (e.g., Prosaposin or sortilin binding domain thereof), and combinations thereof.

In certain example embodiments, the one or more cysteine-rich motifs are independently selected from DNAJC5, CYSRT1, a native cysteine-rich domain of a protein set forth in Table 2, or a protein set forth in Table 2.

In certain example embodiments, one or more nucleotides or amino acids of the engineered biomolecule are modified, wherein the modification reduces biomolecule immunogenicity, increases biomolecule stability, or both.

In certain example embodiments, the modification at each modified nucleotide is independently selected from methylpseudouridine, a phosphorothioate linkage, a locked nucleic acid (LNA) nucleotides comprising a methylene bridge between the 2′ and 4′ carbons of the ribose ring, or bridged nucleic acids (BNA), 2′-O-methyl analogs, 2′-deoxy analogs, or 2′-fluoro analogs, 2-aminopurine, 5-bromo-uridine, pseudouridine, (Ψ), N1-methylpseudouridine (me1Ψ), 5-methoxyuridine (5moU), inosine, 7-methylguanosine, inosine, 7-methylguanosine. Examples of guide RNA chemical modifications include, without limitation, incorporation of 2′-O-methyl (M), 2′-O-methyl 3′phosphorothioate (MS), S-constrained ethyl (cEt), or 2′-O-methyl 3′thioPACE (MSP) at one or more terminal nucleotides

In certain example embodiments, the modification at each modified amino acid is independently selected from phosphorylation, acetylation, ubiquitylation, methylation, glycosylation, SUMOylation, palmitoylation, myristoylation, prenylation, sulfation, any of those presented in Muller, M. Biochemistry 2018, 57, 2, 177-185 incorporated by reference herein); Ramazi et al., 2021. Database. https://doi.org/10.1093/database/baab012 (incorporated by reference herein), any of those described in any of the following post-translational modification databases: dbPTM, BioGRID. Phosphosite Plus, PTMCodev2, qPTM, PLMD, CPLM, YAAM, HPRD, PHOSIDA, PTM-SD, WERAM, EPSD, PhosphoNET, RegPhos, Phospho.ELM, Phospho3D, dbPSP, pTestis, LymPHOS. P3 DB, UniPep, GlycoFly, GlycoFish, mUbiSiDa, SwissPalm, dbSNO (see also e.g., Ramazi et al., 2021.), those described in Narita et al. Nature Reviews Molecular Cell Biology volume 20, pages 156-174 (2019) (incorporated by reference herein), or any combination thereof.

In certain example embodiments, the engineered biomolecule is effective to inhibit ATF4 expression induction, reduce cytosolic cysteine, increase lysosomal cysteine, inhibit a cyst(e)ine stress response, or any combination thereof in a cell.

In certain example embodiments, the engineered biomolecule is effective to induce and/or potentiate ferroptosis.

Described in certain example embodiments herein are vectors and/or vector systems comprising an engineered biomolecule of any one of the preceding claims, wherein the engineered biomolecule is an engineered polynucleotide; and optionally, a regulatory element, wherein the engineered polynucleotide is operably coupled to the regulatory element.

Described in certain example embodiments herein are delivery vehicles comprising an engineered biomolecule as described herein, a vector or vector system as described herein, or both.

In certain example embodiments, the delivery vehicle comprises a micelle, nanoparticle (e.g., lipid nanoparticles, polymer nanoparticles, metal nanoparticles, inorganic nanoparticles), lipid particles (e.g., liposomes, lipid nanoparticles, stable-nucleic-acid-lipid particles), polymer-based particles, stroptolysin-O, an exosome, an extracellular vesicle, dendrimers, a nanoclew, cell penetrating peptides, a multifunctional envelope-type nanodevice, virus and virus like particles, vectors, vector systems, naked polynucleotides, and any combination thereof.

Described in certain example embodiments herein are pharmaceutical formulations comprising (a) an engineered biomolecule of any one of any one of the preceding paragraphs or as described in greater detail elsewhere herein; (b) a vector as in any one of any one of the preceding paragraphs or as described in greater detail elsewhere herein; (c) a delivery particle as in any one of any one of the preceding paragraphs or as described in greater detail elsewhere herein; or (d) any combination of a-c; and a pharmaceutically acceptable carrier.

In certain example embodiments, the pharmaceutical formulation further comprises an additional active agent.

In certain example embodiments, the additional active agent is effective to induce ferroptosis in a cell.

In certain example embodiments, the additional active agent inhibits the X_{c}⁻ antiporter.

In certain example embodiments, the additional active agent is selected from erastin, Ras-selective lethal small molecule 3, sulfasalazine, lanperisone, sorafenib, fenugreek (trigonelline), acetaminophen, cisplatin, artesunate, combination of siramesine and lapatinib, ferumoxytol, and salinomycin (ironomycin), artesunate, dihydroartemisnin, gemcitabine, paclitaxel, temozolomide, buthionine sulfoximine (BSO), solasonine, siramesine, laptinib, BAY 87-2243, Molecule 1 of Taylor et al. 2019. Sci. Rep., 9: 5926 and its derivatives or metabolites (specifically molecule 4 of Taylor et al. 2019. Sci. Rep., 9: 5926) any others set forth in e.g., Su et al., Cancer. Lett. 2020. Jul. 28; 483:127-136. doi: 10.1016/j.canlet.2020.02.015; Wu et al., 2020. Front. Oncol. | https://doi.org/10.3389/fonc.2020.571127; Lin et al., Front. Pharmacol. 2020. 11: 1061; Taylor et al., 2019. Sci. Reports. 9:5926, all of which are incorporated by reference herein), or any combination thereof.

In certain example embodiments, the additional active agent is selected from erastin, Ras-selective lethal small molecule 3, sulfasalazine or analogue thereof, lanperisone, sorafenib, fenugreek (trigonelline), acetaminophen, cisplatin, artesunate, siramesine, lapatinib, a combination of siramesine and lapatinib, ferumoxytol, salinomycin (ironomycin), dihydroartemisnin, gemcitabine, paclitaxel, temozolomide, buthionine sulfoximine (BSO), solasonine, siramesine, laptinib, BAY 87-2243,

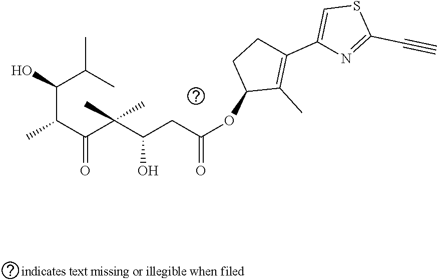

or a derivative or metabolite thereof, optionally

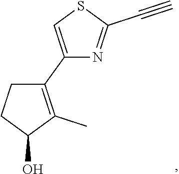

BLZ945, Dyclonine, Oxyfedrine, RSL3, ML210, SSZ, RSL3, ML162, FIN56, Anti-PD1, gefitnib, erlotinib, TMZ, docetaxel, DAT, vemurafenib, lovastatin, atorvastatin, simvastatin, prominin2, artemisinin or a derivative thereof, vitamin E, baicalein, beta-elemene, gallic acid, buthionine sulfoximine, DP17, FIN56, FINO₂, a statin, deferoxamine mesylate, or any combination thereof.

Described in certain example embodiments herein are kits comprising (a) an engineered biomolecule of any one of the preceding claims; (b) a vector of any one of the preceding claims; (c) a delivery vehicle of any one of the preceding claims; (d) a pharmaceutical formulation as in any one of the preceding claims; or (e) any combination thereof.

In certain example embodiments, the kit further comprises an additional active agent.

In certain example embodiments, the additional active agent is effective to induce ferroptosis in a cell.

In certain example embodiments, the additional active agent inhibits the X_{c}⁻ antiporter.

In certain example embodiments, the additional active agent is selected from selected from erastin, Ras-selective lethal small molecule 3, sulfasalazine, lanperisone, sorafenib, fenugreek (trigonelline), acetaminophen, cisplatin, artesunate, combination of siramesine and lapatinib, ferumoxytol, and salinomycin (ironomycin), artesunate, dihydroartemisnin, gemcitabine, paclitaxel, temozolomide, buthionine sulfoximine (BSO), solasonine, siramesine, laptinib, BAY 87-2243, Molecule 1 of Taylor et al., 2019 Sci. Reports. 9:5926 and its derivatives or metabolites (specifically molecule 4 of Taylor et al. 2019) any others set forth in e.g., Su et al., Cancer. Lett. 2020. Jul. 28; 483:127-136. doi: 10.1016/j.canlet.2020.02.015; Wu et al., 2020. Front. Oncol. https://doi.org/10.3389/fonc.2020.571127; Lin et al., Front. Pharmacol. 2020. 11: 1061; Taylor et al., 2019. Sci. Reports. 9:5926, all of which are incorporated by reference herein), or any combination thereof.

In certain example embodiments, the additional active agent is selected from erastin, Ras-selective lethal small molecule 3, sulfasalazine or analogue thereof, lanperisone, sorafenib, fenugreek (trigonelline), acetaminophen, cisplatin, artesunate, siramesine, lapatinib, a combination of siramesine and lapatinib, ferumoxytol, salinomycin (ironomycin), dihydroartemisnin, gemcitabine, paclitaxel, temozolomide, buthionine sulfoximine (BSO), solasonine, siramesine, laptinib, BAY 87-2243,

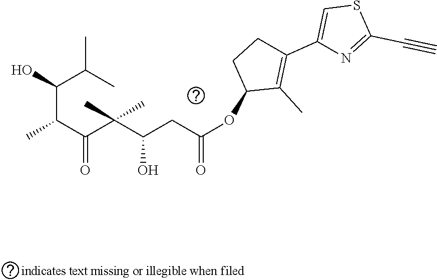

or a derivative or metabolite thereof, optionally

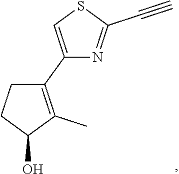

BLZ945, Dyclonine, Oxyfedrine, RSL3, ML210, SSZ, RSL3, ML162, FIN56, Anti-PD1, gefitnib, erlotinib, TMZ, docetaxel, DAT, vemurafenib, lovastatin, atorvastatin, simvastatin, prominin2, artemisinin or a derivative thereof, vitamin E, baicalein, beta-elemene, gallic acid, buthionine sulfoximine, DP17, FIN56, FINO₂, a statin, deferoxamine mesylate, or any combination thereof

Described in certain example embodiments herein are methods comprising delivering to a cell or cell population (a) an engineered biomolecule of any one of the preceding paragraphs or as described in greater detail elsewhere herein; (b) a vector of any one of any one of the preceding paragraphs or as described in greater detail elsewhere herein; (c) a delivery vehicle of any one of any one of the preceding paragraphs or as described in greater detail elsewhere herein; (d) a pharmaceutical formulation as in any one of any one of the preceding paragraphs or as described in greater detail elsewhere herein; or (e) any combination thereof.

In certain example embodiments, ferroptosis is induced and/or potentiated in the cell or cell population.

In certain example embodiments, cytosolic cysteine is decreased, lysosomal cysteine is increased, or both.

In certain example embodiments, ATF4 expression is decreased and/or ATF4 expression induction is decreased.

In certain example embodiments, the method further comprises delivering to the cell an additional active agent.

In certain example embodiments, the additional active agent is effective to induce ferroptosis in the cell or cell population.

In certain example embodiments, the additional active agent is effective to inhibit the X_{c}⁻ antiporter.

In certain example embodiments, the additional active agent is selected from selected from erastin, Ras-selective lethal small molecule 3, sulfasalazine, lanperisone, sorafenib, fenugreek (trigonelline), acetaminophen, cisplatin, artesunate, combination of siramesine and lapatinib, ferumoxytol, and salinomycin (ironomycin), artesunate, dihydroartemisnin, gemcitabine, paclitaxel, temozolomide, buthionine sulfoximine (BSO), solasonine, siramesine, laptinib, BAY 87-2243, Molecule 1 of Taylor et al., 2019 Sci. Reports. 9:5926 and its derivatives or metabolites (specifically molecule 4 of Taylor et al. 2019) any others set forth in e.g., Su et al., Cancer. Lett. 2020. Jul. 28; 483:127-136. doi: 10.1016/j.canlet.2020.02.015; Wu et al., 2020. Front. Oncol. | https://doi.org/10.3389/fonc.2020.571127; Lin et al., Front. Pharmacol. 2020. 11: 1061; Taylor et al., 2019. Sci. Reports. 9:5926, all of which are incorporated by reference herein), or any combination thereof.

In certain example embodiments, the additional active agent is selected from erastin, Ras-selective lethal small molecule 3, sulfasalazine or analogue thereof, lanperisone, sorafenib, fenugreek (trigonelline), acetaminophen, cisplatin, artesunate, siramesine, lapatinib, a combination of siramesine and lapatinib, ferumoxytol, salinomycin (ironomycin), dihydroartemisnin, gemcitabine, paclitaxel, temozolomide, buthionine sulfoximine (BSO), solasonine, siramesine, laptinib, BAY 87-2243,

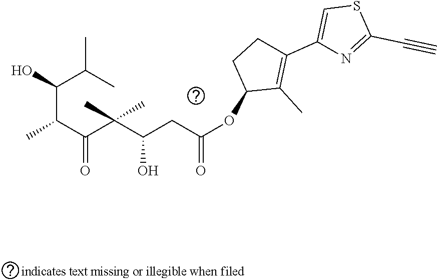

or a derivative or metabolite thereof, optionally

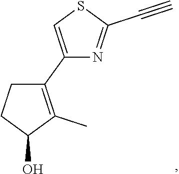

BLZ945, Dyclonine, Oxyfedrine, RSL3, ML210, SSZ, RSL3, ML162, FIN56, Anti-PD1, gefitnib, erlotinib, TMZ, docetaxel, DAT, vemurafenib, lovastatin, atorvastatin, simvastatin, prominin2, artemisinin or a derivative thereof, vitamin E, baicalein, beta-elemene, gallic acid, buthionine sulfoximine, DP17, FIN56, FINO₂, a statin, deferoxamine mesylate, or any combination thereof.

In certain example embodiments, the cell is a cancer cell.

Described in certain example embodiments herein are methods of treating a proliferative disease in a subject in need thereof, the method comprising administering to the subject (a) an engineered biomolecule of any one of any one of the preceding paragraphs or as described in greater detail elsewhere herein; (b) a vector of any one of any one of the preceding paragraphs or as described in greater detail elsewhere herein; (c) a delivery vehicle of any one of any one of the preceding paragraphs or as described in greater detail elsewhere herein; (d) a pharmaceutical formulation as in any one of any one of the preceding paragraphs or as described in greater detail elsewhere herein; or any combination thereof.

In certain example embodiments, ferroptosis is induced and/or potentiated in a cell or cell population in the subject.

In certain example embodiments, cytosolic cysteine is decreased in and/or lysosomal cysteine is increased, in a cell or cell population in the subject.

In certain example embodiments, ATF4 expression is decreased and/or ATF4 expression induction is decreased in a cell or cell population in the subject.

In certain example embodiments, the cell or cell population is a cancer cell or cancer cell population.

In certain example embodiments, the method further comprises administering an additional active agent to the subject.

In certain example embodiments, the additional active agent is administered simultaneously, contemporaneously, or serially with (a)-(e).

In certain example embodiments, the additional active agent is effective to induce ferroptosis in a cell or cell population in the subject.

In certain example embodiments, the additional active agent is effective to inhibit the X_{c}⁻ antiporter in a cell or cell population in the subject.

In certain example embodiments, the additional active agent is selected from selected from erastin, Ras-selective lethal small molecule 3, sulfasalazine, lanperisone, sorafenib, fenugreek (trigonelline), acetaminophen, cisplatin, artesunate, combination of siramesine and lapatinib, ferumoxytol, and salinomycin (ironomycin), artesunate, dihydroartemisnin, gemcitabine, paclitaxel, temozolomide, buthionine sulfoximine (BSO), solasonine, siramesine, laptinib, BAY 87-2243, Molecule 1 of Taylor et al., 2019 and its derivatives or metabolites (specifically molecule 4 of Taylor et al. 2019) any others set forth in e.g., Su et al., Cancer. Lett. 2020. Jul. 28; 483:127-136. doi: 10.1016/j.canlet.2020.02.015; Wu et al., 2020. Front. Oncol. | https://doi.org/10.3389/fonc.2020.571127; Lin et al., Front. Pharmacol. 2020. 11: 1061; Taylor et al., 2019. Sci. Reports. 9:5926, all of which are incorporated by reference herein), or any combination thereof.

In certain example embodiments, the additional active agent is selected from erastin, Ras-selective lethal small molecule 3, sulfasalazine or analogue thereof, lanperisone, sorafenib, fenugreek (trigonelline), acetaminophen, cisplatin, artesunate, siramesine, lapatinib, a combination of siramesine and lapatinib, ferumoxytol, salinomycin (ironomycin), dihydroartemisnin, gemcitabine, paclitaxel, temozolomide, buthionine sulfoximine (BSO), P solasonine, siramesine, laptinib, BAY 87-2243,

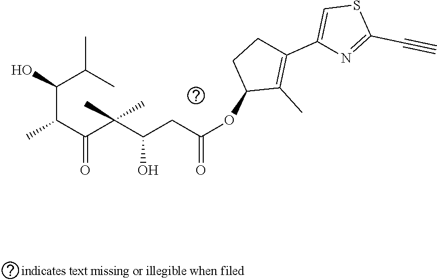

or a derivative or metabolite thereof, optionally

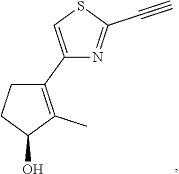

BLZ945, Dyclonine, Oxyfedrine, RSL3, ML210, SSZ, RSL3, ML162, FIN56, Anti-PD1, gefitnib, erlotinib, TMZ, docetaxel, DAT, vemurafenib, lovastatin, atorvastatin, simvastatin, prominin2, artemisinin or a derivative thereof, vitamin E, baicalein, beta-elemene, gallic acid, buthionine sulfoximine, DP17, FIN56, FINO₂, a statin, deferoxamine mesylate, or any combination thereof.

In certain example embodiments, cancer cell growth, cancer tumor growth, or both is inhibited, slowed, and/or stopped.

Described in certain example embodiments herein are methods of inhibiting a cysteine stress response in a cell or cell population, the method comprising delivering to the cell or cell population (a) an engineered biomolecule of any one of any one of the preceding paragraphs or as described in greater detail elsewhere herein; (b) a vector of any one of any one of the preceding paragraphs or as described in greater detail elsewhere herein; (c) a delivery vehicle of any one of any one of the preceding paragraphs or as described in greater detail elsewhere herein; (d) a pharmaceutical formulation as in any one of any one of the preceding paragraphs or as described in greater detail elsewhere herein; or (e) any combination thereof.

In certain example embodiments, ferroptosis is induced and/or potentiated in a cell or cell population.

In certain example embodiments, cytosolic cysteine is decreased in and/or lysosomal cysteine is increased in the cell or cell population.

In certain example embodiments, ATF4 expression is decreased and/or ATF4 expression induction is decreased in the cell or cell population.

In certain example embodiments, the cell or cell population is a cancer cell or cancer cell population.

In certain example embodiments, the method further comprises delivering an additional active agent cell or cell population.

In certain example embodiments, the additional active agent is effective to induce ferroptosis in the cell or cell population.

In certain example embodiments, the additional active agent is effective to inhibit the X_{c}⁻ antiporter in the cell or cell population.

In certain example embodiments, the additional active agent is delivered simultaneously, contemporaneously, or serially with (a)-(e).

In certain example embodiments, the additional active agent is selected from selected from erastin, Ras-selective lethal small molecule 3, sulfasalazine, lanperisone, sorafenib, fenugreek (trigonelline), acetaminophen, cisplatin, artesunate, combination of siramesine and lapatinib, ferumoxytol, and salinomycin (ironomycin), artesunate, dihydroartemisnin, gemcitabine, paclitaxel, temozolomide, buthionine sulfoximine (BSO), solasonine, siramesine, laptinib, BAY 87-2243, Molecule 1 of Taylor et al., 2019 and its derivatives or metabolites (specifically molecule 4 of Taylor et al. 2019) any others set forth in e.g., Su et al., Cancer. Lett. 2020. Jul. 28; 483:127-136. doi: 10.1016/j.canlet.2020.02.015; Wu et al., 2020. Front. Oncol. | https://doi.org/10.3389/fonc.2020.571127; Lin et al., Front. Pharmacol. 2020. 11: 1061; Taylor et al., 2019. Sci. Reports. 9:5926, all of which are incorporated by reference herein), or any combination thereof.

In certain example embodiments, the additional active agent is selected from erastin, Ras-selective lethal small molecule 3, sulfasalazine or analogue thereof, lanperisone, sorafenib, fenugreek (trigonelline), acetaminophen, cisplatin, artesunate, siramesine, lapatinib, a combination of siramesine and lapatinib, ferumoxytol, salinomycin (ironomycin), dihydroartemisnin, gemcitabine, paclitaxel, temozolomide, buthionine sulfoximine (BSO), solasonine, siramesine, laptinib, BAY 87-2243,

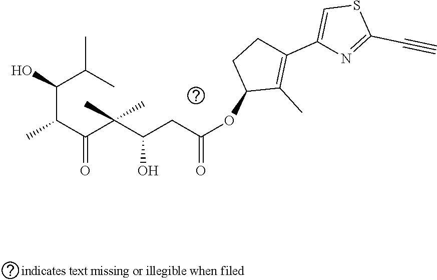

or a derivative or metabolite thereof, optionally

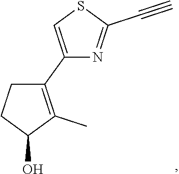

BLZ945, Dyclonine, Oxyfedrine, RSL3, ML210, SSZ, RSL3, ML162, FIN56, Anti-PD1, gefitnib, erlotinib, TMZ, docetaxel, DAT, vemurafenib, lovastatin, atorvastatin, simvastatin, prominin2, artemisinin or a derivative thereof, vitamin E, baicalein, beta-elemene, gallic acid, buthionine sulfoximine, DP17, FIN56, FINO₂, a statin, deferoxamine mesylate, or any combination thereof.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1F-Cystine deprivation induces ATF4 expression independent of the eIF2α signaling pathway. (FIG. 1A) The top panel shows the western blotting of eIF2a(S/S) and eIF2a(A/A) MEF cells after 8 hrs of amino acid starvation. The bottom panel shows RT-qPCR results of Atf4 (normalized to Gapdh) in eIF2a(S/S) and eIF2a(A/A) MEF cells after 8 hrs of amino acid starvations. Error bars, mean±SEM; two-tailed t-test, **p<0.01, n=3 biological replicates. (FIG. 1B) Total cysteine (left) and cystine (right) levels in MEF cells after 12 hrs of amino acid starvation. Both measurements are normalized to protein levels. Error bars, mean±SEM; two-tailed 1-test, *p<0.05, **p<0.01, ***p<0.001, n=3 biological replicates. (FIG. 1C) The top bar graph shows the RT-qPCR results of Slc7a11 (normalized to Gapdh) in MEF cells after 8 hrs of amino acid starvation. Error bars, mean±SEM; two-tailed t-test, **p<0.01, n=3 biological replicates. The bottom panel shows the representative western blotting of SLC7A11 in starved MEF cells. (FIG. 1D) Total GSH levels in MEF cells after 12 hrs of amino acid starvations are normalized to protein levels. Error bars, mean±SEM; two-tailed 1-test, **p<0.01, ***p<0.001, n=3 biological replicates. (FIG. 1E) The top bar graph shows the RT-qPCR results of Nrf2 (normalized to Gapdh) in MEF cells after 8 hrs of amino acid starvation. Error bars, mean±SEM; two-tailed 1-test, *p<0.05, **p<0.01, n=3 biological replicates. The bottom panel shows the representative western blotting of NRF2 in starved MEF cells. (FIG. 1F) MEF cells with or without Nrf2 knockdown were subject to either 8 hrs of BSO (10 μM) treatment or 8 hrs of cystine starvation. The top bar graph shows the RT-qPCR results of Atf4 (normalized to Gapdh). Error bars, mean±SEM; two-tailed 1-test, **p<0.01, n=3 biological replicates. The bottom panel shows the representative western blotting of ATF4 in starved MEF cells.

FIG. 2A-2H-Lysosomal cystine controls transcriptional regulation of ATF4 via the AhR signaling pathway. (FIG. 2A) Schematic of intracellular cysteine and cystine metabolism, storage, and function. (FIG. 2B) Total cysteine (top) and cystine (bottom) levels in MEF cells treated with BafilomycinA (50 nM for 12 hrs) with or without cystine deprivation. Both measurements are normalized to protein levels. Error bars, mean±SEM; two-tailed 1-test, *p<0.05, ***p<0.001, n=3 biological replicates. (FIG. 2C) The top bar graph shows the RT-qPCR results of Atf4 (normalized to Gapdh) in MEF cells treated with BafilomycinA (50 nM for 8 hrs) with or without cystine deprivation. Error bars, mean±SEM; two-tailed t-test, *p<0.05, n=3 biological replicates. The bottom panel shows the representative western blotting of ATF4 in MEF cells. (FIG. 2D) The top panel shows the illustration of a Fluc reporter driven by the Atf4 promoter. The bar graph shows the relative Fluc activities normalized to Renilla luciferase in transfected MEF cells treated with BafilomycinA (50 nM for 8 hrs) with or without cystine deprivation. Error bars, mean±SEM; two-tailed t-test, *p<0.05, n=3 biological replicates. (FIG. 2E) Total cysteine (top) and cystine (bottom) levels in MEF cells with or without CTNS knockdown. Both measurements are normalized to protein levels. Error bars, mean±SEM; two-tailed 1-test, *p<0.05, **p<0.01, ***p<0.001, n=3 biological replicates. (FIG. 2F) The top bar graph shows the RT-qPCR results of Atf4 (normalized to Gapdh) in MEF cells with or without CTNS knockdown before and after cystine deprivation. Error bars, mean±SEM; two-tailed 1-test, ***p<0.001, n=3 biological replicates. The bottom panel shows the representative western blotting of ATF4 in MEF cells. (FIG. 2G) The top panel shows the illustration of the Atf4 promoter region with highlighted AhR consensus sequences. The bottom panel shows the chemicals of AhR modulators. (FIG. 2H) The top bar graph shows the RT-qPCR results of Atf4 (normalized to Gapdh) in MEF cells treated with SR1 (200 nM) or Indirubin (2 μM) before and after cystine deprivation. Error bars, mean±SEM; two-tailed t-test, **p<0.01, n=3 biological replicates. The bottom panel shows the representative western blotting of ATF4 in MEF cells.

FIG. 3A-3D-A cysteine/cystine ratio determines the cellular sensitivity to ferroptosis. (FIG. 3A) The left panel shows the cell viability in MEF cells treated with BafilomycinA (50 nM) for 24 hrs with or without cystine deprivation. Error bars, mean±SEM; two-tailed t-test, *p<0.05, n=4 biological replicates. The right panel shows lipid ROS levels in MEF cells treated with BafilomycinA (50 nM for 18 hrs) with or without cystine deprivation. The lipid ROS levels were quantified by C11-BODIPY staining and flow cytometry. Error bars, mean±SEM; two-tailed t-test, ***p<0.001, n=3 biological replicates. (FIG. 3B) The left panel shows the cell viability in MEF cells treated with Cysteamine (0.5 mM for 24 hrs) with or without cystine deprivation. Error bars, mean±SEM; two-tailed t-test, *p<0.05, n=4 biological replicates. The right panel shows lipid ROS levels in MEF cells treated with Cysteamine (0.5 mM for 18 hrs) with or without cystine deprivation. Error bars, mean±SEM; two-tailed t-test, ***p<0.001, n=3 biological replicates. (FIG. 3C) The left panel shows the cell viability in MEF cells treated with SR1 (200 nM) or Indirubin (2 μM) for 24 hrs with or without cystine deprivation. Error bars, mean±SEM; two-tailed t-test, *p<0.05, ****p<0.0001, n=4 biological replicates. The right panel shows lipid ROS levels in MEF cells treated with (SR1 (200 nM) or Indirubin (2 μM) for 18 hrs with or without cystine deprivation. Error bars, mean±SEM; two-tailed t-test, ***p<0.001, n=3 biological replicates. (FIG. 3D) The left panel shows the cell viability in MEF cells with or without CTNS knockdown before and after 24 hrs cystine deprivation. Error bars, mean±SEM; two-tailed t-test, ***p<0.001, n=4 biological replicates. The right panel shows lipid ROS levels in MEF cells with or without CTNS knockdown before and after 18 hrs cystine deprivation. Error bars, mean±SEM; two-tailed t-test, ***p<0.001, n=3 biological replicates.

FIG. 4A-4I. Modulating cysteine/cystine ratio in cancer cells by CysRx. (FIG. 4A) Western blotting of UMRC6 cells subjected to CTNS and/or SLC7A11 knockdown. (FIG. 4B) The cell viability in UMRC6 cells subjected to CTNS and/or SLC7A11 knockdown after 24 hr cystine deprivation. Error bars, mean±SEM; two-tailed t-test, *p<0.05, **p<0.01, ****p<0.0001, n=4 biological replicates. (FIG. 4C) The bar graph shows the colony formation of UMRC6 cells subject to CTNS and/or SLC7A11 knockdown in soft agar. Error bars, mean±SEM; two-tailed 1-test, ****p<0.0001, n=5 biological replicates. (FIG. 4D) Tumor growth curves from UMRC6 cells subject to CTNS and/or SLC7A11 knockdown in SCID-Beige mice after subcutaneous injection. Error bars, mean±SEM; two-way ANOVA test, **p<0.01, ****p<0.0001, n=10 mice per group. (FIG. 4E) Schematic of how CysRx mRNA cycles cysteine and cystine between the cytosol and lysosome. (FIG. 4F) Total cysteine (top) and cystine (bottom) levels in UMRC6 cells after 24 hrs of CysRx transfection (5 ug). Both measurements were normalized to protein levels. Error bars, mean±SEM; two-tailed t-test, *p<0.05, n=3 biological replicates. (FIG. 4G) The top panel shows the cell viability in UMRC6 cells after 24 hrs of CysRx (5 ug) transfection with or without cystine deprivation. Error bars, mean±SEM; two-tailed 1-test, **p<0.01, n=4 biological replicates. The bottom panel shows the lipid ROS levels in UMRC6 cells after 18 hrs of CysRx (5 ug) transfection with or without cystine deprivation. Error bars, mean±SEM; two-tailed 1-test, *p<0.05, **p<0.01, n=3 biological replicates. (FIG. 4H) The top bar graph shows the RT-qPCR results of Atf4 (normalized to Gapdh) in (UMRC6 cells after 12 hrs of CysRx (5 ug) transfection with or without cystine deprivation. Error bars, mean±SEM; two-tailed 1-test, **p<0.01, n=3 biological replicates. The bottom panel shows the representative western blotting of ATF4 in UMRC6 cells after 12 hrs of CysRx (5 ug) transfection with or without cystine deprivation. (FIG. 4I) Tumor growth curves from UMRC6 cells implanted bilaterally in SCID-Beige mice. Mice were treated with IKE or vehicle control (10 mg/kg/2d) for 25 days. CysRx or HiBit mRNA (0.5 mg/kg/7d) were intratumorally injected into each flank in the form of LNP. Representative MRI images are shown with tumors in green treated with CysRx-TT3 and tumors in red treated with HiBit-TT3. Error bars, mean±SEM; two-way ANOVA test, **p<0.01, ***p<0.001, n=6 mice per group.

FIG. 5A-5F—Cystine deprivation induces ATF4 upregulation independent of the ISR. (FIG. 5A) Western blotting of GCN2 WT and KO MEF cells after 8 hrs of amino acid starvation. (FIG. 5B) Western blotting of eIF2a(S/S) and eIF2a(A/A) MEF cells after 8 hrs of amino acid starvation in the presence or absence of ISRIB (100 nM). (FIG. 5C) MEF cells transfected with the reporter plasmid ATF4-Fluc (left) were subjected to amino acid starvations for 8 hr. Fluc activities were measured by luminometry and normalized to Renilla luciferase. Error bars, mean±SEM; two-tailed t-test, *p<0.05, n=3 biological replicates. (FIG. 5D) MEF cells transfected with the Fluc reporter driven by the Atf4 Promoter (left) were subjected to amino acid starvation for 8 hr. Fluc activities were measured by luminometry and normalized to Renilla luciferase. Error bars, mean±SEM; two-tailed t-test, *p<0.05, ***p<0.001, n=3 biological replicates. (FIG. 5E) Total cysteine (left) and cystine (right) levels in MEF cells treated with erastin (20 μM). Both measurements are normalized to protein levels. Error bars, mean±SEM; two-tailed t-test, ***p<0.001, n=3 biological replicates. (FIG. 5F) The top bar graph shows the RT-qPCR results of Atf4 (normalized to Gapdh) in eIF2a(S/S) and eIF2a(A/A) MEF cells treated with Erastin (20 μM). Error bars, mean±SEM; two-tailed t-test, ****p<0.0001, n=3 biological replicates. The bottom panel shows the representative western blotting of ATF4 in MEF cells.

FIG. 6A-6F—Control of intracellular cyst(e)ine levels by translation and lysosome V-ATPase. (FIG. 6A) Puromycin labeling assay in eIF2a(S/S) and eIF2a(A/A) MEF cells after 8 hrs of amino acid starvation. (FIG. 6B) Total cysteine (left) and cystine (right) levels in MEF cells after 12 hrs of amino acid starvation with or without Cycloheximide (50 μM) treatment. Both measurements are normalized to protein levels. Error bars, mean±SEM; two-tailed t-test, *p<0.05, **p<0.01, n=3 biological replicates. (FIG. 6C) The top bar graph shows the RT-qPCR results of Atf4 (normalized to Gapdh) in eIF2a(S/S) and eIF2a(A/A) MEF cells with or without 8 hrs of cystine starvation, presence or absence of HCA (10 mM) and Ascorbic Acid (20 μM). Error bars, mean±SEM; two-tailed t-test, ***p<0.001, n=3 biological replicates. The bottom panel shows the representative western blotting of ATF4 in MEF cells. (FIG. 6D) Total cysteine (top) and cystine (bottom) levels in MEF cells after 12 hrs of cystine starvation in the presence or absence of ConcanamycinA (100 nM). Both measurements are normalized to protein levels. Error bars, mean±SEM; two-tailed t-test, *p<0.05, ****p<0.0001, n=3 biological replicates. (FIG. 6E) The top bar graph shows the RT-qPCR results of Atf4 (normalized to Gapdh) in MEF cells after 12 hrs of cystine starvation in the presence or absence of ConcanamycinA (100 nM). Error bars, mean±SEM; two-tailed t-test, **p<0.01, n=3 biological replicates. The bottom panel shows the representative western blotting of ATF4 in MEF cells. (FIG. 6F) MEF cells transfected with the Fluc reporter driven by the ARE element (top) were subjected to cystine starvations for 8 hr in the presence or absence of BafilomycinA (50 nM) or ConcanamycinA (100 nM). Fluc activities were measured by luminometry and normalized to Renilla luciferase. Error bars, mean±SEM; two-tailed t-test, **p<0.01, n=3 biological replicates.

FIG. 7A-7E—Characterization of MEF cells with CTNS knockdown. (FIG. 7A-7E) Western blotting of MEF cells with or without CTNS knockdown. (FIG. 7B) MEF cells with or without CTNS knockdown were transfected with the Fluc reporter driven by the Atf4 Promoter (top) followed by amino acid starvations for 8 hr. Fluc activities were measured by luminometry and normalized to Renilla luciferase. Error bars, mean±SEM; two-tailed t-test, *p<0.05, n=3 biological replicates. (FIG. 7C) MEF cells with or without CTNS knockdown were transfected with the Fluc reporter driven by the ARE element (top) followed by amino acid starvations for 8 hr. Fluc activities were measured by luminometry and normalized to Renilla luciferase. Error bars, mean±SEM; two-tailed 1-test, **p<0.01, n=3 biological replicates. (FIG. 7D) Puromycin labeling assay in MEF cells with or without CTNS knockdown before and after cystine starvation (8 hrs). (FIG. 7E) Polysome profiles of MEF cells with or without CTNS knockdown before and after cystine starvation (8 hrs).

FIG. 8A-8E—Lysosomal cystine shortage-induced ATF4 upregulation involves the AhR signaling pathway. (FIG. 8A) Schematic of lysosomal cystine influx and efflux as well as the role of cysteamine in cystine recycling. (FIG. 8B) Total cysteine (top) and cystine (bottom) levels in MEF cells after 12 hrs of cystine starvation in the presence or absence of Cysteamine (0.5 mM). Both measurements are normalized to protein levels. Error bars, mean±SEM; two-tailed t-test, *p<0.05, **p<0.01, ***p<0.001, ****p<0.0001, n=3 biological replicates. (FIG. 8C) The top bar graph shows the RT-qPCR results of Atf4 (normalized to Gapdh) in MEF cells after 12 hrs of cystine starvation in the presence or absence of Cysteamine (0.5 mM). Error bars, mean±SEM; two-tailed t-test, *p<0.05, n=3 biological replicates. The bottom panel shows the representative western blotting of ATF4 in MEF cells. (FIG. 8D) MEF cells transfected with the Fluc reporter driven by the Atf4 Promoter (top) were treated with Cysteamine (0.5 mM). Fluc activities were measured by luminometry and normalized to Renilla luciferase. Error bars, mean±SEM; two-tailed t-test, *p<0.05, n=3 biological replicates. (FIG. 8E) MEF cells transfected with Fluc reporters driven by truncated Atf4 promoter regions (left) were subjected to Cysteamine (0.5 mM) treatment (blue bars) for 8 hrs followed by luminometry. Fluc activities are normalized to Renilla luciferase. Error bars, mean±SEM; two-tailed t-test, **p<0.01, n=3 biological replicates.

FIG. 9A-9G—Intracellular cysteine deprivation induces ferroptosis. (FIG. 9A) Cell viability in MEF cells after 24 hrs amino acid deprivations. Error bars, mean±SEM; two-tailed t-test, ***p<0.001, ****p<0.0001, n=4 biological replicates. (FIG. 9B) Cell viability in MEF cells after 24 hrs full amino acid starvation (left) or cystine deprivation (right) were treated with Z-VAD (20 μM), Necrostatin (5 μM), or Ferrostatin (1 μM). Error bars, mean±SEM; two-tailed t-test, n=4 biological replicates. Error bars, mean±SEM; two-tailed t-test, **p<0.01, ****p<0.0001, n=4 biological replicates. (FIG. 9C) Lipid ROS levels in MEF cells after 18 hrs amino acid deprivations or Erastin (20 μM) treatment. The lipid ROS levels were quantified by C11-BODIPY staining and flow cytometry. Error bars, mean±SEM; two-tailed 1-test, ***p<0.001, n=3 biological replicates. (FIG. 9D) Western blotting of MEF cells with or without Slc7a11 knockdown. (FIG. 9E) The left panel shows the cell viability in MEF cells with or without Slc7a11 knockdown before and after 24 hrs cystine deprivation. Error bars, mean±SEM; two-tailed t-test, ****p<0.0001, n=4 biological replicates. The right panel shows lipid ROS levels in MEF cells with or without Slc7a11 knockdown before and after 18 hrs cystine deprivation. Error bars, mean±SEM; two-tailed t-test, **p<0.01, ***p<0.001, n=3 biological replicates. (FIG. 9F) The left panel shows the cell viability in MEF cells with or without 24 hrs cystine deprivation in the presence or absence of Cycloheximide (50 μM). Error bars, mean±SEM; two-tailed t-test, **p<0.01, n=4 biological replicates. The right panel shows lipid ROS levels in MEF cells with or without 24 hrs cystine deprivation in the presence or absence of Cycloheximide (50 μM). Error bars, mean±SEM; two-tailed t-test, ***p<0.001, n=3 biological replicates. (FIG. 9G) Cell viability of MEF cells after 24 hrs treatment with Erastin (20 μM) or Sulfsalazine (200 μM) in the presence or absence of Cycloheximide (50 μM). Error bars, mean±SEM; two-tailed t-test, *p<0.05, **p<0.01, n=4 biological replicates.

FIG. 10A-10C—Silencing CTNS sensitizes cells to ferroptosis. (FIG. 10A) The left panel shows the cell viability in MEF cells treated with ConcanamycinA (100 nM) for 24 hrs with or without cystine deprivation. Error bars, mean±SEM; two-tailed t-test, *p<0.05, n=4 biological replicates. The right panel shows lipid ROS levels in MEF cells treated with BafilomycinA (50 nM for 18 hrs) with or without cystine deprivation. The lipid ROS levels were quantified by C11-BODIPY staining and flow cytometry. Error bars, mean±SEM; two-tailed 1-test, ***p<0.001, n=3 biological replicates. (FIG. 10B) The cell viability in MEF cells with or without CTNS knockdown after treatment with Erastin (20 μM) (left) or Sulfsalazine (200 μM) (right). Error bars, mean±SEM; two-tailed t-test, **p<0.01, n=4 biological replicates. (FIG. 10C) The left panel shows the cell viability in MEF cells with or without CTNS knockdown before and after 24 hrs cystine deprivation in the presence or absence of Cysteamine (0.5 mM). Error bars, mean±SEM; two-tailed t-test, **p<0.01, ****p <0.0001, n=4 biological replicates. The right panel shows lipid ROS levels in MEF cells. Error bars, mean±SEM; two-tailed t-test, **p<0.01, ***p<0.001, n=3 biological replicates.

FIG. 11A-11D—A synergistic role between CTNS and Slc7a11 in tumor cell growth. (FIG. 11A) Kaplan-Meier curves comparing the overall survival of patients with high and low expression of CTNS. (FIG. 11B) Representative images of soft agar assay for UMRC6 cells with CTNS and/or Slc7a11 knockdown. Images were taken 21 days post plating. (FIG. 11C) The top panel shows the western blotting of 786-O cells with or without CTNS knockdown. The bottom bar graph shows the cell viability in 786-O cells with or without CTNS knockdown after 24 hrs cystine deprivation. Error bars, mean±SEM; two-tailed t-test, *p<0.05, n=4 biological replicates. (FIG. 11D) The left panel shows the representative images of soft agar assay for 786-O cells with or without CTNS knockdown. Images were taken 21 days post plating. The right panel shows the quantification of colony number of 786-O cells with or without CTNS knockdown. Error bars, mean±SEM; two-tailed t-test, **p<0.01, n=5 biological replicates.

FIG. 12A-12E—Characterization of CysRx. (FIG. 12A) The top panel shows the schematic of CysRx design. The bottom panels show western blotting of fractionated MEF cells transfected with CysRx in the presence or absence of BafilomycinA (50 nM) for 12 hrs. Cell fractions are cytoplasmic (Cyto), lysosomal (Lyso), and mitochondria (Mito). (FIG. 12B) The top bar graph shows the RT-qPCR results of Atf4 (normalized to Gapdh) in MEF cells transfected with CysRx with or without 12 hrs of cystine starvation. Error bars, mean±SEM; two-tailed t-test, *p<0.05, n=3 biological replicates. The bottom panel shows the representative western blotting of ATF4 in transfected MEF cells. (FIG. 12C) The left panel shows the cell viability in MEF cells transfected with CysRx with or without 12 hrs of cystine starvation. Error bars, mean±SEM; two-tailed t-test, ***p<0.001, n=4 biological replicates. The right panel shows lipid ROS levels in transfected MEF cells. Error bars, mean±SEM; two-tailed t-test, **p<0.01, n=3 biological replicates. (FIG. 12D) Quantified colony formation of UMRC6 cells transfected with CysRx treatment in soft agar. Error bars, mean±SEM; two-tailed t-test, **p<0.01, ***p<0.001, n=5 biological replicates. Images were taken 21 days post plating and treatment. (FIG. 12E) Quantified colony formation of 786-O cells transfected with CysRx treatment in soft agar. Error bars, mean±SEM; two-tailed t-test, ***p<0.001, n=5 biological replicates. Images were taken 21 days post plating and treatment.

FIG. 13A-13E-Therapeutic assessment of CysRx in vivo. (FIG. 13A) Chemical structure of TT3 used in LNP formulation. (FIG. 13B) Real time HiBit-Luc activity in UMRC6 cells transfected with CysRx mRNA (5 ug) containing UTP or N1-methlpseudo-UTP. (FIG. 13C) Representative images of haematoxylin & eosin and 4HNE immunohistochemical staining of xenograft tumors collected from mice treated with CysRx-TT3 or HiBit-TTS for 25 days. (FIG. 13D) Quantification of 4HNE-positive stained cells per field for samples in (C). Error bars, mean±SEM; two-tailed t-test, **p<0.01, ****p<0.0001, n=5 randomly selected high-power fields per group. (FIG. 13E) Body weight of SCID-Beige mice with intraperitoneal administration of IKE (10 mg/kg/2d) or equal volume of vehicle for the duration of the study. Error bars, mean±SEM; two-tailed t-test, **p<0.01, n=6 mice per group.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlett, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2^{nd} ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

As used herein, “agent” refers to any substance, compound, molecule, and the like, which can be biologically active or otherwise can induce a biological and/or physiological effect on a subject to which it is administered to.

As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An active agent can be a secondary agent, or in other words, the component(s) of a composition to which an additional part and/or other effect of the composition is attributed.

As used herein, “administering” refers to any suitable administration for the agent(s) being delivered and/or subject receiving said agent(s) and can be oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g., by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

As used herein, “antibody” refers to a protein or glycoprotein containing at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. Each light chain is comprised of a light chain variable region and a light chain constant region. The VH and VL regions retain the binding specificity to the antigen and can be further subdivided into regions of hypervariability, termed complementarity determining regions (CDR). The CDRs are interspersed with regions that are more conserved, termed framework regions (FR). Each VH and VL is composed of three CDRs and four framework regions, arranged from amino-terminus to carboxy-terminus in the following order: FR1, CDR1, FR2, CDR2, FR3, CDR3, and FR4. The variable regions of the heavy and light chains contain a binding domain that interacts with an antigen. “Antibody” includes single valent, bivalent and multivalent antibodies

As used herein, “anti-infective” refers to compounds or molecules that can either kill an infectious agent and/or modulate or inhibit its activity, infectivity, replication, and/or spreading such that its infectivity is reduced or eliminated and/or the disease or symptom thereof that it is associated is less severe or eliminated. Anti-infectives include, but are not limited to, antibiotics, antibacterials, antifungals, antivirals, and antiprotozoals.

As used herein, “antigen” refers to a molecule or a portion of a molecule capable of being bound by an antibody, or by a T cell receptor (TCR) when presented by MHC molecules. At the molecular level, an antigen is characterised by its ability to be bound at the antigen-binding site of an antibody. The specific binding denotes that the antigen will be bound in a highly selective manner by its cognate antibody and not by the multitude of other antibodies which may be evoked by other antigens. An antigen is additionally capable of being recognised by the immune system. In some instances, an antigen is capable of eliciting a humoral immune response in a subject. In some instances, an antigen is capable of eliciting a cellular immune response in a subject, leading to the activation of B- and/or T-lymphocytes.

As used herein, “aptamer” refers to single-stranded DNA or RNA molecules that can bind to pre-selected targets including proteins with high affinity and specificity. Their specificity and characteristics are not directly determined by their primary sequence, but instead by their tertiary structure.

As used herein, “biomolecule” refers to any compound, composition, molecule and the like that is made by or present in a living organism, and includes, without limitation, polynucleotides (e.g., DNA, RNA), peptides and polypeptides, and chemical compounds (e.g., hormones, chemokines, and cytokines). It will be appreciated that biomolecules can be de novo and/or chemically synthesized outside of a living organism and still be a biomolecule as the term is used herein.

As used herein “cancer” refers to one or more types of cancer including, but not limited to, acute lymphoblastic leukemia, acute myeloid leukemia, adrenocortical carcinoma, Kaposi Sarcoma, AIDS-related lymphoma, primary central nervous system (CNS) lymphoma, anal cancer, appendix cancer, astrocytomas, atypical teratoid/Rhabdoid tumors, basal cell carcinoma of the skin, bile duct cancer, bladder cancer, bone cancer (including but not limited to Ewing Sarcoma, osteosarcomas, and malignant fibrous histiocytoma), brain tumors, breast cancer, bronchial tumors, Burkitt lymphoma, carcinoid tumor, cardiac tumors, germ cell tumors, embryonal tumors, cervical cancer, cholangiocarcinoma, chordoma, chronic lymphocytic leukemia, chronic myelogenous leukemia, chronic myeloproliferative neoplasms, colorectal cancer, craniopharyngioma, cutaneous T-Cell lymphoma, ductal carcinoma in situ, endometrial cancer, ependymoma, esophageal cancer, esthesioneuroblastoma, extracranial germ cell tumor, extragonadal germ cell tumor, eye cancer (including, but not limited to, intraocular melanoma and retinoblastoma), fallopian tube cancer, gallbladder cancer, gastric cancer, gastrointestinal carcinoid tumor, gastrointestinal stromal tumors, central nervous system germ cell tumors, extracranial germ cell tumors, extragonadal germ cell tumors, ovarian germ cell tumors, testicular cancer, gestational trophoblastic disease, Hairy cell leukemia, head and neck cancers, hepatocellular (liver) cancer, Langerhans cell histiocytosis, Hodgkin lymphoma, hypopharyngeal cancer, islet cell tumors, pancreatic neuroendocrine tumors, kidney (renal cell) cancer, laryngeal cancer, leukemia, lip cancer, oral cancer, lung cancer (non-small cell and small cell), lymphoma, melanoma, Merkel cell carcinoma, mesothelioma, metastatic squamous cell neck cancer, midline tract carcinoma with and without NUT gene changes, multiple endocrine neoplasia syndromes, multiple myeloma, plasma cell neoplasms, mycosis fungoides, myelodysplastic syndromes, myelodysplastic/myeloproliferative neoplasms, chronic myelogenous leukemia, nasal cancer, sinus cancer, non-Hodgkin lymphoma, pancreatic cancer, paraganglioma, paranasal sinus cancer, parathyroid cancer, penile cancer, pharyngeal cancer, pheochromocytoma, pituitary cancer, peritoneal cancer, prostate cancer, rectal cancer, Rhabdomyosarcoma, salivary gland cancer, uterine sarcoma, Sézary syndrome, skin cancer, small intestine cancer, large intestine cancer (colon cancer), soft tissue sarcoma, T-cell lymphoma, throat cancer, oropharyngeal cancer, nasopharyngeal cancer, hypopharyngeal cancer, thymoma, thymic carcinoma, thyroid cancer, transitional cell cancer of the renal pelvis and ureter, urethral cancer, uterine cancer, vaginal cancer, cervical cancer, vascular tumors and cancer, vulvar cancer, and Wilms Tumor.

As used herein, “cDNA” refers to a DNA sequence that is complementary to a RNA transcript in a cell. It is a man-made molecule. Typically, cDNA is made in vitro by an enzyme called reverse-transcriptase using RNA transcripts as templates.

As used herein, “cell type” refers to the more permanent aspects (e.g., a hepatocyte typically can't on its own turn into a neuron) of a cell's identity. Cell type can be thought of as the permanent characteristic profile or phenotype of a cell. Cell types are often organized in a hierarchical taxonomy, types may be further divided into finer subtypes; such taxonomies are often related to a cell fate map, which reflect key steps in differentiation or other points along a development process. Wagner et al., 2016. Nat Biotechnol. 34(111): 1145-1160.

As used herein, “chemotherapeutic agent” or “chemotherapeutic” refers to a therapeutic agent utilized to prevent or treat cancer.

As used herein, “coating” refers to any temporary, semi-permanent or permanent layer, covering or surface. A coating can be applied as a gas, vapor, liquid, paste, semi-solid, or solid. In addition, a coating can be applied as a liquid and solidified into a hard coating. Elasticity can be engineered into coatings to accommodate pliability, e.g., swelling or shrinkage, of the substrate or surface to be coated.

As used herein, “control” refers to an alternative subject or sample used in an experiment for comparison purpose and included to minimize or distinguish the effect of variables other than an independent variable.

As used herein with reference to the relationship between DNA, cDNA, CRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein “reduced expression”, “decreased expression”, or “underexpression” refers to a reduced or decreased expression of a gene, such as a gene relating to an antigen processing pathway, or a gene product thereof in sample as compared to the expression of said gene or gene product in a suitable control. As used throughout this specification, “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed. In one embodiment, said control is a sample from a healthy individual or otherwise normal individual. By way of a non-limiting example, if said sample is a sample of a lung tumor and comprises lung tissue, said control is lung tissue of a healthy individual. The term “reduced expression” preferably refers to at least a 25% reduction, e.g., at least a 30%, 40%, 50%, 60%, 70%, 75%, 80%, 85%, 90%, 95%, 98% or 99% reduction, relative to such control.

As used herein, “deoxyribonucleic acid (DNA)” and “ribonucleic acid (RNA)” can generally refer to any polyribonucleotide or polydeoxribonucleotide, which may be unmodified RNA or DNA or modified RNA or DNA. RNA can be in the form of non-coding RNA such as tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers, guide RNA (gRNA) or coding mRNA (messenger RNA).

As used herein, “DNA molecule” can include nucleic acids/polynucleotides that are made of DNA.

As used herein, the terms “disease” or “disorder” are used interchangeably throughout this specification, and refer to any alternation in state of the body or of some of the organs, interrupting or disturbing the performance of the functions and/or causing symptoms such as discomfort, dysfunction, distress, or even death to the person afflicted or those in contact with a person. A disease or disorder can also be related to a distemper, ailing, ailment, malady, disorder, sickness, illness, complaint, indisposition, or affliction.

As used herein, “dose,” “unit dose,” or “dosage” can refer to physically discrete units suitable for use in a subject, each unit containing a predetermined quantity of the active agent(s) and/or a pharmaceutical formulation thereof calculated to produce the desired response or responses in association with its administration.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins. In some instances, “expression” can also be a reflection of the stability of a given RNA. For example, when one measures RNA, depending on the method of detection and/or quantification of the RNA as well as other techniques used in conjunction with RNA detection and/or quantification, it can be that increased/decreased RNA transcript levels are the result of increased/decreased transcription and/or increased/decreased stability and/or degradation of the RNA transcript. One of ordinary skill in the art will appreciate these techniques and the relation “expression” in these various contexts to the underlying biological mechanisms.

The term “fragment” with reference to a nucleic acid (polynucleotide) generally denotes a 5′- and/or 3′-truncated form of a nucleic acid. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the nucleic acid sequence length of said nucleic acid. For example, insofar not exceeding the length of the full-length nucleic acid, a fragment may include a sequence of ≥5 consecutive nucleotides, or ≥10 consecutive nucleotides, or ≥20 consecutive nucleotides, or ≥30 consecutive nucleotides, e.g., ≥40 consecutive nucleotides, such as for example ≥50 consecutive nucleotides, e.g., ≥60, ≥70, ≥80, ≥90, ≥100, ≥200, ≥300, ≥400, ≥500 or ≥600 consecutive nucleotides of the corresponding full-length nucleic acid. The terms encompass fragments arising by any mechanism, in vivo and/or in vitro, such as, without limitation, by alternative transcription or translation, exo- and/or endo-proteolysis, exo- and/or endo-nucleolysis, or degradation of the peptide, polypeptide, protein, or nucleic acid, such as, for example, by physical, chemical and/or enzymatic proteolysis or nucleolysis.

As used herein, “immunomodulator,” refers to an agent, such as a therapeutic agent, which is capable of modulating or regulating one or more immune function or response.

As used herein, “implanting” “Implanting,” refers to the insertion or grafting into the body of a subject of a product or material.

As used herein “increased expression” or “overexpression” are both used to refer to an increased expression of a gene, such as a gene relating to an antigen processing and/or presentation pathway, or gene product thereof in a sample as compared to the expression of said gene or gene product in a suitable control. The term “increased expression” preferably refers to 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000%, 1010%, 1020%, 1030%, 1040%, 1050%, 1060%, 1070%, 1080%, 1090%, 1100%, 1110%, 1120%, 1130%, 1140%, 1150%, 1160%, 1170%, 1180%, 1190%, 1200%, 1210%, 1220%, 1230%, 1240%, 1250%, 1260%, 1270%, 1280%, 1290%, 1300%, 1310%, 1320%, 1330%, 1340%, 1350%, 1360%, 1370%, 1380%, 1390%, 1400%, 1410%, 1420%, 1430%, 1440%, 1450%, 1460%, 1470%, 1480%, 1490%, or/to 1500% or more increased expression relative to a suitable control.

As used herein, “mammal,” for the purposes of treatments, can refer to any animal classified as a mammal, including human, domestic and farm animals, nonhuman primates, and zoo, sports, or pet animals, such as, but not limited to, dogs, horses, cats, and cows.

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

As used herein, “nanoparticle” refers to a nanoscale deposit of a homogenous or heterogeneous material. Nanoparticles may be regular or irregular in shape and may be formed from a plurality of co-deposited particles that form a composite nanoscale particle. Nanoparticles may be generally spherical in shape or have a composite shape formed from a plurality of co-deposited generally spherical particles. Exemplary shapes for the nanoparticles include, but are not limited to, spherical, rod, elliptical, cylindrical, disc, and the like. In some embodiments, the nanoparticles have a substantially spherical shape.

As used herein, “nucleic acid,” “nucleotide sequence,” and “polynucleotide” can be used interchangeably herein and can generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used interchangeably herein, “operatively linked” and “operably linked” in the context of recombinant or engineered polynucleotide molecules (e.g. DNA and RNA) vectors, and the like refers to the regulatory and other sequences useful for expression, stabilization, replication, and the like of the coding and transcribed non-coding sequences of a nucleic acid that are placed in the nucleic acid molecule in the appropriate positions relative to the coding sequence so as to effect expression or other characteristic of the coding sequence or transcribed non-coding sequence. This same term can be applied to the arrangement of coding sequences, non-coding and/or transcription control elements (e.g., promoters, enhancers, and termination elements), and/or selectable markers in an expression vector. “Operatively linked” can also refer to an indirect attachment (i.e., not a direct fusion) of two or more polynucleotide sequences or polypeptides to each other via a linking molecule (also referred to herein as a linker).

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

As used herein, a “population” of cells is any number of cells greater than 1, but is preferably at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, or at least 1×10¹⁰ cells.

As used herein, “preventative” and “prevent” refers to hindering or stopping a disease or condition before it occurs, even if undiagnosed, or while the disease or condition is still in the sub-clinical phase.

As used herein, “proliferative disease” generally refers to any disease or disorder characterized by neoplastic cell growth and proliferation, whether benign, pre-malignant, or malignant. The term proliferative disease generally includes all transformed cells and tissues and all cancerous cells and tissues. Proliferative diseases or disorders include, but are not limited to abnormal cell growth, benign tumors, premalignant or precancerous lesions, malignant tumors, and cancer.

As used herein, the term “radiation sensitizer” refers to agents that can selectively enhance the cell killing from irradiation in a desired cell population, such as tumor cells, while exhibiting no single agent toxicity on tumor or normal cells.

As used herein, the term “recombinant” or “engineered” can generally refer to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc. Recombinant or engineered can also refer to the polypeptide encoded by the recombinant nucleic acid. Non-naturally occurring nucleic acids or polypeptides include nucleic acids and polypeptides modified by man.

As used herein, the term “specific binding” refers to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, Kd, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

As used interchangeably herein, the terms “sufficient” and “effective,” can refer to an amount (e.g., mass, volume, dosage, concentration, and/or time period) needed to achieve one or more desired result(s). For example, a therapeutically effective amount refers to an amount needed to achieve one or more therapeutic effects.

As used herein “suitable control” refers a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g. a web interface.

As used herein, “therapeutic” can refer to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as a proliferative disease The effect can be therapeutic in terms of a partial or complete cure of a disease, condition, symptom or adverse effect attributed to the disease, disorder, or condition. The term “treatment” as used herein covers any treatment of a proliferative disease, in a subject, particularly a human, and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein, the terms “weight percent,” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Cancer cells rely on a constant supply of nutrients such as amino acids to satisfy the increased anabolic demands. The ability of cancer cells to adapt to nutrient shortage is also critical for tumorigenesis. Upon amino acid restriction, the integrated stress response (ISR) is induced via GCN2 kinase. Activated GCN2 phosphorylates eIF2a, resulting in translational reprogramming that inhibits general protein synthesis but paradoxically increases the translation of a subset of mRNAs. The most-notable example of selective translation is activating transcription factor 4 (ATF4), a bZip transcription factor that promotes the expression of genes involved in antioxidant response and amino acid biosynthesis and transport. The ATF4-mediated adaptative program is thus crucial during tumor progression. It is widely believed that the primary regulation of ATF4 expression is through translational control of pre-existing mRNA. The transcriptional regulation of ATF4, however, remains surprisingly obscure.

The current understanding of amino acid response is largely based on full amino acid starvation. It remains unclear whether single amino acid deprivation triggers the common ISR or elicits a unique cellular response. The Working Examples herein identify a unique cellular response to cyst(e)ine depravation within cells that results in a cyst(e)ine stress response that can rescue a cell from ferroptosis. The Working Examples herein further demonstrate that the cyst(e)ine stress response is mediated by transcriptional upregulation of the global transcription factor ATF4. Without being bound by theory and as is also demonstrated in the Working Examples herein (see e.g., Attachment A to the specification), the adaptive ATF4 upregulation in response to cyst(e)ine depravation results in inter alia upregulation of SLC7A11 and upregulation of the system X_{c}⁻ antiporter, which in turn results in increased cyst(e)ine uptake by the cell and rescue from cyst(e)ine depravation induced ferroptosis.

As is demonstrated in the Working Examples herein (see e.g., Attachment A to the specification) ATF4 transcriptional induction in response to cyst(e)ine depravation is governed by lysosomal levels of cystine. Cystine stored in the lysosome serves as a reservoir that can be tapped into to increase cytosolic cysteine via an active lysosomal efflux system when cytosolic levels drop and/or extracellular cystine is limited. As is demonstrated in the Working Examples herein (see e.g., Attachment A to the specification) ATF4 transcriptional induction in response to cyst(e)ine depravation is attenuated with an accumulation of lysosomal cystine, even under a shortage of cytosolic cysteine, such as that which occurs during ferroptosis.

With that said, embodiments disclosed herein describe engineered biomolecules that can be capable of nutrient reprogramming in a cell such that lysosomal cyst(e)ine is elevated and ATF4 induction is attenuated. In certain example embodiments, the engineered biomolecules include a lysosomal targeting moiety, and one or more cysteine-rich motifs, where each of the one or more cysteine-rich motifs is coupled to the lysosomal targeting moiety. In some embodiments, the engineered biomolecule is a polynucleotide (e.g., a DNA or RNA molecule). In some embodiments, the engineered biomolecule is a polypeptide. Also described in exemplary embodiments herein are vectors, such as expression vectors, that can include an engineered biomolecule described herein. Also described in exemplary embodiments herein are delivery vehicles that can include an engineered biomolecule, vector, or both as described herein. Also described herein are pharmaceutical formulations that include an engineered biomolecule, vector, a delivery vehicle, or any combination thereof.

Some exemplary embodiments herein describe methods that include, delivering to a cell, a cell population, and/or a subject in need thereof an engineered biomolecule, a vector, a delivery vehicle, and/or a pharmaceutical formulation as described herein. Some exemplary embodiments herein describe methods of treating cancer that include delivering to a cell, a cell population, and/or a subject in need thereof an engineered biomolecule, a vector, a delivery vehicle, and/or a pharmaceutical formulation as described herein. Also described in exemplary embodiments herein are methods of inhibiting a cysteine stress response in a cell or cell population that include delivering to a cell, a cell population, and/or a subject in need thereof an engineered biomolecule, a vector, a delivery vehicle, and/or a pharmaceutical formulation as described herein.

Other compositions, compounds, methods, kits, systems, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Engineered Biomolecules

Described in several exemplary embodiments herein are engineered biomolecules that contains or is composed entirely of one or more lysosomal targeting moieties and one or more cysteine-rich motifs, where each of the one or more cysteine-rich motifs is coupled to the one or more lysosomal targeting moiety. In some embodiments, the engineered biomolecule is a polynucleotide (e.g., DNA or RNA). In some embodiments, the engineered biomolecule is a polypeptide.

In some embodiments, the engineered biomolecule is effective to inhibit ATF4 expression induction, reduce cytosolic cysteine, increase lysosomal cysteine, or any combination thereof in a cell. In some embodiments, the engineered biomolecule is effective to induce and/or potentiate ferroptosis. In some embodiments, the engineered biomolecule is effective to inhibit a cysteine stress response in a cell or cell population.

As used in this context, “coupled to” refers to direct coupling through fusion (e.g., where a cysteine rich motif is directly adjacent to a lysosome targeting moiety in the polynucleotide or amino acid sequence), indirect coupling (e.g., one or more amino acids or polynucleotides that are not part of a cysteine rich motif or a lysosome targeting moiety are between the cysteine rich motif and a lysosome targeting moiety in the engineered polynucleotide), and linkages/linkers that are not part of the translated and/or transcribed engineered polynucleotide sequence or polypeptide sequence). The cysteine-rich motif(s) and lysosome targeting moiety(ies) can be included in the engineered biomolecule in any order/location.

In some embodiments, the engineered biomolecule contains 1-50 or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more) cysteine rich motifs. In some embodiments, the cysteine-rich motif is a cysteine-rich domain or region thereof of a native protein or polypeptide. Exemplary polypeptides with native cysteine-rich motifs that can be incorporated into the engineered biomolecules described herein include, without limitation, any of those set forth in Table 2.

TABLE 2

| | | | | | | |
|---|---|---|---|---|---|---|
| RBAK- | TMEM130 | ERVV-2 | TENM2 | SOS2 | INPP4B | SLC35C1 |
| RBAKDN | | | | | | |
| GPR37L1 | OXA1L | USP40 | NOP16 | SPDYC | NRG2 | SHC2 |
| KCNK12 | GEN1 | ZNF792 | SNX6 | CKLF | FASTKD2 | SKIL |
| TXK | TRPM3 | CHORDC1 | KIF12 | TSPAN5 | TMEM190 | ZFYVE9 |
| FAM155B | GML | MAP1S | ZP3 | TSC1 | EBF4 | TLE2 |
| TTC13 | NRN1L | TUBGCP6 | TACC2 | FIGF | GRIK5 | ADGRD1 |
| GPR37 | ZNRF3 | TCAIM | MRPS30 | SHISA4 | CDY1 | ATR |
| CRACR2B | TMEM184C | KLB | PADI2 | EPG5 | TRERF1 | GABRE |
| PTTG1IP | ILDR2 | SLC6A14 | EDC4 | ZMYM1 | SCEL | KEAP1 |
| CECR6 | KRTAP5-1 | GPCPD1 | AGTPBP1 | DUSP5 | NT5C1B | STARD4 |
| XKR4 | LAPTM5 | CCDC82 | EPHA5 | MYH13 | MYBPC1 | ADSL |
| FGF10 | LTBP2 | PARP8 | USP47 | USP17L3 | MTMR12 | ADAM30 |
| SLC51A | CD164L2 | OR52A5 | KCNG2 | MSTN | MSL2 | SYNJ2 |
| PCDH10 | AJUBA | ACKR2 | HSD11B2 | C21orf33 | SYT14 | AGTR2 |
| GPR157 | HS3ST2 | CYFIP1 | GPR107 | IL37 | PTPN5 | ACSL1 |
| MDFI | ODF1 | RFFL | MMS19 | SENP3 | TNKS | ANKEF1 |
| DNAJC5G | CERK | IGFBP3 | DTL | DSG2 | OR2V2 | FES |
| MDFIC | IL27RA | TGFA | PHTF1 | FANCI | KRTAP9-1 | RPGRIP1L |
| SLC34A2 | SMAD7 | ERVMER34-1 | NLRP2 | RBL1 | ABCA5 | ZNF346 |
| GSC2 | CNFN | GHR | FHL1 | GLYCTK | RHOQ | GPR155 |
| TREML4 | KRTAP4-6 | RALGAPA1 | ZNF804A | METTL24 | NAA35 | SMC5 |
| IGSF5 | HCRTR2 | KDM4E | DMXL2 | LTBR | CBWD3 | CD27 |
| CYSRT1 | SLC26A11 | BEX2 | FBXO3 | CENPE | ATP5SL | ANAPC11 |
| SPIRE2 | SCARF2 | NPIPA3 | TRIM38 | CHAF1B | ADAM2 | CD151 |
| SLC12A4 | PLEC | RIT1 | TRIM41 | RAB9B | CCR9 | AWAT1 |
| CLCN6 | LTBP1 | CD37 | HAVCR2 | HBP1 | DEFB118 | MAP4K3 |
| DAGLA | HHIP | LRRC32 | SAP30L | KIF27 | BTBD1 | VNN3 |
| DNAJC5B | ATRX | DYNC2H1 | CHRDL1 | HPS4 | MFSD5 | FAM160A1 |
| KIAA1147 | HELB | TM4SF5 | FBXO40 | CXCR3 | ZBTB48 | TNMD |
| ENDOV | ALDH2 | ATMIN | ORC3 | WDR37 | ZNF175 | AVPR1A |
| GPA33 | KRTAP5-3 | SLCO6A1 | POMGNT2 | CUL9 | CNBP | FHAD1 |
| PCNXL3 | PLSCR4 | PRAMEF10 | MEGF9 | LARS | IL19 | RASL11B |
| SMIM18 | TMEM65 | SETD2 | DSG1 | CCDC68 | IFT172 | RFX5 |
| RGS19 | COL6A6 | MDM1 | GBF1 | C11orf16 | LAMC2 | C4B |
| MREG | KRTAP2-3 | TMEM86B | FAM181A | STK38 | GRB14 | SHISA6 |
| PCNXL2 | OR51Q1 | LYPD1 | ATP10A | CCL26 | RRNAD1 | TTLL7 |
| RFPL3 | CCDC142 | ZNF660 | FGGY | VWC2 | KISSIR | FAM60A |
| C14orf93 | KRTAP5-6 | PANK2 | ARAP2 | EXOSC10 | LYPD3 | DEDD |
| SLC12A6 | RFPL2 | TMEM219 | RP11- | OR2T5 | OR51B5 | GDF5 |
| | | | 446E24.4 | | | |
| FAM174A | KCNQ3 | U2AF1L4 | RAB40AL | OPN1MW3 | RAPGEF5 | ZNF644 |
| CHIC1 | CNNM2 | TBL3 | GZF1 | SLC37A4 | TNFSF15 | ROBO3 |
| ACBD5 | KIAA0319L | EPPIN | DNAH1 | MSMB | ENPP2 | ADGRA1 |
| LRRC15 | TRPM1 | SMPD1 | GRIN3B | AMMECR1L | GNB2 | SLC45A3 |
| PLSCR2 | CDHR5 | SEL1L3 | LEFTY2 | ST8SIA1 | HTRA3 | CEACAM18 |
| MCAM | SPDYA | PITPNM1 | KRT32 | ZDHHC15 | C19orf35 | TTC37 |
| EFR3A | KRTAP2-1 | AGR2 | SLC22A11 | KCTD2 | OR1L3 | LMO4 |
| SYT8 | WDFY3 | SLC36A2 | PRUNE2 | TAB3 | TGFB1 | CH507-9B2.3 |
| ADAMTS19 | COL6A5 | ASPSCR1 | FBXL7 | CRYGB | ANKRD11 | VAMP3 |
| SMIM22 | RP11-613M10.9 | OXT | MYO9A | NISCH | SCN4A | COX17 |
| CPEB2 | ANTXRL | KBTBD2 | SMPD3 | FBXW12 | GABRG2 | C16orf95 |
| EDNRA | KRTAP5-4 | ZNF468 | PRICKLE2 | HGSNAT | MYO18A | CROT |
| DNAJC5 | CASP5 | NUTM2E | TEX14 | LY75 | ACKR3 | GPRIN2 |
| RGS20 | TMPRSS6 | LRRC53 | KAZALD1 | SLC52A2 | TUBA1C | SERTAD4 |
| CRCT1 | KCNQ2 | AASDH | IFITM5 | FAM188B | TNFRSF19 | ZIK1 |
| CHIC2 | KRTAP23-1 | RNF151 | KRTAP9-2 | ZSCAN20 | PDZD2 | IQSEC3 |
| VSIG8 | LMX1A | POP1 | KLHL11 | LRRK1 | USP18 | APPBP2 |
| RFPL1 | MYO16 | HEATR9 | POLRMT | CD180 | CDKN3 | CDPF1 |
| C11orf21 | PLAA | ATRAID | FAM205A | BTBD18 | ATP11C | STRIP2 |
| RGS17 | WDR60 | ABCA1 | SLC2A10 | EDEM2 | MCM3AP | TTC27 |
| KRTAP12-4 | CLCN5 | ZNF727 | SH3PXD2A | SLFN12L | TRRAP | BRWD3 |
| KCNQ4 | FBN3 | IGF2 | ADRA1D | AL109927.1 | RASA4B | MT1M |
| C1orf68 | ILDR1 | RFC3 | PI4K2B | KLRG2 | LECT1 | OR2M5 |
| ERGIC1 | ACVR2A | ATP11A | NLRP3 | CCDC85A | LRRC37A3 | COL14A1 |
| UBR5 | KRTAP5-5 | SOS1 | NUP205 | KIF20A | TSPAN18 | PTX3 |
| ZNF416 | CD163L1 | CDC42BPA | PRDM13 | KIAA1841 | DYM | TMEM55A |
| AQP11 | SMIM5 | MAP1B | CTDSPL | BRF2 | LGR4 | IL18RAP |
| SERINC4 | LRRC55 | ARSH | GSTP1 | SPPL3 | PRAP1 | CD38 |
| P2RY11 | KCTD16 | TIGD6 | LRRC1 | CDCA7L | PDHA1 | EYS |
| ABCA7 | KIAA0319 | ZDHHC21 | GPR183 | LAPTM4A | TC2N | HCFC1 |
| COL26A1 | COL4A6 | CCL4 | MEGF8 | YIF1B | UROD | SGCA |
| DDX11 | KIF4A | INTS2 | SERPINB9 | CYHR1 | MAP3K13 | KMT2E |
| CLCN3 | MCOLN3 | CLEC4C | SLC35G1 | TRIM16 | BTNL8 | ACBD3 |
| CYP7A1 | FBN2 | FBXO15 | ATE1 | RMND5A | ATP13A3 | ABHD2 |
| BRIP1 | CCER1 | TUBA1A | MAS1L | NCR1 | FITM1 | TECPR1 |
| SHISA5 | TSEN2 | OR10W1 | ATP8B2 | COL22A1 | RP11-468E2.1 | KCNK15 |
| GNA14 | ZMIZ1 | SUSD2 | RMND5B | P3H1 | TINAGL1 | MAP3K1 |
| TNFRSF10A | GPATCH3 | ST6GAL1 | ERV3-1 | ESPL1 | ATF7IP2 | ZNF428 |
| SYT2 | KRTAP4-8 | USP32 | C1QTNF8 | TMEM184B | TYW3 | RAG1 |
| TCTN2 | KRTAP5-11 | VWCE | SCN5A | NINL | CGA | EDA |
| MCOLN1 | WBP1L | ARHGAP36 | SLIT3 | SLA2 | CUX2 | DHH |
| ZNF507 | KRTAP5-7 | GCA | ICOS | C15orf59 | SCGB1D1 | CCL13 |
| NOSIP | ZDHHC23 | GRIP2 | UBXN8 | UGT1A10 | COL5A2 | FAM153B |
| TRIM42 | IER5 | LHFPL1 | SEP15 | C12orf79 | ACVR1B | ANTXR1 |
| FAM227A | SERINC5 | TMEM189 | AMER1 | MPP4 | MZT2B | UHRF1BP1L |
| INTS1 | FBXO38 | PDE9A | WDR24 | CBLC | SERAC1 | SEPT1 |
| PROM2 | SLC29A3 | NPAS2 | CASP8 | DPYS | MYOM2 | S1PR5 |
| C3orf17 | TCTN3 | CAPN12 | PODXL | C2CD5 | CCT8 | TICRR |
| LTBP4 | LRSAM1 | EXOC3L2 | IGFBP4 | RAD9A | ACRC | RAPGEF3 |
| STARD9 | SLC25A17 | IFT122 | ALDH1A1 | SLC16A7 | PADI4 | CHRNA5 |
| TDRD1 | VAC14 | CD81 | SLC35A5 | CWC22 | ZFYVE21 | KCNJ14 |
| KRTAP4-5 | CCKAR | OR6A2 | YY1AP1 | WRN | ROBO1 | GCKR |
| KRTAP5-10 | LIMS1 | SLC16A10 | DISP1 | CHD5 | RPRML | RHD |
| BZRAP1 | TRAF3IP1 | SPRED3 | RERE | SLC25A15 | CCL16 | WFIKKN1 |
| CAMK1 | LRIG2 | DLGAP4 | EMP3 | RANBP9 | LY6D | MARCH2 |
| DAGLB | MTMR4 | SRGAP1 | ZMYND12 | AMMECR1 | ALDH9A1 | SPAG17 |
| PDE4DIP | NFIX | PGPEP1 | NELFCD | VOPP1 | NRIP2 | GNS |
| KIAA0232 | POLA1 | UNC13B | NLRP6 | GPR148 | PRKCSH | TRAF7 |
| ACVR2B | FAM172A | OR2M7 | ITK | COX10 | CCR1 | TARBP2 |
| HIRA | SEPT9 | SMARCA5 | PDP1 | HEG1 | KRTAP9-4 | MAP3K14 |
| SCARF1 | CCDC64 | KRTAP10-11 | DEAF1 | BTC | ZNF225 | EFCAB14 |
| ANKRD40 | TMEM245 | NEO1 | DKK2 | GAN | OR10C1 | C1QTNF1 |
| KRTAP2-2 | CDKL5 | GPM6B | TDRD5 | ACOT4 | C10orf12 | MICAL1 |
| CLDN15 | PPP1R36 | LRRC4 | GNB1 | FHL3 | FOLR3 | KAT6B |
| ZNF843 | GPAT2 | MPDU1 | ISPD | SHPRH | ADRB1 | RXFP3 |
| EML5 | ABHD17B | KCNN4 | PTPN2 | CATIP | ZNF414 | TRIM35 |
| STT3B | GIT1 | RPGRIP1 | TRIM77 | CCDC24 | CCDC62 | UBE2Q2L |
| IQSEC2 | KHDC1 | ERCC6L | NFIB | SRPK3 | ADAM20 | ZIM2 |
| SPTLC2 | CARNS1 | GADD45B | PLA2G2A | ZMYND10 | LCE5A | SLFN13 |
| PHLDB3 | ADPRM | ANKRD9 | LCE1B | MYRF | GABRB1 | ALG3 |
| KRTAP2-4 | SHISA8 | TNFRSF9 | TP53TG3 | PTCHD2 | RNF34 | C11orf96 |
| GEMIN4 | MAP3K15 | PCGF1 | IER5L | CHODL | ADCY1 | RNF150 |
| PPAN-P2RY11 | CTNND2 | GNE | IGFL2 | GRIN1 | ZDHHC22 | ERVV-1 |
| TCTN1 | ZNF280C | KIAA1033 | PAQR8 | CCNK | PKDREJ | TGFB3 |
| PHLPP1 | HIRIP3 | GPR75 | LDLRAD1 | OR10P1 | PROC | UNC80 |
| PPP1R11 | CNOT10 | SIAH2 | GABRG3 | RECQL | FST | F2R |
| LYST | ATP10D | MELK | KDELR3 | LRFN5 | ANKRD28 | CNIH2 |
| KRTAP4-11 | SIRT1 | WNT5B | MAGI3 | KIAA0195 | KRTAP9-7 | PRICKLE4 |
| NPFFR2 | MADD | PPP1R15A | ARL8A | HSH2D | FAM163B | TNK1 |
| SHISA2 | CD200R1L | BANF2 | CD47 | RASAL1 | PCLO | ZZZ3 |
| KRTAP5-9 | DCT | SLC25A16 | DNAH14 | WDR17 | SLAMF7 | PKDIL1 |
| KRTAP5-8 | FAM120B | ISM2 | GPR26 | POMZP3 | C21orf140 | ZNF717 |
| ADGRG2 | DNAH8 | DENND2A | AHRR | PSMD1 | DENND1A | TRIM43 |
| TBC1D8 | KIAA1429 | CPNE6 | CRELD2 | KATNBL1 | CHD6 | HPS5 |
| KRTAP4-12 | IKBKAP | TSPAN16 | SLC6A8 | FANCC | GIMAP8 | GDF7 |
| ADGRG6 | GPR22 | SLC30A10 | TBPL2 | SCARA5 | PDDC1 | PIGQ |
| ZDHHC7 | SMG6 | PPP2R5C | CACNA1C | LETM1 | PPP1R3G | CHFR |
| KRTAP4-16P | BMP8A | TENM3 | OR5L2 | TRIM6-TRIM34 | HMHA1 | TMEM99 |
| HDAC11 | GAA | DDIAS | ZNF827 | HSPBAP1 | KCNC4 | NRBP2 |
| PCDH17 | HTR1A | DRC7 | SP140 | OPRL1 | PEX11G | RAD51L3- |
| | | | | | | RFFL |
| AOAH | ZNF664 | NWD2 | PKD2L1 | PASD1 | REST | RECK |
| TMEM52B | ADGRL2 | LIX1 | WDR27 | ITPRIPL2 | CRIM1 | ALDH1A3 |
| LYSMD4 | METTL17 | TPRA1 | SEMA3F | NOP2 | AMN1 | KDM2A |
| KRTAP17-1 | UBXN11 | ZNF587B | NRROS | OASL | NPSR1 | NPIPA7 |
| KCNQ5 | FAT1 | RBMXL3 | OR2AK2 | STARD13 | WDR70 | CACNG2 |
| BMPR2 | LPPR2 | TMEM201 | C19orf84 | WNT2B | DNMT3A | TRIM40 |
| WDSUB1 | COL19A1 | CNNM1 | PDK1 | MBTPS2 | PHKA2 | CAMKK1 |
| TMEM64 | GLIS2 | USP35 | DUSP10 | AK8 | CD1E | PER3 |
| ADGRG3 | PKN2 | ATP13A1 | LRRC14B | HRH2 | ARMC8 | LPPR4 |
| WBP1 | ARMCX6 | STAT5B | GP2 | KLHL22 | NCAPD2 | ANAPC15 |
| ULK2 | INSRR | LRRC36 | CAMTA1 | GLIS3 | ACER3 | FASTKD3 |
| KRTAP4-4 | MS4A4E | MCHR2 | RNF133 | PLK1 | KLHL34 | RXFP2 |
| FAM221B | RNF222 | ACOT11 | HSF5 | KMT2A | FANCB | ZNF185 |
| SNRPB | KCNV1 | ANKRD44 | EBLN1 | FAM193A | ZCCHC6 | ALG13 |
| MMS22L | CREG2 | OR4M2 | OR7A17 | CCKBR | CCDC136 | TMEM183A |
| ALDH1B1 | PTK7 | CELF1 | CHD1 | TRPV5 | SHROOM4 | RP3-509I19.11 |
| KRTAP10-10 | PAOX | WDR72 | RHO | MANSC4 | ZAR1L | PRR5- |
| | | | | | | ARHGAP8 |
| DSG4 | SLC10A6 | KLHL26 | KMT2C | PITPNM2 | OR1M1 | MT1A |
| APAF1 | ZNF800 | TLL1 | LRRC57 | ABCA13 | GPR27 | BBS2 |
| SCUBE2 | PLEKHG5 | CHRFAM7A | GTF3C1 | ADGRL4 | ZNF213 | WDR45 |
| TTYH1 | TEX15 | TRMT2B | RALGAPB | AUTS2 | MTHFR | NLRP8 |
| TMEM63A | SERINC3 | RIC3 | OR812 | DICER1 | BTBD9 | FTMT |
| LMX1B | METTL3 | NHLRC2 | ERI2 | METTL8 | PPP2R2D | RMI1 |
| CLCN4 | PIEZO1 | BCL2L14 | GUCY1B3 | SEMA3A | IGHMBP2 | GRM7 |
| FZD5 | ASH1L | EGFL8 | PAQR6 | NFYC | IRAK2 | MTMR8 |
| HTT | SETX | RPS8 | TRIM34 | SKAP2 | CHRM1 | SCGB1A1 |
| PIK3C2B | CBWD1 | MRPS5 | DPAGT1 | FBXL17 | NLN | SRGAP3 |
| FBN1 | EBF3 | LRRIQ1 | ZDHHC14 | SACS | RAF1 | SLX1B |
| RHOBTB3 | FEZF2 | PIK3C2G | C12orf79 | PROKR1 | TMEM144 | PRAMEF26 |
| PLSCR1 | LCE2C | KRTAP3-3 | CCL18 | MFSD12 | RNF219 | C1QTNF3 |
| TMEM63B | CEP250 | JMJD1C | ASAH1 | KRT83 | ZNF257 | FAM178B |
| ALG6 | LYPD6B | KCP | INTS5 | PXDNL | XIAP | PGAP3 |
| BTAF1 | COL12A1 | MTCL1 | RRP9 | SORCS1 | AMH | RSBN1 |
| KRTAP4-1 | OR2T29 | FBXO4 | BTD | SLC10A2 | TMEM184A | IL20 |
| LRWD1 | GDPD2 | TUBAL3 | TMEM232 | TSPAN1 | FAM19A3 | XKR3 |
| CLDN17 | KIAA0226 | MUC5AC | DHX58 | ASTE1 | SULF2 | RP11- |
| | | | | | | 520P18.5 |
| LSR | C7orf55-LUC7L2 | HNRNPK | KIT | ABCA2 | C2CD3 | NLRP11 |
| PADI3 | CHST15 | PCDH15 | EIF2B4 | SLC40A1 | AMBRA1 | SFMBT2 |
| FOXD2 | NSD1 | SLFN5 | PRICKLE3 | RRAGD | HHIPL2 | TEC |
| MARCH4 | GBP2 | CDRT15 | SALL1 | EXOSC5 | WWC2 | FOLR1 |
| NPC1L1 | C17orf104 | ARHGEF28 | UNC5C | SGK223 | KIAA1210 | MYO7B |
| ZFYVE26 | CLDN6 | TPCN1 | MAP4K1 | TMBIM6 | USP51 | ZNF695 |
| KIAA1875 | PARP10 | SLC52A3 | FAM26E | HELLS | OR2T10 | KCTD10 |
| IRAK1 | GCLM | SDCCAG8 | LRRC8E | WFDC9 | EXOC3L1 | RAB40B |
| VAV1 | SEPT2 | HCFC2 | KRTAP15-1 | G2E3 | GPR108 | ADAR |
| TTF2 | AGRN | LRRC4B | CAPN8 | RNF145 | TSPAN17 | EXOC2 |
| IL4R | WNT6 | KIF21B | PIK3CB | TNFRSF11A | NEK4 | EPB42 |
| LTBP3 | CLOCK | ZNF775 | SMG8 | SEPT5 | MOB2 | CHD9 |
| KRTAP4-7 | BRMS1 | DLEC1 | GRIA1 | ZDHHC8 | CEP152 | COLQ |
| CHAMP1 | GRM5 | DDX20 | ZNF513 | MALRD1 | LAMA2 | CSF3R |
| KRTAP4-9 | IFITM1 | KCND2 | ATP8B1 | XRN1 | MYLIP | IFRD1 |
| TTYH3 | CASR | CTC1 | TNN | INTU | RP11- | AP1S1 |
| | | | | | 1212A22.4 | |
| COQ3 | PTP4A1 | SVIL | KCNC1 | ADAP2 | KIF11 | GPC5 |
| PCSK5 | TRAPPC13 | MRPS12 | ANKRD39 | CENPL | SH3GL3 | GMIP |
| SDHA | TRIM64 | EBLN2 | NT5C1B- | LIMS2 | ZDHHC3 | MYO18B |
| | | | RDH14 | | | |
| WDFY4 | ACACA | CTHRC1 | ADGRG4 | TINAG | WDR78 | CCNI2 |
| KLHDC3 | KRTAP21-1 | DRGX | CLPS | PRMT7 | TCP11 | RNF183 |
| DOPEY2 | IRF2BPL | GABRG1 | THAP7 | TRAF2 | TMEM253 | CCL22 |
| KRTAP5-2 | SLC25A51 | PTCHD1 | AUNIP | ADAMTSL1 | PI4K2A | RPS6KL1 |
| SLC16A12 | PRAMEF17 | VWA3A | WNT3 | PSMB6 | OR52N5 | GSTCD |
| TCF19 | RAD54L2 | TMEM116 | MTL5 | SLC6A15 | SNX7 | SLC4A3 |
| SPAG5 | TMEM74 | RP11-257K9.8 | ZDHHC20 | KIAA0922 | ZNF462 | RP11- |
| | | | | | | 766F14.2 |
| TSHR | FANCE | HSP90AB1 | FNIP1 | RNASE11 | MTF2 | KLHL3 |
| TXNRD3NB | ERICH6 | SYNE2 | OR6K2 | TMEM102 | ZNF438 | POLD2 |
| TRPM8 | PRIMI | UHRF2 | NOXRED1 | TSGA10IP | COL6A2 | CCL15 |
| CCDC15 | TKT | CD8B | ERVW-1 | POLR2F | ID3 | MAP1A |
| SHB | SLC30A5 | PCNT | CTSB | KRT82 | NOD2 | FAM193B |
| SLC39A14 | C22orf42 | P2RX4 | SLC12A7 | WNT16 | SSPN | NOC3L |
| PCNX | PTGS1 | METTL2B | OR2G6 | RBBP5 | CDAN1 | KRTAP13-4 |
| MEGF11 | DEPDC1 | TRIM61 | NPIPA8 | CLDN5 | GIMAP2 | FBXL5 |
| DCAF16 | ATG4A | KRTAP12-1 | ACSM5 | ASTN1 | RAPGEF4 | DEFB113 |
| PRAMEF5 | FMO2 | ASAP2 | OR2T4 | TLE1 | TDRD7 | DEFB125 |
| SPATA32 | TFF3 | GTF2IRD2 | ABHD17A | WFDC1 | ZMYM3 | RARRES3 |
| ARL13B | SPHK2 | CBARP | HAVCR1 | RP11-864I4.1 | SYMPK | HTR1E |
| TLR7 | ICAM4 | MORC2 | TRIP12 | FKRP | IL25 | KRT78 |
| ZFP69 | POLQ | TCF20 | KRTAP4-3 | CDC23 | PINLYP | SYNE1 |
| ZDBF2 | ADAMTS14 | IRF2BP1 | PTCHD3 | SFXN3 | RASA2 | PRKCB |
| FAM111B | DEFB112 | AC124312.1 | RNF14 | PPM1A | CTSA | ITGB4 |
| LRRC14 | RFTN1 | MANSC1 | SMU1 | TNFRSF11B | SDHB | CPNE8 |
| HCRT | KLHL12 | SWAP70 | TMEM259 | OR6C68 | WDR48 | B3GALT2 |
| WDR87 | MOV10L1 | AP5B1 | KIRREL2 | CR1 | EPHA1 | TRIM72 |
| TAF6L | RAB7A | ZNF500 | RBM47 | KLHL15 | OGFOD2 | MARCH11 |
| SPRY1 | MBD2 | FBLN2 | ANKRD36C | STK36 | ATP10B | SDF4 |
| MIER3 | MICAL2 | HOXD10 | DPYSL3 | DHX32 | SYT1 | SEMA6B |
| ESRP1 | TMTC3 | RNASEH1 | KRTAP10-4 | NRK | PRG3 | RGS3 |
| ANXA8 | PLCG2 | RAET1G | ZMYM2 | INHBC | NUTM2A | CADM3 |
| EXOSC8 | FAM136A | TEKT5 | PXDN | KPRP | CLDN1 | PRDM14 |
| ADAM33 | TESPA1 | RNF7 | DCAF5 | PGBD2 | WDR63 | VWA5B2 |
| JAK2 | KRTAP1-3 | ZFHX3 | ICAM2 | NLRP14 | PLCH1 | DNAJC18 |
| PFN3 | CAND1 | COG1 | ZSCAN22 | RNF44 | FN3K | DPYSL4 |
| GSC | OPN5 | PPT1 | CTD- | CCRL2 | PLA2G5 | TMEM55B |
| | | | 2006C1.13 | | | |
| ZXDB | MTSS1 | HTATIP2 | ALDH1A2 | IGF1R | SLC17A5 | BNC2 |
| WNT8B | TCFL5 | RAP1GAP2 | FGF17 | ADH1B | CXCR4 | KRTAP1-5 |
| DUSP8 | PHTF2 | KIR3DL3 | HMGCR | ATP2A1 | POTEB3 | PLA2G12A |
| BSN | POTED | THNSL1 | FMR1 | L3MBTL3 | CCL5 | CYFIP2 |
| PPP6R3 | NDUFAF6 | FZD2 | MS4A4A | SERPINA1 | IRX6 | ZNF282 |
| DPH5 | ANKRD36B | PKMYT1 | CLEC6A | MB21D1 | RNF103 | TTLL6 |
| C5AR2 | TRIM58 | UTP20 | TGFBR2 | KLRD1 | LAMB3 | HCAR3 |
| OR4M1 | TYR | SNX32 | DEFB132 | WWP1 | SLC39A5 | MYRIP |
| TCAF1 | NOTCH2 | ERCC6L2 | ARHGEF3 | TTC39A | COL18A1 | ZKSCAN2 |
| TTK | UGT1A9 | SPINT1 | FKBP5 | FRS3 | UGT1A8 | FAM155A |
| POSTN | OTOP3 | PTER | VEGFB | CCL21 | NUP153 | SMC4 |
| NBEAL1 | TARBP1 | CLPSL1 | PRIMA1 | RNF40 | C14orf39 | BIRC3 |
| ATAD5 | FAM20A | WFDC5 | SAP30 | ENAM | SPNS2 | UNC13A |
| MICB | NAA25 | NNT | PDLIM5 | ZNF211 | CDC27 | FAM26D |
| STARD10 | HLTF | CILP | NOX3 | TRIM24 | POTEJ | C12orf49 |
| PIGB | PTGFRN | LPAR1 | TACR1 | RHOV | TBC1D13 | SMYD5 |
| CCR4 | C1orf112 | GRK4 | IGFL4 | VPS13D | NALCN | SLX1A |
| CPAMD8 | TPMT | USP17L1 | DPP8 | DRD3 | EDAR | SALL3 |
| KCNF1 | PAQR9 | CFAP54 | MAP6 | RTL1 | GEMIN5 | DGCR2 |
| ECT2L | C9orf3 | FOLR2 | FAM20C | SP140L | PVRIG | PIGG |
| TRPM6 | PTPRO | ZBTB7A | POTEH | ZKSCAN5 | DPH2 | MTX2 |
| CCNYL1 | DGKK | LRPPRC | FAM117A | POTEI | SOHLH1 | ATP2C2 |
| OSMR | ADRB2 | ERRFI1 | NEGR1 | DENND1C | FBXO30 | GUCD1 |
| CCDC84 | BTLA | ABCA8 | LPPR5 | GPR20 | SMARCA1 | TRAF5 |
| STAC2 | VNN2 | DEFB129 | MC1R | LHX9 | LPAR3 | GRIN2A |
| KCNB1 | TSNARE1 | MYO5A | ZNF132 | AIRE | WDR83 | NT5C |
| TUBGCP3 | SLC15A3 | LRCOL1 | FAM198A | ZFYVE19 | LRRC4C | ZNF496 |
| TLE6 | KDM4B | AGPAT9 | BTG4 | KCNJ4 | PIWIL2 | MYO10 |
| CFAP61 | RASGEF1A | ARFGEF3 | DDB2 | TCTE1 | MACF1 | SCN11A |
| NCDN | SPPL2C | FAAH | SCN2A | MALT1 | XKR9 | GPR4 |
| SFXN1 | GPR55 | TDGF1 | WDR90 | BCAM | C6orf89 | HEATR5A |
| THEMIS2 | SPRY4 | RTP5 | PSMD12 | STXBP2 | TUBA3D | FOXE3 |
| SDAD1 | TRIM2 | ASCC3 | NCAPG | HIPK3 | PPP2R5D | INSC |
| ZNF707 | IGFBP2 | UBE2J1 | EPAS1 | SLC27A3 | TAAR9 | MLH3 |
| BTBD8 | RHCE | CDC42SE1 | PGAP1 | CEP85L | CALM2 | NLRC4 |
| FAM117B | CDON | AQP12A | FAM181B | TRAF3 | HRH1 | HTRA4 |
| SLC25A52 | PHIP | PIH1D3 | NUB1 | GPRASP1 | SOCS7 | ITGA8 |
| ST8SIA6 | OR10X1 | LZTR1 | PHF12 | NOX1 | ZC3H10 | ADAM11 |
| SEC16B | RNF111 | RP11-35N6.1 | HSP90B1 | SPAG16 | RABGGTB | SPON1 |
| UMOD | ST8SIA5 | ZNF516 | SPPL2A | MXRA7 | SLITRK5 | RBFOX1 |
| OR1D5 | CX3CR1 | DCSTAMP | TGM7 | PLA2G2D | CLEC10A | NNAT |
| SHC3 | BMP5 | C4A | TTYH2 | USP48 | TOP1MT | PTGFR |
| WDR13 | SPNS3 | P2RY10 | CDCP1 | WFDC6 | VPS13B | TKTL2 |
| PDE2A | MROH1 | ZNF592 | BCL2L15 | ACSL3 | PDS5A | C1GALT1C1 |
| AIM1 | ZFHX2 | TRIM45 | RP11- | TBCD | TNFRSF13B | AC138969.4 |
| | | | 548K23.11 | | | |
| UGDH | KCNJ5 | CRLS1 | CFAP46 | NT5DC1 | USP17L2 | ZNF572 |
| CRMP1 | CMC4 | NODAL | FAM153C | TNC | ESPNL | C3orf84 |
| SLC39A2 | NADK2 | CAPN15 | GC | SEMA3E | XKR8 | KCND3 |
| HTR3E | DNMT3B | PAPPA2 | SUV39H1 | PGBD5 | GRAMD4 | RGS21 |
| FTSJ3 | WNT4 | TENM4 | HSD17B4 | FNIP2 | ZNF69 | CCDC125 |
| ADCY6 | CCL7 | STAT6 | ZNF724P | ARL6IP6 | MAGEC2 | TSPAN3 |
| CTNS | C5orf45 | LHX2 | FRAS1 | RFX6 | DOCK4 | NKAIN3 |
| ADAMDEC1 | STAT5A | THRB | PPP6R2 | GPR78 | ITGA2 | C1orf159 |
| ZDHHC13 | CIT | CELSR3 | ZNF449 | FOCAD | NDST3 | PGAP2 |
| PTCHD4 | MTOR | SLC34A3 | CSTF1 | TMEM186 | COL17A1 | MYT1L |
| LATS2 | PTHIR | TGS1 | SPATA2L | BAAT | LAMA1 | PRAMEF12 |
| PKHD1 | FAM170A | PRAMEF26 | THAP11 | PHOSPHO1 | MFAP3 | AHCYL1 |
| ISX | CALHM3 | DGKE | CLEC4E | FAHD2A | CMTR2 | ZNF273 |
| KRTAP10-3 | USP13 | AXDND1 | ADCK1 | SLC26A8 | ZNF770 | WNT2 |
| STXBP5 | APOBEC4 | ZNF678 | LAMTOR1 | ZNF442 | IFITM10 | GDF15 |
| TRPV6 | RP11-540D14.8 | PNPLA7 | ERCC8 | CCNC | TDRD15 | NCAPG2 |
| ADH1A | RNF144B | SEMA3B | KCNA2 | ANKHD1 | MCF2 | TYK2 |
| RNF138 | ZBED6 | USP34 | TTC3 | ANKMY1 | HGH1 | SYNE3 |
| SYNGAP1 | IRF2BP2 | ARHGEF17 | ZP1 | CD82 | DPYSL2 | ARL5A |
| STS | SLC24A4 | TMEM61 | UVSSA | SMYD4 | IVNS1ABP | FYN |
| KRTAP9-8 | CLCN2 | ADAM8 | GRM6 | CPSF2 | DPT | DNAH10 |
| ZCWPW2 | ACAP2 | SEMA3G | PLD1 | OVOL3 | RNASEK | ZNF236 |
| ZDHHC6 | MKKS | WNT1 | SCN3A | PDE4B | ARL15 | CCDC47 |
| IL20RB | IQCF3 | TIMD4 | ANGEL1 | MAP3K6 | GLRA4 | DSC2 |
| GAPVD1 | KCNIP3 | EGFL6 | THAP6 | RIMBP2 | USP41 | ADRA1A |
| PIGN | DNAH9 | CNGB3 | GNA15 | CYP4F3 | NFATC4 | NANOS1 |
| SP100 | RYR1 | LRRC58 | RAG2 | JAG1 | GRINA | CHD7 |
| BHMT | RRAGC | MS4A18 | SLC35E2B | H6PD | SMPD2 | SETDB2 |
| KIF5A | TGFB1I1 | RNF186 | SLC5A6 | C11orf52 | CUL5 | ZNF235 |
| ATP8A1 | CEP120 | ASB1 | TMEM91 | NSUN5 | NLRP9 | DSTYK |
| INTS7 | RANBP10 | C6orf163 | ATG2A | SPSB3 | HECTD4 | PRADC1 |
| ZFP36 | WHSC1 | CEP68 | KMT2D | ARMC5 | NSUN4 | ZNF274 |
| KRT36 | ADAM15 | MPC1 | STXBP3 | KBTBD6 | DENND3 | OSCAR |
| CSRNP1 | TMEM189- | CTDSP2 | EIF4EBP3 | WDR44 | SEC24C | EFCAB2 |
| | UBE2V1 | | | | | |
| GRM2 | CCL3 | LRRC66 | GPSM2 | UGT1A4 | EPB41L4B | DOCK3 |
| JRKL | KIAA0100 | OR52N2 | IL1F10 | CCL2 | IL34 | BMPR1B |
| OSGIN2 | OTOGL | UBR4 | WDR5 | SPACA4 | TENM1 | KIAA0825 |
| MAD2L 1BP | EMP2 | ADGRL3 | MTMR10 | CEP192 | ICAM3 | ZNF446 |
| PPP2R5E | LRRC9 | FAM47A | KRT37 | FAM159B | CCL3L3 | KALRN |
| FFAR4 | RREB1 | ROS1 | TRANK1 | SP110 | ALB | PNMA5 |
| TXNDC11 | MAP6D1 | BLM | TUBB3 | ADAM22 | ZNF865 | TP53TG3C |
| ERGIC3 | PKD1L3 | STAB2 | NMBR | CHRNA3 | OR51T1 | PMP22 |
| GTF2IRD1 | RPL8 | ZFP36L2 | ASGR2 | UBE3C | NOC4L | C1orf53 |
| GADD45G | MOV10 | SPRY3 | AMFR | COL3A1 | SEPT12 | FAM188A |
| KRTAP1-4 | PLA2G1B | NBEAL2 | DCC | LY6H | KRTAP3-1 | CEP170B |
| SNTG2 | FAM24A | SLC47A1 | ISLR2 | WISP2 | SLC13A4 | MRPS21 |
| C3orf80 | ADAM12 | KIF14 | ZNF556 | GLDC | VWF | OR10AD1 |
| SLC15A4 | FNTB | SYDE1 | GFM2 | EIF2D | PDHA2 | KCNB2 |
| ZNF268 | KRT86 | SELL | C5 | PHLPP2 | EFCAB6 | NUS1 |
| PPP1R16A | SCGB1D2 | ZDHHC18 | KRTAP4-2 | ZNF10 | SLC17A4 | CLDN9 |
| RYK | TIGD2 | MTHFD1L | SBF1 | AHCTF1 | KCNA3 | METTL13 |
| THAP5 | FAAP100 | NXPE3 | SLFN12 | ADNP | REC114 | RIF1 |
| CCR8 | SYNDIG1 | C12orf79 | ERBB3 | EFR3B | SALL2 | MTR |
| RNF223 | DEFB115 | CHRNA9 | HERC4 | LPAR6 | ARL5C | XAF1 |
| XPO5 | RPE65 | TRIOBP | AXIN2 | CACNA1H | KRT38 | STAG3 |
| TES | TAB2 | GDF1 | SCN1A | TSPAN6 | SLC24A5 | SHANK2 |
| ZFAT | KIF5C | GREB1L | IQCB1 | GON4L | EMILIN1 | HERC2 |
| KATNB1 | CCDC169- | CTSW | SMYD2 | RAE1 | DGKG | FAAP24 |
| | SOHLH2 | | | | | |
| CCM2 | HEATR1 | RASSF7 | WDR86 | OR5K3 | SYT4 | EDNRB |
| TMEM158 | ABCC8 | C15orf27 | SYT5 | CCR6 | RNF112 | KLHL1 |
| MYEOV | PATE1 | PML | GIT2 | IGFBP7 | KCTD13 | CD200R1 |
| ADCY8 | FNDC3B | KIAA1644 | ARTN | PPP1R18 | PPA1 | ATP9A |
| GPR39 | TMEM41A | GDF10 | OR5K2 | TNFRSF14 | USP24 | IL32 |
| UGT1A5 | NUGGC | TAF2 | TMEM231 | SH3GL2 | TEPP | PPAP2B |
| GAS2L3 | SLC38A9 | FBXO27 | MSMP | WDR5B | SETD1A | RGS22 |
| VSIG10L | ZRANB1 | GPR150 | SLC9A3 | CDY2B | FOXP3 | NPC1 |
| PER2 | ADAM18 | PRG4 | ATG9A | PDZD3 | SUV420H1 | KDM3A |
| KLHDC7A | CRBN | CFAP43 | YAE1D1 | RNF224 | SLC16A3 | THOC2 |
| MKL1 | GPR68 | HAUS7 | GRM1 | ARSF | KDM4A | FAM45A |
| CLN3 | ZNF551 | TTLL4 | DAO | IZUMO1R | KCNA6 | OSGIN1 |
| MCTP1 | PPP1R3D | KCNK17 | TOP1 | LMTK2 | MRPL43 | CFP |
| PLEKHH3 | SALL4 | THEMIS | RBKS | ADGRA3 | FAM65B | NELL2 |
| EN2 | NOXO1 | CTU2 | ADAM29 | RAB40C | DIAPH3 | RNF168 |
| CCDC129 | HTR2C | NMNAT2 | UPP2 | PRICKLE1 | ABCC5 | RTFDC1 |
| IL31 | SCN8A | CLEC4A | DZIP3 | LTN1 | FAM171A1 | GATA5 |
| SLC30A1 | TGDS | KRTAP3-2 | TCF15 | STOX2 | PGLS | FRYL |
| TSPAN31 | SYNDIG1L | TMIE | PIGF | KLRC4-KLRK1 | PFN2 | ANGPTL7 |
| DHX57 | UHRF1 | EMILIN3 | TMEM92 | CACNG8 | VPS36 | A2ML1 |
| ESM1 | PGS1 | MMP24 | CHD3 | AQR | SPRYD7 | ARHGAP33 |
| C17orf99 | MT3 | OR6X1 | GRIN2B | BRCA1 | CLPSL2 | GPR143 |
| CLEC9A | ASB11 | C1orf35 | AP1AR | INTS4 | RASSF1 | ODF2L |
| LY6G6E | HAUS6 | BMPR1A | ZNF362 | SOGA1 | WNT9B | C1GALT1 |
| HYDIN | DPM1 | POLD1 | NRGN | PLEKHH2 | RP11- | WI2- |
| | | | | | 240B13.2 | 3308P17.2 |
| OSBP | ATP11B | RAD54L | RLF | CYP1A2 | CEP72 | FOXD4L5 |
| TSTD2 | TPRKB | SFTPC | ZKSCAN4 | PROCA1 | AL589743.1 | DGKI |
| RTN4RL1 | ACVR1C | C7orf31 | CSRP2 | FRY | RTBDN | PLP1 |
| TRMT13 | CCL28 | LRRK2 | CCND1 | NGLY1 | SERPINA7 | CST7 |
| PDGFA | CENPJ | PER1 | ABHD12B | ZZEF1 | GPR179 | ST5 |
| MSH3 | CAMSAP1 | GGNBP2 | FEM1A | TUT1 | ZMYND11 | MYO15A |
| ATAT1 | NBAS | PAPPA | ATG2B | MAN2A2 | LACC1 | SLC27A5 |
| JAG2 | CCR2 | PPM1J | EPHX3 | RTKN | LCE1A | ENGASE |
| FNDC7 | UBR1 | MROH2B | PTBP2 | NEK10 | COL6A3 | SBSPON |
| ARHGEF10L | PMS1 | PSD4 | C9orf89 | ADAD2 | CYBA | PPP1CA |
| KBTBD4 | TSPAN15 | MAP3K4 | FZD7 | HTR1F | USP17L7 | FAT2 |
| ZNF674 | PROK2 | ADAM9 | DNAJA1 | WRAP53 | ZNF316 | SULT4A1 |
| ACADM | CCL4L2 | ARFGEF2 | IPO4 | PAPLN | GATA2 | PRKDC |
| GPR25 | TLE4 | DDO | WFDC2 | ZDHHC9 | PEAK1 | SEPT7 |
| GPR35 | FASTK | PRR20A | RCAN2 | C10orf55 | GPC3 | ZDHHC17 |
| TM9SF1 | GPATCH2L | TGFB2 | EIF2B5 | PSME4 | GREB1 | TBC1D32 |
| AMDHD2 | DUSP2 | PRAMEF8 | ERICH3 | HPN | FSTL3 | OPN1MW2 |
| STPG1 | ACSBG1 | KRTAP10-12 | AOX1 | CHURC1-FNTB | GRIN2C | AL022578.1 |
| TDRKH | CYB5RL | ESPN | SEC61A1 | CD300LG | ADGRL1 | LRRD1 |
| EGR4 | TNFRSF4 | AKAP2 | C14orf177 | MB21D2 | TNFRSF8 | BAMBI |
| CASD1 | CRYGC | TEX10 | ZNF441 | AP1S2 | GK5 | TAS1R2 |
| LPAR4 | ZNF70 | RP11-166B2.1 | FYCO1 | FAM19A4 | SLC35G6 | KLHL31 |
| HELZ | RUFY4 | CYP4F11 | MOXD1 | LAMB4 | PGM2L1 | GIMAP7 |
| TGM1 | KDM5B | ISOC1 | LY6G6C | LCE3A | SLC25A47 | SLC26A1 |
| TMEM213 | GPC6 | ZFHX4 | LGR6 | CFI | FPGT- | NEK5 |
| | | | | | TNNI3K | |
| EFNA4 | THBS1 | MAST4 | ZNF547 | CREBBP | NMRK2 | SLC37A2 |
| INSR | STAB1 | ZFP64 | PRAME | DOPEY1 | BMP4 | FAM102A |
| FSHR | HACE1 | FAM131C | TAAR8 | SERINC1 | TAMM41 | NEK11 |
| KCNV2 | CDIP1 | CHRNA6 | TMEM52 | RFNG | ENDOD1 | CYP46A1 |
| TACR2 | COL1A1 | TRIM8 | MYCBPAP | C17orf80 | BTK | AMN |
| ZC3HAV1 | LHX5 | C1QTNF3- | C2CD4D | CCND3 | TEX35 | ZFYVE16 |
| | | AMACR | | | | |
| TM4SF20 | IGF2BP1 | ARHGAP6 | TUBA8 | BFAR | ANTXR2 | BRD4 |
| AP4E1 | EFNA1 | BTBD7 | CRELD1 | HCAR1 | NME4 | WI2- |
| | | | | | | 2610K16.2 |
| TMEM260 | CDRT15L2 | TRIM67 | USP49 | FBXO34 | NEURL1B | APP |
| GDF6 | MEGF10 | TUBA4A | PHF1 | SEPP1 | FAM198B | TOR1B |
| HAGHL | LITAF | KDM4C | NXPH3 | CD276 | FAM214A | BTN2A2 |
| NID1 | DNAJB13 | PTGES3L- | VSIG4 | TNNI3K | ENPP1 | SEPT4 |
| | | AARSD1 | | | | |
| ADORA2B | CNGB1 | SLC28A1 | WDR36 | CRYBG3 | ZSWIM6 | HNRNPL |
| CTAG1A | MS4A7 | DLC1 | ZMAT4 | ADA | BCL11B | XCR1 |
| C4BPB | TTC21A | PLA2G2F | TESK2 | DAAM1 | IGF1 | ARSK |
| DLL1 | USP15 | NCR3LG1 | ZHX3 | GPC2 | VPS9D1 | C10orf142 |
| TOR3A | TRIM28 | SLC17A1 | USP9Y | OAZ1 | SNX31 | C5AR1 |
| TAS1R3 | ZP4 | DNAH7 | ZNF280D | AFAP1L1 | HERC1 | RCE1 |
| FAM73A | CCNO | KIF2B | EBF2 | QRFPR | IRS2 | LRP11 |
| SLC25A10 | ARMC4 | CNIH4 | ZFP37 | AMIGO3 | MYCBP2 | CD96 |
| VEGFA | OR13J1 | KCND1 | TSPAN8 | CLDN11 | TMC1 | IFI27L1 |
| PREPL | GPR176 | SLC35F4 | SCN10A | TFF2 | ALDH8A1 | KBTBD8 |
| MCCC2 | WIZ | ZKSCAN3 | OR2T27 | ZCCHC11 | POTEE | PRKCH |
| SLC6A17 | ZNF536 | NPEPPS | ZNF511 | SOWAHB | KIAA1551 | PGM3 |
| DDX43 | PRR16 | AC012005.4 | ACTR8 | GRIN2D | SLC22A16 | PALB2 |
| SMAD6 | COL16A1 | LCE1E | TMEM265 | VTN | VWDE | PTGS2 |
| TBC1D30 | GPR180 | ZXDA | TMEM11 | LY6G5B | IGFBPL1 | RXFP1 |
| CYBB | CAAP1 | SLC2A5 | ACYP2 | SLC35B3 | IL17RA | ATP9B |
| STIL | NKPD1 | SLC26A9 | ZNF646 | TGFBI | MDN1 | DNAH6 |
| OR10J3 | OR2M4 | FRS2 | PLEKHG4B | ASIP | BMP2 | EPHA10 |
| CHRNA4 | PLAT | USP2 | HOXB13 | C3orf20 | HOXB2 | DPYSL5 |
| ADCY7 | WDR11 | DBF4 | DOCK8 | PXT1 | DPH1 | TGM2 |
| KLHL17 | FOXD4 | ZNF425 | SEPT3 | PLAC8 | PHF7 | ARL13A |
| CFAP47 | ASB4 | TEP1 | OR2T12 | ITPK1 | CNKSR1 | MYOC |
| LRRC37A | DOCK6 | THUMPD1 | ZNF232 | LIFR | PRDM9 | RABEP2 |
| DRD1 | LEPR | FAM153A | ZNF814 | CANX | ADCY4 | PRM1 |
| STXBP1 | AGAP2 | FAM19A2 | HTATSF1 | C5orf34 | APLNR | KIAA1107 |
| C21orf2 | DEFB121 | OIT3 | AKAP9 | TRIM64B | DENND1B | ADAMTS2 |
| CBWD2 | FBXW2 | PSCA | LRRTM2 | EPM2A | SLC9C1 | ADGRF3 |
| PHF3 | NR0B2 | GNB4 | PLK2 | ACOT8 | NPIPA5 | SLC35F3 |
| PINK1 | DCTN6 | LRRC7 | KRT85 | HIPK1 | USP44 | BTBD11 |
| YWHAE | ACOT1 | PAMR1 | OR4M2 | AGXT2 | RAD51AP2 | MYH9 |
| MYBPHL | CCR3 | FAM98C | PLEKHG2 | RB1CC1 | FAM212A | APBB3 |
| KRTAP7-1 | PRAMEF25 | TNFRSF1B | HINFP | EI24 | OR1L1 | KRTAP6-3 |
| ALPK2 | SNAP23 | DOCK1 | LIPE | ARHGEF15 | MYO7A | D2HGDH |
| GRIP1 | SLC22A7 | ARHGAP44 | POLR1A | SLC2A14 | OTOL1 | DGKB |
| OR14A16 | C10orf54 | XKR7 | RAB9A | OPN1LW | LYPD5 | ZNF716 |
| BMP6 | DRAXIN | UCHL5 | EXD3 | ZBTB40 | ABCA3 | KIAA0430 |
| COASY | SGCE | KLHL2 | CDT1 | FBXO28 | RHBDL3 | INHA |
| POTEF | WNT10B | C10orf67 | DEPDC5 | TMPRSS11E | GLMN | CSNK2B |
| IPP | MCM9 | APOL3 | DDX1 | CAD | C9orf92 | AGAP5 |
| PGF | RCBTB2 | BBS12 | UMODL1 | EHMT2 | KDF1 | DMXL1 |
| TNR | SYTL3 | AKAP13 | AHCYL2 | GAREM | EBF1 | CPNE7 |
| CCNF | CEL | OC90 | CFC1B | SEMA6A | POLM | CDCA2 |
| DHX36 | PLD2 | RCHY1 | HHIPL1 | LY6K | CXADR | SLIT1 |
| ZFYVE27 | MARCH8 | AKIRIN2 | PPM1E | PCDH7 | DLG3 | KCNA1 |
| CCL11 | GLTSCR1L | ANKRD36 | DAAM2 | CBR1 | OTOF | SPAG11B |
| SLC7A10 | DEFB130 | CSF1R | SAE1 | TWSG1 | GPAM | PERP |
| ABCB8 | CYSLTR1 | ATP2C1 | FOXD4L3 | MET | ERBB4 | IGFBP1 |
| DHX8 | MAN2A1 | WDR4 | TEKT3 | FGF13 | MEIKIN | CDY2A |
| BUB1B | EGLN1 | OR10Q1 | ARHGAP21 | ELF5 | CTAG1B | GPC1 |
| FIGNL1 | TIMELESS | ABHD3 | SRGAP2 | TMBIM1 | LCE3B | ADAM32 |
| C1QTNF6 | CYP4A22 | KIAA2026 | MEGF6 | TSPAN2 | C2orf54 | FBLN1 |
| UGT1A6 | ZBTB6 | NOX4 | GP1BA | FLT3 | ALS2 | PPARA |
| TNS2 | FMO1 | PRR20E | KLHL5 | SLC26A3 | RASL10B | IFITM3 |
| OR2M2 | LHFP | CHL1 | NGFR | AVP | SLFN14 | OR5L1 |
| APLP1 | SPPL2B | SLC25A29 | ACAP1 | BRICD5 | SLC6A13 | PDCD2 |
| GOLGA4 | ZNF850 | SI | DOCK9 | CACHD1 | ALG1 | C4BPA |
| CCNG2 | SLC12A1 | KIF23 | SLC28A3 | UPF2 | MIB1 | CHRNA2 |
| SERPINB4 | SNX21 | SGSM3 | GBA2 | GRK6 | FAM189A2 | FKBPL |
| TLL2 | DBF4B | TGFBRAP1 | RP11- | FLG2 | IFIT3 | KRTAP9-6 |
| | | | 574F21.3 | | | |
| TTLL5 | KIAA0226L | GPM6A | CACNG3 | NUDT7 | OR2V1 | AMZ1 |
| LCMT2 | RNF20 | AFP | CLCN1 | KIAA1024 | HSP90AA1 | FAM160A2 |
| XPNPEP1 | TTN | DEFB128 | ERMP1 | SLA | SCNM1 | DKK1 |
| GDF2 | PIGM | FASTKD5 | PLCD4 | OR6J1 | TSR3 | MUC5B |
| C2CD2L | CCL8 | RPRM | ADAT1 | ATP8B3 | SEC14L1 | MCCD1 |
| RASA4 | KRTAP12-3 | UPRT | VAV2 | BAHCC1 | SLIT2 | KCNK13 |
| LIMS3 | NYX | ELMO3 | WDR47 | C12orf29 | HSPB9 | BCHE |
| NFKBIE | CCR7 | SLPI | ATG7 | C10orf99 | ASGR1 | MGAM |
| BACH2 | MOSPD1 | ZNF598 | PRAMEF15 | TNFSF13B | BLK | SIAH1 |
| SQSTM1 | WBP4 | NMUR1 | ARRDC5 | ZSCAN21 | C17orf50 | CLUL1 |
| OR14K1 | RUSC2 | THAP3 | PRAMEF20 | LPCAT2 | FAM72C | PEX6 |
| XPO7 | ACSS2 | KCNIP2 | TEX29 | TEFM | INSIG1 | PROSER2 |
| TBCEL | C3orf70 | RNF169 | VPS45 | PLCE1 | KRTAP10-8 | LRRC42 |
| RFPL4A | VWC2L | PCCB | FETUB | SLC22A31 | EGFL7 | TMEM62 |
| PLEKHH1 | TEX11 | RFWD2 | PRDM11 | TGM4 | EDRF1 | C15orf41 |
| FBXO46 | DUOX2 | OPN4 | PROM1 | PLD5 | CDY1B | RP1-66C13.4 |
| OR5C1 | OR5K4 | TCAF2 | RAB40A | ANKHD1- | SIM2 | TSPAN10 |
| | | | | EIF4EBP3 | | |
| ICE1 | KLHL18 | THNSL2 | HOGA1 | CBR3 | TONSL | ALG8 |
| IER2 | UPK1A | SPOP | OR8H1 | ZNF229 | VPRBP | GFOD1 |
| NOTCH4 | BBS10 | XKRX | MFSD6L | KCNK9 | RYR3 | ZNF16 |
| SCRG1 | ARL8B | RANBP17 | MUSK | RNF122 | PADI1 | ZSCAN32 |
| PEMT | RP2 | IL12RB1 | FMR1NB | MUC6 | POTEM | NPRL2 |
| CBLN2 | DNMT3L | KRTCAP3 | SEC61A2 | VAV3 | SPATA18 | PTGES3 |
| AC006328.4 | ANXA8L1 | TSPAN19 | ZMYM4 | ATP8A2 | RHBDF2 | TTC39B |
| ZNF487 | TNFRSF1A | ZFYVE28 | POMT1 | ZNF28 | JAM3 | BTBD6 |
| B3GALT4 | FAM43A | EPRS | PDGFC | TM9SF3 | MED16 | ABCC11 |
| TYRP1 | SWT1 | CARS | CBWD5 | TIAM1 | PCSK9 | PGM2 |
| CLDN3 | HPS3 | TMEM59L | BTBD16 | JARID2 | PPP1CB | SAP25 |
| DCLK2 | CD63 | TSPAN7 | IGF2R | SRM | FAT3 | SLX4 |
| ASPM | DEFB116 | TMEM150A | PRMT2 | ZNF17 | PRAMEF6 | SCN1B |
| WSB2 | OVCH1 | DAB2IP | CACNA1G | RGS7BP | UBE3B | NPIPA1 |
| SVEP1 | ZNF407 | ZNF862 | TMEM255B | NADK | SENP5 | CXCL10 |
| TRMT2A | C12orf71 | CTGF | AMHR2 | BMP10 | KCNS2 | RELT |
| CATSPERD | CLN3 | GPR88 | CAMSAP2 | PRR20C | CACNA1I | EVC2 |
| NUDT19 | ATP1A1 | PRPF6 | CD53 | PGK1 | NDUFS7 | NBPF6 |
| BTBD2 | RASAL2 | PRR20D | IDI1 | SLC47A2 | RASA3 | GDNF |
| LIMK1 | RINT1 | CCL15-CCL14 | YTHDC2 | NKX3-2 | AF196779.12 | CCL17 |
| DAZAP2 | CLEC17A | MAGEB2 | CHUK | CMTM1 | REXO1 | PCDHGC5 |
| C1orf111 | FGG | FAM20B | USP17L8 | ICOSLG | ZNF302 | SLC9A3R1 |
| USP3 | ALG1L | MDM2 | MYO3B | CAPN3 | MDC1 | TTBK2 |
| PCDHAC1 | GSG2 | TLR2 | ZXDC | BRWD1 | GPR101 | HTR3D |
| NFIA | ZMYND8 | ZMYM5 | CNOT1 | ASUN | KCNG4 | STK11IP |
| NOP14 | R3HDM1 | SLC6A1 | TSPAN13 | ZFYVE1 | FIZ1 | POPDC2 |
| TCP11X2 | CLDN19 | NLRC5 | SPTLC1 | RABGEF1 | GRN | CLDN8 |
| FOXD4L4 | AP1S3 | USP9X | OR2T8 | MCM7 | TMEM221 | RP11-244H3.4 |
| PIK3C2A | C12orf4 | PLAC8L1 | URB1 | TXNDC8 | ADGRV1 | ADCY5 |
| RNF126 | ANOS1 | VSTM4 | MED12L | ANKRD50 | FBXW4 | PHF20L1 |
| ITGB1 | MMRN1 | PLA2G6 | RFPL4AL1 | ANGEL2 | WHSC1L1 | GRM3 |
| HUWE1 | HTR6 | FRMPD3 | RNF39 | SYT16 | ACTL10 | PRR14L |
| NIPBL | RP11-146B14.1 | PALM2-AKAP2 | FN3KRP | UBR2 | TRMT44 | SYCN |
| MLLT10 | THBS2 | INTS8 | ZNF395 | KRTAP13-3 | KRTAP10-7 | TMPRSS9 |
| SLFN11 | DZANK1 | DEPDC1B | SLC35B4 | PDE8B | PDE3A | LYRM9 |
| SULF1 | PDGFB | SSUH2 | SF3B1 | RNASE8 | FAM189B | C7orf60 |
| SCARB2 | RANBP2 | CCL24 | IGFN1 | MROH8 | RBBP6 | OR51B6 |
| AC108938.5 | PRORY | TMEM178B | MTFR1L | OR6C2 | TMC5 | KIAA1328 |
| MT1F | CYP4F12 | BRPF3 | KCNC3 | ISM1 | KRTAP12-2 | MT1HL1 |
| UGT1A7 | RP1L1 | COL6A1 | MC3R | LYPD2 | RBSN | TSPAN12 |
| ACOT2 | ASNS | RETSAT | PRKCG | TGFBR1 | ESCO1 | SLC44A2 |
| FAM72D | CIAO1 | ZPBP2 | ACSL5 | SBK1 | SLC44A4 | RAPH1 |
| BMP3 | OSBPL1A | C6orf49 | SLC22A23 | TMEM196 | SLC28A2 | FAIM2 |
| TMEM59 | IGFLR1 | SMCP | TBC1D12 | CTIF | CAPN6 | CTCFL |
| CCL27 | RNF24 | FAM160B2 | TUSC1 | DSC3 | CYR61 | ADAM23 |
| SLC16A8 | CCDC138 | C1orf95 | KDM2B | RBM44 | TMF1 | HFM1 |
| NUTM2B | NLK | PTGER2 | PTCH2 | PHGDH | POMC | LAX1 |
| IFT140 | LAPTM4B | CSH1 | MCPH1 | LCE1F | SCN7A | TESK1 |
| RLTPR | DNHD1 | FAM120A | LDLRAD2 | UGT1A1 | ZNF165 | KIF24 |
| SLC35E2 | URB2 | MFSD4 | IL17RE | IGSF1 | ZNF670 | NELL1 |
| EPB41L2 | RAB3GAP1 | ADAM10 | NOM1 | POGZ | CTDSP1 | MRGPRG |
| AATK | TMEM136 | ADAM19 | GNA11 | TMC2 | CD72 | DBX2 |
| AAAS | ASAP3 | RAD50 | C19orf68 | BRAF | UPK1B | PGBD1 |
| CAPN1 | SPG11 | PTPRM | TSPAN33 | PSMB10 | COL2A1 | COG8 |
| PDXDC1 | INHBE | BPTF | WNT7B | GPR15 | GGA2 | FYB |
| SLC27A2 | PCDHAC2 | BMP1 | PHLDA1 | KMT2B | ELL3 | KRTAP21-2 |
| HAUS5 | KIF18A | TP53TG3B | TM4SF1 | CPD | LHX1 | GSG1L |
| NME7 | FBXO22 | RALGAPA2 | TMEM241 | PI4KA | JAK1 | ANKRD52 |
| SIAE | DOCK10 | TOE1 | TMPRSS11B | PHKA1 | OR2T1 | PPP4R1 |
| GRM4 | EP300 | FZD8 | HAP1 | ADGRF5 | KRTAP10-6 | HELQ |
| CENPI | ASIC1 | PPP1R3A | GRIN3A | KRTAP16-1 | DENND2D | ATM |
| RTN4R | MBD1 | CCBE1 | OR5K1 | CHRNA1 | XYLB | DFNA5 |
| CAMK1G | KIF17 | ANKIB1 | KIF13A | C3 | PPP1CC | CCDC85B |
| KANSL2 | RNH1 | GADL1 | MCM6 | NXF1 | SMPDL3A | PROKR2 |
| CPM | LHCGR | ATP8B4 | AKAP1 | ACVR1 | ARAP1 | WFIKKN2 |
| VPS8 | KLHL36 | GPIHBP1 | ABCA12 | POTEG | KBTBD3 | MMP9 |
| NLRP4 | SSPO | SNPH | DPRX | U2AF1L5 | PARD3B | GCLC |
| SLCO1C1 | AC004381.6 | BTG3 | SHISA7 | PRAMEF19 | LRRC16B | SPATA31E1 |
| GALR3 | STOX1 | EML6 | TACR3 | MS4A8 | CDC14B | AIP |
| WWOX | OR51F1 | NANP | PRAMEF4 | EFNA3 | SLC29A2 | OTUD7B |
| MN1 | LAMA3 | KIAA0355 | GH2 | FAM161B | RAB3GAP2 | CCL19 |
| MIB2 | TUBA1B | ENDOU | ECM1 | TBC1D15 | TUBGCP5 | CCL20 |
| DNAH12 | ADAM7 | KRTAP10-5 | KLF17 | KIRREL3 | INMT | ADAM21 |
| SUMF1 | INHBA | PIM2 | PSMB7 | BUD31 | LIMD2 | NR0B1 |
| TLE3 | RBCK1 | SMC6 | AKAP6 | CIDEC | OXLD1 | PGR |
| CLDN16 | TMEM44 | BMP7 | APLP2 | RNF182 | TP53TG3D | ZNFX1 |
| SPRY2 | ARL5B | KCNS3 | TM4SF4 | CNBD1 | PPY | PRRT1 |
| SLC17A2 | ZDHHC5 | RNASE11 | KLHL10 | LYSMD3 | LY75-CD302 | HECA |
| BRD8 | HOXC10 | ZNF510 | PTBP1 | RTTN | S1PR4 | RYR2 |
| SYDE2 | GPR135 | EHMT1 | TMEM120A | SEPW1 | SCP2 | SGIP1 |
| CLDN4 | SLC12A2 | SMAD9 | PSMG1 | C14orf159 | PROK1 | EFCAB13 |
| VNN1 | CNR2 | KRTAP29-1 | ZSWIM2 | CNTFR | CD80 | NFXL1 |
| ZNF479 | MST1R | FAM72A | KRT39 | OR2T35 | SCGB1D4 | PRAMEF14 |
| PJA2 | ARL14EP | AEBP2 | LPAR2 | WFDC3 | LRCH2 | GLO1 |
| CDK2 | KIDINS220 | AARS2 | ARID5B | ADAMTS12 | PLA2G3 | SLC6A5 |
| EIF2AK4 | ZNF700 | ADAM17 | THAP4 | OTUD5 | PNPLA5 | RNF141 |
| GADD45A | ACTR6 | FBXL16 | TMEM108 | BMPER | FAM24B | PRR7 |
| ARHGEF4 | ACVRL1 | SEC23B | ELP2 | FBXO16 | PKHD1L1 | PLCH2 |
| MRGPRE | ZMYM6 | KCNMA1 | ARPC1A | LRIT1 | PDLIM2 | INPP4A |
| PGBD4 | IFI44 | C2orf81 | GPR132 | GPC4 | ELP4 | FSHB |
| DDX51 | CARD18 | WISP3 | OR14A2 | CCL25 | TMC6 | USP46 |
| PADI6 | PTGIR | PPM1D | LCMT1 | TRPM4 | SYT11 | C2CD4C |
| TRIM47 | C10orf82 | CMC1 | C7orf26 | NBEA | NTMT1 | PRF1 |
| WNT8A | KRT81 | AJAP1 | BHMT2 | RNF135 | TRAF1 | PKP4 |
| TRMT1 | DEF6 | GPR34 | MCM4 | ZFP57 | TOPAZ1 | SKOR2 |
| PLA2G10 | PSTPIP1 | ARHGAP23 | LRRC8B | NPY5R | ADAMTS3 | MUC4 |
| OR2T6 | TUBE1 | KCNC2 | LRRC37A2 | NEU4 | HERC5 | TMEM176A |
| PRAMEF11 | CSGALNACT1 | PRAMEF7 | PERM1 | CCDC108 | ATP6V0A1 | ZNF197 |
| FAM200A | NIN | SERPINE3 | TIAM2 | BMX | ADNP2 | HTR7 |
| SPATA6 | CTU1 | TG | FCGRT | SLC25A32 | SLC26A4 | SERINC2 |
| KLHL8 | DCAF17 | ITGA1 | GDF9 | METTL25 | PPP2R5A | SYT17 |
| MAP3K5 | C17orf62 | GALK2 | PAAF1 | OXTR | METTL4 | IKBKG |
| NRTN | TCEAL4 | C17orf74 | ANKS3 | LIMK2 | TLR10 | AGRP |
| IGFBP5 | FCER2 | WNT7A | PRR4 | IFIT2 | N4BP2 | HTR4 |
| ATP5G1 | VCPKMT | CEPT1 | KIF5B | IFITM2 | MARCH1 | RGS12 |
| SPACA7 | SLC44A3 | TMEM256- | ADAM28 | REPIN1 | TLR4 | PI4K2B |
| | | PLSCR3 | | | | |
| VRK2 | FANCA | SLC36A1 | DMWD | ANKRD17 | NMD3 | SLC22A14 |
| PJA1 | PDS5B | UHMK1 | RNF146 | RORA | POLR2L | PLXNA3 |
| CHST13 | AK1 | ADAMTS18 | PTPDC1 | LMBRD1 | THADA | C22orf31 |
| NAT10 | ADH1C | FAM124A | SUV39H2 | FMO5 | ANKRD55 | WFDC12 |
| PLAUR | PLEKHM2 | UPF1 | KRTAP10-1 | C16orf93 | MED12 | VIL1 |
| GNAQ | BEND7 | NRXN1 | WNK2 | PLA2G12B | ASNSD1 | INSIG2 |
| CKLF-CMTM1 | TSPAN11 | SUSD3 | RSPRY1 | UBR3 | DPCR1 | NAA40 |
| CAMSAP3 | PGLYRP1 | HUNK | DEPDC4 | C10orf90 | AARSD1 | CACNA1D |
| ADGRE1 | BCO2 | TRIO | TMEM222 | ABHD16A | CLEC4G | XPNPEP2 |
| HECW1 | HEPACAM2 | RP11-1277H1.5 | FGFR4 | BCR | TRAF6 | SEC16A |
| PRKCA | RASGRP4 | SLC6A16 | ALGIL2 | TOR1A | SPIRE1 | WNT11 |
| C5orf42 | SLC17A3 | MYO15B | C12orf76 | SIGLEC9 | CEP76 | CCL14 |
| OTUB1 | GPR45 | DNAH17 | CASP9 | TRAPPC10 | WNT5A | DUOX1 |
| LCE2D | SLC38A8 | EMID1 | APBB1 | ADCY9 | SLC52A1 | RHBDF1 |
| TMEM255A | ZNF181 | GTPBP2 | LMO1 | HEXDC | INSL6 | CILP2 |
| SHISA9 | ABHD17C | MRGPRF | WWC3 | ARMC2 | ZYG11A | QTRTD1 |
| TAAR6 | CD40 | CNTROB | HARBI1 | NBPF4 | SGMS2 | TSPAN4 |
| OR10A4 | NRG3 | HNRNPLL | AVPR2 | MED13L | MROH6 | PANK1 |
| EML2 | CAPN10 | PPARGC1B | KCNT2 | GNGT1 | SNX5 | CDYL |
| SCAP | RNF26 | NFX1 | DKK4 | LYPD6 | ERVFRD-1 | C9orf24 |
| MTNR1B | ZNF512B | ZSCAN30 | FBXL6 | PRPH2 | RGR | GPR137 |
| TRPM7 | LIMS3L | FBXL4 | ZPBP | ZNF544 | CHST11 | TFF1 |
| PTBP3 | NLRP1 | SMAD1 | LAMA5 | SLC17A8 | KRTAP13-1 | C5orf56 |
| IL1RAPL2 | PNPT1 | KCTD17 | HSDL1 | CACNG4 | RBM5 | ATAD3A |
| FUT6 | CHN2 | ABHD16B | DDC | TNFAIP1 | SOHLH2 | PHF10 |
| ARRDC4 | PELI3 | ZNRF4 | LBH | OR4C11 | KIAA1468 | KLRK1 |
| SFMBT1 | MXD3 | TTLL3 | TISP43 | ATXN1 | SSTR4 | BIRC2 |
| MSRB2 | HIP1 | SPAM1 | OR4P4 | SMG1 | WFDC13 | COQ10A |
| RP11-23E10.6 | ZBTB5 | LIME1 | S1PR1 | SSH1 | DISP2 | LCE1C |
| CDK12 | OSBPL2 | ARHGAP8 | DIS3L | HEATR3 | BMF | CCL1 |
| PID1 | KIR3DX1 | SLC9A6 | ARRDC3 | TAS1R1 | OR4C3 | SLC25A34 |
| SLC16A5 | SLC1A5 | CD59 | TRAPPC11 | SEMA6D | DKK3 | PLEKHG1 |
| CASC10 | SMCR8 | SLC44A1 | KRTAP13-2 | PWP1 | ZDHHC19 | VN1R1 |
| GNB3 | CFAP69 | DSG3 | | | | |
| TMEM145 | ITGB1BP2 | TYMP | | | | |
| KCNA7 | TDRD6 | ACSM6 | | | | |
| CCDC18 | KAT6A | CABIN1 | | | | |
| FMO4 | SVOP | TRIM23 | | | | |
| ZBTB38 | DUSP15 | ERGIC2 | | | | |
| GCAT | PIKFYVE | TSHZ2 | | | | |
| PTCH1 | CELF2 | TMEM225 | | | | |
| NUP160 | RGN | NAALADL1 | | | | |
| LUC7L2 | ETV3L | C2orf68 | | | | |
| KRTAP9-3 | HTRA1 | PLA2G4E | | | | |
| UNC79 | ARHGAP26 | VENTX | | | | |
| SLC11A1 | TROAP | TTC34 | | | | |
| OR1D2 | ZNF781 | OBSCN | | | | |
| TNFRSF18 | TH | KCNT1 | | | | |
| ZC3H13 | KPNB1 | LRIG3 | | | | |
| TSPAN9 | LCE4A | IRS4 | | | | |
| NEBL | SNX11 | HR | | | | |
| TOP3A | SPRED1 | DIDO1 | | | | |
| CFC1 | ICOSLG | SLC2A6 | | | | |
| FAM72B | FREM1 | ADRB3 | | | | |
| C4orf19 | POLH | TIMM23B | | | | |
| USH2A | NAIP | TRMT61B | | | | |
| SEMA3C | MYRFL | S1PR2 | | | | |
| CASP4 | ROM1 | OTOG | | | | |
| DCLRE1C | MT4 | VEGFC | | | | |
| TXNRD3 | ARMC6 | TRA2B | | | | |
| RAB11FIP4 | SZT2 | KRTAP9-9 | | | | |
| FAM170B | CCAR1 | ARHGEF1 | | | | |
| DSC1 | NOV | KY | | | | |
| ATP2B2 | ACOT7 | CHRNE | | | | |
| MIS18A | TM4SF18 | TMEM246 | | | | |
| RNF217 | WNT3A | MPL | | | | |
| LCE3C | TNPO3 | FCRL5 | | | | |
| PDPR | CHD4 | RBL2 | | | | |
| TMCO5A | TNFRSF25 | MTERF4 | | | | |
| TUBA3C | PRAMEF2 | ZER1 | | | | |
| TUBA3E | AFM | DAPK1 | | | | |
| TRIM64C | IKBKB | HTR5A | | | | |
| AK7 | CHAT | PRAMEF1 | | | | |
| U2AF1 | CSRNP3 | PPP2R2A | | | | |
| RNF216 | RAPSN | ZNF641 | | | | |
| TMEFF1 | CACFD1 | NADSYN1 | | | | |
| C22orf29 | PGK2 | DQX1 | | | | |
| EMILIN2 | FBXL18 | NAA50 | | | | |
| OR2T2 | GABBR2 | GRPR | | | | |
| ATP6V0A4 | DRD5 | ATP6V0E1 | | | | |
| SH2D2A | OR2T3 | TRIM3 | | | | |
| C1orf94 | TSPAN14 | MTMR3 | | | | |
| TRIM66 | SEMA3D | SLC6A11 | | | | |
| ZNF311 | NEURL1 | EIF3D | | | | |
| KRTAP10-9 | ANLN | CERS1 | | | | |
| CDC14A | CHRD | SLC44A5 | | | | |
| TAF1 | BCAS3 | IGFL3 | | | | |
| CHI3L1 | TRIM55 | TTC21B | | | | |
| HRG | CLEC4D | NPNT | | | | |
| DOCK11 | LCE2A | FSIP2 | | | | |
| NKAIN1 | P2RX7 | HAL | | | | |
| TAF1L | DNAH3 | UVRAG | | | | |
| KCNG3 | LYPD8 | KRT34 | | | | |
| KCTD9 | PEAR1 | OR2T33 | | | | |
| CCNY | ARHGEF10 | DBR1 | | | | |
| ACTL7A | EPS8L2 | SGSH | | | | |
| PDZRN3 | KCNG1 | CNST | | | | |
| FAM71C | NLRP13 | POTEB2 | | | | |
| ADRA1B | SLC16A1 | ZNF691 | | | | |
| IDH3B | PMFBP1 | WFDC8 | | | | |
| IRX4 | SPATA3 | TMEM198 | | | | |
| LRRC37B | FOXD4L6 | TRIM37 | | | | |
| SYNE4 | SFRP2 | TMEM134 | | | | |
| UBE3D | IGFL1 | KRTAP6-2 | | | | |
| WNT10A | PLEKHG4 | CLUH | | | | |
| SLC12A5 | USP50 | FAM57B | | | | |
| DDHD1 | MED13 | APIP | | | | |
| ADGRA2 | RP11-449H3.3 | CHRNA10 | | | | |
| EME1 | ALAS2 | GPRC6A | | | | |
| UTP6 | DCAF8 | OSBPL5 | | | | |
| WISP1 | C10orf120 | C3AR1 | | | | |
| CHID1 | HTR1D | MPDZ | | | | |
| RAD18 | CCDC79 | ATP6V0E2 | | | | |
| GRM8 | OR2M3 | HTR3C | | | | |
| CMTR1 | ZNF804B | DRICH1 | | | | |
| CD9 | PPP2R4 | ZNF292 | | | | |
| USP17L4 | TRIM33 | ARHGAP4 | | | | |
| PGD | SMG5 | UTS2 | | | | |
| PRR20B | FAM163A | PIEZO2 | | | | |
| OR10R2 | MKRN2OS | MAP4K5 | | | | |
| RFWD3 | BMP8B | SLC34A1 | | | | |
| PLEKHJ1 | OAZ2 | FAHD2B | | | | |
| CARD14 | THRA | TNXB | | | | |
| FAM120C | FAM64A | CCAR2 | | | | |
| LMO3 | GSG1 | PLBD1 | | | | |
| LRP1B | KRTAP6-1 | ADAMTS7 | | | | |
| WDPCP | KLHL28 | HCRTR1 | | | | |
| WDR88 | TP53I11 | TMEM39A | | | | |
| DENND6B | CD160 | FAM189A1 | | | | |
| PASK | TSC22D3 | SPTBN2 | | | | |
| ECM2 | ACCSL | NTRK1 | | | | |
| HCAR2 | DLGAP3 | WSB1 | | | | |
| TSSC1 | PPP1R16B | TRIM27 | | | | |
| FAM19A1 | IDH3G | CHD2 | | | | |
| KRTAP11-1 | APBB1IP | GLI1 | | | | |
| LMTK3 | GLIS1 | PRKCE | | | | |
| RALGDS | ZSCAN29 | LCE1D | | | | |
| ARL14 | AOC3 | AGL | | | | |
| NPHP4 | CHRNA7 | PKD1 | | | | |
| POTEB | ACSL4 | PRDM10 | | | | |
| PUM2 | KIAA1324L | STYK1 | | | | |
| TGM5 | RP5-972B16.2 | NID2 | | | | |
| OR2T11 | DEFB130 | CCDC85C | | | | |
| SNRNP40 | CHRDL2 | EXOSC9 | | | | |
| C22orf46 | CCL23 | EXTL2 | | | | |
| PKLR | DNAH5 | ZNF444 | | | | |
| RPP40 | LEFTY1 | GTF2IRD2B | | | | |
| CBLB | RAI1 | SEC23A | | | | |
| CPNE5 | MMRN2 | SHANK1 | | | | |
| GALR2 | RGS14 | MOBP | | | | |
| ST6GALNAC4 | TMEM139 | WDR73 | | | | |
| FBXO21 | DCTN5 | | | | | |
| POTEC | SOD3 | | | | | |
| DR1 | TRIM13 | | | | | |
| COG6 | ARHGAP30 | | | | | |
| INADL | SLU7 | | | | | |
| RPS24 | ZCCHC13 | | | | | |
| CYYR1 | METTL2A | | | | | |
| GPSM1 | FAM26F | | | | | |
| INHBB | FAM159A | | | | | |
| SRGAP2C | POLG | | | | | |
| RNF213 | NDP | | | | | |
| HELZ2 | CRIP2 | | | | | |
| LUC7L | DAP | | | | | |
| UGT1A3 | SHISA3 | | | | | |
| KLHL20 | HECTD2 | | | | | |
| CD22 | MLLT6 | | | | | |
| IL10 | CXCR5 | | | | | |
| CCR5 | EPPIN-WFDC6 | | | | | |
| CFAP57 | SV2B | | | | | |
| GPR85 | MGAM2 | | | | | |
| DEFB126 | ACO1 | | | | | |
| TIMM23 | NAA30 | | | | | |
| NLRP5 | FAM86C1 | | | | | |
| RUSC1 | NUTM2D | | | | | |
| HTR1B | DYSF | | | | | |
| LGR5 | TRPC1 | | | | | |
| KCNK18 | MMADHC | | | | | |
| WDR12 | ITIH2 | | | | | |
| MTO1 | DLK2 | | | | | |
| ENPP3 | KRTAP1-1 | | | | | |
| N4BP1 | FLOT2 | | | | | |
| GPR160 | GPHA2 | | | | | |
| ZNF683 | BCDIN3D | | | | | |
| PLA2G2E | METRN | | | | | |
| PHF14 | PABPC1L | | | | | |
| DROSHA | SLC23A1 | | | | | |
| ABCE1 | CHD8 | | | | | |
| TMEM39B | ETNK2 | | | | | |
| FZD1 | ACTL7B | | | | | |
| OR10D3 | COPRS | | | | | |
| ERBB2 | DUS1L | | | | | |
| BRS3 | OR4C6 | | | | | |
| RMDN2 | POLE4 | | | | | |
| SCN9A | HSPA12B | | | | | |
| PDE3B | GSAP | | | | | |
| WNT9A | SLC37A3 | | | | | |
| AVPR1B | BOC | | | | | |
| MYBPC3 | MT1X | | | | | |
| BRPF1 | HHLA2 | | | | | |
| CDC42SE2 | GDF11 | | | | | |
| ANKRD18B | BRD1 | | | | | |
| TBL1X | CBWD7 | | | | | |
| SRI | SEMA4A | | | | | |
| SPRED2 | SRGAP2B | | | | | |
| COL20A1 | DEFB127 | | | | | |
| TTC8 | ABCG8 | | | | | |
| METTL7B | KCNA4 | | | | | |
| RFT1 | FAM120AOS | | | | | |
| XDH | XKR6 | | | | | |
| WDR75 | IL11RA | | | | | |
| LCE2B | OR2T34 | | | | | |
| TRIM9 | TSPAN32 | | | | | |
| KCNS1 | PTP4A2 | | | | | |
| RP11-385D13.1 | SUCO | | | | | |
| IKBKE | KRTAP10-2 | | | | | |
| RNF152 | C9 | | | | | |
| ARAF | IL1RAP | | | | | |
| CST9 | POLR3A | | | | | |
| NLRP10 | METAP1 | | | | | |
| ZSCAN10 | DEFB119 | | | | | |
| TMEM135 | OPN1MW | | | | | |
| SLC10A4 | PTF1A | | | | | |
| PRRT4 | JAK3 | | | | | |
| MRI1 | SSH2 | | | | | |
| ZNF735 | ZNF541 | | | | | |
| CNKSR2 | ZNF831 | | | | | |
| DPF2 | SLC32A1 | | | | | |
| RIMKLB | ASAP1 | | | | | |
| ABHD8 | EGFR | | | | | |
| GLYAT | WBSCR16 | | | | | |
| FBXO18 | SLC5A5 | | | | | |
| RTKN2 | ZBTB1 | | | | | |

In some embodiments, the cysteine-rich motif is a synthetic non-native polypeptide.

Cysteine-rich motifs are described in greater detail elsewhere herein, e.g., with respect to the engineered polynucleotides and engineered polypeptides.

In some embodiments, the engineered biomolecule contains 1-20 or more (e.g., 1, 2, 3, 4, 5, 6, 7, 8, 9, 10, 11, 12, 13, 14, 15, 16, 17, 18, 19, or 20 or more) lysosome targeting moieties. Exemplary lysosome targeting moieties are described in greater detail elsewhere herein with respect to e.g., the engineered polynucleotides and engineered polypeptides and targeted delivery.

In some embodiments, the engineered biomolecule contains one or more additional targeting moieties, such as one or more cell-type targeting moieties. In some embodiments, the one or more additional targeting moieties target a cancer cell. Exemplary additional targeting moieties that can be incorporated into the engineered biomolecules are described with respect to targeted delivery.

The engineered biomolecules (e.g., engineered polynucleotides and/or polypeptides) can be generated by any suitable method or technique. For example, polynucleotides can be recombinantly produced and/or chemically synthesis using automated, solid-phase oligonucleotide synthesis machines with 2′-acetoxyethyl orthoester (2′-ACE) (Scaringe et al., J. Am. Chem. Soc. (1998) 120: 11820-11821; Scaringe, Methods Enzymol. (2000) 317: 3-18) or 2′-thionocarbamate (2′-TC) chemistry (Dellinger et al., J. Am. Chem. Soc. (2011) 133: 11540-11546; Hendel et al., Nat. Biotechnol. (2015) 33:985-989). Likewise, polypeptides can be recombinantly produced and/or chemically synthesized. Exemplary methods of chemical synthesis of polypeptides are described in e.g., Borgia and Fields. 2000. Trend. Biotechnol 18(6):243-251; Tan et al., J. Am. Chem. Soc. 2020, 142, 48, 20288-20298. Such methods are described elsewhere herein and/or are generally known to one of ordinary skill in the art.

### Engineered Polynucleotides

In some embodiments, the engineered biomolecule is an engineered polynucleotide. In some embodiments, the engineered polynucleotide is engineered DNA. In some embodiments, the engineered DNA encodes an RNA, such as an mRNA, and/or polypeptide engineered biomolecule of the present disclosure.

In some embodiments, the engineered polynucleotide is engineered RNA. In some embodiments, the engineered RNA is engineered mRNA.

In some embodiments, the engineered polynucleotide includes one or more cysteine-rich motifs and one or more lysosome targeting moieties. In some embodiments, one or more of the cysteine-rich motifs are located at the 5′ end of the lysosome targeting moiety polynucleotide sequence. In some embodiments, one or more of the cysteine-rich motifs are located at the 3′ end of the lysosome targeting moiety polynucleotide sequence. In some embodiments, one or more cysteine-rich motifs are located at the 5′ end and the 3′ end of the lysosome targeting moiety polynucleotide sequence. In some embodiments, the cysteine-rich motif(s) are only located at the 5′ end of the lysosome targeting moiety polynucleotide sequence. In some embodiments, the cysteine-rich motif(s) are only located at the 3′ end of the lysosome targeting moiety polynucleotide sequence.

In some embodiments, the engineered polynucleotide contains 3 to 1000 or more nucleotides (e.g., 3, to/or 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000 or more nucleotides).

Cysteine-Rich (polynucleotide) Motifs

In the context of embodiments of the engineered polynucleotides herein, “cysteine-rich motifs” refers to regions or polynucleotide sequences that are composed of about 10-100 percent (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 percent) codons for cysteine (e.g., DNA: TGT or TGC; RNA: UGU, UGC). In some embodiments, a cysteine-rich polynucleotide motif is composed of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 99 percent to 100 percent codons for cysteine.

In some embodiments, a cysteine-rich polynucleotide motif is composed of about 10 percent to about 95 percent, about 10 percent to about 90 percent, about 10 percent to about 85 percent, about 10 percent to about 80 percent, about 10 percent to about 75 percent, about 10 percent to about 70 percent, about 10 percent to about 65 percent, about 10 percent to about 60 percent, about 10 percent to about 55 percent, about 10 percent to about 50 percent, about 10 percent to about 45 percent, about 10 percent to about 40 percent, about 10 percent to about 35 percent, about 10 percent to about 30 percent, about 10 percent to about 25 percent, about percent to about 20 percent, or about 10 percent to about 15 percent codons for cysteine.

In some embodiments, a cysteine-rich polynucleotide motif is composed of about 20 percent to about 95 percent, about 20 percent to about 90 percent, about 20 percent to about 85 percent, about 20 percent to about 80 percent, about 20 percent to about 75 percent, about 20 percent to about 70 percent, about 20 percent to about 65 percent, about 20 percent to about 60 percent, about 20 percent to about 55 percent, about 20 percent to about 50 percent, about 20 percent to about 45 percent, about 20 percent to about 40 percent, about 20 percent to about 35 percent, about 20 percent to about 30 percent, or about 20 percent to about 25 percent codons for cysteine.

In some embodiments, a cysteine-rich polynucleotide motif is composed of about percent to about 95 percent, about 20 percent to about 90 percent, about 20 percent to about 85 percent, about 20 percent to about 80 percent, about 20 percent to about 75 percent, about 20 percent to about 70 percent, about 20 percent to about 65 percent, about 20 percent to about 60 percent, about 20 percent to about 55 percent, about 20 percent to about 50 percent, about 20 percent to about 45 percent, about 20 percent to about 40 percent, about 20 percent to about 35 percent, about 20 percent to about 30 percent, or about 20 percent to about 25 percent codons for cysteine.

In some embodiments, a cysteine-rich polynucleotide motif is composed of about 30 percent to about 95 percent, about 30 percent to about 90 percent, about 30 percent to about 85 percent, about 30 percent to about 80 percent, about 30 percent to about 75 percent, about percent to about 70 percent, about 30 percent to about 65 percent, about 30 percent to about 60 percent, about 30 percent to about 55 percent, about 30 percent to about 50 percent, about 30 percent to about 45 percent, about 30 percent to about 40 percent, or about 30 percent to about 35 percent codons for cysteine.

In some embodiments, a cysteine-rich polynucleotide motif is composed of about 40 percent to about 95 percent, about 40 percent to about 90 percent, about 40 percent to about 85 percent, about 40 percent to about 80 percent, about 40 percent to about 75 percent, about 40 percent to about 70 percent, about 40 percent to about 65 percent, about 40 percent to about 60 percent, about 40 percent to about 55 percent, about 40 percent to about 50 percent, or about 40 percent to about 45 percent codons for cysteine.

In some embodiments, a cysteine-rich polynucleotide motif is composed of about 50 percent to about 95 percent, about 50 percent to about 90 percent, about 50 percent to about 85 percent, about 50 percent to about 80 percent, about 50 percent to about 75 percent, about 50 percent to about 70 percent, about 50 percent to about 65 percent, about 50 percent to about 60 percent, or about 50 percent to about 55 percent codons for cysteine.

In some embodiments, a cysteine-rich polynucleotide motif is composed of about 60 percent to about 95 percent, about 60 percent to about 90 percent, about 60 percent to about 85 percent, about 60 percent to about 80 percent, about 60 percent to about 75 percent, about 60 percent to about 70 percent, or about 60 percent to about 65 percent codons for cysteine.

In some embodiments, a cysteine-rich polynucleotide motif is composed of about 70 percent to about 95 percent, about 70 percent to about 90 percent, about 70 percent to about 85 percent, about 70 percent to about 80 percent, or about 70 percent to about 75 percent codons for cysteine.

In some embodiments, a cysteine-rich polynucleotide motif is composed of about 80 percent to about 95 percent, about 80 percent to about 90 percent, or about 80 percent to about 85 percent codons for cysteine.

In some embodiments, a cysteine-rich polynucleotide motif is composed of about 90 percent to about 95 percent codons for cysteine.

In embodiments where the cysteine-rich motif contains less than 100 percent cysteine codons, the cysteine rich motif can contain one or more additional nucleotides that are in addition to those coding for a cysteine. In some embodiments, the one or more additional nucleotides is at least 3 additional nucleotides which can optionally form one or more additional codons for one or more non-cysteine amino acids.

In some embodiments, the cysteine-rich motif can contain 1-500 (e.g., 1 to/or, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 4999, or 500) nucleotides in addition to those coding for one or more cysteines.

In some embodiments, the cysteine-rich motif contains 1-100 or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 or more) cysteine codons.

In some embodiments, the cysteine-rich motif contains 1-100 or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 or more) non-cysteine codons.

In some embodiments, a cysteine-rich motif contains 2-25 (e.g., 3, 4, 5, 6, 7, 8, 9, 10, 11, 12, 13, 14, 15, 16, 17, 18, 19, 20, 21, 22, 23, 24, or 25) or more contiguous cysteine codons. In some embodiments, a cysteine-rich motif contains 2-25 (e.g., 3, 4, 5, 6, 7, 8, 9, 10, 11, 12, 13, 14, 15, 16, 17, 18, 19, 20, 21, 22, 23, 24, or 25) or more non-contiguous cysteine codons.

In some embodiments, a cysteine-rich motif contains 2-25 (e.g., 3, 4, 5, 6, 7, 8, 9, 10, 11, 12, 13, 14, 15, 16, 17, 18, 19, 20, 21, 22, 23, 24, or 25) or more contiguous non-cysteine codons. In some embodiments, a cysteine-rich motif contains 2-25 (e.g., 3, 4, 5, 6, 7, 8, 9, 10, 11, 12, 13, 14, 15, 16, 17, 18, 19, 20, 21, 22, 23, 24, or 25) or more non-contiguous non-cysteine codons.

A cysteine-rich motif can be composed of 3 to about 500 or more nucleotides (e.g., 3 to/or 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 or more nucleotides).

In some embodiments, a cysteine-rich motif is 3 to about 500 nucleotides, 3 to about 475 nucleotides, 3 to about 450 nucleotides, 3 to about 425 nucleotides, 3 to about 400 nucleotides, 3 to about 375 nucleotides, 3 to about 350 nucleotides, 3 to about 325 nucleotides, 3 to about 300 nucleotides, 3 to about 275 nucleotides, 3 to about 250 nucleotides, 3 to about 225 nucleotides, 3 to about 200 nucleotides, 3 to about 175 nucleotides, 3 to about 150 nucleotides, 3 to about 125 nucleotides, 3 to about 100 nucleotides, 3 to about 75 nucleotides, 3 to about 50 nucleotides, 3 to about 25 nucleotides, 3 to about 20 nucleotides, 3 to about 15 nucleotides, or 3 to about 10 nucleotides in length.

In some embodiments, a cysteine-rich motif is about 10 to about 500 nucleotides about 10 to about 475 nucleotides, about 10 to about 450 nucleotides, about 10 to about 425 nucleotides, about 10 to about 400 nucleotides, about 10 to about 375 nucleotides, about 10 to about 350 nucleotides, about 10 to about 325 nucleotides, about 10 to about 300 nucleotides, about 10 to about 275 nucleotides, about 10 to about 250 nucleotides, about 10 to about 225 nucleotides, about 10 to about 200 nucleotides, about 10 to about 175 nucleotides, about 10 to about 150 nucleotides, about 10 to about 125 nucleotides, about 10 to about 100 nucleotides, about 10 to about 75 nucleotides, about 10 to about 50 nucleotides, about 10 to about 25 nucleotides, about 10 to about 20 nucleotides, or about 10 to about 15 nucleotides in length.

In some embodiments, a cysteine-rich motif is about 20 to about 500 nucleotides, about 20 to about 475 nucleotides, about 20 to about 450 nucleotides, about 20 to about 425 nucleotides, about 20 to about 400 nucleotides, about 20 to about 375 nucleotides, about 20 to about 350 nucleotides, about 20 to about 325 nucleotides, about 20 to about 300 nucleotides, about 20 to about 275 nucleotides, about 20 to about 250 nucleotides, about 20 to about 225 nucleotides, about 20 to about 200 nucleotides, about 20 to about 175 nucleotides, about 20 to about 150 nucleotides, about 20 to about 125 nucleotides, about 20 to about 100 nucleotides, about 20 to about 75 nucleotides, about 20 to about 50 nucleotides, or about 20 to about 25 nucleotides in length.

In some embodiments, a cysteine-rich motif is about 25 to about 500 nucleotides, about 25 to about 475 nucleotides, about 25 to about 450 nucleotides, about 25 to about 425 nucleotides, about 25 to about 400 nucleotides, about 25 to about 375 nucleotides, about 25 to about 350 nucleotides, about 25 to about 325 nucleotides, about 25 to about 300 nucleotides, about 25 to about 275 nucleotides, about 25 to about 250 nucleotides, about 25 to about 225 nucleotides, about 25 to about 200 nucleotides, about 25 to about 175 nucleotides, about 25 to about 150 nucleotides, about 25 to about 125 nucleotides, about 25 to about 100 nucleotides, about 25 to about 75 nucleotides, or about 25 to about 50 nucleotides in length.

In some embodiments, a cysteine-rich motif is about 50 to about 500 nucleotides, about 50 to about 475 nucleotides, about 50 to about 450 nucleotides, about 50 to about 425 nucleotides, about 50 to about 400 nucleotides, about 50 to about 375 nucleotides, about 50 to about 350 nucleotides, about 50 to about 325 nucleotides, about 50 to about 300 nucleotides, about 50 to about 275 nucleotides, about 50 to about 250 nucleotides, about 50 to about 225 nucleotides, about 50 to about 200 nucleotides, about 50 to about 175 nucleotides, about 50 to about 150 nucleotides, about 50 to about 125 nucleotides, about 50 to about 100 nucleotides, or about 50 to about 75 nucleotides in length.

In some embodiments, a cysteine-rich motif is about 75 to about 500 nucleotides, about 75 to about 475 nucleotides, 75 to about 450 nucleotides, about 75 to about 425 nucleotides, about 75 to about 400 nucleotides, about 75 to about 375 nucleotides, about 75 to about 350 nucleotides, about 75 to about 325 nucleotides, about 75 to about 300 nucleotides, about 75 to about 275 nucleotides, about 75 to about 250 nucleotides, about 75 to about 225 nucleotides, about 75 to about 200 nucleotides, about 75 to about 175 nucleotides, about 75 to about 150 nucleotides, about 75 to about 125 nucleotides, or about 75 to about 100 nucleotides in length.

In some embodiments, a cysteine-rich motif is about 100 to about 500 nucleotides, about 100 to about 475 nucleotides, about 100 to about 450 nucleotides, about 100 to about 425 nucleotides, about 100 to about 400 nucleotides, about 100 to about 375 nucleotides, about 100 to about 350 nucleotides, about 100 to about 325 nucleotides, about 100 to about 300 nucleotides, about 100 to about 275 nucleotides, about 100 to about 250 nucleotides, about 100 to about 225 nucleotides, about 100 to about 200 nucleotides, about 100 to about 175 nucleotides, about 100 to about 150 nucleotides, or about 100 to about 125 nucleotides in length.

In some embodiments, a cysteine-rich motif is about 125 to about 500 nucleotides, about 125 to about 475 nucleotides, about 125 to about 450 nucleotides, about 125 to about 425 nucleotides, about 125 to about 400 nucleotides, about 125 to about 375 nucleotides, about 125 to about 350 nucleotides, about 125 to about 325 nucleotides, about 125 to about 300 nucleotides, about 125 to about 275 nucleotides, about 125 to about 250 nucleotides, about 125 to about 225 nucleotides, about 125 to about 200 nucleotides, about 125 to about 175 nucleotides, or about 125 to about 150 nucleotides in length.

In some embodiments, a cysteine-rich motif is about 150 to about 500 nucleotides, about 150 to about 475 nucleotides, about 150 to about 450 nucleotides, about 150 to about 425 nucleotides, about 150 to about 400 nucleotides, about 150 to about 375 nucleotides, about 150 to about 350 nucleotides, about 150 to about 325 nucleotides, about 150 to about 300 nucleotides, about 150 to about 275 nucleotides, about 150 to about 250 nucleotides, about 150 to about 225 nucleotides, about 150 to about 200 nucleotides, or about 150 to about 175 nucleotides in length.

In some embodiments, a cysteine-rich motif is about 175 to about 500 nucleotides, about 175 to about 475 nucleotides, about 175 to about 450 nucleotides, about 175 to about 425 nucleotides, about 175 to about 400 nucleotides, about 175 to about 375 nucleotides, about 175 to about 350 nucleotides, about 175 to about 325 nucleotides, about 175 to about 300 nucleotides, about 175 to about 275 nucleotides, about 175 to about 250 nucleotides, about 175 to about 225 nucleotides, or about 175 to about 200 nucleotides in length.

In some embodiments, a cysteine-rich motif is about 200 to about 500 nucleotides, about 200 to about 475 nucleotides, about 200 to about 450 nucleotides, about 200 to about 425 nucleotides, about 200 to about 400 nucleotides, about 200 to about 375 nucleotides, about 200 to about 350 nucleotides, about 200 to about 325 nucleotides, about 200 to about 300 nucleotides, about 200 to about 275 nucleotides, about 200 to about 250 nucleotides, about 200 to about 225 nucleotides in length.

In some embodiments, a cysteine-rich motif is about 250 to about 500 nucleotides, about 250 to about 475 nucleotides, about 250 to about 450 nucleotides, about 250 to about 425 nucleotides, about 250 to about 400 nucleotides, about 250 to about 375 nucleotides, about 250 to about 350 nucleotides, about 250 to about 325 nucleotides, about 250 to about 300 nucleotides, or about 250 to about 275 nucleotides in length.

In some embodiments, a cysteine-rich motif is about 275 to about 500 nucleotides, about 275 to about 475 nucleotides, about 275 to about 450 nucleotides, about 275 to about 425 nucleotides, about 275 to about 400 nucleotides, about 275 to about 375 nucleotides, about 275 to about 350 nucleotides, about 275 to about 325 nucleotides, or about 275 to about 300 nucleotides in length.

In some embodiments, a cysteine-rich motif is about 300 to about 500 nucleotides, about 300 to about 475 nucleotides, about 300 to about 450 nucleotides, about 300 to about 425 nucleotides, about 300 to about 400 nucleotides, about 300 to about 375 nucleotides, about 300 to about 350 nucleotides, or about 300 to about 325 nucleotides in length.

In some embodiments, a cysteine-rich motif is about 325 to about 500 p nucleotides, about 325 to about 475 nucleotides, about 325 to about 450 nucleotides, about 325 to about 425 nucleotides, about 325 to about nucleotides, about 325 to about 375 nucleotides, or about 325 to about 350 nucleotides in length.

In some embodiments, a cysteine-rich motif is about 350 to about 500 nucleotides, about 350 to about 475 nucleotides, about 350 to about 450 nucleotides, about 350 to about 425 nucleotides, about 350 to about 400 nucleotides, or about 350 to about 375 nucleotides in length.

In some embodiments, a cysteine-rich motif is about 375 to about 500 nucleotides, about 375 to about 475 nucleotides, about 375 to about 450 nucleotides, about 375 to about 425 nucleotides, or about 375 to about 400 nucleotides in length.

In some embodiments, a cysteine-rich motif is about 400 to about 500 nucleotides, about 400 to about 475 nucleotides, about 400 to about 450 nucleotides, or about 400 to about 425 nucleotides in length.

In some embodiments, a cysteine-rich motif is about 425 to about 500 nucleotides, about 425 to about 475 nucleotides, or about 425 to about 450 nucleotides in length.

In some embodiments, a cysteine-rich motif is about 450 to about 500 nucleotides or about 450 to about 475 nucleotides in length.

### Lysosome Targeting Moieties

The engineered polynucleotide can include one or more lysosome targeting moieties as previously described. In some embodiments, a lysosome targeting moiety included in an engineered polynucleotide is a polynucleotide encoding a polypeptide lysosome targeting moiety such that lysosome targeting does not occur until the engineered polynucleotide is translated. This can be advantageous as it keeps the engineered polynucleotide in the nucleus and/or cytoplasm of the cell where it can be transcribed and/or translated but allows for translocation of the engineered polypeptide translated from an engineered polynucleotide into the lysosome. Thus, in some embodiments, translation of an engineered polynucleotide described herein incorporates cytosolic cysteine into an engineered polypeptide, which is then targeted (via the lysosome targeting moiety(ies), to the lysosome and therefore moves cytosolic cysteine into the lysosome. This can deplete cytosolic cyst(e)ine and increase lysosome cyst(e)ine.

Exemplary lysosome targeting moieties include, but are not limited to any one or more of the following:
a. IGF2 (insulin like growth factor 2) or an M6PR binding domain thereof
b. A LIMP-2 ligand-Lysosomal integral membrane protein LIMP-2 transports the cargo β-glucocerebrosidase into lysosome:
   i. It is predicted that the following specific regions are important for LIMP-2 binding: helix 1a (residues T86-L96), helix 1b (residues P99-S110), and helix 2 (P150-A168). Below is the sequence from T86-A168.
   ii. Protein: β-glucocerebrosidase
   iii. Amino Acid Sequence:

(SEQ ID NO: 1)
RRMELSMGPIQANHTGTGLLLTLQPEQKFQKVKGFGGAMTDAAALNILAL
SPPAQNLLLKSYFSEEGIGYNIIRVPMASCDFS

iv. mRNA sequence:

(SEQ ID NO: 2)
cgccgcauggaacugagcaugggcccgauucaggcgaaccauaccggcac
cggccugcugcugacccugcagccggaacagaaauuucagaaagugaaag
gcuuuggggcgcgaugaccgaugcggcggcgcugaacauucuggcgcuga
gcccgccggcgcagaaccugcugcugaaaagcuauuuuagcgaagaaggc
auuggcuauaacauuauucgcgugccgauggcgagcugcgauuuuagc

c. A sortilin ligand-Sortilin transports the cargo prosaposin into lysosome:
   i. Residues 521-557 of the C terminus of prosaposin is necessary for sending prosaposin to sortilin.
   ii. Protein: Prosaposin
   iii. Amino Acid Sequence:

| (SEQ ID NO: 3) | |
|---|---|
| | LLLGTEKCVWGPSYWCQNMETAARCNAVDHCKRHVWN |

iv. mRNA sequence:

(SEQ ID NO: 4)
cugcugcugggcaccgaaaaaugcguguggggcccgagcuauuggugcca
gaacauggaaaccgcggcgcgcugcaacgogguggaucauugcaaacgcc
auguguggaac

d. Additional lysosomal proteins that are sent to the lysosome after translation in the cytoplasm using dileucine-based and tyrosine-based sorting signals are in the following table (Table 1).

TABLE 1
Exemplary Lysosome targeting polypeptides.

| | Amino Acid | |
|---|---|---|
| Protein | Sequence | mRNA Sequence |
| MPR300/ | SFHDDSDEDLL | uccuuucaugaugau |
| CI-MPR | (SEQ ID NO: | uccgaugaagauctg |
| | 5) | cta (SEQ ID NO: |
| | | 6) |
| MPR46/ | EESEERDDHLL | gaagaauccgaagaa |
| CD-MPR | (SEQ ID NO: | cgcgaugaucaucug |
| | 7) | (SEQ ID NO: 8) |
| Sortilin | GYHDDSDEDLL | ggcuaucaugaugau |
| | (SEQ ID NO: | agcgaugaagaucug |
| | 9) | cug (SEQ ID NO: |
| | | 10) |
| SorLA/ | ITGFSDDVPMV | auuaccggcuuuagc |
| SORL1 | (SEQ ID NO: | gaugaugugccgaug |
| | 11) | gug (SEQ ID NO: |
| | | 12) |
| GGA1 (1) | ASVSLLDDELM | gcgagcgugagccug |
| | (SEQ ID NO: | cuggaugaugaacug |
| | 13) | aug (SEQ ID NO: |
| | | 14) |
| GGA1 (2) | ASSGLDDLDLL | gcgagcagcggccug |
| | (SEQ ID NO: | gaugaucuggaucug |
| | 15) | cug (SEQ ID NO: |
| | | 16) |
| GGA2 | VQNPSADRNLL | gugcagaacccgagc |
| | (SEQ ID NO: | gcggaucgcaaccug |
| | 17) | cug (SEQ ID NO: |
| | | 18) |
| GGA3 | NALSWLDEELL | aacgcgcugagcugg |
| | (SEQ ID NO: | cuggaugaagaacug |
| | 19) | cug (SEQ ID NO: |
| | | 20) |
| LIMP-II | DERAPLI (SEQ | gaugaacgcgcgccg |
| | ID NO: 21) | cugauu (SEQ ID |
| | | NO: 22) |
| NPC1 | TERERLL (SEQ | accgaacgegaacgc |
| | ID NO: 23) | cugcug (SEQ ID |
| | | NO: 24) |
| Mucolipin-1 | SETERLL (SEQ | agcgaaaccgaacgc |
| | ID NO: 25) | cugcug (SEQ ID |
| | | NO: 26) |
| Sialin | TDRTPLL (SEQ | accgaucgcaccccg |
| | ID NO: 27) | cugcug (SEQ ID |
| | | NO: 28) |
| GLUT8 | EETQPLL (SEQ | gaagaaacccagccg |
| | ID NO: 29) | cugcug (SEQ ID |
| | | NO: 30) |
| Invariant | DDORDLI (SEQ | gaugaucagcgcgau |
| chain (Ii) | ID NO: 31) | cugauu (SEQ ID |
| (1) | | NO: 32) |
| Invariant | NEQLPML (SEQ | aacgaacagcugccg |
| chain (Ii) | ID NO: 33) | augcug (SEQ ID |
| (2) | | NO: 34) |
| LAMP-1 | GYQTI (SEQ | ggcuaucagaccauu |
| | ID NO: 35) | (SEQ ID NO: 36) |
| LAMP-2A | GYEQF (SEQ | ggcuaugaacaguuu |
| | ID NO: 37) | (SEQ ID NO: 38) |
| LAMP-2B | GYQTL (SEQ | ggcuaucagacccug |
| | ID NO: 39) | (SEQ ID NO: 40) |
| LAMP-2C | GYQSV (SEQ | ggcuaucagagcgug |
| | ID NO: 41) | (SEQ ID NO: 42) |
| CD63 | GYEVM (SEQ | ggcuaugaagugaug |
| | ID NO: 43) | (SEQ ID NO: 44) |
| CD68 | AYQAL (SEQ | gcguaucaggcgcug |
| | ID NO: 45) | (SEQ ID NO: 46) |
| Endolyn | NYHTL (SEQ | aacuaucauacccug |
| | ID NO: 47) | (SEQ ID NO: 48) |
| DC-LAMP | GYORI (SEQ | ggcuaucagcgcauu |
| | ID NO: 49) | (SEQ ID NO: 50) |
| Cystinosin | GYDQL (SEQ | ggcuaugaucagcug |
| | ID NO: 51) | (SEQ ID NO: 52) |
| Sugar | GYKEI (SEQ | ggcuauaaagaaauu |
| phosphate | ID NO: 53) | (SEQ ID NO: 54) |
| exchanger | | |
| 2 | | |
| Acid | GYRHV (SEQ | ggcuaucgccaugug |
| phosphatase | ID NO: 55) | (SEQ ID NO: 56) |

e. any combination thereof.

### Polynucleotide Modifications

In some embodiments, one or more polynucleotides in the engineered polynucleotide are modified. In some embodiments, the engineered polynucleotide includes one or more non-naturally occurring nucleotides, which can be the result of modifying a naturally occurring nucleotide. In some embodiments, the modification is selected independently for each polynucleotide modified. In some embodiments, the modification(s) increase or decrease the stability of the polynucleotide, reduce the immunogenicity of the polynucleotide, increase or decrease the rate of transcription and/or translation, or any combination thereof. Non-naturally occurring nucleotides and/or nucleotide analogs may be modified at the ribose, phosphate, and/or base moiety.

Suitable modifications include, without limitation, methylpseudouridine, a phosphorothioate linkage, a locked nucleic acid (LNA) nucleotide comprising a methylene bridge between the 2′ and 4′ carbons of the ribose ring, or bridged nucleic acids (BNA), 2′-O-methyl analogs, 2′-deoxy analogs, or 2′-fluoro analogs, 2-aminopurine, 5-bromo-uridine, pseudouridine, (Ψ), N1-methylpseudouridine (me1Ψ), 5-methoxyuridine (5moU), inosine, 7-methylguanosine, inosine, 7-methylguanosine. Examples of guide RNA chemical modifications include, without limitation, incorporation of 2′-O-methyl (M), 2′-O-methyl 3′phosphorothioate (MS), S-constrained ethyl(cEt), or 2′-O-methyl 3′thioPACE (MSP) at one or more terminal nucleotides.

In certain embodiments, 1-100 (e.g., 1, to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100) or more nucleotides are modified.

In some embodiments, one or more nucleotides of one or more portions of the engineered polynucleotides are modified with one or more functional groups. In some embodiments, the one or more functional groups can facilitate linking one or more portions of the engineered polynucleotide together so as to form the complete engineered polynucleotide and/or can otherwise facilitated synthesis of the engineered polynucleotide. In some embodiments, one or more portions of the engineered biomolecule can be synthesized, e.g., using the standard phosphoramidite synthetic protocol (Herdewijn, P., ed., Methods in Molecular Biology Col 288, Oligonucleotide Synthesis: Methods and Applications, Humana Press, New Jersey (2012)). Functional groups may be optionally added to facilitate e.g., ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sulfonyl, ally, propargyl, diene, alkyne, and azide. Once functionalized, a covalent chemical bond or linkage can be formed between the sequence of one portion and another. Examples of chemical bonds include, but are not limited to, those based on carbamates, ethers, esters, amides, imines, amidines, aminotriazines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

### Polypeptides

In some embodiments, the engineered biomolecule is a polypeptide. As previously described, an engineered polynucleotide can be transcribed and/or translated into an engineered polypeptide of the present disclosure. In some embodiments, this occurs in vivo, in vitro, or ex vivo after administration or delivery of an engineered polynucleotide to a subject and/or cell(s). In some embodiments, an engineered polypeptide is generated de novo based on an amino acid sequence. In some embodiments, an engineered polypeptide is prepared outside of a recipient cell or subject in need thereof and subsequently delivered to the recipient cell or subject in need thereof. Delivery is described in greater detail elsewhere herein.

In some embodiments, the engineered polynucleotide contains 1 to 1000 or more amino acids (e.g., 1, to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000 or more amino acids).

### Cysteine-Rich (Polypeptide) Motifs

In the context of embodiments of the engineered polypeptides herein, “cysteine-rich motifs” refers to regions or amino acid sequences that are composed of about 10-100 percent (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 percent) cysteine residues. In some embodiments, a cysteine-rich polynucleotide motif is composed of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 99 percent to 100 percent cysteine residues.

In some embodiments, a cysteine-rich polynucleotide motif is composed of about 10 percent to about 95 percent, about 10 percent to about 90 percent, about 10 percent to about 85 percent, about 10 percent to about 80 percent, about 10 percent to about 75 percent, about 10 percent to about 70 percent, about 10 percent to about 65 percent, about 10 percent to about 60 percent, about 10 percent to about 55 percent, about 10 percent to about 50 percent, about 10 percent to about 45 percent, about 10 percent to about 40 percent, about 10 percent to about 35 percent, about 10 percent to about 30 percent, about 10 percent to about 25 percent, about 10 percent to about 20 percent, or about 10 percent to about 15 percent cysteine residues.

In some embodiments, a cysteine-rich polynucleotide motif is composed of about 20 percent to about 95 percent, about 20 percent to about 90 percent, about 20 percent to about 85 percent, about 20 percent to about 80 percent, about 20 percent to about 75 percent, about 20 percent to about 70 percent, about 20 percent to about 65 percent, about 20 percent to about 60 percent, about 20 percent to about 55 percent, about 20 percent to about 50 percent, about 20 percent to about 45 percent, about 20 percent to about 40 percent, about 20 percent to about 35 percent, about 20 percent to about 30 percent, or about 20 percent to about 25 percent cysteine residues.

In some embodiments, a cysteine-rich polynucleotide motif is composed of about 20 percent to about 95 percent, about 20 percent to about 90 percent, about 20 percent to about 85 percent, about 20 percent to about 80 percent, about 20 percent to about 75 percent, about 20 percent to about 70 percent, about 20 percent to about 65 percent, about 20 percent to about 60 percent, about 20 percent to about 55 percent, about 20 percent to about 50 percent, about 20 percent to about 45 percent, about 20 percent to about 40 percent, about 20 percent to about 35 percent, about 20 percent to about 30 percent, or about 20 percent to about 25 percent cysteine residues.

In some embodiments, a cysteine-rich polynucleotide motif is composed of about 30 percent to about 95 percent, about 30 percent to about 90 percent, about 30 percent to about 85 percent, about 30 percent to about 80 percent, about 30 percent to about 75 percent, about 30 percent to about 70 percent, about 30 percent to about 65 percent, about 30 percent to about 60 percent, about 30 percent to about 55 percent, about 30 percent to about 50 percent, about 30 percent to about 45 percent, about 30 percent to about 40 percent, or about 30 percent to about 35 percent codons for cysteine.

In some embodiments, a cysteine-rich polynucleotide motif is composed of about 40 percent to about 95 percent, about 40 percent to about 90 percent, about 40 percent to about 85 percent, about 40 percent to about 80 percent, about 40 percent to about 75 percent, about 40 percent to about 70 percent, about 40 percent to about 65 percent, about 40 percent to about 60 percent, about 40 percent to about 55 percent, about 40 percent to about 50 percent, or about 40 percent to about 45 percent cysteine residues.

In some embodiments, a cysteine-rich polynucleotide motif is composed of about 50 percent to about 95 percent, about 50 percent to about 90 percent, about 50 percent to about 85 percent, about 50 percent to about 80 percent, about 50 percent to about 75 percent, about 50 percent to about 70 percent, about 50 percent to about 65 percent, about 50 percent to about 60 percent, or about 50 percent to about 55 percent cysteine residues.

In some embodiments, a cysteine-rich polynucleotide motif is composed of about 60 percent to about 95 percent, about 60 percent to about 90 percent, about 60 percent to about 85 percent, about 60 percent to about 80 percent, about 60 percent to about 75 percent, about 60 percent to about 70 percent, or about 60 percent to about 65 percent cysteine residues.

In some embodiments, a cysteine-rich polynucleotide motif is composed of about 70 percent to about 95 percent, about 70 percent to about 90 percent, about 70 percent to about 85 percent, about 70 percent to about 80 percent, or about 70 percent to about 75 percent cysteine residues.

In some embodiments, a cysteine-rich polynucleotide motif is composed of about 80 percent to about 95 percent, about 80 percent to about 90 percent, or about 80 percent to about 85 percent cysteine residues.

In some embodiments, a cysteine-rich polynucleotide motif is composed of about 90 percent to about 95 percent cysteine residues.

In some embodiments, the cysteine-rich motif can contain 1-500 (e.g., 1, to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500) or more cysteine amino acids.

In embodiments where the cysteine-rich motif contains less than 100 percent cysteine residues, the cysteine-rich motif can contain one or more non-cysteine amino acids. In some embodiments, the cysteine-rich motif can contain 1-500 (e.g., 1, to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500) or more non-cysteine amino acids.

In some embodiments, a cysteine-rich motif contains 2-25 (e.g., 3, 4, 5, 6, 7, 8, 9, 10, 11, 12, 13, 14, 15, 16, 17, 18, 19, 20, 21, 22, 23, 24, or 25) or more contiguous cysteine residues. In some embodiments, a cysteine-rich motif contains 2-25 (e.g., 3, 4, 5, 6, 7, 8, 9, 10, 11, 12, 13, 14, 15, 16, 17, 18, 19, 20, 21, 22, 23, 24, or 25) or more non-contiguous cysteine residues.

A cysteine-rich motif can be composed of 1 to about 500 or more amino acids (e.g., 1 to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 or more amino acids).

In some embodiments, a cysteine-rich motif is 1 to about 500 amino acids, 1 to about 475 amino acids, 1 to about 450 amino acids, 1 to about 425 amino acids, 1 to about 400 amino acids, 1 to about 375 nucleotides, 1 to about 350 amino acids, 1 to about 325 amino acids, 1 to about 300 amino acids, 1 to about 275 amino acids, 1 to about 250 amino acids, 1 to about 225 amino acids, 1 to about 200 amino acids, 1 to about 175 amino acids, 1 to about 150 amino acids, 1 to about 125 amino acids, 1 to about 100 amino acids, 1 to about 75 amino acids s, 1 to about 50 amino acids, 1 to about 25 amino acids, 1 to about 20 amino acids, 1 to about 15 amino acids, 1 to about 10 amino acids or 1 to about 5 amino acids in length.

In some embodiments, a cysteine-rich motif is about 10 to about 500 amino acids about 10 to about 475 amino acids, about 10 to about 450 amino acids, about 10 to about 425 amino acids, about 10 to about 400 amino acids, about 10 to about 375 amino acids, about 10 to about 350 amino acids, about 10 to about 325 amino acids, about 10 to about 300 amino acids, about 10 to about 275 amino acids, about 10 to about 250 amino acids, about 10 to about 225 amino acids, about 10 to about 200 amino acids, about 10 to about 175 amino acids, about 10 to about 150 amino acids, about 10 to about 125 amino acids, about 10 to about 100 amino acids, about 10 to about 75 amino acids, about 10 to about 50 amino acids, about 10 to about 25 amino acids, about 10 to about 20 amino acids, or about 10 to about 15 amino acids in length.

In some embodiments, a cysteine-rich motif is about 20 to about 500 amino acids, about 20 to about 475 amino acids, about 20 to about 450 amino acids, about 20 to about 425 amino acids, about 20 to about 400 nucleotides, about 20 to about 375 nucleotides, about 20 to about 350 amino acids, about 20 to about 325 amino acids, about 20 to about 300 amino acids, about 20 to about 275 amino acids, about 20 to about 250 amino acids, about 20 to about 225 amino acids, about 20 to about 200 amino acids, about 20 to about 175 amino acids, about 20 to about 150 amino acids, about 20 to about 125 amino acids, about 20 to about 100 amino acids, about 20 to about 75 amino acids, about 20 to about 50 amino acids, or about 20 to about 25 amino acids s in length.

In some embodiments, a cysteine-rich motif is about 25 to about 500 nucleotides, about 25 to about 475 amino acids, about 25 to about 450 amino acids, about 25 to about 425 amino acids, about 25 to about 400 amino acids, about 25 to about 375 amino acids, about 25 to about 350 amino acids, about 25 to about 325 amino acids, about 25 to about 300 amino acids, about 25 to about 275 amino acids, about 25 to about 250 amino acids, about 25 to about 225 amino acids, about 25 to about 200 amino acids, about 25 to about 175 amino acids, about 25 to about 150 amino acids, about 25 to about 125 amino acids, about 25 to about 100 amino acids, about 25 to about 75 amino acids, or about 25 to about 50 amino acids in length.

In some embodiments, a cysteine-rich motif is about 50 to about 500 amino acids, about 50 to about 475 amino acids, about 50 to about 450 amino acids, about 50 to about 425 amino acids, about 50 to about 400 amino acids, about 50 to about 375 amino acids, about 50 to about 350 amino acids, about 50 to about 325 amino acids, about 50 to about 300 amino acids, about 50 to about 275 amino acids, about 50 to about 250 amino acids, about 50 to about 225 amino acids, about 50 to about 200 amino acids, about 50 to about 175 amino acids, about 50 to about 150 amino acids, about 50 to about 125 amino acids s, about 50 to about 100 amino acids, or about 50 to about 75 amino acids in length.

In some embodiments, a cysteine-rich motif is about 75 to about 500 amino acids, about 75 to about 475 amino acids, 75 to about 450 amino acids, about 75 to about 425 amino acids, about 75 to about 400 amino acids, about 75 to about 375 amino acids, about 75 to about 350 amino acids, about 75 to about 325 amino acids, about 75 to about 300 amino acids, about 75 to about 275 amino acids, about 75 to about 250 amino acids, about 75 to about 225 amino acids, about 75 to about 200 amino acids, about 75 to about 175 amino acids, about 75 to about 150 amino acids, about 75 to about 125 amino acids, or about 75 to about 100 amino acids in length.

In some embodiments, a cysteine-rich motif is about 100 to about 500 amino acids, about 100 to about 475 amino acids, about 100 to about 450 amino acids, about 100 to about 425 amino acids, about 100 to about 400 amino acids, about 100 to about 375 amino acids, about 100 to about 350 amino acids, about 100 to about 325 amino acids, about 100 to about 300 amino acids, about 100 to about 275 amino acids, about 100 to about 250 amino acids, about 100 to about 225 amino acids, about 100 to about 200 amino acids, about 100 to about 175 amino acids, about 100 to about 150 amino acids, or about 100 to about 125 amino acids in length.

In some embodiments, a cysteine-rich motif is about 125 to about 500 amino acids, about 125 to about 475 amino acids, about 125 to about 450 amino acids, about 125 to about 425 amino acids, about 125 to about 400 amino acids, about 125 to about 375 amino acids, about 125 to about 350 amino acids, about 125 to about 325 amino acids, about 125 to about 300 amino acids, about 125 to about 275 amino acids, about 125 to about 250 amino acids, about 125 to about 225 amino acids, about 125 to about 200 amino acids, about 125 to about 175 amino acids, or about 125 to about 150 amino acids in length.

In some embodiments, a cysteine-rich motif is about 150 to about 500 amino acids, about 150 to about 475 amino acids, about 150 to about 450 amino acids, about 150 to about 425 amino acids, about 150 to about 400 amino acids, about 150 to about 375 amino acids, about 150 to about 350 amino acids, about 150 to about 325 amino acids, about 150 to about 300 amino acids, about 150 to about 275 amino acids, about 150 to about 250 amino acids, about 150 to about 225 amino acids, about 150 to about 200 amino acids, or about 150 to about 175 amino acids in length.

In some embodiments, a cysteine-rich motif is about 175 to about 500 amino acids, about 175 to about 475 amino acids, about 175 to about 450 amino acids, about 175 to about 425 amino acids, about 175 to about 400 amino acids, about 175 to about 375 amino acids, about 175 to about 350 amino acids, about 175 to about 325 amino acids, about 175 to about 300 amino acids, about 175 to about 275 amino acids, about 175 to about 250 amino acids, about 175 to about 225 amino acids, or about 175 to about 200 amino acids in length.

In some embodiments, a cysteine-rich motif is about 200 to about 500 amino acids, about 200 to about 475 amino acids, about 200 to about 450 amino acids, about 200 to about 425 amino acids, about 200 to about 400 amino acids, about 200 to about 375 amino acids, about 200 to about 350 amino acids, about 200 to about 325 amino acids, about 200 to about 300 amino acids, about 200 to about 275 amino acids, about 200 to about 250 amino acids, about 200 to about 225 amino acids in length.

In some embodiments, a cysteine-rich motif is about 250 to about 500 amino acids, about 250 to about 475 amino acids, about 250 to about 450 amino acids, about 250 to about 425 amino acids, about 250 to about 400 amino acids, about 250 to about 375 amino acids, about 250 to about 350 amino acids, about 250 to about 325 amino acids, about 250 to about 300 amino acids, or about 250 to about 275 amino acids in length.

In some embodiments, a cysteine-rich motif is about 275 to about 500 amino acids, about 275 to about 475 amino acids, about 275 to about 450 amino acids, about 275 to about 425 amino acids, about 275 to about 400 amino acids, about 275 to about 375 amino acids, about 275 to about 350 amino acids, about 275 to about 325 amino acids, or about 275 to about 300 amino acids in length.

In some embodiments, a cysteine-rich motif is about 300 to about 500 amino acids, about 300 to about 475 amino acids, about 300 to about 450 amino acids, about 300 to about 425 nucleotides, about 300 to about 400 amino acids, about 300 to about 375 amino acids, about 300 to about 350 amino acids, or about 300 to about 325 amino acids in length.

In some embodiments, a cysteine-rich motif is about 325 to about 500 amino acids, about 325 to about 475 amino acids, about 325 to about 450 amino acids, about 325 to about 425 amino acids, about 325 to about amino acids, about 325 to about 375 amino acids, or about 325 to about 350 amino acids in length.

In some embodiments, a cysteine-rich motif is about 350 to about 500 amino acids, about 350 to about 475 amino acids, about 350 to about 450 amino acids, about 350 to about 425 amino acids, about 350 to about 400 amino acids, or about 350 to about 375 amino acids in length.

In some embodiments, a cysteine-rich motif is about 375 to about 500 amino acids, about 375 to about 475 amino acids, about 375 to about 450 amino acids, about 375 to about 425 amino acids, or about 375 to about 400 amino acids in length.

In some embodiments, a cysteine-rich motif is about 400 to about 500 amino acids, about 400 to about 475 amino acids, about 400 to about 450 amino acids, or about 400 to about 425 amino acids in length.

In some embodiments, a cysteine-rich motif is about 425 to about 500 amino acids, about 425 to about 475 amino acids, or about 425 to about 450 amino acids in length.

In some embodiments, a cysteine-rich motif is about 450 to about 500 amino acids or about 450 to about 475 amino acids in length.

### Lysosome Targeting Moieties

The engineered polypeptide can include one or more lysosome targeting moieties as previously described. In some embodiments, a lysosome targeting moiety included in an engineered polynucleotide a polypeptide lysosome targeting moiety. The polypeptide targeting moiety can be an antibody or fragment thereof, a ligand for a lysosome receptor, or other moiety that can specifically bind, specifically associate with, and/or facilitate influx or uptake of the engineered polypeptide into the lysosome.

Exemplary lysosome targeting moieties include, but are not limited to any one or more of the following:

IGF2 (insulin like growth factor 2) or an M6PR binding domain thereof

A LIMP-2 ligand-Lysosomal integral membrane protein LIMP-2 transports the cargo β-glucocerebrosidase into lysosome:
a. It is predicted that the following specific regions are important for LIMP-2 binding: helix 1a (residues T86-L96), helix 1b (residues P99-S110), and helix 2 (P150-A168). Below is the sequence from T86-A168.
b. Protein: β-glucocerebrosidase
c. Amino Acid Sequence:

(SEQ ID NO: 1)
RRMELSMGPIQANHTGTGLLLTLQPEQKFQKVKGFGGAMTDAAALNILAL
SPPAQNLLLKSYFSEEGIGYNIIRVPMASCDFS

d. mRNA sequence:

(SEQ ID NO: 2)
cgccgcauggaacugagcaugggcccgauucaggcgaaccauaccggcac
cggccugcugcugacccugcagccggaacagaaauuucagaaagugaaag
gcuuuggggcgcgaugaccgaugcggcggcgcugaacauucuggcgcuga
gcccgccggcgcagaaccugcugcugaaaagcuauuuuagcgaagaaggc
auuggcuauaacauuauucgcgugccgauggcgagcugegauuuuagc

A sortilin ligand-Sortilin transports the cargo prosaposin into lysosome:
a. Residues 521-557 of the C terminus of prosaposin is necessary for sending prosaposin to sortilin.
b. Protein: Prosaposin
c. Amino Acid Sequence:

| (SEQ ID NO: 3) | |
|---|---|
| | LLLGTEKCVWGPSYWCQNMETAARCNAVDHCKRHVWN |

d. mRNA sequence:

(SEQ ID NO: 4)
cugcugcugggcaccgaaaaaugcguguggggcccgagcuauuggugcca
gaacauggaaaccgcggcgcgcugcaacgcgguggaucauugcaaacgcc
auguguggaac

Additional lysosomal proteins that are sent to the lysosome after translation in the cytoplasm using dileucine-based and tyrosine-based sorting signals are in Table 1.
a. any combination thereof.

### Polypeptide Modifications

In some embodiments, one or more amino acids in the engineered polypeptide are modified with one or more post-translation modifications. In some embodiments, the modification is selected independently for each amino acids modified. In some embodiments, the modification(s) increase or decrease the stability of the polypeptide, increase or decrease the half-life, reduce the immunogenicity of the polypeptide, target the polypeptide to a target, allow for monitoring, detection, or visualization of the polypeptide, facilitate polypeptide loading into a delivery vehicle, or any combination thereof. More than 400 post-translational modifications are known in the art, any of which may be incorporated in one or more amino acids of the engineered prolylpeptides described herein.

Suitable modifications include, without limitation, phosphorylation, acetylation, ubiquitylation, methylation, glycosylation, SUMOylation, palmitoylation, myristoylation, prenylation, sulfation, any of those presented in Muller, M. Biochemistry 2018, 57, 2, 177-185 incorporated by reference herein); Ramazi et al., 2021. Database. https://doi.org/10.1093/database/baab012 (incorporated by reference herein), any of those described in any of the following post-translational modification databases: dbPTM, BioGRID. Phosphosite Plus, PTMCodev2, qPTM, PLMD, CPLM, YAAM, HPRD, PHOSIDA, PTM-SD, WERAM, EPSD, PhosphoNET, RegPhos, Phospho.ELM, Phospho3D, dbPSP, pTestis, LymPHOS. P3 DB, UniPep, GlycoFly, GlycoFish, mUbiSiDa, SwissPalm, dbSNO (see also e.g., Ramazi et al., 2021.), those described in Narita et al. Nature Reviews Molecular Cell Biology volume 20, pages 156-174 (2019) (incorporated by reference herein), and combinations thereof. Other suitable post-translational modifications will be appreciated by those of ordinary skill in the art in view of the description herein.

### Vectors and Vector Systems

Also described herein are vectors and vector systems that can contain one or more of the engineered polynucleotides described herein. In some embodiments, the vector can contain one or more engineered polynucleotides encoding one or more elements of engineered polypeptide described herein. The vectors can be useful, inter alia, in producing bacterial, fungal, yeast, plant cells, animal cells, and transgenic animals that can express one or more engineered biomolecules described herein. In some embodiments, the vectors and/or vector systems can be used, for example, to express one or more of the polynucleotides in a cell, such as a producer cell, to produce e.g., engineered polypeptides, delivery vehicles containing an engineered biomolecule of the present disclosure which are described in greater detail elsewhere herein. Other uses for the vectors and vector systems described herein are also within the scope of this disclosure and include producing an engineered biomolecule in a recipient cell (i.e., a cell which is in need of the engineered biomolecule but is not necessarily used to produce an engineered biomolecule of the present disclosure) and/or subject in need thereof. In general, and throughout this specification, the term “vector” refers to a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can be composed of a nucleic acid (e.g., a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and further defined elsewhere herein. In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells. These and other aspects of the vectors and vector systems are described elsewhere herein.

In some embodiments, the vector can be a bicistronic vector. In some embodiments, a bicistronic vector can be used for one or more engineered polynucleotides described herein. In some embodiments, expression of one or more engineered polynucleotides described herein can be driven by a Pol II promoter. In some embodiments, expression of one or more engineered polynucleotides described herein can be driven by a ubiquitous or constitutive promoter. In some embodiments, expression of one or more engineered polynucleotides described herein can be driven by a tissue-specific and/or inducible promoter. In some embodiments, expression of one or more engineered polynucleotides described herein can be driven by a tumor specific promoter. Where the engineered biomolecule is an RNA, its expression can be driven by a Pol III promoter, such as a U6 promoter.

### Cell-Based Vector Amplification and Expression

Vectors can be designed for expression of one or more engineered polynucleotides described herein (e.g., nucleic acid transcripts, proteins, enzymes, and combinations thereof) in a suitable host cell. In some embodiments, the suitable host cell is a prokaryotic cell. Suitable host cells include, but are not limited to, bacterial cells, yeast cells, insect cells, and mammalian cells. The vectors can be viral-based or non-viral based. In some embodiments, the suitable host cell is a eukaryotic cell. In some embodiments, the suitable host cell is a suitable bacterial cell. Suitable bacterial cells include, but are not limited to, bacterial cells from the bacteria of the species Escherichia coli. Many suitable strains of E. coli are known in the art for expression of vectors. These include, but are not limited to Pir1, Stb12, Stb13, Stb14, TOP10, XL1 Blue, and XL10 Gold. In some embodiments, the host cell is a suitable insect cell. Suitable insect cells include those from Spodoptera frugiperda. Suitable strains of S. frugiperda cells include, but are not limited to, Sf9 and Sf21. In some embodiments, the host cell is a suitable yeast cell. In some embodiments, the yeast cell can be from Saccharomyces cerevisiae. In some embodiments, the host cell is a suitable mammalian cell. Many types of mammalian cells have been developed to express vectors. Suitable mammalian cells include, but are not limited to, HEK293, Chinese Hamster Ovary Cells (CHOs), mouse myeloma cells, HeLa, U2OS, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif. (1990).

In some embodiments, the vector can be a yeast expression vector. Examples of vectors for expression in yeast Saccharomyces cerevisiae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2nd edition, Xiao, W., ed. (Humana Press, New York, 2007) and Buckholz, R. G. and Gleeson, M. A. (1991) Biotechnology (NY) 9(11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2μ plasmids, yeast integrative plasmids, yeast replicative plasmids, shuttle vectors, and episomal plasmids.

In some embodiments, the vector is a baculovirus vector or expression vector and can be suitable for expression of polynucleotides and/or proteins in insect cells. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31⁻³⁹). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, the vector is a mammalian expression vector. In some embodiments, the mammalian expression vector is capable of expressing one or more polynucleotides and/or polypeptides in a mammalian cell. Examples of mammalian expression vectors include, but are not limited to, pCDM8 (Seed, 1987. Nature 329: 840) and pMT2PC (Kaufman, et al., 1987. EMBO J. 6: 187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More detail on suitable regulatory elements are described elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546). With regards to these prokaryotic and eukaryotic vectors, mention is made of U.S. Pat. No. 6,750,059, the contents of which are incorporated by reference herein in their entirety. Other aspects can utilize viral vectors, with regards to which mention is made of U.S. patent application Ser. No. 13/092,085, the contents of which are incorporated by reference herein in their entirety. Tissue-specific regulatory elements are known in the art and in this regard, mention is made of U.S. Pat. No. 7,776,321, the contents of which are incorporated by reference herein in their entirety. In some embodiments, a regulatory element can be operably linked to one or more of the engineered polynucleotides so as to drive expression of the one or more of the engineered polynucleotides described herein.

Vectors may be introduced and propagated in a prokaryote or prokaryotic cell. In some embodiments, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). In some embodiments, a prokaryote is used to amplify copies of a vector and express one or more nucleic acids, such as to provide a source of one or more proteins for delivery to a host cell or host organism.

In some embodiments, the vector can be a fusion vector or fusion expression vector. In some embodiments, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In some embodiments, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In some embodiments, the fusion expression vector can include a proteolytic cleavage site, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

### Vector Features

The vectors can include additional features that can confer one or more functionalities to the vector, the polynucleotide to be delivered, a virus particle produced there from, or polypeptide expressed thereof. Such features include, but are not limited to, regulatory elements, selectable markers, molecular identifiers (e.g., molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

In some embodiments, the engineered polynucleotides and/or vectors thereof of the present disclosure described herein include one or more regulatory elements that can be operatively linked to an engineered polynucleotide. The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981).

In some embodiments, the regulatory sequence can be a regulatory sequence described in U.S. Pat. No. 7,776,321, U.S. Pat. Pub. No. 2011/0027239, and International Publication No. WO 2011/028929, the contents of which are incorporated by reference herein in their entirety. In some embodiments, the vector contains a minimal promoter. In some embodiments, the minimal promoter is the Mecp2 promoter, tRNA promoter, or U6. In a further embodiment, the minimal promoter is tissue specific. In some embodiments, the length of the vector polynucleotide the minimal promoters and polynucleotide sequences is less than 4.4 Kb.

To express a polynucleotide, the vector can include one or more transcriptional and/or translational initiation regulatory sequences, e.g., promoters, that direct the transcription of the gene and/or translation of the encoded protein in a cell. In some embodiments a constitutive promoter may be employed. Suitable constitutive promoters for mammalian cells are generally known in the art and include, but are not limited to SV40, CAG, CMV, EF-1α, β-actin, RSV, and PGK. Suitable constitutive promoters for bacterial cells, yeast cells, and fungal cells are generally known in the art, such as a T-7 promoter for bacterial expression and an alcohol dehydrogenase promoter for expression in yeast.

In some embodiments, the regulatory element can be a regulated promoter. “Regulated promoter” refers to promoters that direct gene expression not constitutively, but in a temporally- and/or spatially-regulated manner, and includes tissue-specific, tissue-preferred and inducible promoters. Regulated promoters include conditional promoters and inducible promoters. In some embodiments, conditional promoters can be employed to direct expression of a polynucleotide in a specific cell type, under certain environmental conditions, and/or during a specific state of development. Suitable tissue specific promoters can include, but are not limited to, liver specific promoters (e.g., APOA2, SERPIN A1 (hAAT), CYP3A4, and MIR122), pancreatic cell promoters (e.g., INS, IRS2, Pdx1, Alx3, Ppy), cardiac specific promoters (e.g., Myh6 (alpha MHC), MYL2 (MLC-2v), TNI3 (cTn1), NPPA (ANF), Slc8a1 (Ncx1)), central nervous system cell promoters (SYN1, GFAP, INA, NES, MOBP, MBP, TH, FOXA2 (HNF3 beta)), skin cell specific promoters (e.g., FLG, K14, TGM3), immune cell specific promoters, (e.g. ITGAM, CD43 promoter, CD14 promoter, CD45 promoter, CD68 promoter), urogenital cell specific promoters (e.g., Pbsn, Upk2, Sbp, Fer114), endothelial cell specific promoters (e.g., ENG), pluripotent and embryonic germ layer cell specific promoters (e.g., Oct4, NANOG, Synthetic Oct4, T brachyury, NES, SOX17, FOXA2, MIR122), and muscle cell specific promoter (e.g., Desmin), or tumor specific promoters (e.g., AFP promoter (hepatocellular carcinoma specific promoter), CCKAR (pancreatic cancer specific promoter), CEA promoter (epithelial cancer specific promoter), c-erbB2 promoter (breast and pancreatic specific promoter), COX-2 promoter, CXCR4 promoter, E2F-1 promoter, HE4 promoter, LP promoter, MUC1 promoter (carcinoma cell specific), PSA promoter (prostate cell and prostate cancer cell specific), surviving promoter, TRP1 promoter (melanocyte and melanoma specific), Tyr promoter (melanocyte and melanoma specific), ran promoter, brms1 promoter, mcm5 promoter, TERT, SPA1, A33 promoter, uPAR promoter, FGF18 promoter, and KDR promoter,). Other tissue and/or cell specific promoters are generally known in the art and are within the scope of this disclosure.

Where expression in a plant cell is desired, such as for the production of engineered RNA and/or polypeptides, the engineered polynucleotides described herein are typically placed under control of a plant promoter, i.e., a promoter operable in plant cells. The use of different types of promoters is envisaged.

A constitutive plant promoter is a promoter that is able to express the open reading frame (ORF) that it controls in all or nearly all of the plant tissues during all or nearly all developmental stages of the plant (referred to as “constitutive expression”). One non-limiting example of a constitutive promoter is the cauliflower mosaic virus 35S promoter. Different promoters may direct the expression of a gene in different tissues or cell types, or at different stages of development, or in response to different environmental conditions. In particular embodiments, one or more of the engineered polynucleotides are expressed under the control of a constitutive promoter, such as the cauliflower mosaic virus 35S promoter issue-preferred promoters can be utilized to target enhanced expression in certain cell types within a particular plant tissue, for instance vascular cells in leaves or roots or in specific cells of the seed. Examples of particular promoters for use in expression of the engineered polynucleotides of the present disclosure are found in Kawamata et al., (1997) Plant Cell Physiol 38:792-803; Yamamoto et al., (1997) Plant J 12:255-65; Hire et al, (1992) Plant Mol Biol 20:207-18, Kuster et al, (1995) Plant Mol Biol 29:759-72, and Capana et al., (1994) Plant Mol Biol 25:681-91.

Inducible/conditional promoters can be positively inducible/conditional promoters (e.g., a promoter that activates transcription of the polynucleotide upon appropriate interaction with an activated activator, or an inducer (compound, environmental condition, or other stimulus) or a negative/conditional inducible promoter (e.g., a promoter that is repressed) (e.g., bound by a repressor) until the repressor condition of the promotor is removed (e.g., inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

Examples of promoters that are inducible and that can allow for spatiotemporal control of gene editing or gene expression may use a form of energy. The form of energy may include but is not limited to sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc.), or light inducible systems (Phytochrome, LOV domains, or cryptochrome)., such as a Light Inducible Transcriptional Effector (LITE) that direct changes in transcriptional activity in a sequence-specific manner. The components of a light inducible system may include one or more engineered polynucleotides described herein, a light-responsive cytochrome heterodimer (e.g., from Arabidopsis thaliana), and a transcriptional activation/repression domain. In some embodiments, the vector can include one or more of the inducible DNA binding proteins provided in PCT publication WO 2014/018423 and US Publications, 2015/0291966, 2017/0166903, 2019/0203212, which describe e.g., aspects of inducible DNA binding proteins and methods of use and can be adapted for use with the present invention.

In some embodiments, transient or inducible expression can be achieved by including, for example, chemical-regulated promotors, i.e., whereby the application of an exogenous chemical induces gene expression. Modulation of gene expression can also be obtained by including a chemical-repressible promoter, where application of the chemical represses gene expression. Chemical-inducible promoters include, but are not limited to, the maize ln2-2 promoter, activated by benzene sulfonamide herbicide safeners (De Veylder et al., (1997) Plant Cell Physiol 38:568-77), the maize GST promoter (GST-11-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Promoters that are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

In some embodiments, the vector or system thereof can include one or more elements capable of translocating and/or expressing an engineered polynucleotide to/in a specific cell component or organelle. Such organelles can include, but are not limited to, nucleus, cytoplasm, ribosome, endoplasmic reticulum, golgi apparatus, chloroplast, mitochondria, vacuole, lysosome, cytoskeleton, plasma membrane, cell wall, peroxisome, centrioles, etc.

### Selectable Markers and Tags

One or more of the engineered polynucleotides can be operably linked, fused to, or otherwise modified to include a polynucleotide that encodes or is a selectable marker or tag, which can be a polynucleotide or polypeptide. In some embodiments, the polypeptide encoding a polypeptide selectable marker can be incorporated in the engineered polynucleotide such that the selectable marker polypeptide, when translated, is inserted between two amino acids between the N- and C-terminus of the engineered polypeptide or at the N- and/or C-terminus of the engineered polypeptide. In some embodiments, the selectable marker or tag is a polynucleotide barcode or unique molecular identifier (UMI).

It will be appreciated that the polynucleotide encoding such selectable markers or tags can be incorporated into a polynucleotide encoding one or more of the engineered polynucleotides described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FLASH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g., GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more of the engineered polynucleotides described herein via suitable linker, such as a glycine or glycine serine linkers as short as GS or GG up to (GGGGG)₃ (SEQ ID NO: 57) or (GGGGS)₃ (SEQ ID NO: 58). Other suitable linkers are described elsewhere herein.

The vector or vector system can include one or more polynucleotides encoding one or more targeting moieties. In some embodiments, the targeting moiety encoding polynucleotides can be included in the vector or vector system, such as a viral vector system, such that they are expressed within and/or on the virus particle(s) produced such that the virus particles can be targeted to specific cells, tissues, organs, etc. In some embodiments, the targeting moiety encoding polynucleotides can be included in the vector or vector system such that the engineered polynucleotide(s) and/or products expressed therefrom include the targeting moiety and can be targeted to specific cells, tissues, organs, etc. In some embodiments, such as non-viral carriers, the targeting moiety can be attached to the carrier (e.g., polymer, lipid, inorganic molecule etc.) and can be capable of targeting the engineered biomolecule and/or delivery vehicle containing the engineered biomolecule and any attached or associated with the engineered polynucleotide(s) and/or polypeptides produced therefrom to specific cells, tissues, organs, etc. In some embodiments, the targeting moiety is a lysosomal targeting moiety (i.e., a targeting moiety that is capable of targeting the engineered biomolecule to a target cell, target cell type, and/or compartment within a cell or cell type. In some embodiments, the targeting moiety targets a cancer cell. In some embodiments, the targeting moiety targets a lysosome. In some embodiments, the engineered biomolecule and/or delivery vehicle includes two or more targeting moiety. In some embodiments, the two or more targeting moieties include at least a lysosome targeting moiety and a cancer or tumor cell targeting moiety.

### Cell-Free Vector and Polynucleotide Expression

In some embodiments, the polynucleotide encoding one or more engineered polynucleotides is/are expressed from a vector or suitable polynucleotide in a cell-free in vitro system. In other words, the polynucleotide can be transcribed and optionally translated in vitro. In vitro transcription/translation systems and appropriate vectors are generally known in the art and commercially available. Generally, in vitro transcription and in vitro translation systems replicate the processes of RNA and protein synthesis, respectively, outside of the cellular environment. Vectors and suitable polynucleotides for in vitro transcription can include T7, SP6, T3, promoter regulatory sequences that can be recognized and acted upon by an appropriate polymerase to transcribe the polynucleotide or vector.

In vitro translation can be stand-alone (e.g., translation of a purified polyribonucleotide) or linked/coupled to transcription. In some embodiments, the cell-free (or in vitro) translation system can include extracts from rabbit reticulocytes, wheat germ, and/or E. coli. The extracts can include various macromolecular components that are needed for translation of exogenous RNA (e.g., 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (creatine phosphate and creatine phosphokinase (eukaryotic systems)) (phosphoenol pyruvate and pyruvate kinase for bacterial systems), and other co-factors (Mg2+, K+, etc.). As previously mentioned, in vitro translation can be based on RNA or DNA starting material. Some translation systems can utilize an RNA template as starting material (e.g., reticulocyte lysates and wheat germ extracts). Some translation systems can utilize a DNA template as a starting material (e.g., E coli-based systems). In these systems transcription and translation are coupled and DNA is first transcribed into RNA, which is subsequently translated. Suitable standard and coupled cell-free translation systems are generally known in the art and are commercially available.

### Codon Optimization of Vector Polynucleotides

As described elsewhere herein, the polynucleotide encoding one or more engineered polynucleotides described herein can be codon optimized. In some embodiments, the vector includes polynucleotides that are in addition to the engineered polynucleotides of the present disclosure and are generally referred to herein as “vector polynucleotides”. In some embodiments, one or more vector polynucleotides are codon optimized. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database available at http://www.yeastgenome.org/community/codon_usage.shtml, or Codon selection in yeast, Bennetzen and Hall, J Biol Chem. 1982 Mar. 25; 257(6):3026⁻³¹. As to codon usage in plants including algae, reference is made to Codon usage in higher plants, green algae, and cyanobacteria, Campbell and Gowri, Plant Physiol. 1990 January; 92(1): 1-11.; as well as Codon usage in plant genes, Murray et al, Nucleic Acids Res. 1989 Jan. 25; 17(2):477-98; or Selection on the codon bias of chloroplast and cyanelle genes in different plant and algal lineages, Morton B R, J Mol Evol. 1998 April; 46(4): 449-59.

The vector polynucleotide(s) and/or engineered polynucleotide(s) can be codon optimized for expression in a specific cell-type, tissue type, organ type, and/or subject type. In some embodiments, a codon optimized sequence is a sequence optimized for expression in a eukaryote, e.g., humans (i.e., being optimized for expression in a human or human cell), or for another eukaryote, such as another animal (e.g., a mammal or avian) as is described elsewhere herein. Such codon optimized sequences are within the ambit of the ordinary skilled artisan in view of the description herein. In some embodiments, the polynucleotide is codon optimized for a specific cell type. Such cell types can include, but are not limited to, epithelial cells (including skin cells, cells lining the gastrointestinal tract, cells lining other hollow organs), nerve cells (nerves, brain cells, spinal column cells, nerve support cells (e.g., astrocytes, glial cells, Schwann cells etc.), muscle cells (e.g., cardiac muscle, smooth muscle cells, and skeletal muscle cells), connective tissue cells (fat and other soft tissue padding cells, bone cells, tendon cells, cartilage cells), blood cells, stem cells and other progenitor cells, immune system cells, germ cells, and combinations thereof. Such codon optimized sequences are within the ambit of the ordinary skilled artisan in view of the description herein. In some embodiments, the engineered polynucleotide is codon optimized for a specific tissue type. Such tissue types can include, but are not limited to, muscle tissue, connective tissue, connective tissue, nervous tissue, and epithelial tissue. Such codon optimized sequences are within the ambit of the ordinary skilled artisan in view of the description herein. In some embodiments, the polynucleotide is codon optimized for a specific organ. Such organs include, but are not limited to, muscles, skin, intestines, liver, spleen, brain, lungs, stomach, heart, kidneys, gallbladder, pancreas, bladder, thyroid, bone, blood vessels, blood, and combinations thereof. Such codon optimized sequences are within the ambit of the ordinary skilled artisan in view of the description herein.

In some embodiments, a vector and/or an engineered polynucleotide of the present disclosure is codon optimized for expression in particular cells, such as prokaryotic or eukaryotic cells. The eukaryotic cells may be those of or derived from a particular organism, such as a plant or a mammal, including but not limited to human, or non-human eukaryote or animal or mammal as discussed herein, e.g., mouse, rat, rabbit, dog, livestock, or non-human mammal or primate.

### Non-Viral Vectors and Carriers

In some embodiments, the vector is a non-viral vector or carrier. In some aspects, non-viral vectors can have the advantage(s) of reduced toxicity and/or immunogenicity and/or increased bio-safety as compared to viral vectors The terms of art “Non-viral vectors and carriers” and as used herein in this context refers to molecules and/or compositions that are not based on one or more component of a virus or virus genome (excluding any nucleotide to be delivered and/or expressed by the non-viral vector) that can be capable of attaching to, incorporating, coupling, and/or otherwise interacting with an engineered polynucleotide of the present disclosure and can be capable of ferrying the polynucleotide to a cell and/or expressing the polynucleotide. It will be appreciated that this does not exclude the inclusion of a virus-based polynucleotide that is to be delivered. Non-viral vectors and carriers include naked polynucleotides, chemical-based carriers, polynucleotide (non-viral) based vectors, and particle-based carriers. It will be appreciated that the term “vector” as used in the context of non-viral vectors and carriers refers to polynucleotide vectors and “carriers” used in this context refers to a non-nucleic acid, polynucleotide molecule, or composition that be attached to or otherwise interact with, encapsulate, and/or associate with a polynucleotide to be delivered, such as an engineered polynucleotide of the present disclosure.

### Naked Polynucleotides

In some embodiments, one or more engineered polynucleotides described elsewhere herein is/are included in a naked polynucleotide. The term of art “naked polynucleotide” as used herein refers to polynucleotides that are not associated with another molecule (e.g., proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the engineered polynucleotides described herein can be delivered directly to a host cell and optionally expressed therein. The naked polynucleotides can have any suitable two- and three-dimensional configurations. By way of non-limiting examples, naked polynucleotides can be single-stranded molecules, double stranded molecules, circular molecules (e.g., plasmids and artificial chromosomes), molecules that contain portions that are single stranded and portions that are double stranded (e.g., ribozymes), and the like. In some embodiments, the naked polynucleotide contains only the engineered polynucleotide(s) of the present invention. In some embodiments, the naked polynucleotide can contain other nucleic acids and/or polynucleotides in addition to the engineered polynucleotide(s) of the present invention. The naked polynucleotides can include one or more elements of a transposon system. Transposons and system thereof are described in greater detail elsewhere herein.

### Non-Viral Polynucleotide Vectors

In some embodiments, one or more of the engineered polynucleotides is/are included in a non-viral polynucleotide vector. Suitable non-viral polynucleotide vectors include, but are not limited to, transposon vectors and vector systems, plasmids, bacterial artificial chromosomes, yeast artificial chromosomes, AR (antibiotic resistance)-free plasmids and miniplasmids, circular covalently closed vectors (e.g., minicircles, minivectors, miniknots,), linear covalently closed vectors (“dumbbell shaped”), MIDGE (minimalistic immunologically defined gene expression) vectors, MiLV (micro-linear vector) vectors, Ministrings, mini-intronic plasmids, PSK systems (post-segregationally killing systems), ORT (operator repressor titration) plasmids, and the like. See e.g., Hardee et al. 2017. Genes. 8(2):65.

In some embodiments, the non-viral polynucleotide vector can have a conditional origin of replication. In some embodiments, the non-viral polynucleotide vector can be an ORT plasmid. In some embodiments, the non-viral polynucleotide vector can have a minimalistic immunologically defined gene expression. In some embodiments, the non-viral polynucleotide vector can have one or more post-segregationally killing system genes. In some embodiments, the non-viral polynucleotide vector is AR-free. In some embodiments, the non-viral polynucleotide vector is a minivector. In some embodiments, the non-viral polynucleotide vector includes a nuclear localization signal. In some embodiments, the non-viral polynucleotide vector can include one or more CpG motifs. In some embodiments, the non-viral polynucleotide vectors can include one or more scaffold/matrix attachment regions (S/MARs). See e.g., Mirkovitch et al. 1984. Cell. 39:223-232, Wong et al. 2015. Adv. Genet. 89:113-152, whose techniques and vectors can be adapted for use in the present invention. S/MARs are AT-rich sequences that play a role in the spatial organization of chromosomes through DNA loop base attachment to the nuclear matrix. S/MARs are often found close to regulatory elements such as promoters, enhancers, and origins of DNA replication. Inclusion of one or S/MARs can facilitate a once-per-cell-cycle replication to maintain the non-viral polynucleotide vector as an episome in daughter cells. In aspects, the S/MAR sequence is located downstream of an actively transcribed polynucleotide (e.g., one or more engineered polynucleotides of the present disclosure) included in the non-viral polynucleotide vector. In some embodiments, the S/MAR can be a S/MAR from the beta-interferon gene cluster. See e.g., Verghese et al. 2014. Nucleic Acid Res. 42:e53; Xu et al. 2016. Sci. China Life Sci. 59:1024-1033; Jin et al. 2016. 8:702-711; Koirala et al. 2014. Adv. Exp. Med. Biol. 801:703-709; and Nehlsen et al. 2006. Gene Ther. Mol. Biol. 10:233-244, whose techniques and vectors can be adapted for use in the present invention.

In some embodiments, the non-viral vector is a transposon vector or system thereof. As used herein, “transposon” (also referred to as transposable element) refers to a polynucleotide sequence that is capable of moving form location in a genome to another. There are several classes of transposons. Transposons include retrotransposons and DNA transposons. Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide. In some embodiments, the non-viral polynucleotide vector can be a retrotransposon vector. In some embodiments, the retrotransposon vector includes long terminal repeats. In some embodiments, the retrotransposon vector does not include long terminal repeats. In some embodiments, the non-viral polynucleotide vector can be a DNA transposon vector. DNA transposon vectors can include a polynucleotide sequence encoding a transposase. In some embodiments, the transposon vector is configured as a non-autonomous transposon vector, meaning that the transposition does not occur spontaneously on its own. In some of these aspects, the transposon vector lacks one or more polynucleotide sequences encoding proteins required for transposition. In some embodiments, the non-autonomous transposon vectors lack one or more Ac elements.

In some embodiments, a non-viral polynucleotide transposon vector system can include a first polynucleotide vector that contains the engineered polynucleotide(s) of the present disclosure flanked on the 5′ and 3′ ends by transposon terminal inverted repeats (TIRs) and a second polynucleotide vector that includes a polynucleotide capable of encoding a transposase coupled to a promoter to drive expression of the transposase. When both are expressed in the same cell the transposase can be expressed from the second vector and can transpose the material between the TIRs on the first vector (e.g., the engineered polynucleotide(s) of the present disclosure) and integrate it into one or more positions in the host cell's genome. In some embodiments, the transposon vector or system thereof can be configured as a gene trap. In some embodiments, the TIRs can be configured to flank a strong splice acceptor site followed by a reporter and/or other gene (e.g., one or more of the engineered polynucleotide(s) of the present invention) and a strong poly A tail. When transposition occurs while using this vector or system thereof, the transposon can insert into an intron of a gene and the inserted reporter or other gene can provoke a mis-splicing process and as a result it in activates the trapped gene.

Any suitable transposon system can be used. Suitable transposon and systems thereof include, but are not limited to, Sleeping Beauty transposon system (Tcl/mariner superfamily) (see e.g., Ivics et al. 1997. Cell. 91(4): 501-510), piggyBac (piggyBac superfamily) (see e.g., Li et al. 2013 110(25): E2279-E2287 and Yusa et al. 2011. PNAS. 108(4): 1531-1536), Tol2 (superfamily hAT), Frog Prince (Tc1/mariner superfamily) (see e.g., Miskey et al. 2003 Nucleic Acid Res. 31(23):6873-6881) and variants thereof.

### Chemical Carriers

In some embodiments, the engineered polynucleotide(s) is/are coupled to a chemical carrier. Chemical carriers that can be suitable for delivery of polynucleotides can be broadly classified into the following classes: (i) inorganic particles, (ii) lipid-based, (iii) polymer-based, and (iv) peptide based. They can be categorized as (1) those that can form condensed complexes with a polynucleotide (such as the engineered polynucleotide(s) of the present disclosure), (2) those capable of targeting specific cells, (3) those capable of increasing delivery of the polynucleotide (such as the engineered polynucleotide(s) of the present disclosure) to the nucleus or cytosol of a host cell, (4) those capable of disintegrating from DNA/RNA in the cytosol of a host cell, and (5) those capable of sustained or controlled release. It will be appreciated that any one given chemical carrier can include features from multiple categories. The term “particle” as used herein, refers to any suitable sized particles for delivery of the engineered polynucleotides and/or engineered polypeptides described herein. Suitable sizes include macro-, micro-, and nano-sized particles.

In some embodiments, the non-viral carrier can be an inorganic particle. In some embodiments, the inorganic particle, can be a nanoparticle. The inorganic particles can be configured and optimized by varying size, shape, and/or porosity. In some embodiments, the inorganic particles are optimized to escape from the reticulo endothelial system. In some aspects, the inorganic particles can be optimized to protect an entrapped molecule from degradation., the Suitable inorganic particles that can be used as non-viral carriers in this context can include, but are not limited to, calcium phosphate, silica, metals (e.g., gold, platinum, silver, palladium, rhodium, osmium, iridium, ruthenium, mercury, copper, rhenium, titanium, niobium, tantalum, and combinations thereof), magnetic compounds, particles, and materials, (e.g., supermagnetic iron oxide and magnetite), quantum dots, fullerenes (e.g., carbon nanoparticles, nanotubes, nanostrings, and the like), and combinations thereof. Other suitable inorganic non-viral carriers are discussed elsewhere herein.

In some embodiments, the non-viral carrier can be lipid-based. Suitable lipid-based carriers are also described in greater detail herein. In some embodiments, the lipid-based carrier includes a cationic lipid or an amphiphilic lipid that is capable of binding or otherwise interacting with a negative charge on the polynucleotide to be delivered (e.g., such as an engineered polynucleotides of the present disclosure). In some embodiments, chemical non-viral carrier systems can include a polynucleotide such as the engineered polynucleotide(s) of the present disclosure) and a lipid (such as a cationic lipid). These are also referred to in the art as lipoplexes. Other aspects of lipoplexes are described elsewhere herein. In some embodiments, the non-viral lipid-based carrier can be a lipid nano emulsion. Lipid nano emulsions can be formed by the dispersion of an immiscible liquid in another stabilized emulsifying agent and can have particles of about 200 nm that are composed of the lipid, water, and surfactant that can contain the polynucleotide to be delivered (e.g., the engineered polynucleotide(s) of the present disclosure). In some embodiments, the lipid-based non-viral carrier can be a solid lipid particle or nanoparticle.

In some embodiments, the non-viral carrier can be peptide-based. In some embodiments, the peptide-based non-viral carrier can include one or more cationic amino acids. In some embodiments, 35 to 40, 45, 50, 55, 60, 65, 70, 75, 80, 85, 90, 95, 99 or 100% of the amino acids are cationic. In some embodiments, peptide carriers can be used in conjunction with other types of carriers (e.g., polymer-based carriers and lipid-based carriers to functionalize these carriers). In some embodiments, the functionalization is targeting a host cell. Suitable polymers that can be included in the polymer-based non-viral carrier can include, but are not limited to, polyethylenimine (PEI), chitosan, poly (DL-lactide) (PLA), poly (DL-Lactide-co-glycoside) (PLGA), dendrimers (see e.g., US Pat. Pub. 2017/0079916 whose techniques and compositions can be adapted for use with the engineered polynucleotides of the present disclosure), polymethacrylate, and combinations thereof.

In some embodiments, the non-viral carrier can be configured to release an engineered polynucleotide that is associated with or attached to the non-viral carrier in response to an external stimulus, such as pH, temperature, osmolarity, concentration of a specific molecule or composition (e.g., calcium, NaCl, and the like), pressure and the like. In some embodiments, the non-viral carrier can be a particle that is configured includes one or more of the engineered polynucleotides described herein and an environmental triggering agent response element, and optimally a triggering agent. In some embodiments, the particle can include a polymer that can be selected from the group of polymethacrylates and polyacrylates. In some embodiments, the non-viral particle can include one or more aspects of the compositions microparticles described in US Pat. Pubs. 20150232883 and 20050123596, whose techniques and compositions can be adapted for use in the present disclosure.

In some embodiments, the non-viral carrier can be a polymer-based carrier. In some embodiments, the polymer is cationic or is predominantly cationic such that it can interact in a charge-dependent manner with the negatively charged polynucleotide to be delivered (such as the engineered polynucleotide(s) of the present disclosure). Polymer-based systems are described in greater detail elsewhere herein.

### Viral Vectors

In some embodiments, the vector is a viral vector. The term of art “viral vector” and as used herein in this context refers to polynucleotide based vectors that contain one or more elements from or based upon one or more elements of a virus that can be capable of expressing and packaging a polynucleotide, such as an engineered polynucleotide of the present disclosure, into a virus particle and producing said virus particle when used alone or with one or more other viral vectors (such as in a viral vector system). Viral vectors and systems thereof can be used for producing viral particles for delivery of and/or expression of one or more engineered polynucleotides described herein. The viral vector can be part of a viral vector system involving multiple vectors. In some embodiments, systems incorporating multiple viral vectors can increase the safety of these systems. Suitable viral vectors can include retroviral-based vectors, lentiviral-based vectors, adenoviral-based vectors, adeno associated vectors, helper-dependent adenoviral (HdAd) vectors, hybrid adenoviral vectors, herpes simplex virus-based vectors, poxvirus-based vectors, and Epstein-Barr virus-based vectors. Other aspects of viral vectors and viral particles produce therefrom are described elsewhere herein. In some embodiments, the viral vectors are configured to produce replication incompetent viral particles for improved safety of these systems.

### Retroviral and Lentiviral Vectors

Retroviral vectors can be composed of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Suitable retroviral vectors for the engineered polynucleotides can include those based upon murine leukemia virus (MuLV), gibbon ape leukemia virus (GaLV), Simian immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). Selection of a retroviral gene transfer system may therefore depend on the target tissue.

The tropism of a retrovirus can be altered by incorporating foreign envelope proteins, expanding the potential target population of target cells. Lentiviral vectors are retroviral vectors that are able to transduce or infect non-dividing cells and are described in greater detail elsewhere herein. A retrovirus can also be engineered to allow for conditional expression of the inserted transgene, such that only certain cell types are infected by the lentivirus.

Lentiviruses are complex retroviruses that have the ability to infect and express their genes in both mitotic and post-mitotic cells. Advantages of using a lentiviral approach can include the ability to transduce or infect non-dividing cells and their ability to typically produce high viral titers, which can increase efficiency or efficacy of production and delivery. Suitable lentiviral vectors include, but are not limited to, human immunodeficiency virus (HIV)-based lentiviral vectors, feline immunodeficiency virus (FIV)-based lentiviral vectors, simian immunodeficiency virus (SIV)-based lentiviral vectors, Moloney Murine Leukaemia Virus (Mo-MLV), Visna.maedi virus (VMV)-based lentiviral vector, carpine arthritis-encephalitis virus (CAEV)-based lentiviral vector, bovine immune deficiency virus (BIV)-based lentiviral vector, and Equine infectious anemia (EIAV)-based lentiviral vector. In some embodiments, an HIV-based lentiviral vector system can be used. In some embodiments, a FIV-based lentiviral vector system can be used.

In some embodiments, the lentiviral vector is an EIAV-based lentiviral vector or vector system. EIAV vectors have been used to mediate expression, packaging, and/or delivery in other contexts, such as for ocular gene therapy (see, e.g., Balagaan, J Gene Med 2006; 8: 275-285). In another embodiment, RetinoStat®, (see, e.g., Binley et al., HUMAN GENE THERAPY 23:980-991 (September 2012)), which describes RetinoStat®, an equine infectious anemia virus-based lentiviral gene therapy vector that expresses angiostatic proteins endostatin and angiostatin that is delivered via a subretinal injection for the treatment of the wet form of age-related macular degeneration. Any of these vectors described in these publications can be modified for the elements of the engineered polynucleotides described herein.

In some embodiments, the lentiviral vector or vector system thereof can be a first-generation lentiviral vector or vector system thereof. First-generation lentiviral vectors can contain a large portion of the lentivirus genome, including the gag and pol genes, other additional viral proteins (e.g., VSV-G) and other accessory genes (e.g., vif, vprm vpu, nef, and combinations thereof), regulatory genes (e.g., tat and/or rev) as well as the gene of interest between the LTRs. First generation lentiviral vectors can result in the production of virus particles that can be capable of replication in vivo, which may not be appropriate for some instances or applications.

In some embodiments, the lentiviral vector or vector system thereof can be a second-generation lentiviral vector or vector system thereof. Second-generation lentiviral vectors do not contain one or more accessory virulence factors and do not contain all components necessary for virus particle production on the same lentiviral vector. This can result in the production of a replication-incompetent virus particle and thus increase the safety of these systems over first-generation lentiviral vectors. In some embodiments, the second-generation vector lacks one or more accessory virulence factors (e.g., vif, vprm, vpu, nef, and combinations thereof). Unlike the first-generation lentiviral vectors, no single second generation lentiviral vector includes all features necessary to express and package a polynucleotide into a virus particle. In some embodiments, the envelope and packaging components are split between two different vectors with the gag, pol, rev, and tat genes being contained on one vector and the envelope protein (e.g., VSV-G) are contained on a second vector. The gene of interest, its promoter, and LTRs can be included on a third vector that can be used in conjunction with the other two vectors (packaging and envelope vectors) to generate a replication-incompetent virus particle.

In some embodiments, the lentiviral vector or vector system thereof can be a third-generation lentiviral vector or vector system thereof. Third-generation lentiviral vectors and vector systems thereof have increased safety over first- and second-generation lentiviral vectors and systems thereof because, for example, the various components of the viral genome are split between two or more different vectors but used together in vitro to make virus particles, they can lack the tat gene (when a constitutively active promoter is included up-stream of the LTRs), and they can include one or more deletions in the 3′LTR to create self-inactivating (SIN) vectors having disrupted promoter/enhancer activity of the LTR. In some aspects, a third-generation lentiviral vector system can include (i) a vector plasmid that contains the polynucleotide of interest and upstream promoter that are flanked by the 5′ and 3′ LTRs, which can optionally include one or more deletions present in one or both of the LTRs to render the vector self-inactivating; (ii) a “packaging vector(s)” that can contain one or more genes involved in packaging a polynucleotide into a virus particle that is produced by the system (e.g., gag, pol, and rev) and upstream regulatory sequences (e.g., promoter(s)) to drive expression of the features present on the packaging vector, and (iii) an “envelope vector” that contains one or more envelope protein genes and upstream promoters. In embodiments, the third-generation lentiviral vector system includes at least two packaging vectors, with the gag-pol being present on a different vector than the rev gene.

In some embodiments, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) can be used/and or adapted to the engineered polynucleotides of the present disclosure.

In some embodiments, the pseudotype and infectivity or tropisim of a lentivirus particle can be tuned by altering the type of envelope protein(s) included in the lentiviral vector or system thereof. As used herein, an “envelope protein” or “outer protein” means a protein exposed at the surface of a viral particle that is not a capsid protein. For example, envelope or outer proteins typically comprise proteins embedded in the envelope of the virus. In some embodiments, a lentiviral vector or vector system thereof can include a VSV-G envelope protein. VSV-G mediates viral attachment to an LDL receptor (LDLR) or an LDLR family member present on a host cell, which triggers endocytosis of the viral particle by the host cell. Because LDLR is expressed by a wide variety of cells, viral particles expressing the VSV-G envelope protein can infect or transduce a wide variety of cell types. Other suitable envelope proteins can be incorporated based on the host cell that a user desires to be infected by a virus particle produced from a lentiviral vector or system thereof described herein and can include, but are not limited to, feline endogenous virus envelope protein (RD114) (see e.g., Hanawa et al. Molec. Ther. 2002 5(3) 242-251), modified Sindbis virus envelope proteins (see e.g., Morizono et al. 2010. J. Virol. 84(14) 6923-6934; Morizono et al. 2001. J. Virol. 75:8016-8020; Morizono et al. 2009. J. Gene Med. 11:549-558; Morizono et al. 2006 Virology 355:71-81; Morizono et al J. Gene Med. 11:655-663, Morizono et al. 2005 Nat. Med. 11:346-352), baboon retroviral envelope protein (see e.g., Girard-Gagnepain et al. 2014. Blood. 124: 1221-1231); Tupaia paramyxovirus glycoproteins (see e.g., Enkirch T. et al., 2013. Gene Ther. 20:16-23); measles virus glycoproteins (see e.g., Funke et al. 2008. Molec. Ther. 16(8): 1427-1436), rabies virus envelope proteins, MLV envelope proteins, Ebola envelope proteins, baculovirus envelope proteins, filovirus envelope proteins, hepatitis E1 and E2 envelope proteins, gp41 and gp120 of HIV, hemagglutinin, neuraminidase, M2 proteins of influenza virus, and combinations thereof.

In some embodiments, the tropism of the resulting lentiviral particle can be tuned by incorporating cell targeting peptides into a lentiviral vector such that the cell targeting peptides are expressed on the surface of the resulting lentiviral particle. In some embodiments, a lentiviral vector can contain an envelope protein that is fused to a cell targeting protein (see e.g., Buchholz et al. 2015. Trends Biotechnol. 33:777-790; Bender et al. 2016. PLOS Pathog. 12(e1005461); and Friedrich et al. 2013. Mol. Ther. 2013. 21: 849-859.

In some embodiments, a split-intein-mediated approach to target lentiviral particles to a specific cell type can be used (see e.g., Chamoun-Emaneulli et al. 2015. Biotechnol. Bioeng. 112:2611-2617, Ramirez et al. 2013. Protein. Eng. Des. Sel. 26:215-233. In these aspects, a lentiviral vector can contain one half of a splicing-deficient variant of the naturally split intein from Nostoc punctiforme fused to a cell targeting peptide and the same or different lentiviral vector can contain the other half of the split intein fused to an envelope protein, such as a binding-deficient, fusion-competent virus envelope protein. This can result in production of a virus particle from the lentiviral vector or vector system that includes a split intein that can function as a molecular Velcro linker to link the cell-binding protein to the pseudotyped lentivirus particle. This approach can be advantageous for use where surface-incompatibilities can restrict the use of, e.g., cell targeting peptides.

In some embodiments, a covalent-bond-forming protein-peptide pair can be incorporated into one or more of the lentiviral vectors described herein to conjugate a cell targeting peptide to the virus particle (see e.g., Kasaraneni et al. 2018. Sci. Reports (8) No. 10990). In some embodiments, a lentiviral vector can include an N-terminal PDZ domain of InaD protein (PDZ1) and its pentapeptide ligand (TEFCA) from NorpA, which can conjugate the cell targeting peptide to the virus particle via a covalent bond (e.g., a disulfide bond). In some embodiments, the PDZ1 protein can be fused to an envelope protein, which can optionally be binding deficient and/or fusion competent virus envelope protein and included in a lentiviral vector. In some embodiments, the TEFCA can be fused to a cell targeting peptide and the TEFCA-CPT fusion construct can be incorporated into the same or a different lentiviral vector as the PDZ1-envelope protein construct. During virus production, specific interaction between the PDZ1 and TEFCA facilitates producing virus particles covalently functionalized with the cell targeting peptide and thus capable of targeting a specific cell-type based upon a specific interaction between the cell targeting peptide and cells expressing its binding partner. This approach can be advantageous for use where surface-incompatibilities can restrict the use of, e.g., cell targeting peptides.

Lentiviral vectors have been disclosed as in the treatment for Parkinson's Disease, see, e.g., US Patent Publication No. 20120295960 and U.S. Pat. Nos. 7,303,910 and 7,351,585. Lentiviral vectors have also been disclosed for the treatment of ocular diseases, see e.g., US Patent Publication Nos. 20060281180, 20090007284, US20110117189; US20090017543; US20070054961, US20,100,317109. Lentiviral vectors have also been disclosed for delivery to the brain, see, e.g., US Patent Publication Nos. US20110293571; US20110293571, US20040013648, US20070025970, US20090111106 and U.S. Pat. No. 7,259,015. Any of these systems or a variant thereof can be used to deliver an engineered polynucleotide described herein to a cell.

In some embodiments, a lentiviral vector system can include one or more transfer plasmids. Transfer plasmids can be generated from various other vector backbones and can include one or more features that can work with other retroviral and/or lentiviral vectors in the system that can, for example, improve safety of the vector and/or vector system, increase virial titers, and/or increase or otherwise enhance expression of the desired insert to be expressed and/or packaged into the viral particle. Suitable features that can be included in a transfer plasmid can include, but are not limited to, 5′LTR, 3′LTR, SIN/LTR, origin of replication (Ori), selectable marker genes (e.g., antibiotic resistance genes), Psi (Ψ), RRE (rev response element), cPPT (central polypurine tract), promoters, WPRE (woodchuck hepatitis post-transcriptional regulatory element), SV40 polyadenylation signal, pUC origin, SV40 origin, F1 origin, and combinations thereof.

### Adenoviral Vectors, Helper-Dependent Adenoviral Vectors, and Hybrid Adenoviral Vectors

In some embodiments, the vector can be an adenoviral vector. In some embodiments, the adenoviral vector can include elements such that the virus particle produced using the vector or system thereof can be serotype 2 or serotype 5. In some embodiments, the polynucleotide to be delivered via the adenoviral particle can be up to about 8 kb. Thus, in some embodiments, an adenoviral vector can include a DNA polynucleotide to be delivered that can range in size from about 0.001 kb to about 8 kb. Adenoviral vectors have been used successfully in several contexts (see e.g., Teramato et al. 2000. Lancet. 355:1911-1912; Lai et al. 2002. DNA Cell. Biol. 21:895-913; Flotte et al., 1996. Hum. Gene. Ther. 7:1145-1159; and Kay et al. 2000. Nat. Genet. 24:257-261.

In some embodiments, the vector can be a helper-dependent adenoviral vector or system thereof. These are also referred to in the art as “gutless” or “gutted” vectors and are a modified generation of adenoviral vectors (see e.g., Thrasher et al. 2006. Nature. 443:E5-7). In aspects of the helper-dependent adenoviral vector system one vector (the helper) can contain all the viral genes required for replication but contains a conditional gene defect in the packaging domain. The second vector of the system can contain only the ends of the viral genome, one or more engineered polynucleotides, and the native packaging recognition signal, which can allow selective packaged release from the cells (see e.g., Cideciyan et al. 2009. N Engl J Med. 361:725-727). Helper-dependent adenoviral vector systems have been successful for gene delivery in several contexts (see e.g., Simonelli et al. 2010. J Am Soc Gene Ther. 18:643-650; Cideciyan et al. 2009. N Engl J Med. 361:725-727; Crane et al. 2012. Gene Ther. 19(4): 443-452; Alba et al. 2005. Gene Ther. 12:18-S27; Croyle et al. 2005. Gene Ther. 12:579-587; Amalfitano et al. 1998. J. Virol. 72:926-933; and Morral et al. 1999. PNAS. 96:12816-12821). The techniques and vectors described in these publications can be adapted for inclusion and delivery of the engineered polynucleotides described herein. In some embodiments, the polynucleotide to be delivered via the viral particle produced from a helper-dependent adenoviral vector or system thereof can be up to about 37 kb. Thus, in some embodiments, an adenoviral vector can include a DNA polynucleotide to be delivered that can range in size from about 0.001 kb to about 37 kb (see e.g., Rosewell et al. 2011. J. Genet. Syndr. Gene Ther. Suppl. 5:001).

In some embodiments, the vector is a hybrid-adenoviral vector or system thereof. Hybrid adenoviral vectors are composed of the high transduction efficiency of a gene-deleted adenoviral vector and the long-term genome-integrating potential of adeno-associated, retroviruses, lentivirus, and transposon based-gene transfer. In some embodiments, such hybrid vector systems can result in stable transduction and limited integration site. See e.g., Balague et al. 2000. Blood. 95:820-828; Morral et al. 1998. Hum. Gene Ther. 9:2709-2716; Kubo and Mitani. 2003. J. Virol. 77(5): 2964-2971; Zhang et al. 2013. PloS One. 8(10) e76771; and Cooney et al. 2015. Mol. Ther. 23(4):667-674), whose techniques and vectors described therein can be modified and adapted for use in with the engineered polynucleotides of the present disclosure. In some embodiments, a hybrid-adenoviral vector can include one or more features of a retrovirus and/or an adeno-associated virus. In some embodiments, the hybrid-adenoviral vector can include one or more features of a spuma retrovirus or foamy virus (FV). See e.g., Ehrhardt et al. 2007. Mol. Ther. 15:146-156 and Liu et al. 2007. Mol. Ther. 15:1834-1841, whose techniques and vectors described therein can be modified and adapted for use with the engineered polynucleotides of the present disclosure. Advantages of using one or more features from the FVs in the hybrid-adenoviral vector or system thereof can include the ability of the viral particles produced therefrom to infect a broad range of cells, a large packaging capacity as compared to other retroviruses, and the ability to persist in quiescent (non-dividing) cells. See also e.g., Ehrhardt et al. 2007. Mol. Ther. 156:146-156 and Shuji et al. 2011. Mol. Ther. 19:76-82, whose techniques and vectors described therein can be modified and adapted for use with the engineered polynucleotides of the present invention.

### Adeno Associated Viral (AAV) Vectors

In an embodiment, the vector can be an adeno-associated virus (AAV) vector. See, e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). Although similar to adenoviral vectors in some of their features, AAVs have some deficiency in their replication and/or pathogenicity and thus can be safer that adenoviral vectors. In some embodiments, the AAV can integrate into a specific site on chromosome 19 of a human cell with no observable side effects. In some embodiments, the capacity of the AAV vector, system thereof, and/or AAV particles can be up to about 4.7 kb.

The AAV vector or system thereof can include one or more regulatory molecules. In some embodiments, the regulatory molecules can be promoters, enhancers, repressors and the like, which are described in greater detail elsewhere herein. In some embodiments, the AAV vector or system thereof can include one or more polynucleotides that can encode one or more regulatory proteins. In some embodiments, the one or more regulatory proteins can be selected from Rep78, Rep68, Rep52, Rep40, variants thereof, and combinations thereof.

The AAV vector or system thereof can include one or more polynucleotides that can encode one or more capsid proteins. The capsid proteins can be selected from VP1, VP2, VP3, and combinations thereof. The capsid proteins can be capable of assembling into a protein shell of the AAV virus particle. In some embodiments, the AAV capsid can contain 60 capsid proteins. In some embodiments, the ratio of VP1:VP2:VP3 in a capsid can be about 1:1:10.

In some embodiments, the AAV vector or system thereof can include one or more adenovirus helper factors or polynucleotides that can encode one or more adenovirus helper factors. Such adenovirus helper factors can include, but are not limited, E1A, E1B, E2A, E4ORF6, and VA RNAs. In some embodiments, a producing host cell line expresses one or more of the adenovirus helper factors.

The AAV vector or system thereof can be configured to produce AAV particles having a specific serotype. In some embodiments, the serotype can be AAV-1, AAV-2, AAV-3, AAV-4, AAV-5, AAV-6, AAV-8, AAV-9 or any combinations thereof. In some embodiments, the AAV can be AAV1, AAV-2, AAV-5 or any combination thereof. One can select the AAV of the AAV with regard to the cells to be targeted; e.g., one can select AAV serotypes 1, 2, 5 or a hybrid capsid AAV-1, AAV-2, AAV-5 or any combination thereof for targeting brain and/or neuronal cells; and one can select AAV-4 for targeting cardiac tissue; and one can select AAV8 for delivery to the liver. Thus, in some embodiments, an AAV vector or system thereof capable of producing AAV particles capable of targeting the brain and/or neuronal cells can be configured to generate AAV particles having serotypes 1, 2, 5 or a hybrid capsid AAV-1, AAV-2, AAV-5 or any combination thereof. In some embodiments, an AAV vector or system thereof capable of producing AAV particles capable of targeting cardiac tissue can be configured to generate an AAV particle having an AAV-4 serotype. In some embodiments, an AAV vector or system thereof capable of producing AAV particles capable of targeting the liver can be configured to generate an AAV having an AAV-8 serotype. In some embodiments, the AAV vector is a hybrid AAV vector or system thereof. Hybrid AAVs are AAVs that include genomes with elements from one serotype that are packaged into a capsid derived from at least one different serotype. For example, if it is the rAAV2/5 that is to be produced, and if the production method is based on the helper-free, transient transfection method discussed above, the 1st plasmid and the 3rd plasmid (the adeno helper plasmid) will be the same as discussed for rAAV2 production. However, the 2nd plasmid, the pRepCap will be different. In this plasmid, called pRep2/Cap5, the Rep gene is still derived from AAV2, while the Cap gene is derived from AAV5. The production scheme is the same as the above-mentioned approach for AAV2 production. The resulting rAAV is called rAAV2/5, in which the genome is based on recombinant AAV2, while the capsid is based on AAV5. It is assumed the cell or tissue-tropism displayed by this AAV2/5 hybrid virus should be the same as that of AAV5.

A tabulation of certain AAV serotypes as to these cells can be found in Grimm, D. et al, J. Virol. 82: 5887-5911 (2008).

In some embodiments, the AAV vector or system thereof is configured as a “gutless” vector, similar to that described in connection with a retroviral vector. In some embodiments, the “gutless” AAV vector or system thereof can have the cis-acting viral DNA elements involved in genome amplification and packaging in linkage with the heterologous sequences of interest (e.g., the engineered polynucleotide(s) of the present disclosure).

### Herpes Simplex Viral Vectors

In some embodiments, the vector can be a Herpes Simplex Viral (HSV)-based vector or system thereof. HSV systems can include the disabled infections single copy (DISC) viruses, which are composed of a glycoprotein H defective mutant HSV genome. When the defective HSV is propagated in complementing cells, virus particles can be generated that are capable of infecting subsequent cells permanently replicating their own genome but are not capable of producing more infectious particles. See e.g., 2009. Trobridge. Exp. Opin. Biol. Ther. 9:1427-1436, whose techniques and vectors described therein can be modified and adapted for use with the engineered polynucleotides of the present disclosure. In some embodiments, where an HSV vector or system thereof is utilized, the host cell can be a complementing cell. In some embodiments, HSV vector or system thereof can be capable of producing virus particles capable of delivering a polynucleotide cargo of up to 150 kb. Thus, in some aspect the engineered polynucleotide(s) included in the HSV-based viral vector or system thereof can sum from about 0.001 to about 150 kb. HSV-based vectors and systems thereof have been successfully used in several contexts including various models of neurologic disorders. See e.g., Cockrell et al. 2007. Mol. Biotechnol. 36:184-204; Kafri T. 2004. Mol. Biol. 246:367-390; Balaggan and Ali. 2012. Gene Ther. 19:145-153; Wong et al. 2006. Hum. Gen. Ther. 2002. 17:1-9; Azzouz et al. J. Neruosci. 22L10302-10312; and Betchen and Kaplitt. 2003. Curr. Opin. Neurol. 16:487-493, whose techniques and vectors described therein can be modified and adapted for use with the engineered polynucleotides of the present disclosure.

### Poxvirus Vectors

In some embodiments, the vector can be a poxvirus vector or system thereof. In some embodiments, the poxvirus vector can result in cytoplasmic expression of one or more engineered polynucleotides of the present invention. In some embodiments, the capacity of a poxvirus vector or system thereof can be about 25 kb or more. In some embodiments, a poxvirus vector or system thereof can include a one or more engineered polynucleotides of the present disclosure.

### Vector Construction

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. Application publication No. US 2004-0171156 A1. Other suitable methods and techniques are described elsewhere herein.

Construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vector described herein. nAAV vectors are discussed elsewhere herein.

In some embodiments, the vector can have one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors.

Delivery vehicles, vectors, particles, nanoparticles, formulations and components thereof for expression of one or more engineered polynucleotides described herein are as used in the foregoing documents, such as WO 2014/093622 (PCT/US2013/074667) and are discussed in greater detail herein.

Virus Particle Production from Viral Vectors

### Retroviral Production

In some embodiments, one or more viral vectors and/or system thereof can be delivered to a suitable cell line for production of virus particles containing the polynucleotide or other payload (e.g., one or more engineered polynucleotides of the present disclosure) to be delivered to a host cell. Suitable host cells for virus production from viral vectors and systems thereof described herein are known in the art and are commercially available. For example, suitable host cells include HEK 293 cells and its variants (HEK 293T and HEK 293TN cells). In some embodiments, the suitable host cell for virus production from viral vectors and systems thereof described herein can stably express one or more genes involved in packaging (e.g., pol, gag, and/or VSV-G) and/or other supporting genes.

In some embodiments, after delivery of one or more viral vectors to the suitable host cells for or virus production from viral vectors and systems thereof, the cells are incubated for an appropriate length of time to allow for viral gene expression from the vectors, packaging of the polynucleotide to be delivered (e.g., an engineered polynucleotide of the present disclosure), and virus particle assembly, and secretion of mature virus particles into the culture media. Various other methods and techniques are generally known to those of ordinary skill in the art.

Mature virus particles can be collected from the culture media by a suitable method. In some embodiments, this can involve centrifugation to concentrate the virus. The titer of the composition containing the collected virus particles can be obtained using a suitable method. Such methods can include transducing a suitable cell line (e.g., NIH 3T3 cells) and determining transduction efficiency, infectivity in that cell line by a suitable method. Suitable methods include PCR-based methods, flow cytometry, and antibiotic selection-based methods. Various other methods and techniques are generally known to those of ordinary skill in the art. The concentration of virus particle can be adjusted as needed. In some embodiments, the resulting composition containing virus particles can contain 1×10¹-1×10²⁰ particles/mL.

### AAV Particle Production

There are two main strategies for producing AAV particles from AAV vectors and systems thereof, such as those described herein, which depend on how the adenovirus helper factors are provided (helper v. helper free). In some embodiments, a method of producing AAV particles from AAV vectors and systems thereof can include adenovirus infection into cell lines that stably harbor AAV replication and capsid encoding polynucleotides along with AAV vector containing the polynucleotide to be packaged and delivered by the resulting AAV particle (e.g., the engineered polynucleotide(s)). In some embodiments, a method of producing AAV particles from AAV vectors and systems thereof can be a “helper free” method, which includes co-transfection of an appropriate producing cell line with three vectors (e.g., plasmid vectors): (1) an AAV vector that contains a polynucleotide of interest (e.g., the engineered polynucleotide(s)) between 2 ITRs; (2) a vector that carries the AAV Rep-Cap encoding polynucleotides; and (helper polynucleotides. One of skill in the art will appreciate various methods and variations thereof that are both helper and -helper free and as well as the different advantages of each system.

### Vector and Virus Particle Delivery

A vector (including non-viral carriers) described herein can be introduced into host cells to thereby produce transcripts, proteins, or peptides, including fusion proteins or peptides encoded by nucleic acids as described herein (e.g., engineered polynucleotides, proteins, etc.), and virus particles (such as from viral vectors and systems thereof).

One or more engineered polynucleotides can be delivered using adeno associated virus (AAV), lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For examples, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus.

For plasmid delivery, the route of administration, formulation and dose can be as in U.S. Pat. No. 5,846,946 and as in clinical studies involving plasmids. In some embodiments, doses can be based on or extrapolated to an average 70 kg individual (e.g., a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into or otherwise delivered to the tissue or cell of interest.

In terms of in vivo delivery, AAV is advantageous over other viral vectors for a couple of reasons such as low toxicity (this may be due to the purification method not requiring ultra-centrifugation of cell particles that can activate the immune response) and a low probability of causing insertional mutagenesis because it doesn't integrate into the host genome.

The vector(s) and virus particles described herein can be delivered into a host cell in vitro, in vivo, and or ex vivo. Delivery can occur by any suitable method including, but not limited to, physical methods, chemical methods, and biological methods. Physical delivery methods are those methods that employ physical force to counteract the membrane barrier of the cells to facilitate intracellular delivery of the vector. Suitable physical methods include, but are not limited to, needles (e.g., injections), ballistic polynucleotides (e.g., particle bombardment, micro projectile gene transfer, and gene gun), electroporation, sonoporation, photoporation, magnetofection, hydroporation, and mechanical massage. Chemical methods are those methods that employ a chemical to elicit a change in the cells membrane permeability or other characteristic(s) to facilitate entry of the vector into the cell. For example, the environmental pH can be altered which can elicit a change in the permeability of the cell membrane. Biological methods are those that rely and capitalize on the host cell's biological processes or biological characteristics to facilitate transport of the vector (with or without a carrier) into a cell. For example, the vector and/or its carrier can stimulate an endocytosis or similar process in the cell to facilitate uptake of the vector into the cell.

Delivery of the engineered polynucleotides to cells can be via particles. In some embodiments, any of the of the engineered polynucleotides can be attached to, coupled to, integrated with, otherwise associated with one or more particles or component thereof as described herein. The particles described herein can then be administered to a cell or organism by an appropriate route and/or technique. In some embodiments, particle delivery can be selected and be advantageous for delivery of the polynucleotide or vector components. It will be appreciated that in embodiments, particle delivery can also be advantageous for other engineered biomolecules and described elsewhere herein.

### Delivery and Delivery Vehicles

The present disclosure also provides delivery systems/vehicles (used interchangeably in this context herein) for introducing components of the engineered biomolecules and/or vectors described herein to cells, tissues, organs, or organisms. A delivery system may comprise one or more delivery vehicles and/or cargos.

In some embodiments, the delivery systems may be used to introduce the components of the systems and compositions to plant cells. For example, the components may be delivered to plant using electroporation, microinjection, aerosol beam injection of plant cell protoplasts, biolistic methods, DNA particle bombardment, and/or Agrobacterium-mediated transformation. Examples of methods and delivery systems for plants include those described in Fu et al., Transgenic Res. 2000 February; 9(1):11-9; Klein R M, et al., Biotechnology. 1992; 24:384-6; Casas A M et al., Proc Natl Acad Sci USA. 1993 Dec. 1; 90(23): 11212-11216; and U.S. Pat. No. 5,563,055, Davey M R et al., Plant Mol Biol. 1989 September; 13(3):273-85, which are incorporated by reference herein in their entireties.

### Cargos

The delivery vehicle can include one or more cargos. In some embodiments, the one or more cargos are one or more engineered biomolecules, vectors, vector systems, co-therapeutic or co-therapy, or any combination thereof of the present disclosure as is described elsewhere herein.

### Physical Delivery

In some embodiments, the cargos may be introduced to cells by physical delivery methods. Examples of physical methods include microinjection, electroporation, and hydrodynamic delivery. Both nucleic acids and proteins may be delivered using such methods. For example, an engineered polypeptide of the present disclosure may be prepared in vitro, isolated, (refolded, purified if needed), and introduced to a cell or cell population.

### Microinjection

Microinjection of the cargo directly to cells can achieve high efficiency, e.g., above 90% or about 100%. In some embodiments, microinjection may be performed using a microscope and a needle (e.g., with 0.5-5.0 μm in diameter) to pierce a cell membrane and deliver the cargo directly to a target site within the cell. Microinjection may be used for in vitro and ex vivo delivery.

Any of the engineered biomolecules, vectors, vector systems, etc. of the present disclosure may be microinjected. In some cases, microinjection may be used i) to deliver DNA directly to a cell nucleus, ii) to deliver mRNA (e.g., in vitro transcribed mRNA) to a cell nucleus or cytoplasm, and/or iii) delivery an engineered polypeptide of the present disclosure to a nucleus, cytoplasm, and/or any compartment thereof of a cell. In certain examples, microinjection may be used to deliver an engineered DNA, engineered mRNA and/or engineered polypeptide directly to the cytoplasm. In some embodiments, delivery of the DNA, engineered mRNA and/or engineered polypeptide is to the nucleus.

Microinjection may be in vitro, ex vivo, or in vivo.

### Electroporation

In some embodiments, the cargos and/or delivery vehicles may be delivered by electroporation. Electroporation may use pulsed high-voltage electrical currents to transiently open nanometer-sized pores within the cellular membrane of cells suspended in buffer, allowing for components with hydrodynamic diameters of tens of nanometers to flow into the cell. In some cases, electroporation may be used on various cell types and efficiently transfer cargo into cells. Electroporation may be used for in vitro and ex vivo delivery.

Electroporation may also be used to deliver the cargo to into the nuclei of mammalian cells by applying specific voltage and reagents, e.g., by nucleofection. Such approaches include those described in Wu Y, et al. (2015). Cell Res 25:67-79; Ye L, et al. (2014). Proc Natl Acad Sci USA 111:9591-6; Choi P S, Meyerson M. (2014). Nat Commun 5:3728; Wang J, Quake S R. (2014). Proc Natl Acad Sci 111:13157-62. Electroporation may also be used to deliver the cargo in vivo, e.g., with methods described in Zuckermann M, et al. (2015). Nat Commun 6:7391.

### Hydrodynamic Delivery

Hydrodynamic delivery may also be used for delivering the cargos, e.g., for in vivo delivery. In some examples, hydrodynamic delivery may be performed by rapidly pushing a large volume (8-10% body weight) solution containing the gene editing cargo into the bloodstream of a subject (e.g., an animal or human), e.g., for mice, via the tail vein. As blood is incompressible, the large bolus of liquid may result in an increase in hydrodynamic pressure that temporarily enhances permeability into endothelial and parenchymal cells, allowing for cargo not normally capable of crossing a cellular membrane to pass into cells. This approach may be used for delivering naked DNA plasmids and proteins. The delivered cargos may be enriched in liver, kidney, lung, muscle, and/or heart.

### Transfection

The cargos, e.g., nucleic acids and/or polypeptides, may be introduced to cells by transfection methods for introducing nucleic acids into cells. Examples of transfection methods include calcium phosphate-mediated transfection, cationic transfection, liposome transfection, dendrimer transfection, heat shock transfection, magnetofection, lipofection, impalefection, optical transfection, proprietary agent-enhanced uptake of nucleic acid.

### Transduction

The cargos, e.g., nucleic acids and/or polypeptides, can be introduced to cells by transduction by a viral or pseudoviral particle. Methods of packaging the cargos in viral particles can be accomplished using any suitable viral vector or vector systems. Such viral vector and vector systems are described in greater detail elsewhere herein. As used in this context herein “transduction” refers to the process by which foreign nucleic acids and/or proteins are introduced to a cell (prokaryote or eukaryote) by a viral or pseudo viral particle. After packaging in a viral particle or pseudo viral particle, the viral particles can be exposed to cells (e.g., in vitro, ex vivo, or in vivo) where the viral or pseudoviral particle infects the cell and delivers the cargo to the cell via transduction. Viral and pseudoviral particles can be optionally concentrated prior to exposure to target cells. In some embodiments, the virus titer of a composition containing viral and/or pseudoviral particles can be obtained and a specific titer be used to transduce cells. Viral vectors and viral particle production are described in greater detail elsewhere herein.

### Biolistics

The cargos, e.g., nucleic acids and/or polypeptides, can be introduced to cells using a biolistic method or technique. The term of art “biolistic”, as used herein refers to the delivery of nucleic acids to cells by high-speed particle bombardment. In some embodiments, the cargo(s) can be attached, associated with, or otherwise coupled to particles, which than can be delivered to the cell via a gene-gun (see e.g., Liang et al. 2018. Nat. Protocol. 13:413-430; Svitashev et al. 2016. Nat. Comm. 7:13274; Ortega-Escalante et al., 2019. Plant. J. 97:661-672). In some embodiments, the particles can be gold, tungsten, palladium, rhodium, platinum, or iridium particles.

### Implantable Devices

In some embodiments, the delivery system can include an implantable device that incorporates, contains, and/or is coated with an engineered biomolecule, vector, vector system, formulation thereof, etc. of the present disclosure as described elsewhere herein. Various implantable devices are described in the art, and include any device, graft, or other composition that can be implanted into a subject. When inserted, the implantable device can release or otherwise elute the engineered biomolecules vector, vector system, formulation thereof, etc. of the present disclosure into the subject to deliver said molecules, vectors, vector systems, formulations, etc. of the present disclosure to the subject or cell(s).

### Delivery Vehicles

The delivery systems may comprise one or more delivery vehicles. The delivery vehicles may deliver the cargo into cells, tissues, organs, or organisms (e.g., animals or plants). The cargos may be packaged, carried, or otherwise associated with the delivery vehicles. The delivery vehicles may be selected based on the types of cargo to be delivered, and/or the delivery is in vitro and/or in vivo. Examples of delivery vehicles include vectors, viruses (e.g., virus particles), non-viral vehicles, and other delivery reagents described herein.

The delivery vehicles in accordance with the present invention may a greatest dimension (e.g., diameter) of less than 100 microns (μm). In some embodiments, the delivery vehicles have a greatest dimension of less than 10 μm. In some embodiments, the delivery vehicles may have a greatest dimension of less than 2000 nanometers (nm). In some embodiments, the delivery vehicles may have a greatest dimension of less than 1000 nanometers (nm). In some embodiments, the delivery vehicles may have a greatest dimension (e.g., diameter) of less than 900 nm, less than 800 nm, less than 700 nm, less than 600 nm, less than 500 nm, less than 400 nm, less than 300 nm, less than 200 nm, less than 150 nm, or less than 100 nm, less than 50 nm. In some embodiments, the delivery vehicles may have a greatest dimension ranging between 25 nm and 200 nm.

In some embodiments, the delivery vehicles may be or comprise particles. For example, the delivery vehicle may be or comprise nanoparticles (e.g., particles with a greatest dimension (e.g., diameter) no greater than 1000 nm. The particles may be provided in different forms, e.g., as solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles).

Nanoparticles may also be used to deliver the compositions and systems to plant cells, e.g., as described in WO 2008042156, US 20130185823, and WO2015089419. In general, a “nanoparticle” refers to any particle having a diameter of less than 1000 nm. In certain preferred embodiments, nanoparticles of the invention have a greatest dimension (e.g., diameter) of 500 nm or less. In other preferred embodiments, nanoparticles of the invention have a greatest dimension ranging between 25 nm and 200 nm. In other preferred embodiments, nanoparticles of the invention have a greatest dimension of 100 nm or less. In other preferred embodiments, nanoparticles of the invention have a greatest dimension ranging between 35 nm and 60 nm. It will be appreciated that reference made herein to particles or nanoparticles can be interchangeable, where appropriate. Nanoparticles made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present invention. Semi-solid and soft nanoparticles have been manufactured, and are within the scope of the present invention. Nanoparticles with one half hydrophilic and the other half hydrophobic are termed Janus particles and are particularly effective for stabilizing emulsions. They can self-assemble at water/oil interfaces and act as solid surfactants.

Particle characterization (including e.g., characterizing morphology, dimension, etc.) is done using a variety of different techniques. Common techniques are electron microscopy (TEM, SEM), atomic force microscopy (AFM), dynamic light scattering (DLS), X-ray photoelectron spectroscopy (XPS), powder X-ray diffraction (XRD), Fourier transform infrared spectroscopy (FTIR), matrix-assisted laser desorption/ionization time-of-flight mass spectrometry (MALDI-TOF), ultraviolet-visible spectroscopy, dual polarization interferometry and nuclear magnetic resonance (NMR). Characterization (dimension measurements) may be made as to native particles (i.e., preloading) or after loading of the cargo (in this context cargo refers to e.g., one or more engineered biomolecules of the present disclosure, vectors, vectors systems, formulations, co-therapies, and the like of the present disclosure or any combination thereof), and may include additional carriers and/or excipients to provide particles of an optimal size for delivery for any in vitro, ex vivo and/or in vivo application of the present disclosure. In certain preferred embodiments, particle dimension (e.g., diameter) characterization is based on measurements using dynamic laser scattering (DLS). Mention is made of U.S. Pat. Nos. 8,709,843; 6,007,845; 5,855,913; 5,985,309; 5,543,158; and the publication by James E. Dahlman and Carmen Barnes et al. Nature Nanotechnology (2014) published online 11 May 2014, doi: 10.1038/nnano.2014.84, describing particles, methods of making and using them and measurements thereof.

### Vectors and Vector Systems

Vectors and vector systems containing and/or capable of expression one or more engineered polynucleotides of the present disclosure are described in greater detail elsewhere herein. As will be appreciated, such vectors and vector systems are suitable delivery vehicles for the engineered biomolecules of the present disclosure described herein.

### Non-Vector Delivery Vehicles

The delivery vehicles may comprise non-viral vehicles. In general, methods and vehicles capable of delivering nucleic acids and/or protein (e.g., engineered biomolecules of the present disclosure) may be used for delivering the systems compositions herein. Examples of non-viral vehicles include lipid nanoparticles, cell-penetrating peptides (CPPs), DNA nanoclews, metal nanoparticles, streptolysin O, multifunctional envelope-type nanodevices (MENDs), lipid-coated mesoporous silica particles, and other inorganic nanoparticles.

### Lipid Particles

The delivery vehicles may comprise lipid particles, e.g., lipid nanoparticles (LNPs) and liposomes. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, International Patent Publication Nos. WO 91/17424 and WO 91/16024. The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

### Lipid Nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations.

In some examples. LNPs may be used for delivering DNA and/or RNA molecules (e.g., engineered polynucleotides of the present disclosure).

Components in LNPs may comprise cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), R-3-[(ro-methoxy-poly(ethylene glycol)2000) carbamoyl]-1,2-dimyristyloxlpropyl-3-amine (PEG-C-DOMG, and any combination thereof. Preparation of LNPs and encapsulation may be adapted from Rosin et al, Molecular Therapy, vol. 19, no. 12, pages 1286-220 Dec. 2011).

In some embodiments, an LNP delivery vehicle can be used to deliver a virus particle containing an engineered polynucleotide of the present disclosure. In some embodiments, the virus particle(s) can be adsorbed to the lipid particle, such as through electrostatic interactions, and/or can be attached to the liposomes via a linker.

In some embodiments, the LNP contains a nucleic acid, wherein the charge ratio of nucleic acid backbone phosphates to cationic lipid nitrogen atoms is about 1: 1.5-7 or about 1:4.

In some embodiments, the LNP also includes a shielding compound, which is removable from the lipid composition under in vivo conditions. In some embodiments, the shielding compound is a biologically inert compound. In some embodiments, the shielding compound does not carry any charge on its surface or on the molecule as such. In some embodiments, the shielding compounds are polyethylenglycoles (PEGs), hydroxyethylglucose (HEG) based polymers, polyhydroxyethyl starch (polyHES) and polypropylene. In some embodiments, the PEG, HEG, polyHES, and a polypropylene weight between about 500 to 10,000 Da or between about 2000 to 5000 Da. In some embodiments, the shielding compound is PEG2000 or PEG5000.

In some embodiments, the LNP can include one or more helper lipids. In some embodiments, the helper lipid can be a phosphor lipid or a steroid. In some embodiments, the helper lipid is between about 20 mol % to 80 mol % of the total lipid content of the composition. In some embodiments, the helper lipid component is between about 35 mol % to 65 mol % of the total lipid content of the LNP. In some embodiments, the LNP includes lipids at 50 mol % and the helper lipid at 50 mol % of the total lipid content of the LNP.

Other non-limiting, exemplary LNP delivery vehicles are described in U.S. Patent Publication Nos. US20160174546, US20140301951, US20150105538, US20150250725, Wang et al., J. Control Release, 2017 Jan. 31. pii: S0168-3659(17)30038-X. doi: 10.1016/j.jconrel.2017.01.037. [Epub ahead of print]; Altinoğlu et al., Biomater Sci., 4(12): 1773-80, Nov. 15, 2016; Wang et al., PNAS, 113(11):2868-73 Mar. 15, 2016; Wang et al., PloS One, 10(11): e0141860. doi: 10.1371/journal.pone.0141860. eCollection 2015 Nov. 3, 2015; Takeda et al., Neural Regen Res. 10(5):689-90, May 2015; Wang et al., Adv. Healthc Mater., 3(9): 1398-403, September 2014; and Wang et al., Agnew Chem Int Ed Engl., 53(11):2893-8, Mar. 10, 2014; James E. Dahlman and Carmen Barnes et al. Nature Nanotechnology (2014) published online 11 May 2014, doi:10.1038/nnano.2014.84; Coelho et al., N Engl J Med 2013; 369:819-29; Aleku et al., Cancer Res., 68(23): 9788-98 (Dec. 1, 2008), Strumberg et al., Int. J. Clin. Pharmacol. Ther., 50(1): 76-8 (January 2012), Schultheis et al., J. Clin. Oncol., 32(36): 4141-48 (Dec. 20, 2014), and Fehring et al., Mol. Ther., 22(4): 811-20 (Apr. 22, 2014); Novobrantseva, Molecular Therapy—Nucleic Acids (2012) 1, e4; doi:10.1038/mtna.2011.3; WO2012135025; US 20140348900; US 20140328759; US 20140308304; WO 2005/105152; WO 2006/069782; WO 2007/121947; US 2015/082080; US 20120251618; U.S. Pat. Nos. 7,982,027; 7,799,565; 8,058,069; 8,283,333; 7,901,708; 7,745,651; 7,803,397; 8,101,741; 8,188,263; 7,915,399; 8,236,943 and 7,838,658 and European Pat. Nos 1766035; 1519714; 1781593 and 1664316.

### Liposomes

In some embodiments, a lipid particle may be liposome. Liposomes are spherical vesicle structures composed of a uni- or multilamellar lipid bilayer surrounding internal aqueous compartments and a relatively impermeable outer lipophilic phospholipid bilayer. In some embodiments, liposomes are biocompatible, nontoxic, can deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

In some embodiments, a liposome delivery vehicle can be used to deliver an engineered biomolecule of the present disclosure and/or a virus particle containing an engineered biomolecule of the present disclosure. In some embodiments, the virus particle(s) can be adsorbed to the liposome, such as through electrostatic interactions, and/or can be attached to the liposomes via a linker.

In some embodiments, the liposome can be a Trojan Horse liposome (also known in the art as Molecular Trojan Horses), see e.g., http://cshprotocols.cshlp.org/content/2010/4/pdb.prot5407.long, the teachings of which can be applied and/or adapted to generated and/or deliver the engineered biomolecules described herein.

Other non-limiting, exemplary liposomes can be those as set forth in Wang et al., ACS Synthetic Biology, 1, 403-07 (2012); Wang et al., PNAS, 113(11) 2868-2873 (2016); Spuch and Navarro, Journal of Drug Delivery, vol. 2011, Article ID 469679, 12 pages, 2011. doi: 10.1155/2011/469679; WO 2008/042973; U.S. Pat. No. 8,071,082; WO 2014/186366; 20160257951; US 20160129120; US 20160244761; 20120251618; WO2013/093648; Lipofectin (a combination of DOTMA and DOPE), Lipofectase, LIPOFECTAMINE® (e.g., LIPOFECTAMINE® 2000, LIPOFECTAMINE® 3000, LIPOFECTAMINE® RNAIMAX, LIPOFECTAMINE® LTX), SAINT-RED (Synvolux Therapeutics, Groningen Netherlands), DOPE, Cytofectin (Gilead Sciences, Foster City, Calif.), and Eufectins (JBL, San Luis Obispo, Calif.).

### Stable Nucleic-Acid-Lipid Particles (SNALPs)

In some embodiments, the lipid particles may be stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycol)2000)carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero-3-phosphocholine, PEG-CDMA, and 1,2-dilinoleyloxy-3-(N;N-dimethyl)aminopropane (DLinDMAo).

Other non-limiting, exemplary SNALPs that can be used to deliver the engineered biomolecules described herein can be any such SNALPs as described in Morrissey et al., Nature Biotechnology, Vol. 23, No. 8, August 2005, Zimmerman et al., Nature Letters, Vol. 441, 4 May 2006; Geisbert et al., Lancet 2010; 375: 1896-905; Judge, J. Clin. Invest. 119:661-673 (2009); and Semple et al., Nature Niotechnology, Volume 28 Number 2 Feb. 2010, pp. 172-177.

### Other Lipids

The lipid particles may also comprise one or more other types of lipids, e.g., cationic lipids, such as amino lipid 2,2-dilinoleyl-4-dimethylaminoethyl-[1,3]-dioxolane (DLin-KC2-DMA), DLin-KC2-DMA4, C12-200 and colipids disteroylphosphatidyl choline, cholesterol, and PEG-DMG.

In some embodiments, the delivery vehicle can be or include a lipidoid, such as any of those set forth in, for example, US 20110293703.

In some embodiments, the delivery vehicle can be or include an amino lipid, such as any of those set forth in, for example, Jayaraman, Angew. Chem. Int. Ed. 2012, 51, 8529-8533.

In some embodiments, the delivery vehicle can be or include a lipid envelope, such as any of those set forth in, for example, Korman et al., 2011. Nat. Biotech. 29:154-157.

### Lipoplexes/Polyplexes

In some embodiments, the delivery vehicles comprise lipoplexes and/or polyplexes. Lipoplexes may bind to negatively charged cell membrane and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2lp (e.g., forming DNA/Ca²⁺ microcomplexes), polyethenimine (PEI) (e.g., branched PEI), and poly(L-lysine) (PLL).

### Sugar-Based Particles

In some embodiments, the delivery vehicle can be a sugar-based particle. In some embodiments, the sugar-based particles can be or include GalNAc, such as any of those described in WO2014118272; US 20020150626; Nair, J K et al., 2014, Journal of the American Chemical Society 136 (49), 16958-16961; Østergaard et al., Bioconjugate Chem., 2015, 26 (8), pp 1451-1455.

### Cell Penetrating Peptides

In some embodiments, the delivery vehicles comprise cell penetrating peptides (CPPs). CPPs are short peptides that facilitate cellular uptake of various molecular cargo (e.g., from nanosized particles to small chemical molecules and large fragments of DNA).

CPPs may be of different sizes, amino acid sequences, and charges. In some examples, CPPs can translocate the plasma membrane and facilitate the delivery of various molecular cargoes to the cytoplasm or an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Another type of CPPs is the trans-activating transcriptional activator (Tat) from Human Immunodeficiency Virus 1 (HIV-1). Examples of CPPs include to Penetratin, Tat (48-60), Transportan, and (R-AhX-R4) (Ahx refers to aminohexanoyl), Kaposi fibroblast growth factor (FGF) signal peptide sequence, integrin ß3 signal peptide sequence, polyarginine peptide Args sequence, Guanine rich-molecular transporters, and sweet arrow peptide. Examples of CPPs and related applications also include those described in U.S. Pat. No. 8,372,951.

CPPs can be used for in vitro and ex vivo work quite readily, and extensive optimization for each cargo and cell type is usually required. In some examples, CPPs may be covalently attached to the Cas protein directly, which is then complexed with the gRNA and delivered to cells. In some examples, separate delivery of CPP-Cas and CPP-gRNA to multiple cells may be performed. CPP may also be used to delivery RNPs.

CPPs may be used to deliver the compositions and systems to plants. In some examples, CPPs may be used to deliver the components to plant protoplasts, which are then regenerated to plant cells and further to plants.

### DNA Nanoclews

In some embodiments, the delivery vehicles comprise DNA nanoclews. A DNA nanoclew refers to a sphere-like structure of DNA (e.g., with a shape of a ball of yarn). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct. 22; 136(42): 14722-5; and Sun W et al, Angew Chem Int Ed Engl. 2015 Oct. 5; 54(41):12029-33. DNA nanoclew may have a palindromic sequences to be partially complementary to the gRNA within the Cas:gRNA ribonucleoprotein complex. A DNA nanoclew may be coated, e.g., coated with PEI to induce endosomal escape.

### Metal Nanoparticles

In some embodiments, the delivery vehicles comprise gold nanoparticles (also referred to AuNPs or colloidal gold). Gold nanoparticles may form complex with cargos. Gold nanoparticles may be coated, e.g., coated in a silicate and an endosomal disruptive polymer, PAsp(DET). Examples of gold nanoparticles include AuraSense Therapeutics' Spherical Nucleic Acid (SNA™) constructs, and those described in Mout R, et al. (2017). ACS Nano 11:2452-8; Lee K, et al. (2017). Nat Biomed Eng 1:889-901. Other metal nanoparticles can also be complexed with cargo(s). Such metal particles include, without limitation, tungsten, palladium, rhodium, platinum, and iridium particles. Other non-limiting, exemplary metal nanoparticles are described in US 20100129793. iTOP

In some embodiments, the delivery vehicles comprise iTOP. iTOP refers to a combination of small molecules drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D'Astolfo D S, Pagliero R J, Pras A, et al. (2015). Cell 161:674-690.

### Polymer-Based Particles

In some embodiments, the delivery vehicles may comprise polymer-based particles (e.g., nanoparticles). In some embodiments, the polymer-based particles may mimic a viral mechanism of membrane fusion. The polymer-based particles may be a synthetic copy of Influenza virus machinery and form transfection complexes with various types of nucleic acids ((siRNA, miRNA, plasmid DNA or shRNA, mRNA, etc.) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In some embodiments, the polymer-based particles may comprise alkylated and carboxyalkylated branched polyethylenimine. In some examples, the polymer-based particles are VIROMER, e.g., VIROMER RNAi, VIROMER RED, VIROMER mRNA. Example methods of delivering the systems and compositions herein include those described in Bawage S S et al., Synthetic mRNA expressed Cas13a mitigates RNA virus infections, www.biorxiv.org/content/10.1101/370460v1.full doi: doi.org/10.1101/370460, Viromer® RED, a powerful tool for transfection of keratinocytes. doi: 10.13140/RG.2.2.16993.61281, Viromer® Transfection-Factbook 2018: technology, product overview, users' data., doi:10.13140/RG.2.2.23912.16642. Other exemplary and non-limiting polymeric particles are described in US 20170079916, US 20160367686, US 20110212179, US 20130302401, U.S. Pat. Nos. 6,007,845, 5,855,913, 5,985,309, 5,543,158, WO2012135025, US 20130,252281, US 20130245107, US 20130244279; US 20050019923, 20080267903;

### Streptolysin O(SLO)

The delivery vehicles may be or include streptolysin O (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc Natl Acad Sci USA 98:3185-90; Teng K W, et al. (2017). Elife 6:e25460.

### Multifunctional Envelope-Type Nanodevice (MEND)

The delivery vehicles may comprise multifunctional envelope-type nanodevice (MENDs). MENDs may comprise condensed plasmid DNA, a PLL core, and a lipid film shell. A MEND may further comprise cell-penetrating peptide (e.g., stearyl octaarginine). The cell penetrating peptide may be in the lipid shell. The lipid envelope may be modified with one or more functional components, e.g., one or more of: polyethylene glycol (e.g., to increase vascular circulation time), ligands for targeting of specific tissues/cells, additional cell-penetrating peptides (e.g., for greater cellular delivery), lipids to enhance endosomal escape, and nuclear delivery tags. In some examples, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Acc Chem Res 45:1113-21.

### Lipid-Coated Mesoporous Silica Particles

The delivery vehicles may comprise lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In some embodiments, pore sizes, pore chemistry, and overall particle sizes may be modified for loading different types of cargos. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

### Inorganic Nanoparticles

The delivery vehicles may comprise inorganic nanoparticles. Examples of inorganic nanoparticles include carbon nanotubes (CNTs) (e.g., as described in Bates K and Kostarelos K. (2013). Adv Drug Deliv Rev 65:2023-33.), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

### Exosomes

The delivery vehicles may comprise exosomes. Exosomes include membrane bound extracellular vesicles, which can be used to contain and delivery various types of biomolecules, such as proteins, carbohydrates, lipids, and nucleic acids, and complexes thereof (e.g., RNPs). Examples of exosomes include those described in Schroeder A, et al., J Intern Med. 2010 January; 267(1):9-21; El-Andaloussi S, et al., Nat Protoc. 2012 December; 7(12):2112-26; Uno Y, et al., Hum Gene Ther. 2011 June; 22(6):711-9; Zou W, et al., Hum Gene Ther. 2011 April; 22(4):465-75.

In some examples, the exosome may form a complex (e.g., by binding directly or indirectly) to one or more components of the cargo. In certain examples, a molecule of an exosome may be fused with first adapter protein and a component of the cargo may be fused with a second adapter protein. The first and the second adapter protein may specifically bind each other, thus associating the cargo with the exosome. Examples of such exosomes include those described in Ye Y, et al., Biomater Sci. 2020 Apr. 28. doi: 10.1039/d0bm00427h.

Other non-limiting, exemplary exosomes include any of those set forth in Alvarez-Erviti et al. 2011, Nat Biotechnol 29: 341; El-Andaloussi et al. (Nature Protocols 7:2112-2126(2012); and Wahlgren et al. (Nucleic Acids Research, 2012, Vol. 40, No. 17 e130).

### Spherical Nucleic Acids (SNAs)

In some embodiments, the delivery vehicle can be or include a SNA. SNAs are three dimensional nanostructures that can be composed of densely functionalized and highly oriented nucleic acids that can be covalently attached to the surface of spherical nanoparticle cores. The core of the spherical nucleic acid can impart the conjugate with specific chemical and physical properties, and it can act as a scaffold for assembling and orienting the oligonucleotides into a dense spherical arrangement that gives rise to many of their functional properties, distinguishing them from all other forms of matter. In some embodiments, the core is a crosslinked polymer. Non-limiting, exemplary SNAs can be any of those set forth in Cutler et al., J. Am. Chem. Soc. 2011 133:9254-9257, Hao et al., Small. 2011 7:3158-3162, Zhang et al., ACS Nano. 2011 5:6962-6970, Cutler et al., J. Am. Chem. Soc. 2012 134:1376-1391, Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495:S14-S16, Choi et al., Proc. Natl. Acad. Sci. USA. 2013 110(19):7625-7630, Jensen et al., Sci. Transl. Med. 5, 209ra152 (2013) and Mirkin, et al., and Small, 10:186-192.

### Self-Assembling Nanoparticles

In some embodiments, the delivery vehicle is a self-assembling nanoparticle. The self-assembling nanoparticles can contain one or more polymers. The self-assembling nanoparticles can be PEGylated. Self-assembling nanoparticles are known in the art. Non-limiting, exemplary self-assembling nanoparticles can any as set forth in Schiffelers et al., Nucleic Acids Research, 2004, Vol. 32, No. 19, Bartlett et al. (PNAS, Sep. 25, 2007, vol. 104, no. 39; Davis et al., Nature, Vol 464, 15 Apr. 2010.

### Supercharged Proteins

In some embodiments, the delivery vehicle can be a supercharged protein. As used herein “Supercharged proteins” are a class of engineered or naturally occurring proteins with unusually high positive or negative net theoretical charge. Non-limiting, exemplary supercharged proteins can be any of those set forth in Lawrence et al., 2007, Journal of the American Chemical Society 129, 10110-10112.

### Targeted Delivery

In some embodiments, the delivery vehicle can allow for targeted delivery to a specific cell, tissue, organ, or system. In some embodiments, the engineered biomolecules are engineered such that when expressed in RNA or polypeptide form within a cell they include a targeting moiety that targets the lysosome. In such embodiments, the delivery vehicle can include one or more targeting moieties that can direct targeted delivery of the cargo(s). In some embodiments, the engineered biomolecule can include both a cell type targeting moiety and lysosome targeting moiety. In some embodiments where the engineered biomolecule is incorporated in a delivery vehicle, the delivery vehicle includes a cell type targeting moiety and the engineered biomolecule includes a lysosome targeting moiety. In some embodiments, the lysosome targeting moiety is only effective as a lysosome targeting moiety when translated into a polypeptide. Thus, for example, an engineered DNA or RNA of the present disclosure can incorporate sequence that encodes a polypeptide lysosome targeting moiety, such that when delivered to the cell, the engineered DNA and/or RNA is transcribed and/or translated into a polypeptide in the nucleus and/or cytosol but is not targeted to the lysosome and only the translated polypeptide is targeted to the lysosome. In some embodiments where a cell type targeting moiety is included in the engineered biomolecule and/or a delivery vehicle, the cell type targeting moiety is a tumor or cancer cell targeting moiety.

### Exemplary Targeting Moieties

In an embodiment, the delivery vehicle comprises a targeting moiety, such as active targeting of a lipid entity of the invention, e.g., lipid particle or nanoparticle or liposome or lipid bilayer of the invention comprising a targeting moiety for active targeting.

With regard to targeting moieties, exemplary methods and targeting moieties are described in e.g., Deshpande et al, “Current trends in the use of liposomes for tumor targeting,” Nanomedicine (Lond). 8(9), doi:10.2217/nnm.13.118 (2013); International Patent Publication No. WO 2016/027264, Lorenzer et al., Journal of Controlled Release, 203: 1-15 (2015) all of which are incorporated herein by reference.

An actively targeting lipid particle or nanoparticle or liposome or lipid bilayer delivery system (generally as to embodiments of the invention, “lipid entity of the invention” delivery systems) are prepared by conjugating targeting moieties, including small molecule ligands, peptides and monoclonal antibodies, on the lipid or liposomal surface; for example, certain receptors, such as folate and transferrin (Tf) receptors (TfR), are overexpressed on many cancer cells and have been used to make liposomes tumor cell specific. Liposomes that accumulate in the tumor microenvironment can be subsequently endocytosed into the cells by interacting with specific cell surface receptors. To efficiently target liposomes to cells, such as cancer cells, it is useful that the targeting moiety have an affinity for a cell surface receptor and to link the targeting moiety in sufficient quantities to have optimum affinity for the cell surface receptors; and determining these embodiments are within the ambit of the skilled artisan. In the field of active targeting, there are a number of cell-, e.g., tumor-, specific targeting ligands.

Also, as to active targeting, with regard to targeting cell surface receptors such as cancer cell surface receptors, targeting ligands on liposomes can provide attachment of liposomes to cells, e.g., vascular cells, via a noninternalizing epitope; and this can increase the extracellular concentration of that which is being delivered, thereby increasing the amount delivered to the target cells. A strategy to target cell surface receptors, such as cell surface receptors on cancer cells, such as overexpressed cell surface receptors on cancer cells, is to use receptor-specific ligands or antibodies. Many cancer cell types display upregulation of tumor-specific receptors. For example, TfRs and folate receptors (FRs) are greatly overexpressed by many tumor cell types in response to their increased metabolic demand. Folic acid can be used as a targeting ligand for specialized delivery owing to its ease of conjugation to nanocarriers, its high affinity for FRs and the relatively low frequency of FRs, in normal tissues as compared with their overexpression in activated macrophages and cancer cells, e.g., certain ovarian, breast, lung, colon, kidney and brain tumors. Overexpression of FR on macrophages is an indication of inflammatory diseases, such as psoriasis, Crohn's disease, rheumatoid arthritis and atherosclerosis; accordingly, folate-mediated targeting of the invention can also be used for studying, addressing or treating inflammatory disorders, as well as cancers. Folate-linked lipid particles or nanoparticles or liposomes or lipid bilayers of the invention (“lipid entity of the invention”) deliver their cargo intracellularly through receptor-mediated endocytosis. Intracellular trafficking can be directed to acidic compartments that facilitate cargo release, and, most importantly, release of the cargo can be altered or delayed until it reaches the cytoplasm or vicinity of target organelles. Delivery of cargo using a lipid entity of the invention having a targeting moiety, such as a folate-linked lipid entity of the invention, can be superior to nontargeted lipid entity of the invention. The attachment of folate directly to the lipid head groups may not be favorable for intracellular delivery of folate-conjugated lipid entity of the invention, since they may not bind as efficiently to cells as folate attached to the lipid entity of the invention surface by a spacer, which may can enter cancer cells more efficiently. A lipid entity of the invention coupled to folate can be used for the delivery of complexes of lipid, e.g., liposome, e.g., anionic liposome and virus or capsid or envelope or virus outer protein, such as those herein discussed such as adenovirus or AAV. Tf is a monomeric serum glycoprotein of approximately 80 KDa involved in the transport of iron throughout the body. Tf binds to the TfR and translocates into cells via receptor-mediated endocytosis. The expression of TfR can be higher in certain cells, such as tumor cells (as compared with normal cells and is associated with the increased iron demand in rapidly proliferating cancer cells. Accordingly, the invention comprehends a TfR-targeted lipid entity of the invention, e.g., as to liver cells, liver cancer, breast cells such as breast cancer cells, colon such as colon cancer cells, ovarian cells such as ovarian cancer cells, head, neck and lung cells, such as head, neck and non-small-cell lung cancer cells, cells of the mouth such as oral tumor cells.

Also, as to active targeting, a lipid entity of the invention can be multifunctional, i.e., employ more than one targeting moiety such as CPP, along with Tf; a bifunctional system; e.g., a combination of Tf and poly-L-arginine which can provide transport across the endothelium of the blood-brain barrier. EGFR, is a tyrosine kinase receptor belonging to the ErbB family of receptors that mediates cell growth, differentiation and repair in cells, especially non-cancerous cells, but EGF is overexpressed in certain cells such as many solid tumors, including colorectal, non-small-cell lung cancer, squamous cell carcinoma of the ovary, kidney, head, pancreas, neck and prostate, and especially breast cancer. The invention comprehends EGFR-targeted monoclonal antibody(ies) linked to a lipid entity of the invention. HER-2 is often overexpressed in patients with breast cancer, and is also associated with lung, bladder, prostate, brain and stomach cancers. HER-2, encoded by the ERBB2 gene. The invention comprehends a HER-2-targeting lipid entity of the invention, e.g., an anti-HER-2-antibody (or binding fragment thereof)-lipid entity of the invention, a HER-2-targeting-PEGylated lipid entity of the invention (e.g., having an anti-HER-2-antibody or binding fragment thereof), a HER-2-targeting-maleimide-PEG polymer-lipid entity of the invention (e.g., having an anti-HER-2-antibody or binding fragment thereof). Upon cellular association, the receptor-antibody complex can be internalized by formation of an endosome for delivery to the cytoplasm.

With respect to receptor-mediated targeting, the skilled artisan takes into consideration ligand/target affinity and the quantity of receptors on the cell surface, and that PEGylation can act as a barrier against interaction with receptors. The use of antibody-lipid entity of the invention targeting can be advantageous. Multivalent presentation of targeting moieties can also increase the uptake and signaling properties of antibody fragments. In practice of the invention, the skilled person takes into account ligand density (e.g., high ligand densities on a lipid entity of the invention may be advantageous for increased binding to target cells). Preventing early by macrophages can be addressed with a sterically stabilized lipid entity of the invention and linking ligands to the terminus of molecules such as PEG, which is anchored in the lipid entity of the invention (e.g., lipid particle or nanoparticle or liposome or lipid bilayer). The microenvironment of a cell mass such as a tumor microenvironment can be targeted; for instance, it may be advantageous to target cell mass vasculature, such as the tumor vasculature microenvironment. Thus, in some embodiments VEGF is targeted. VEGF and its receptors are well-known proangiogenic molecules and are well-characterized targets for antiangiogenic therapy. Many small-molecule inhibitors of receptor tyrosine kinases, such as VEGFRs or basic FGFRs, have been developed as anticancer agents and the invention comprehends coupling any one or more of these peptides to a lipid entity of the invention, e.g., phage IVO peptide(s) (e.g., via or with a PEG terminus), tumor-homing peptide APRPG such as APRPG-PEG-modified. VCAM, the vascular endothelium plays a key role in the pathogenesis of inflammation, thrombosis and atherosclerosis. CAMs are involved in inflammatory disorders, including cancer, and are a logical target, E- and P-selectins, VCAM-1 and ICAMs. Can be used to target a lipid entity of the invention., e.g., with PEGylation.

Matrix metalloproteases (MMPs) belong to the family of zinc-dependent endopeptidases. They are involved in tissue remodeling, tumor invasiveness, resistance to apoptosis and metastasis. There are four MMP inhibitors called TIMP1-4, which determine the balance between tumor growth inhibition and metastasis; a protein involved in the angiogenesis of tumor vessels is MT1-MMP, expressed on newly formed vessels and tumor tissues. The proteolytic activity of MT1-MMP cleaves proteins, such as fibronectin, elastin, collagen and laminin, at the plasma membrane and activates soluble MMPs, such as MMP-2, which degrades the matrix.

In some embodiments, MT1-MMP is targeted. An exemplary MT1-MMP1 targeting moiety is an antibody or fragment thereof, such as a Fab′ fragment, is included in the delivery vehicles as a targeting moiety to target MT1-MMP. Exemplary delivery vehicles that include an MT1-MMP antibodies include in an anti MT1-MMP monoclonal antibody (e.g., antihuman MT1-MMP or anti mammalian MT1-MMP monoclonal antibody) linked to a lipid entity of the invention, e.g., via a spacer such as a PEG spacer.

αβ-integrins or integrins are a group of transmembrane glycoprotein receptors that mediate attachment between a cell and its surrounding tissues or extracellular matrix. Integrins contain two distinct chains (heterodimers) called α- and β-subunits. The tumor tissue-specific expression of integrin receptors can be utilized for targeted delivery in the invention, e.g., whereby the targeting moiety can be an RGD peptide such as a cyclic RGD.

Aptamers are ssDNA or RNA oligonucleotides that impart high affinity and specific recognition of the target molecules by electrostatic interactions, hydrogen bonding and hydrophobic interactions as opposed to the Watson-Crick base pairing, which is typical for the bonding interactions of oligonucleotides. Aptamers as a targeting moiety can have advantages over antibodies: aptamers can demonstrate higher target antigen recognition as compared with antibodies; aptamers can be more stable and smaller in size as compared with antibodies; aptamers can be easily synthesized and chemically modified for molecular conjugation; and aptamers can be changed in sequence for improved selectivity and can be developed to recognize poorly immunogenic targets. Such moieties as a sgc8 aptamer can be used as a targeting moiety (e.g., via covalent linking to the lipid entity of the invention, e.g., via a spacer, such as a PEG spacer).

Also, as to active targeting, the invention also comprehends intracellular delivery. Since liposomes follow the endocytic pathway, they are entrapped in the endosomes (pH 6.5-6) and subsequently fuse with lysosomes (pH <5), where they undergo degradation that results in a lower therapeutic potential. The low endosomal pH can be taken advantage of to escape degradation. Fusogenic lipids or peptides, which destabilize the endosomal membrane after the conformational transition/activation at a lowered pH. Amines are protonated at an acidic pH and cause endosomal swelling and rupture by a buffer effect Unsaturated dioleoylphosphatidylethanolamine (DOPE) readily adopts an inverted hexagonal shape at a low pH, which causes fusion of liposomes to the endosomal membrane. This process destabilizes a lipid entity containing DOPE and releases the cargo into the cytoplasm; fusogenic lipid GALA, cholesteryl-GALA and PEG-GALA may show a highly efficient endosomal release; a pore-forming protein listeriolysin O may provide an endosomal escape mechanism; and, histidine-rich peptides have the ability to fuse with the endosomal membrane, resulting in pore formation, and can buffer the proton pump causing membrane lysis.

The invention comprehends a lipid entity of the invention modified with CPP(s), for intracellular delivery that may proceed via energy dependent macropinocytosis followed by endosomal escape. The invention further comprehends organelle-specific targeting. A lipid entity of the invention surface-functionalized with the triphenylphosphonium (TPP) moiety or a lipid entity of the invention with a lipophilic cation, rhodamine 123 can be effective in delivery of cargo to mitochondria. DOPE/sphingomyelin/stearyl-octa-arginine can delivers cargos to the mitochondrial interior via membrane fusion. A lipid entity of the invention surface modified with a lysosomotropic ligand, octadecyl rhodamine B can deliver cargo to lysosomes. Ceramides are useful in inducing lysosomal membrane permeabilization; the invention comprehends intracellular delivery of a lipid entity of the invention having a ceramide. The invention further comprehends a lipid entity of the invention targeting the nucleus, e.g., via a DNA-intercalating moiety. The invention also comprehends multifunctional liposomes for targeting, i.e., attaching more than one functional group to the surface of the lipid entity of the invention, for instance to enhances accumulation in a desired site and/or promotes organelle-specific delivery and/or target a particular type of cell and/or respond to the local stimuli such as temperature (e.g., elevated), pH (e.g., decreased), respond to externally applied stimuli such as a magnetic field, light, energy, heat or ultrasound and/or promote intracellular delivery of the cargo. All of these are considered actively targeting moieties.

It should be understood that as to each possible targeting or active targeting moiety herein-discussed, there is an embodiment of the invention wherein the delivery system comprises such a targeting or active targeting moiety. Likewise, the following Table 3 provides exemplary targeting moieties that can be incorporated into the engineered biomolecules and/or delivery vehicles of the present disclosure.

TABLE 3

| Targeting Moiety | Target Molecule | Target Cell or Tissue |
|---|---|---|
| folate | folate receptor | cancer cells |
| transferrin | transferrin receptor | cancer cells |
| Antibody CC52 | CC531 | colon adenocarcinoma CC531 |
| anti-HER2 antibody | HER2 | HER2-overexpressing tumors |
| anti-GD2 | GD2 | neuroblastoma, melanoma |
| anti-EGFR | EGFR | tumor cells overexpressing EGFR |
| pH-dependent fusogenic | | ovarian carcinoma |
| peptide diINF-7 | | |
| anti-VEGFR | VEGF Receptor | tumor vasculature |
| anti-CD19 | CD19 (B cell marker) | leukemia, lymphoma |
| cell-penetrating peptide | | blood-brain barrier |
| cyclic arginine-glycine- | avβ3 | glioblastoma cells, human |
| aspartic acid-tyrosine- | | umbilical vein endothelial |
| cysteine peptide (c(RGDyC)-LP) | | cells, tumor angiogenesis |
| ASSHN peptide | | endothelial progenitor |
| | | cells; anti-cancer |
| PR_b peptide | α₅β₁ integrin | cancer cells |
| AG86 peptide | α₆β₄ integrin | cancer cells |
| KCCYSL (P6.1 peptide) | HER-2 receptor | cancer cells |
| affinity peptide LN | Aminopeptidase N | APN-positive tumor |
| (YEVGHRC) | (APN/CD13) | |
| synthetic somatostatin | Somatostatin receptor 2 | breast cancer |
| analogue | (SSTR2) | |
| anti-CD20 monoclonal | B-lymphocytes | B cell lymphoma |
| antibody | | |

Thus, in an embodiment of the delivery system, the targeting moiety comprises a receptor ligand, such as, for example, hyaluronic acid for CD44 receptor, galactose for hepatocytes, or antibody or fragment thereof such as a binding antibody fragment against a desired surface receptor, and as to each of a targeting moiety comprising a receptor ligand, or an antibody or fragment thereof such as a binding fragment thereof, such as against a desired surface receptor, there is an embodiment of the invention wherein the delivery system comprises a targeting moiety comprising a receptor ligand, or an antibody or fragment thereof such as a binding fragment thereof, such as against a desired surface receptor, or hyaluronic acid for CD44 receptor, galactose for hepatocytes (see, e.g., Surace et al, “Lipoplexes targeting the CD44 hyaluronic acid receptor for efficient transfection of breast cancer cells,” J. Mol Pharm 6(4):1062-73; doi: 10.1021/mp800215d (2009); Sonoke et al, “Galactose-modified cationic liposomes as a liver-targeting delivery system for small interfering RNA,” Biol Pharm Bull. 34(8): 1338-42 (2011); Torchilin, “Antibody-modified liposomes for cancer chemotherapy,” Expert Opin. Drug Deliv. 5 (9), 1003-1025 (2008); Manjappa et al, “Antibody derivatization and conjugation strategies: application in preparation of stealth immunoliposome to target chemotherapeutics to tumor,” J. Control. Release 150 (1), 2-22 (2011); Sofou S “Antibody-targeted liposomes in cancer therapy and imaging,” Expert Opin. Drug Deliv. 5 (2): 189-204 (2008); Gao J et al, “Antibody-targeted immunoliposomes for cancer treatment,” Mini. Rev. Med. Chem. 13(14): 2026-2035 (2013); Molavi et al, “Anti-CD30 antibody conjugated liposomal doxorubicin with significantly improved therapeutic efficacy against anaplastic large cell lymphoma,” Biomaterials 34(34):8718-25 (2013), each of which and the documents cited therein are hereby incorporated herein by reference), the teachings of which can be applied and/or adapted for targeted delivery of one or more engineered biomolecules, vectors, vector systems, etc. of the present disclosure described herein.

Exemplary lysosome targeting moieties include, but are not limited to any one or more of the following:
a. IGF2 (insulin like growth factor 2) or an M6PR binding domain thereof
b. A LIMP-2 ligand-Lysosomal integral membrane protein LIMP-2 transports the cargo β-glucocerebrosidase into lysosome:
   i. It is predicted that the following specific regions are important for LIMP-2 binding: helix 1a (residues T86-L96), helix 1b (residues P99-S110), and helix 2 (P150-A168). Below is the sequence from T86-A168.
   ii. Protein: β-glucocerebrosidase
   iii. Amino Acid Sequence:

(SEQ ID NO: 1)
RRMELSMGPIQANHTGTGLLLTLQPEQKFQKVKGFGGAMTDAAALNILAL
SPPAQNLLLKSYFSEEGIGYNIIRVPMASCDFS

iv. mRNA sequence:

(SEQ ID NO: 2)
cgccgcauggaacugagcaugggcccgauucaggcgaaccauaccggcac
cggccugcugcugacccugcagccggaacagaaauuucagaaagugaaag
gcuuuggggcgcgaugaccgaugcggggcgcugaacauucuggcgcugag
cccgccggcgcagaaccugcugcugaaaagcuauuuuagcgaagaaggca
uuggcuauaacauuauucgcgugccgauggcgagcugcgauuuuagc

c. A sortilin ligand-Sortilin transports the cargo prosaposin into lysosome:
   i. Residues 521-557 of the C terminus of prosaposin is necessary for sending prosaposin to sortilin.
   ii. Protein: Prosaposin
   iii. Amino Acid Sequence:

| (SEQ ID NO: 3) | |
|---|---|
| | LLLGTEKCVWGPSYWCQNMETAARCNAVDHCKRHVWN |

iv. mRNA sequence:

(SEQ ID NO: 4)
cugcugcugggcaccgaaaaaugcguguggggcccgagcuauuggugcca
gaacauggaaaccgcggcgcgcugcaacgogguggaucauugcaaacgcc
auguguggaac

d. Additional lysosomal proteins that are sent to the lysosome after translation in the cytoplasm using dileucine-based and tyrosine-based sorting signals are in Table 1.
e. any combination thereof.

Other exemplary targeting moieties are described elsewhere herein, such as epitope tags and the like.

### Responsive Delivery

In some embodiments, the delivery vehicle can allow for responsive delivery of the cargo(s). Responsive delivery, as used in this context herein, refers to delivery of cargo(s) by the delivery vehicle in response to an external stimuli. Examples of suitable stimuli include, without limitation, an energy (light, heat, cold, and the like), a chemical stimuli (e.g., chemical composition, etc.), and a biologic or physiologic stimuli (e.g., environmental pH, osmolarity, salinity, biologic molecule, etc.). In some embodiments, the targeting moiety can be responsive to an external stimuli and facilitate responsive delivery. In other embodiments, responsiveness is determined by a non-targeting moiety component of the delivery vehicle.

The delivery vehicle can be stimuli-sensitive, e.g., sensitive to an externally applied stimuli, such as magnetic fields, ultrasound or light; and pH-triggering can also be used, e.g., a labile linkage can be used between a hydrophilic moiety such as PEG and a hydrophobic moiety such as a lipid entity of the invention, which is cleaved only upon exposure to the relatively acidic conditions characteristic of the a particular environment or microenvironment such as an endocytic vacuole or the acidotic tumor mass. pH-sensitive copolymers can also be incorporated in embodiments of the invention can provide shielding; diortho esters, vinyl esters, cysteine-cleavable lipopolymers, double esters and hydrazones are a few examples of pH-sensitive bonds that are quite stable at pH 7.5, but are hydrolyzed relatively rapidly at pH 6 and below, e.g., a terminally alkylated copolymer of N-isopropylacrylamide and methacrylic acid that copolymer facilitates destabilization of a lipid entity of the invention and release in compartments with decreased pH value; or, the invention comprehends ionic polymers for generation of a pH-responsive lipid entity of the invention (e.g., poly(methacrylic acid), poly(diethylaminoethyl methacrylate), poly(acrylamide) and poly(acrylic acid)).

In some embodiments, the delivery is temperature-triggered delivery. Many pathological areas, such as inflamed tissues and tumors, show a distinctive hyperthermia compared with normal tissues. Utilizing this hyperthermia is an attractive strategy in cancer therapy since hyperthermia is associated with increased tumor permeability and enhanced uptake. This technique involves local heating of the site to increase microvascular pore size and blood flow, which, in turn, can result in an increased extravasation of embodiments of the invention. Temperature-sensitive lipid entity of the invention can be prepared from thermosensitive lipids or polymers with a low critical solution temperature. Above the low critical solution temperature (e.g., at site such as tumor site or inflamed tissue site), the polymer precipitates, disrupting the liposomes to release. Lipids with a specific gel-to-liquid phase transition temperature are used to prepare these lipid entities of the invention; and a lipid for a thermosensitive embodiment can be dipalmitoylphosphatidylcholine. Thermosensitive polymers can also facilitate destabilization followed by release, and a useful thermosensitive polymer is poly (N-isopropylacrylamide). Another temperature triggered system can employ lysolipid temperature-sensitive liposomes.

In some embodiments, delivery is redox-triggered delivery. The difference in redox potential between normal and inflamed or tumor tissues, and between the intra- and extra-cellular environments has been exploited for delivery, e.g., GSH is a reducing agent abundant in cells, especially in the cytosol, mitochondria and nucleus. The GSH concentrations in blood and extracellular matrix are just one out of 100 to one out of 1000 of the intracellular concentration, respectively. This high redox potential difference caused by GSH, cysteine and other reducing agents can break the reducible bonds, destabilize a lipid entity of the invention and result in release of payload. The disulfide bond can be used as the cleavable/reversible linker in a lipid entity of the invention, because it causes sensitivity to redox owing to the disulfideto-thiol reduction reaction; a lipid entity of the invention can be made reduction sensitive by using two (e.g., two forms of a disulfide-conjugated multifunctional lipid as cleavage of the disulfide bond (e.g., via tris(2-carboxyethyl)phosphine, dithiothreitol, L-cysteine or GSH), can cause removal of the hydrophilic head group of the conjugate and alter the membrane organization leading to release of payload. Calcein release from reduction-sensitive lipid entity of the invention containing a disulfide conjugate can be more useful than a reduction-insensitive embodiment.

Enzymes can also be used as a trigger to release payload. Enzymes, including MMPs (e.g., MMP2), phospholipase A2, alkaline phosphatase, transglutaminase or phosphatidylinositol-specific phospholipase C, have been found to be overexpressed in certain tissues, e.g., tumor tissues. In the presence of these enzymes, specially engineered enzyme-sensitive lipid entity of the invention can be disrupted and release the payload. an MMP2-cleavable octapeptide (Gly-Pro-Leu-Gly-Ile-Ala-Gly-Gln (SEQ ID NO: 59)) can be incorporated into a linker, and can have antibody targeting, e.g., a cancer cell and/or lysosome targeting moiety.

In some embodiments, delivery is light- or energy-triggered delivery, e.g., the lipid entity of the invention can be light-sensitive, such that light or energy can facilitate structural and conformational changes, which lead to direct interaction of the lipid entity of the invention with the target cells via membrane fusion, photo-isomerism, photofragmentation or photopolymerization; such a moiety therefor can be benzoporphyrin photosensitizer. Ultrasound can be a form of energy to trigger delivery; a lipid entity of the invention with a small quantity of particular gas, including air or perfluorated hydrocarbon can be triggered to release with ultrasound, e.g., low-frequency ultrasound (LFUS). Magnetic delivery: A lipid entity of the invention can be magnetized by incorporation of magnetites, such as Fe₃O₄ or γ-Fe₂O₃, e.g., those that are less than 10 nm in size. Responsive delivery can be then by exposure to a magnetic field.

### Pharmaceutical Formulations

Also described herein are pharmaceutical formulations that can contain an amount, effective amount, and/or least effective amount, and/or therapeutically effective amount of one or more engineered biomolecules, vectors, vector systems, cells, delivery vehicles, or any combination thereof (which are also referred to as the primary active agent or ingredient elsewhere herein) described in greater detail elsewhere herein a pharmaceutically acceptable carrier or excipient. In some embodiments, the active ingredient is present as a pharmaceutically acceptable salt of the active ingredient.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s). Other suitable administration routes will be appreciated by those of ordinary skill in the art in view of the present disclosure.

Where appropriate, compounds, molecules, compositions, vectors, vector systems, cells, delivery vehicles, or a combination thereof described in greater detail elsewhere herein can be provided to a subject in need thereof as an ingredient, such as an active ingredient or agent, in a pharmaceutical formulation. As such, also described are pharmaceutical formulations containing one or more of the compounds and salts thereof, or pharmaceutically acceptable salts thereof described herein. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

In some embodiments, the subject is in need of inhibition of a cyst(e)ine stress response in a cell or cell population. In some embodiments, the subject is in need of inhibition of ATF4 induction, particularly cyst(e)ine depletion induced ATF4 induction, in a cell or cell population. In some embodiments, the subject in need thereof has or is suspected of having a disease or disorder, such as a proliferative disease. In some embodiments, the subject in need thereof has or is suspected of having a cancer.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

### Secondary (or Additional Active Agents)

In some embodiments, the pharmaceutical formulation also includes an effective amount of one or more secondary active agents. In some embodiments the optional secondary active agent, is included in the pharmaceutical formulation as a pharmaceutically acceptable salt. Suitable secondary active agents include, but not limited to, biologic agents or molecules including, but not limited to, e.g., polynucleotides, amino acids, peptides, polypeptides, antibodies, aptamers, ribozymes, hormones, immunomodulators, antipyretics, anxiolytics, antipsychotics, analgesics, antispasmodics, anti-inflammatoires, anti-histamines, anti-infectives, chemotherapeutics, radiation or other sensitizers, and combinations thereof.

In some embodiments, the secondary active agent is a compound, molecule, or other composition capable of selectively depleting cyst(e)ine, particularly cytosolic cyst(e)ine, in a cell. In some embodiments, the secondary active agent is a compound, molecule, or other composition effective to induce ferroptosis in a cell. In some embodiments, the secondary active agent is effective to inhibit the X_{c}⁻ antiporter in the cell or cell population. In some embodiments, the secondary active agent is selected from erastin, Ras-selective lethal small molecule 3, sulfasalazine, lanperisone, sorafenib, fenugreek (trigonelline), acetaminophen, cisplatin, artesunate, combination of siramesine and lapatinib, ferumoxytol, and salinomycin (ironomycin), artesunate, dihydroartemisnin, gemcitabine, paclitaxel, temozolomide, buthionine sulfoximine (BSO), solasonine, siramesine, laptinib, BAY 87-2243, Molecule 1 of Taylor et al., 2019. Sci Reports. 9:5926, and its derivatives or metabolites (specifically molecule 4 of Taylor et al. 2019) any others set forth in e.g., Su et al., Cancer. Lett. 2020. Jul. 28; 483:127-136. doi: 10.1016/j.canlet.2020.02.015; Wu et al., 2020. Front. Oncol. | https://doi.org/10.3389/fonc.2020.571127; Lin et al., Front. Pharmacol. 2020. 11: 1061; Taylor et al., 2019. Sci. Reports. 9:5926, all of which are incorporated by reference herein), and any combination thereof.

In some embodiments, the additional active agent is selected from erastin, Ras-selective lethal small molecule 3, sulfasalazine or analogue thereof, lanperisone, sorafenib, fenugreek (trigonelline), acetaminophen, cisplatin, artesunate, siramesine, lapatinib, a combination of siramesine and lapatinib, ferumoxytol, salinomycin (ironomycin), dihydroartemisnin, gemcitabine, paclitaxel, temozolomide, buthionine sulfoximine (BSO), solasonine, siramesine, laptinib, BAY 87-2243,

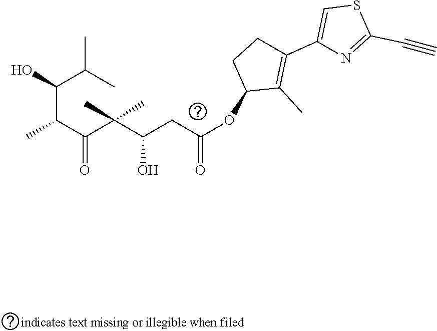

or a derivative or metabolite thereof, optionally

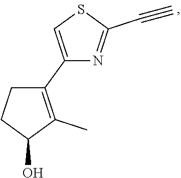

BLZ945, Dyclonine, Oxyfedrine, RSL3, ML210, SSZ, RSL3, ML162, FIN56, Anti-PD1, gefitnib, erlotinib, TMZ, docetaxel, DAT, vemurafenib, lovastatin, atorvastatin, simvastatin, prominin2, artemisinin or a derivative thereof, vitamin E, baicalein, beta-elemene, gallic acid, buthionine sulfoximine, DP17, FIN56, FINO₂, a statin, deferoxamine mesylate, or any combination thereof.

### Effective Amounts

In some embodiments, the amount of the primary active agent and/or optional secondary agent can be an effective amount, least effective amount, and/or therapeutically effective amount. As used herein, “effective amount” refers to the amount of the primary and/or optional secondary agent included in the pharmaceutical formulation that achieve one or more therapeutic effects or desired effect. As used herein, “least effective” amount refers to the lowest amount of the primary and/or optional secondary agent that achieves the one or more therapeutic or other desired effects. As used herein, “therapeutically effective amount” refers to the amount of the primary and/or optional secondary agent included in the pharmaceutical formulation that achieves one or more therapeutic effects. In some embodiments, the one or more therapeutic effects are inducing and/or potentiating ferroptosis in a cell or cell population, inhibiting a cyst(e)ine stress response in a cell or cell population, reducing induction of ATF4 expression, particularly reducing cyst(e)ine depletion induced ATF4 expression, in a cell or cell population, increasing lysosomal cyst(e)ine in a cell or cell population, inhibiting growth and/or proliferation of a cell, particularly a cancer cell, or any combination thereof.

The effective amount, least effective amount, and/or therapeutically effective amount of the primary and optional secondary active agent described elsewhere herein contained in the pharmaceutical formulation can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value with any of these ranges.

In some embodiments, the effective amount, least effective amount, and/or therapeutically effective amount can be an effective concentration, least effective concentration, and/or therapeutically effective concentration, which can each range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value with any of these ranges.

In other embodiments, the effective amount, least effective amount, and/or therapeutically effective amount of the primary and optional secondary active agent can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value with any of these ranges.

In some embodiments, the primary and/or the optional secondary active agent present in the pharmaceutical formulation can range from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation.

In some embodiments where a cell population is present in the pharmaceutical formulation (e.g., as a primary and/or or secondary active agent), the effective amount of cells can range from about 2 cells to 1×10¹/mL, 1×10²⁰/mL or more, such as about 1×10¹/mL, 1×10²/mL, 1×10³/mL, 1×10⁴/mL, 1×10⁵/mL, 1×10⁶/mL, 1×10⁷/mL, 1×10⁸/mL, 1×10⁹/mL, 1×10¹⁰/mL, 1×10¹¹/mL, 1×10¹²/mL, 1×10¹³/mL, 1×10¹⁴/mL, 1×10¹⁵/mL, 1×10¹⁶/mL, 1×10¹⁷/mL, 1×10¹⁸/mL, 1×10¹⁹/mL, to/or about 1×10²⁰/mL.

In some embodiments, the amount or effective amount, particularly where an infective particle is being delivered (e.g., a virus particle having the primary or secondary agent as a cargo), the effective amount of virus particles can be expressed as a titer (plaque forming units per unit of volume) or as a MOI (multiplicity of infection). In some embodiments, the effective amount can be 1×10¹ particles per pL, nL, μL, mL, or L to 1×10²⁰/particles per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L. In some embodiments, the effective titer can be about 1×10¹ transforming units per pL, nL, μL, mL, or L to 1×10²⁰/transforming units per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ transforming units per pL, nL, μL, mL, or L. In some embodiments, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more.

In some embodiments, the amount or effective amount of the one or more of the active agent(s) described herein contained in the pharmaceutical formulation can range from about 1 pg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total secondary active agent in the pharmaceutical formulation. In additional embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation.

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, intratumor, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, M D, 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g., micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, mannitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In some embodiments, the pharmaceutical formulation(s) described herein can be part of a combination treatment or combination therapy. The combination treatment can include the pharmaceutical formulation described herein and an additional treatment agent or modality. The additional treatment modality can be a second active agent, a chemotherapeutic, a biological therapeutic, surgery, radiation, diet modulation, environmental modulation, a physical activity modulation, and combinations thereof.

In some embodiments, the co-therapy or combination therapy can additionally include but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, radiation sensitizers, or any combinations thereof.

In some embodiments, the secondary active agent that is administered as a combination or co-therapy with an engineered biomolecule described herein is a compound, molecule, or other composition capable of selectively depleting cyst(e)ine, particularly cytosolic cyst(e)ine, in a cell. In some embodiments, the secondary active agent is a compound, molecule, or other composition effective to induce ferroptosis in a cell. In some embodiments, the secondary active agent is effective to inhibit the X_{c}⁻ antiporter in the cell or cell population. In some embodiments, the secondary active agent is selected from erastin, Ras-selective lethal small molecule 3, sulfasalazine, lanperisone, sorafenib, fenugreek (trigonelline), acetaminophen, cisplatin, artesunate, combination of siramesine and lapatinib, ferumoxytol, and salinomycin (ironomycin), artesunate, dihydroartemisnin, gemcitabine, paclitaxel, temozolomide, buthionine sulfoximine (BSO), solasonine, siramesine, laptinib, BAY 87-2243, Molecule 1 of Taylor et al., 2019 Sci. Reports. 9:5926 and its derivatives or metabolites (specifically molecule 4 of Taylor et al. 2019) any others set forth in e.g., Su et al., Cancer. Lett. 2020. Jul. 28; 483:127-136. doi: 10.1016/j.canlet.2020.02.015; Wu et al., 2020. Front. Oncol. | https://doi.org/10.3389/fonc.2020.571127; Lin et al., Front. Pharmacol. 2020. 11: 1061; Taylor et al., 2019. Sci. Reports. 9:5926, all of which are incorporated by reference herein), or any combination thereof.

In some embodiments, the additional active agent is selected from erastin, Ras-selective lethal small molecule 3, sulfasalazine or analogue thereof, lanperisone, sorafenib, fenugreek (trigonelline), acetaminophen, cisplatin, artesunate, siramesine, lapatinib, a combination of siramesine and lapatinib, ferumoxytol, salinomycin (ironomycin), dihydroartemisnin, gemcitabine, paclitaxel, temozolomide, buthionine sulfoximine (BSO), solasonine, siramesine, laptinib, BAY 87-2243,

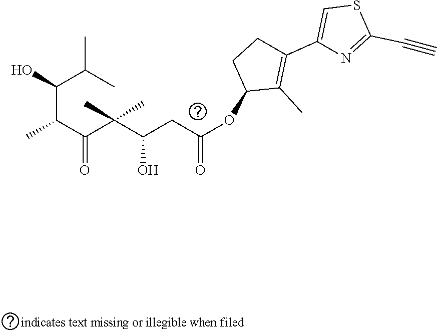

or a derivative or metabolite thereof, optionally

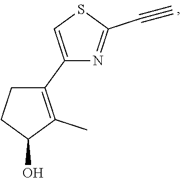

BLZ945, Dyclonine, Oxyfedrine, RSL3, ML210, SSZ, RSL3, ML162, FIN56, Anti-PD1, gefitnib, erlotinib, TMZ, docetaxel, DAT, vemurafenib, lovastatin, atorvastatin, simvastatin, prominin2, artemisinin or a derivative thereof, vitamin E, baicalein, beta-elemene, gallic acid, buthionine sulfoximine, DP17, FIN56, FINO₂, a statin, deferoxamine mesylate, or any combination thereof.

### Administration of the Pharmaceutical Formulations

The pharmaceutical formulations or dosage forms thereof described herein can be administered one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g., 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time.

### Kits

Any of the engineered biomolecules, vectors, vector systems, delivery vehicles, compounds, compositions, formulations, particles, cells, described herein or a combination thereof can be presented as a combination kit. As used herein, the terms “combination kit” or “kit of parts” refers to the compounds, compositions, formulations, particles, cells and any additional components that are used to package, sell, market, deliver, and/or administer the combination of elements or a single element, such as the active ingredient, contained therein. Such additional components include, but are not limited to, packaging, syringes, blister packages, bottles, and the like. When one or more of the c engineered biomolecules, vectors, vector systems, delivery vehicles, compounds, compositions, formulations, particles, cells, described herein or a combination thereof (e.g., agents) contained in the kit are administered simultaneously, the combination kit can contain the active agents in a single formulation, such as a pharmaceutical formulation, (e.g., a tablet a solution, etc.) or in separate formulations that can be optionally combined prior to administration or optionally mix upon or after administration. When the engineered biomolecules, vectors, vector systems, delivery vehicles, compounds, compositions, formulations, particles, cells, described herein or a combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the c engineered biomolecules, vectors, vector systems, delivery vehicles, compounds, compositions, formulations, particles, cells, described herein or a combination thereof contained therein, safety information regarding the content of the engineered biomolecules, vectors, vector systems, delivery vehicles, compounds, compositions, formulations, particles, cells, described herein or a combination thereof contained therein, information regarding the dosages, indications for use, and/or recommended treatment regimen(s), applications, and/or use for the engineered biomolecules, vectors, vector systems, delivery vehicles, compounds, compositions, formulations, particles, cells, described herein contained therein. In some embodiments, the instructions can provide directions for administering the engineered biomolecules, vectors, vector systems, delivery vehicles, compounds, compositions, formulations, particles, cells, described herein or a combination thereof to a subject and/or cell(s) in need thereof. In some embodiments the cell has depleted cyst(e)ine, particularly cytosolic cyst(e)ine. In some embodiments, the cell and/or subject has been treated with an agent that is capable of specifically depleting intracellular cyst(e)ine. In some embodiments, the cell has been treated with an agent that inhibits the X_{c}⁻ antiporter. In some embodiments, the cell has been treated with an agent that induces ferroptosis. In some embodiments, the cell has a cyst(e)ine stress response. In some embodiments the cell has or is susceptible to induced ATF4 expression as a result of specific depletion of cyst(e)ine within the cytoplasm and/or lysosome.

In some embodiments, the subject in need thereof can be in need of a treatment or prevention for a cancer or a symptom thereof. In some embodiments the cell is a cancer cell.

In some embodiments the cell(s) is/are cancer or tumor cells. In some embodiments, the cancer cell is a hepatocellular carcinoma cell, a gastric carcinoma cell, ovarian carcinoma cell, pancreatic carcinoma cell, breast carcinoma cell, colorectal carcinoma cell, melanoma cell, head and neck cancer cell, kidney carcinoma cell, lung carcinoma cell, glioblastoma, lymphoma, or retinoblastoma cell.

### Methods of Using the Engineered Biomolecules

The engineered biomolecules, vectors, vectors systems, formulations thereof and the like can be used to, reallocate nutrients within a cell, induce and/or potentiate ferroptosis in a cell, inhibit cyst(e)ine depletion induced ATF4 expression in a cell, increase lysosomal cyst(e)ine, deplete cytosolic cyst(e)ine, inhibit a cyst(e)ine stress response in a cell, treat a disease or disorder in a subject and/or cell, or any combination thereof.

Described in certain example embodiments herein are methods that include delivering to a cell or cell population (a) an engineered biomolecule of the present disclosure; (b) a vector as described elsewhere herein; (c) a delivery vehicle as described elsewhere herein; (d) a pharmaceutical formulation as described elsewhere herein; or (e) any combination thereof. In certain example embodiments, ferroptosis is induced and/or potentiated in the cell or cell population. In certain example embodiments, cytosolic cysteine is decreased, lysosomal cysteine is increased, or both. In certain example embodiments, ATF4 expression is decreased and/or ATF4 expression induction is decreased. In certain example embodiments, the method further includes delivering to the cell an additional active agent. In certain example embodiments, the additional active agent is effective to induce ferroptosis in the cell or cell population. In certain example embodiments, the additional active agent is effective to inhibit the X_{c}⁻ antiporter. In certain example embodiments, the additional active agent is selected from erastin, Ras-selective lethal small molecule 3, sulfasalazine, lanperisone, sorafenib, fenugreek (trigonelline), acetaminophen, cisplatin, artesunate, combination of siramesine and lapatinib, ferumoxytol, and salinomycin (ironomycin), artesunate, dihydroartemisnin, gemcitabine, paclitaxel, temozolomide, buthionine sulfoximine (BSO), solasonine, siramesine, laptinib, BAY 87-2243, Molecule 1 of Taylor et al., 2019. Sci. Reports. 9:5926 and its derivatives or metabolites (specifically molecule 4 of Taylor et al. 2019) any others set forth in e.g., Su et al., Cancer. Lett. 2020. Jul. 28; 483:127-136. doi: 10.1016/j.canlet.2020.02.015; Wu et al., 2020. Front. Oncol. | https://doi.org/10.3389/fonc.2020.571127; Lin et al., Front. Pharmacol. 2020. 11: 1061; Taylor et al., 2019. Sci. Reports. 9:5926, all of which are incorporated by reference herein), or any combination thereof. In some embodiments, the additional active agent is selected from erastin, Ras-selective lethal small molecule 3, sulfasalazine or analogue thereof, lanperisone, sorafenib, fenugreek (trigonelline), acetaminophen, cisplatin, artesunate, siramesine, lapatinib, a combination of siramesine and lapatinib, ferumoxytol, salinomycin (ironomycin), dihydroartemisnin, gemcitabine, paclitaxel, temozolomide, buthionine sulfoximine (BSO), solasonine, siramesine, laptinib, BAY 87-2243,

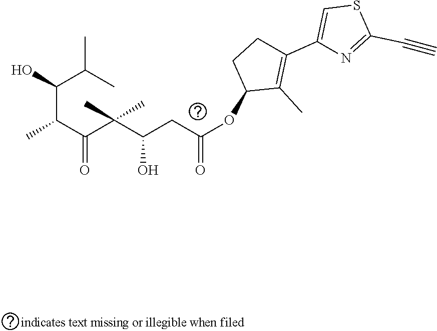

or a derivative or metabolite thereof, optionally

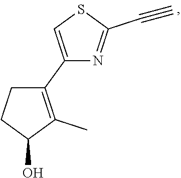

BLZ945, Dyclonine, Oxyfedrine, RSL3, ML210, SSZ, RSL3, ML162, FIN56, Anti-PD1, gefitnib, erlotinib, TMZ, docetaxel, DAT, vemurafenib, lovastatin, atorvastatin, simvastatin, prominin2, artemisinin or a derivative thereof, vitamin E, baicalein, beta-elemene, gallic acid, buthionine sulfoximine, DP17, FIN56, FINO₂, a statin, deferoxamine mesylate, or any combination thereof.

In certain example embodiments, the cell(s) is/are a cancer cell(s), neuron(s) (nerve cells), neuronal support cell(s) (e.g., astrocyte(s), microglial cell(s), Schwan cell(s), and the etc.), muscle cell(s) (including, smooth, cardiac, and/or skeletal muscle cell (s)), skin cell(s), hair follicle cell(s), hair cell(s) (ear), retinal cell(s), bone cell(s), blood cell(s), fat cell(s), gamete(s), endothelial cell(s), liver cell(s), kidney cell(s), lung cell(s), adrenal cell(s), bladder cell(s), pancreatic cell(s), intestinal cell(s), blood vessel cell(s), stomach cell(s), stem cell(s) (e.g., mesenchymal stem cells, adipose stem cells, hematopoetic stem cells, etc.), hormone secreting cell(s), exocrine secretory epithelial ell(s), oral cell(s), epithelial cell(s), immune cell(s) (e.g., monocytes, macrophages, T-cells, B-cells, neutrophils, eosinophils, lymphocytes, etc), and combinations thereof. In some embodiments, the cancer cell is a hepatocellular carcinoma cell, a gastric carcinoma cell, ovarian carcinoma cell, pancreatic carcinoma cell, breast carcinoma cell, colorectal carcinoma cell, melanoma cell, head and neck cancer cell, kidney carcinoma cell, lung carcinoma cell, glioblastoma, lymphoma, or retinoblastoma cell.

Described in certain embodiments are methods of treating a proliferative disease in a subject in need thereof that include administering to the subject (a) an engineered biomolecule of the present disclosure; (b) a vector as described elsewhere herein; (c) a delivery vehicle as described elsewhere herein; (d) a pharmaceutical formulation as described elsewhere herein; or (e) any combination thereof. In certain example embodiments, ferroptosis is induced and/or potentiated in a cell or cell population in the subject. In certain example embodiments, cytosolic cysteine is decreased in and/or lysosomal cysteine is increased, in a cell or cell population in the subject. In certain example embodiments, ATF4 expression is decreased and/or ATF4 expression induction is decreased in a cell or cell population in the subject. In certain example embodiments, the cell or cell population is a cancer cell or cancer cell population. In certain example embodiments, the method further includes administering an additional active agent to the subject. In certain example embodiments, the additional active agent is administered simultaneously, contemporaneously, or serially with (a)-(e). In certain example embodiments, the additional active agent is effective to induce ferroptosis in a cell or cell population in the subject. In certain example embodiments, the additional active agent is effective to inhibit the X_{c}⁻ antiporter in a cell or cell population in the subject. In certain example embodiments, the additional active agent is selected from erastin, Ras-selective lethal small molecule 3, sulfasalazine, lanperisone, sorafenib, fenugreek (trigonelline), acetaminophen, cisplatin, artesunate, combination of siramesine and lapatinib, ferumoxytol, and salinomycin (ironomycin), artesunate, dihydroartemisnin, gemcitabine, paclitaxel, temozolomide, buthionine sulfoximine (BSO), solasonine, siramesine, laptinib, BAY 87-2243, Molecule 1 of Taylor et al., 2019. Sci. Reports. 9:5926, and its derivatives or metabolites (specifically molecule 4 of Taylor et al. 2019) any others set forth in e.g., Su et al., Cancer. Lett. 2020. Jul. 28; 483:127-136. doi: 10.1016/j.canlet.2020.02.015; Wu et al., 2020. Front. Oncol. | https://doi.org/10.3389/fonc.2020.571127; Lin et al., Front. Pharmacol. 2020. 11: 1061; Taylor et al., 2019. Sci. Reports. 9:5926, all of which are incorporated by reference herein), and any combination thereof. In some embodiments, the additional active agent is selected from erastin, Ras-selective lethal small molecule 3, sulfasalazine or analogue thereof, lanperisone, sorafenib, fenugreek (trigonelline), acetaminophen, cisplatin, artesunate, siramesine, lapatinib, a combination of siramesine and lapatinib, ferumoxytol, salinomycin (ironomycin), dihydroartemisnin, gemcitabine, paclitaxel, temozolomide, buthionine sulfoximine (BSO), solasonine, siramesine, laptinib, BAY 87-2243,

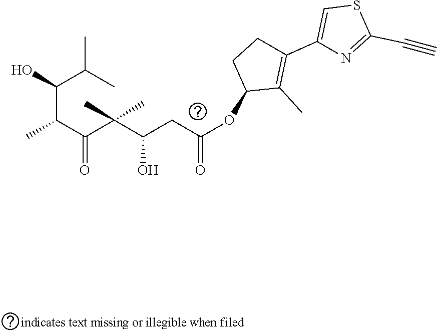

or a derivative or metabolite thereof, optionally

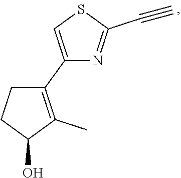

BLZ945, Dyclonine, Oxyfedrine, RSL3, ML210, SSZ, RSL3, ML162, FIN56, Anti-PD1, gefitnib, erlotinib, TMZ, docetaxel, DAT, vemurafenib, lovastatin, atorvastatin, simvastatin, prominin2, artemisinin or a derivative thereof, vitamin E, baicalein, beta-elemene, gallic acid, buthionine sulfoximine, DP17, FIN56, FINO₂, a statin, deferoxamine mesylate, or any combination thereof.

In certain example embodiments, cancer cell growth, cancer tumor growth, or both is inhibited, slowed, and/or stopped. In some embodiments, the cancer cell is a hepatocellular carcinoma cell, a gastric carcinoma cell, ovarian carcinoma cell, pancreatic carcinoma cell, breast carcinoma cell, colorectal carcinoma cell, melanoma cell, head and neck cancer cell, kidney carcinoma cell, lung carcinoma cell, glioblastoma, lymphoma, or retinoblastoma cell.

Described in exemplary embodiments herein are methods of inhibiting a cysteine stress response in a cell or cell population that include delivering to the cell or cell population (a) an engineered biomolecule of the present disclosure; (b) a vector as described elsewhere herein; (c) a delivery vehicle as described elsewhere herein; (d) a pharmaceutical formulation as described elsewhere herein; or (e) any combination thereof. In some embodiments, ferroptosis is induced and/or potentiated in a cell or cell population. In some embodiments, cytosolic cysteine is decreased in and/or lysosomal cysteine is increased in the cell or cell population. In some embodiments, ATF4 expression is decreased and/or ATF4 expression induction is decreased in the cell or cell population. In certain example embodiments, the cell(s) is/are a cancer cell(s), neuron(s) (nerve cells), neuronal support cell(s) (e.g., astrocyte(s), microglial cell(s), Schwan cell(s), and the etc.), muscle cell(s) (including, smooth, cardiac, and/or skeletal muscle cell (s)), skin cell(s), hair follicle cell(s), hair cell(s) (ear), retinal cell(s), bone cell(s), blood cell(s), fat cell(s), gamete(s), endothelial cell(s), liver cell(s), kidney cell(s), lung cell(s), adrenal cell(s), bladder cell(s), pancreatic cell(s), intestinal cell(s), blood vessel cell(s), stomach cell(s), stem cell(s) (e.g., mesenchymal stem cells, adipose stem cells, hematopoetic stem cells, etc.), hormone secreting cell(s), exocrine secretory epithelial ell(s), oral cell(s), epithelial cell(s), immune cell(s) (e.g., monocytes, macrophages, T-cells, B-cells, neutrophils, eosinophils, lymphocytes, etc), and combinations thereof. In some embodiments, the method further includes delivering an additional active agent to the cell or cell population. In some embodiments, the additional active agent is effective to induce ferroptosis in the cell or cell population. In some embodiments, the additional active agent is effective to inhibit the X_{c}⁻ antiporter in the cell or cell population. In some embodiments, the additional active agent is delivered simultaneously, contemporaneously, or serially with (a)-(e). the additional active agent is selected from erastin, Ras-selective lethal small molecule 3, sulfasalazine, lanperisone, sorafenib, fenugreek (trigonelline), acetaminophen, cisplatin, artesunate, combination of siramesine and lapatinib, ferumoxytol, and salinomycin (ironomycin), artesunate, dihydroartemisnin, gemcitabine, paclitaxel, temozolomide, buthionine sulfoximine (BSO), solasonine, siramesine, laptinib, BAY 87-2243, Molecule 1 of Taylor et al., 2019. Sci. Reports. 9:5926 and its derivatives or metabolites (specifically molecule 4 of Taylor et al. 2019) any others set forth in e.g., Su et al., Cancer. Lett. 2020. Jul. 28; 483:127-136. doi: 10.1016/j.canlet.2020.02.015; Wu et al., 2020. Front. Oncol. | https://doi.org/10.3389/fonc.2020.571127; Lin et al., Front. Pharmacol. 2020. 11: 1061; Taylor et al., 2019. Sci. Reports. 9:5926, all of which are incorporated by reference herein), and any combination thereof. In some embodiments, the additional active agent is selected from erastin, Ras-selective lethal small molecule 3, sulfasalazine or analogue thereof, lanperisone, sorafenib, fenugreek (trigonelline), acetaminophen, cisplatin, artesunate, siramesine, lapatinib, a combination of siramesine and lapatinib, ferumoxytol, salinomycin (ironomycin), dihydroartemisnin, gemcitabine, paclitaxel, temozolomide, buthionine sulfoximine (BSO), solasonine, siramesine, laptinib, BAY 87-2243,

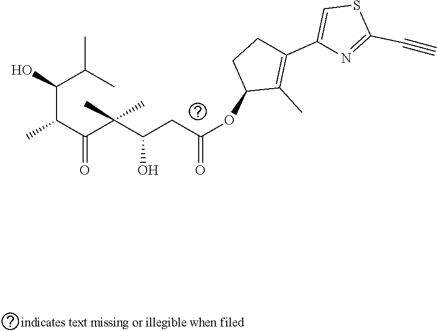

or a derivative or metabolite thereof, optionally

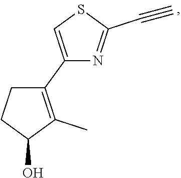

BLZ945, Dyclonine, Oxyfedrine, RSL3, ML210, SSZ, RSL3, ML162, FIN56, Anti-PD1, gefitnib, erlotinib, TMZ, docetaxel, DAT, vemurafenib, lovastatin, atorvastatin, simvastatin, prominin2, artemisinin or a derivative thereof, vitamin E, baicalein, beta-elemene, gallic acid, buthionine sulfoximine, DP17, FIN56, FINO₂, a statin, deferoxamine mesylate, or any combination thereof.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1

Cancer cells rely on a constant supply of nutrients such as amino acids to satisfy the increased anabolic demands. The ability of cancer cells to adapt to nutrient shortage is also critical for tumorigenesis. Upon amino acid restriction, the integrated stress response (ISR) is induced via GCN2 kinase (1, 2). Activated GCN2 phosphorylates eIF2α, resulting in translational reprogramming that inhibits general protein synthesis but paradoxically increases the translation of a subset of mRNAs (3, 4). The most-notable example of selective translation is activating transcription factor 4 (ATF4), a bZip transcription factor that promotes the expression of genes involved in antioxidant response and amino acid biosynthesis and transport (5). The ATF4-mediated adaptative program is thus crucial during tumor progression (5, 6). It is widely believed that the primary regulation of ATF4 expression is through translational control of pre-existing mRNA (7, 8). The transcriptional regulation of ATF4, however, remains surprisingly obscure.

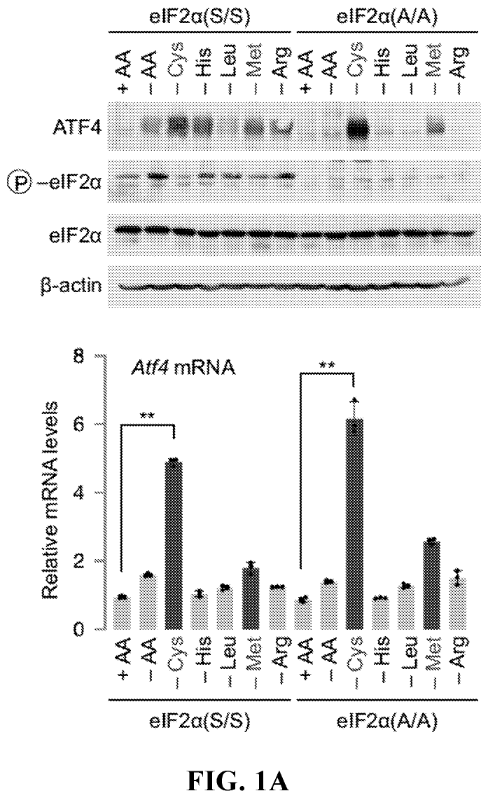

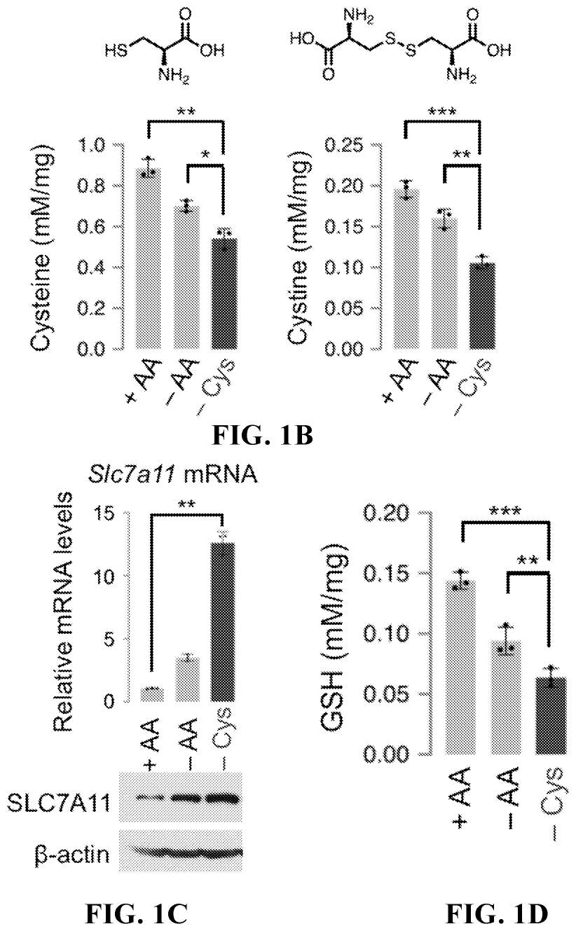

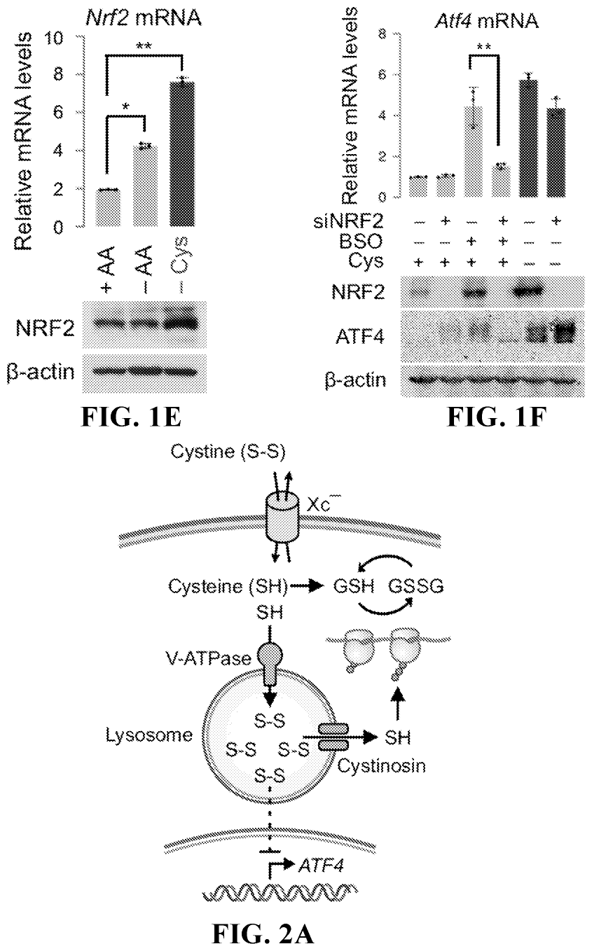

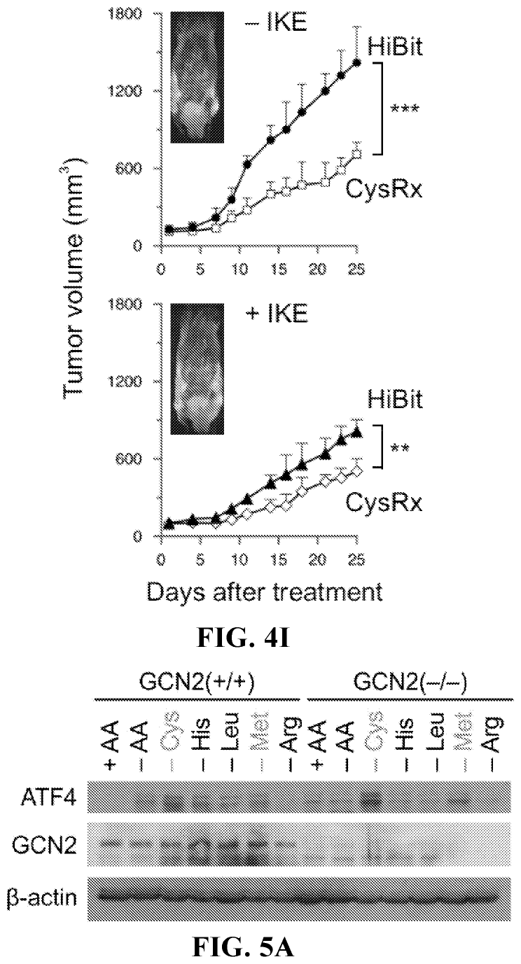

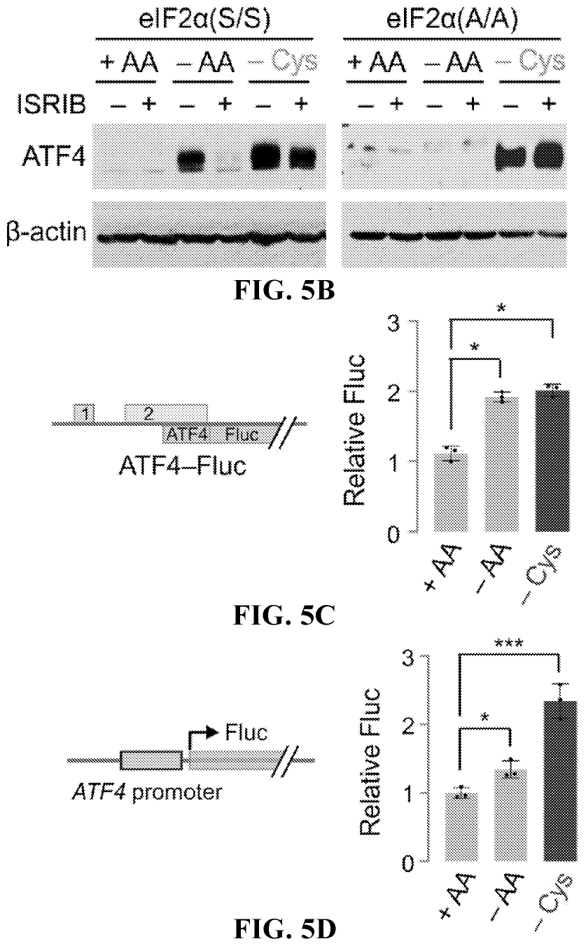

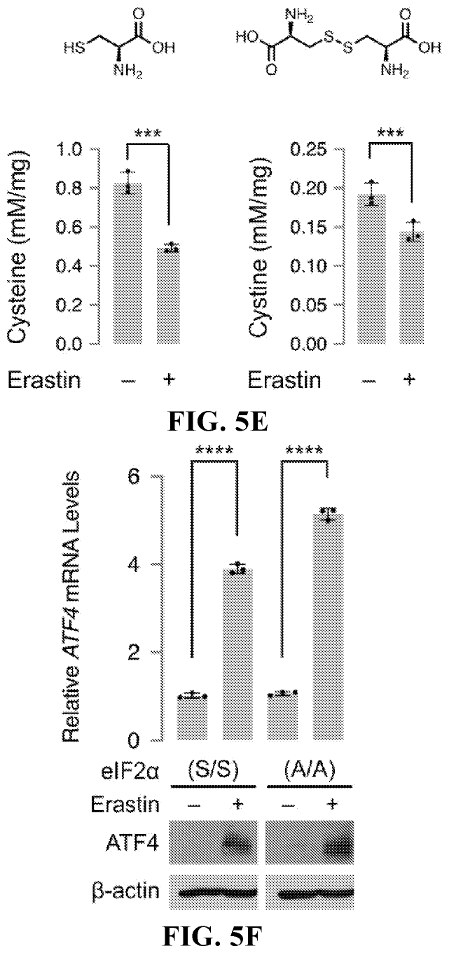

The current understanding of amino acid response is largely based on full amino acid starvation. It remains unclear whether single amino acid deprivation triggers the common ISR or elicits a unique cellular response. Applicant took advantage of a mouse embryonic fibroblast (MEF) cell line harboring a non-phosphorylatable eIF2α in which the serine 51 (S/S) was mutated to an alanine (A/A) (9). Wild type eIF2α (S/S) cells readily responded to whole amino acid starvation by showing increased eIF2a phosphorylation and robust ATF4 induction (FIG. 1A), which were abolished in eIF2α (A/A) cells. Single amino acid withdrawal, however, resulted in varied ATF4 induction. Notably, cystine starvation triggered the strongest ATF4 expression despite the minimal effect on eIF2α phosphorylation. Strikingly, a potent ATF4 induction was maintained in eIF2α (A/A) cells upon withdrawal of cystine, but not histidine, leucine, or arginine from the medium (FIG. 1A). The eIF2α-independent ATF4 induction was also seen after removal of methionine, a cysteine precursor, albeit to a lower extent. The similar finding holds true in MEF cells lacking GCN2 (FIG. 5A). Further confirming the eIF2α independency, the cystine shortage-induced ATF4 upregulation was insensitive to ISRIB, a small-molecule ISR inhibitor (FIG. 5B). Using a firefly luciferase (Fluc) reporter bearing the 5′ UTR of Atf4, Applicant found comparable Fluc levels between whole amino acid starvation and cystine deprivation (FIG. 5C), suggesting that mechanisms beyond translational control likely contribute to the ATF4 induction upon cystine shortage. Applicant next measured Atf4 mRNA levels in starved cells using RT-qPCR. In comparison to full amino acid starvation that induced Atf4 expression by ˜2 fold, cystine deprivation increased Atf4 mRNA levels by >5 fold (FIG. 1A, bottom panel). The transcriptional upregulation of ATF4 was confirmed by a Fluc reporter driven by the Atf4 promoter (FIG. S1D). Therefore, cystine shortage induces a unique transcriptional induction of ATF4, which surprisingly overcomes the translational control of ATF4 mediated by eIF2α phosphorylation.

The extracellular cystine is actively transported into cells via the cystine-glutamate antiporter system X_{c}⁻ (10). Once inside cells, each cystine is reduced to two molecules of cysteine. Not surprisingly, cystine depletion from the medium lowered intracellular levels of both cystine and cysteine (FIG. 1B). Similar to cystine starvation, treatment with erastin, a potent system X_{c}⁻ inhibitor (11), decreased intracellular cystine and cysteine levels (FIG. 5E).

Likewise, erastin treatment led to a robust induction of ATF4 independent of eIF2α phosphorylation (FIG. 5F). Notably, cystine withdrawal led to a marked increase of SLC7A11, an essential subunit of the system X_{c}⁻ antiporter (FIG. 1C). Since SLC7A11 is a downstream target of ATF4 (6), the upregulated X_{c}⁻ represents an adaptive cellular response to mitigate cystine shortage.

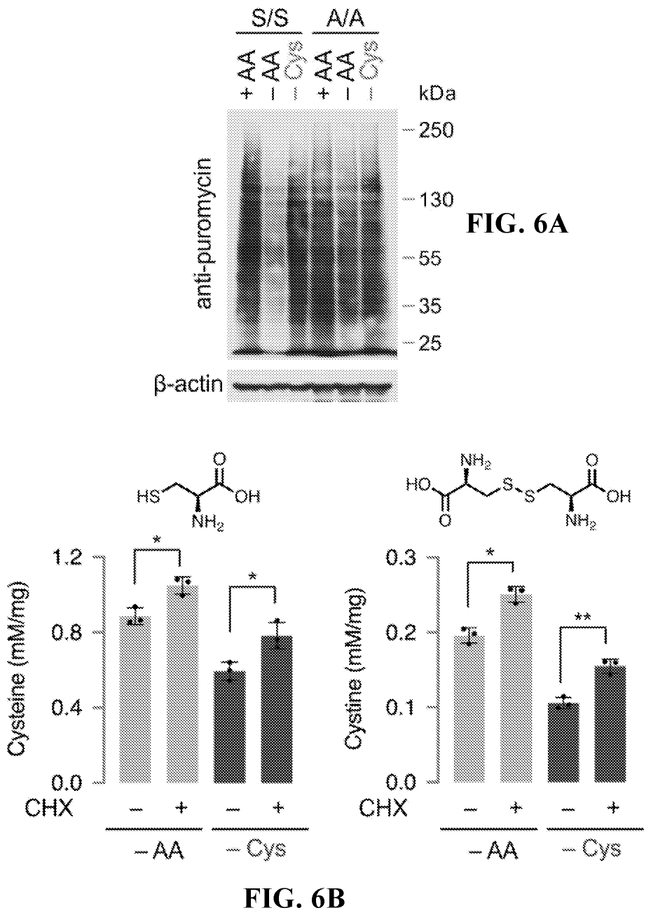

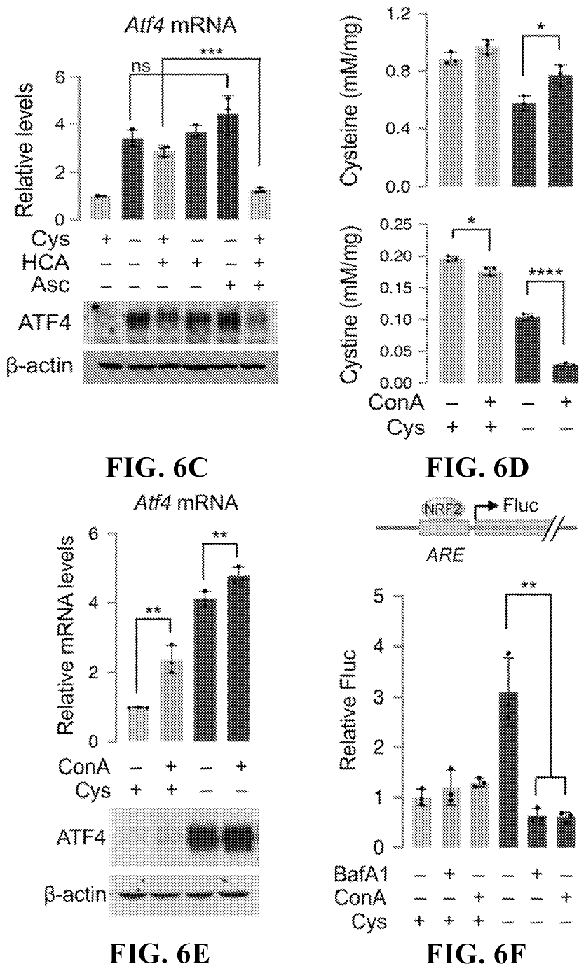

It is intriguing to find that full amino acid starvation is less potent in ATF4 induction despite the similar cystine shortage. Applicant found that withdrawal of cystine alone resulted in a more significant decrease of intracellular cyst(e)ine levels than full amino acid starvation (FIG. 1B). This is likely due to their differential effects on global protein synthesis. Indeed, puromycin labeling revealed a rapid inhibition of global protein synthesis upon full amino acid starvation but not upon cystine depletion (FIG. 6A). This is in line with the notion that cystine starvation minimally induces ISR. The continuous protein synthesis under cystine restriction is expected to further deplete the intracellular cyst(e)ine pool. Supporting this notion, translation inhibition by cycloheximide (CHX) largely restored the cyst(e)ine levels (FIG. 6B), indicating that translation is a major consumer of intracellular cysteine.

In addition to protein synthesis, intracellular cysteine is utilized for the synthesis of metabolites such as glutathione (GSH), a primary cellular antioxidant (12). Indeed, there was a 50% reduction of GSH in cystine starved cells (FIG. 1D). As expected, a decline in intracellular GSH led to oxidative stress response as evidenced by the elevated NRF2 (13) (FIG. 1E). Given the much higher NRF2 induction under cystine starvation, Applicant wondered whether the robust ATF4 upregulation was a result of excessive oxidative stress response (14). However, the antioxidant ascorbic acid had little effects on ATF4 induction upon cystine starvation (FIG. 6C). By contrast, the same treatment attenuated ATF4 expression induced by homocysteic acid (HCA), a potent oxidative stress inducer (14). To substantiate this finding further, Applicant induced oxidative stress response by inhibiting GSH biosynthesis using buthionine sulfoximine (BSO). Silencing NRF2 suppressed ATF4 expression induced by BSO but had little effect on cystine-starved cells (FIG. 1F). Therefore, other mechanisms are likely in place to mediate the transcriptional activation of ATF4 in response to cystine shortage.

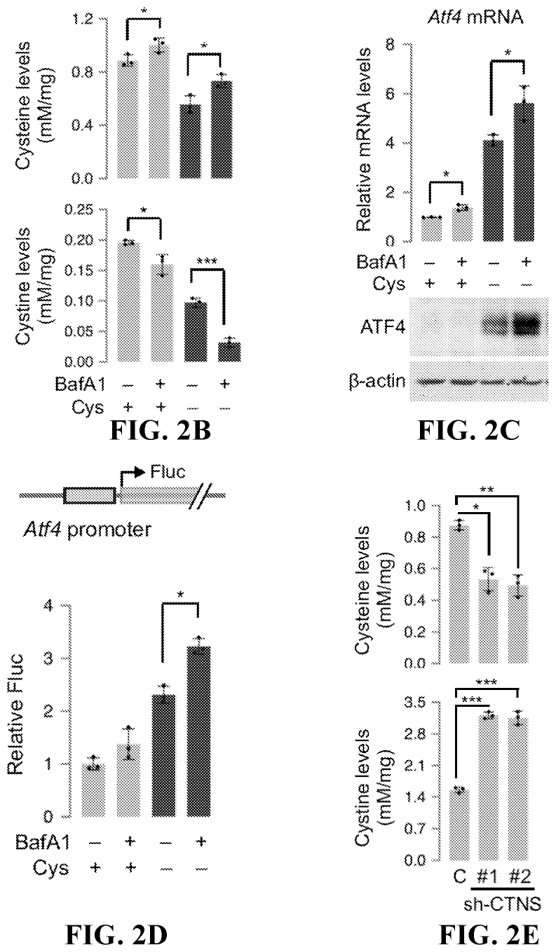

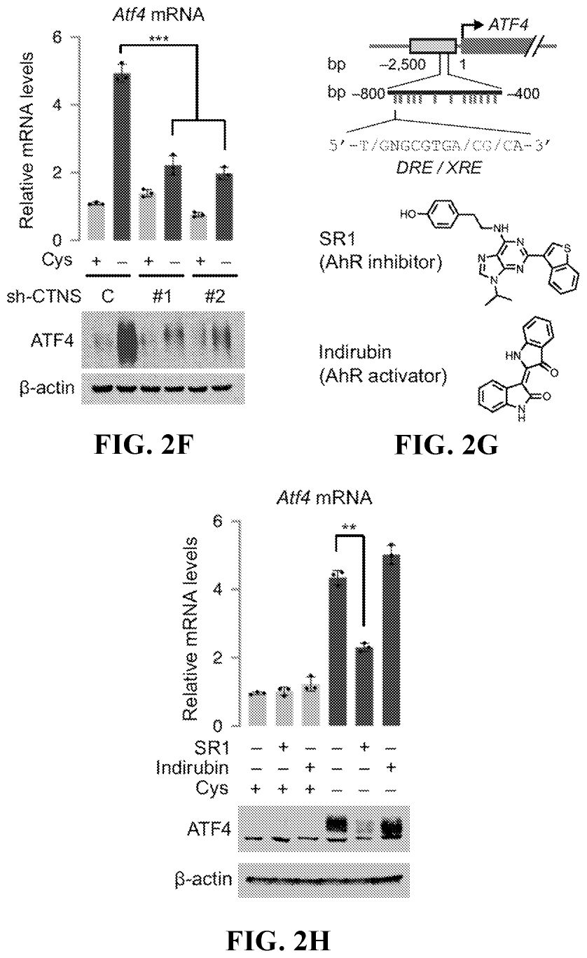

A prior study demonstrated that intracellular cysteine is mostly stored in lysosomes as cystine by a process involving the V-ATPase pump (15) (FIG. 2A). Indeed, V-ATPase inhibition by bafilomycin A1 (BafA1) resulted in an increase of cysteine and a corresponding decrease in cystine (FIG. 2B). Remarkably, blocking the influx of cysteine into the lysosome greatly potentiated ATF4 induction in cystine-starved cells (FIG. 2C). Since the cytosolic cysteine level was partially restored in the presence of BafA1, this result suggests that it is the shortage of lysosomal cystine that triggers Atf4 gene expression. The same result was obtained by applying another V-ATPase inhibitor concanamycin A (ConA) (FIGS. 6D and 6E). The transcriptional upregulation of ATF4 upon V-ATPase inhibition was further confirmed by the Atf4 promoter-driven reporter assay (FIG. 2D). Applicant next examined NRF2 activity using a Fluc reporter containing the antioxidant responsive element (ARE). V-ATPase inhibition evidently prevented NRF2 activation (FIG. 6F), which is in line with the elevated cysteine levels in the cytosol. Applicant concluded that the transcriptional regulation of ATF4 is governed by lysosomal cystine in an ISR- and NRF2-independent manner.

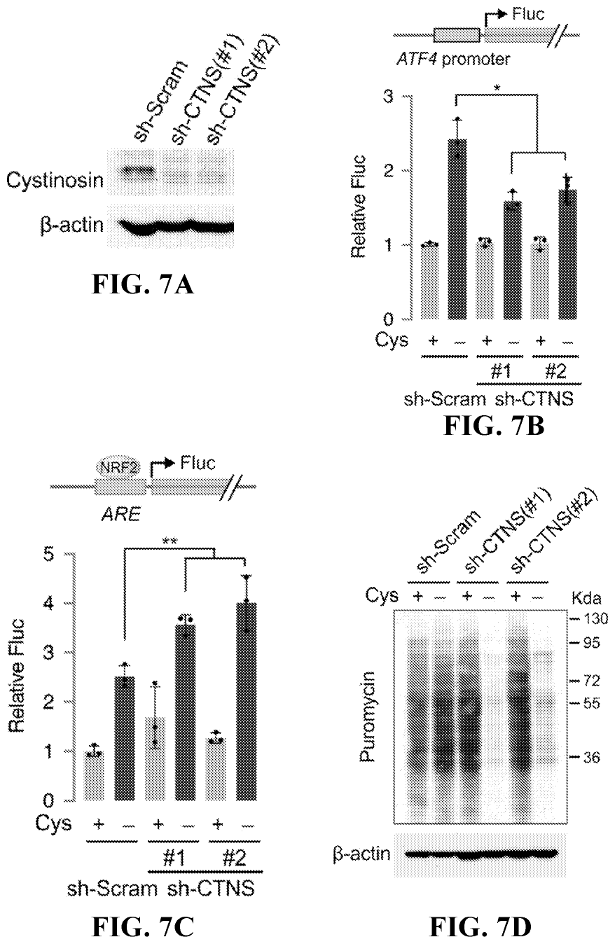

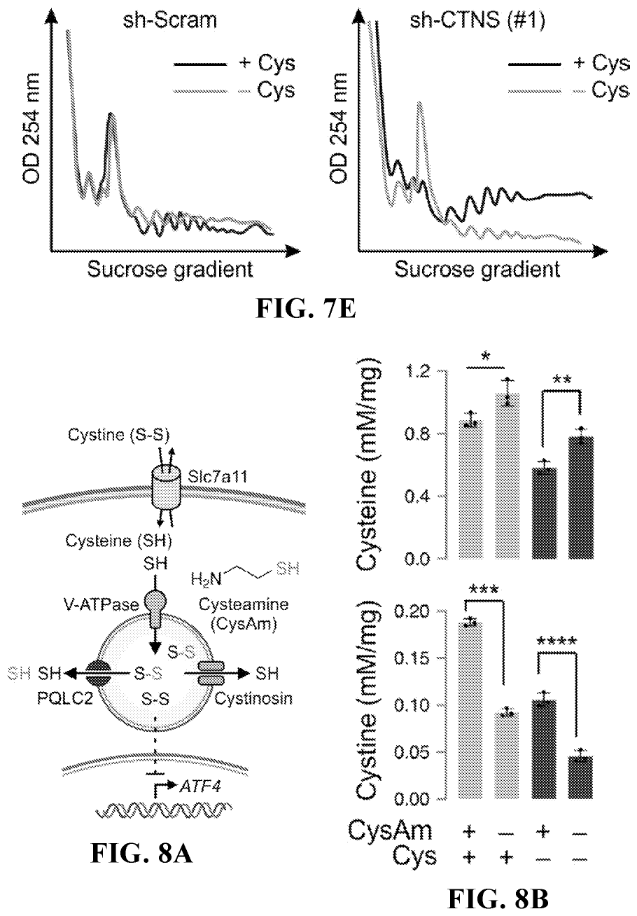

Besides the V-ATPase-mediated cystine storage in lysosome, there is an active lysosomal efflux system that supplies intracellular cysteine when extracellular cystine is limited (FIG. 2A). This lysosomal cystine transporter known as cystinosin is encoded by CTNS, a gene mutated in the lysosomal storage disorder cystinosis (16). As expected, silencing CTNS led to an increase of cystine and a decrease of cysteine levels (FIG. 7A and FIG. 2E). Remarkably, ATF4 induction upon cystine withdrawal was largely blunted in the absence of cystinosin (FIG. 2F), and this was further confirmed by the Atf4 promoter-driven reporter assay (FIG. 7B). Therefore, an accumulation of lysosomal cystine appears to attenuate ATF4 expression. Notably, cells lacking cystinosin exhibited increased NRF2 activation (FIG. S3C), which is in line with the lowered cysteine levels. In comparison to control cells, CTNS silencing readily suppressed global protein synthesis in cystine-starved cells (FIGS. S3D and S3E), a strong indication of depleted cytosolic cysteine. Therefore, even under the shortage of cytosolic cysteine, an accumulation of lysosomal cystine attenuates the adaptive ATF4 response.

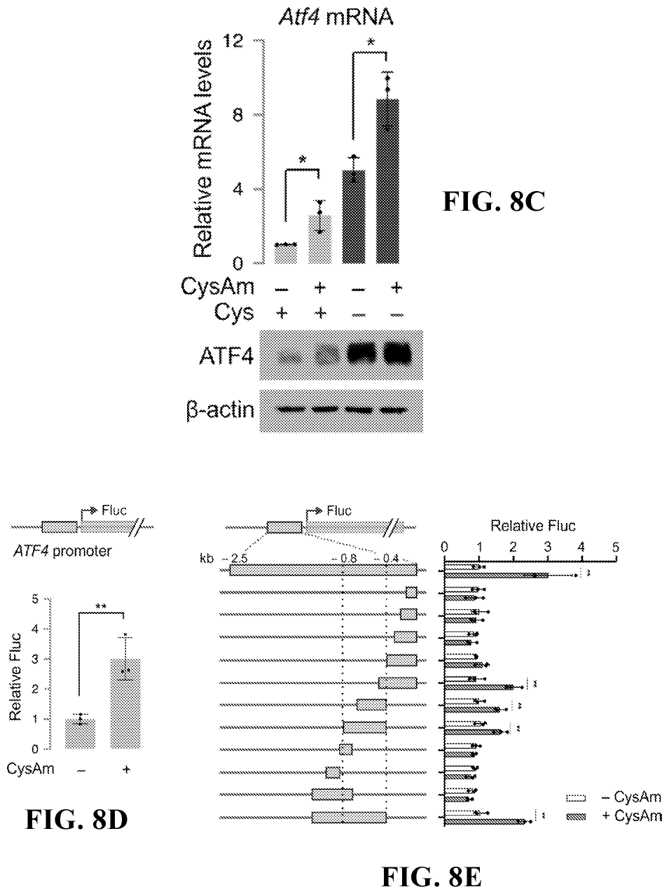

To further confirm that the attenuated ATF4 expression in cells lacking cystinosin is attributed to the accumulated lysosomal cystine, Applicant treated cells with cysteamine to resolve cystine accumulation independent of cystinosin (FIG. 8A) (17). As expected, cysteamine treatment readily depleted lysosomal cystine with a corresponding increase of cysteine in the cytosol (FIG. 8B). Importantly, cysteamine treatment led to a pronounced ATF4 induction even under the cystine rich condition (FIG. 8C). Without absolute cyst(e)ine depletion, a decrease in lysosomal cystine is sufficient to trigger ATF4 induction as evidenced by the Atf4 promoter-driven reporter assay (FIG. 8D). Altogether, Applicant demonstrate the existence of a signaling pathway linking lysosomal cystine and nuclear Atf4 gene expression.

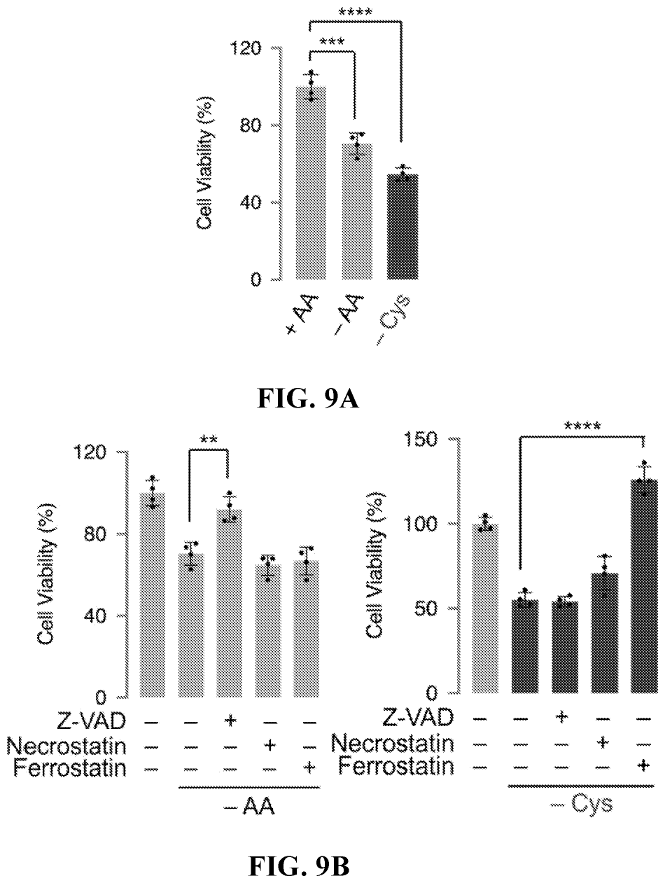

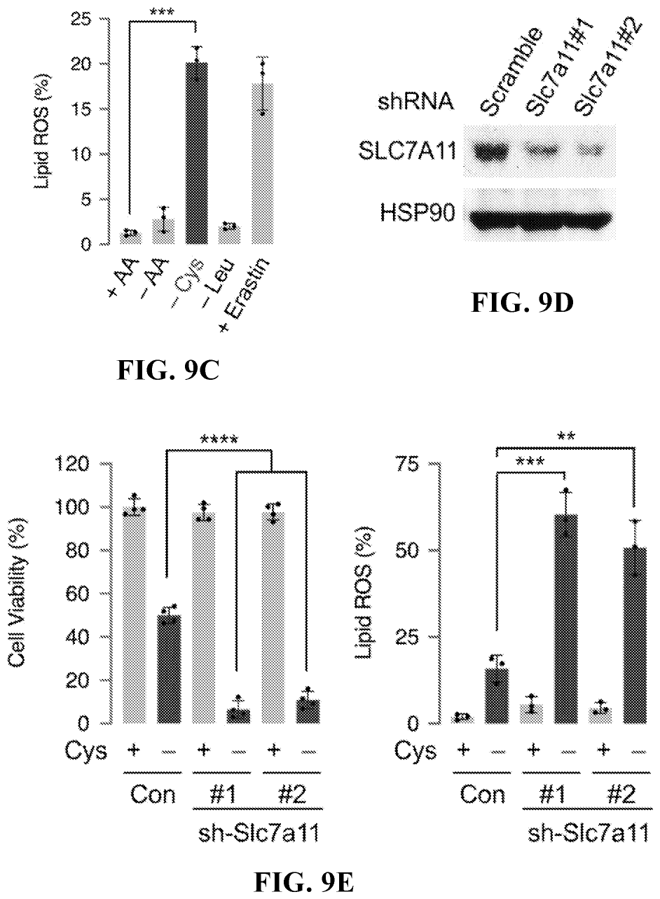

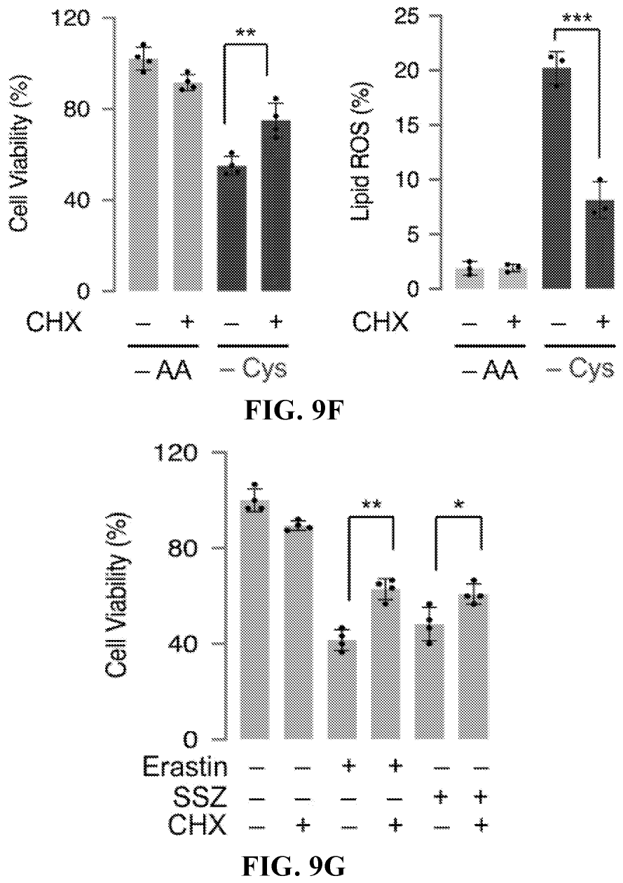

To probe how a shortage of lysosomal cystine leads to transcriptional response of ATF4, Applicant dissected the Atf4 promoter region using the Fluc reporter assay. Like the endogenous Atf4, a reporter containing the 2.5-kb region upstream of the Atf4 transcription start site showed a robust response to cysteamine treatment (FIG. 8D). Targeted deletion of this upstream region revealed a sub-region between −400 and −800 bp that was critical for maintaining the cysteamine sensitivity (FIG. 2G and FIG. 9E). Analysis of this critical sub-region identified 13 motifs with similarity to dioxin response element (DRE), which is also referred to as xenobiotic response element (XRE) (FIG. 2G). The DRE/XRE is recognized by activated aryl hydrocarbon receptor (AhR) (18), which sense a wide range of environmental stimuli as well as intracellular metabolites (19). It appears that lysosomal cystine serves as an antagonist to AhR signaling pathways. To confirm the participation of AhR in the transcriptional regulation of ATF4, Applicant treated cystine-starved cells with AhR modulators (FIG. 2G, bottom panel). While the AhR inhibitor SR1 significantly reduced the ATF4 induction, the AhR activator indirubin further boosted the ATF4 levels (FIG. 2H). The modest effect of indirubin suggests that activated AhR signaling is nearly maximal upon cystine deprivation.

It has been well-established that cyst(e)ine depletion leads to ferroptosis, a peroxidation-driven and iron-catalyzed form of non-apoptotic cell death (11, 20). Indeed, the cell death under cystine restriction was prevented by ferrostatin-1 (a ferroptosis inhibitor) but not Z-VAD (an apoptosis inhibitor) or necrostatin (a necrosis inhibitor) (FIGS. 9A and 9B). By contrast, the reduced cell viability under full amino acid starvation was rescued by Z-VAD only. One hallmark of ferroptosis is lipid peroxidation, which can be quantified by C11-BODIPY staining. Cystine withdrawal, but not full amino acid starvation or leucine restriction, caused a large increase in lipid oxidation (FIG. 9C). Additionally, genetic silencing of Slc7a11 promoted ferroptosis of cystine-starved cells (FIGS. 9D and 9E). Since cystine starvation does not trigger ISR, the continuous protein synthesis is expected to exacerbate cysteine depletion and subsequent ferroptosis. Supporting this notion, the increased ferroptosis susceptibility was prevented by CHX treatment (FIG. 9F). Likewise, CHX also partially rescued ferroptosis induced by X_{c}⁻ inhibitors erastin and sulfasalazine (SSZ) (FIG. 9G). These results confirm that the cytosolic cysteine pool influences the ferroptosis susceptibility.

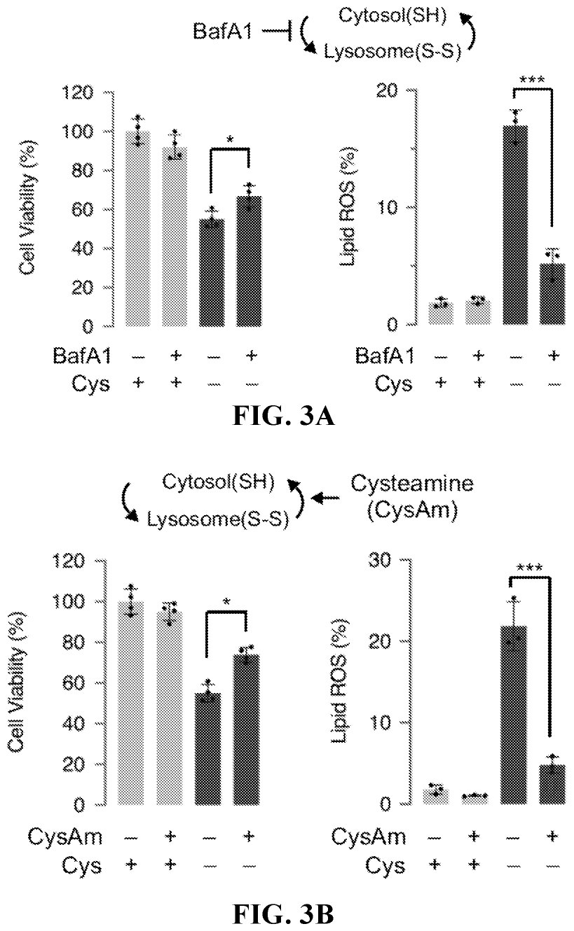

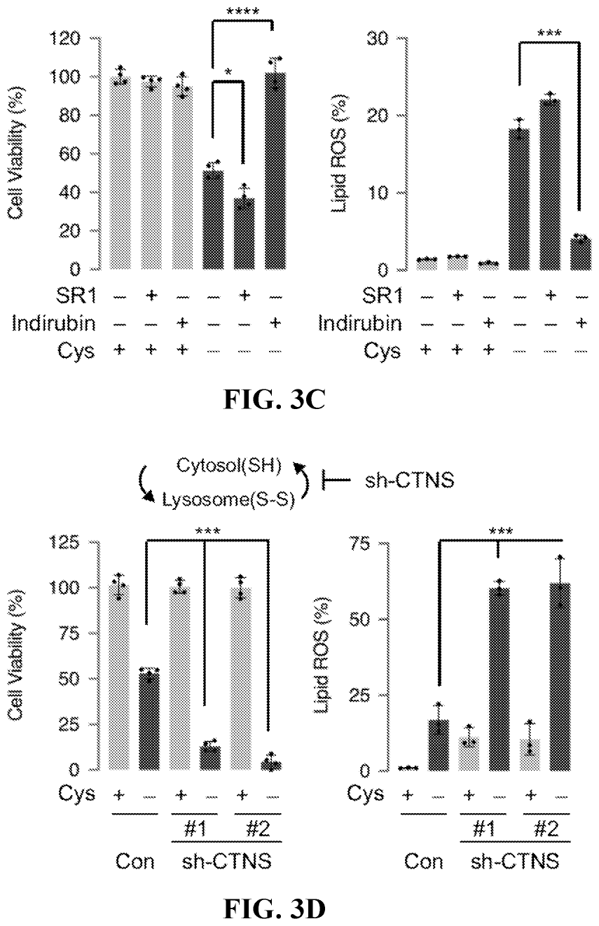

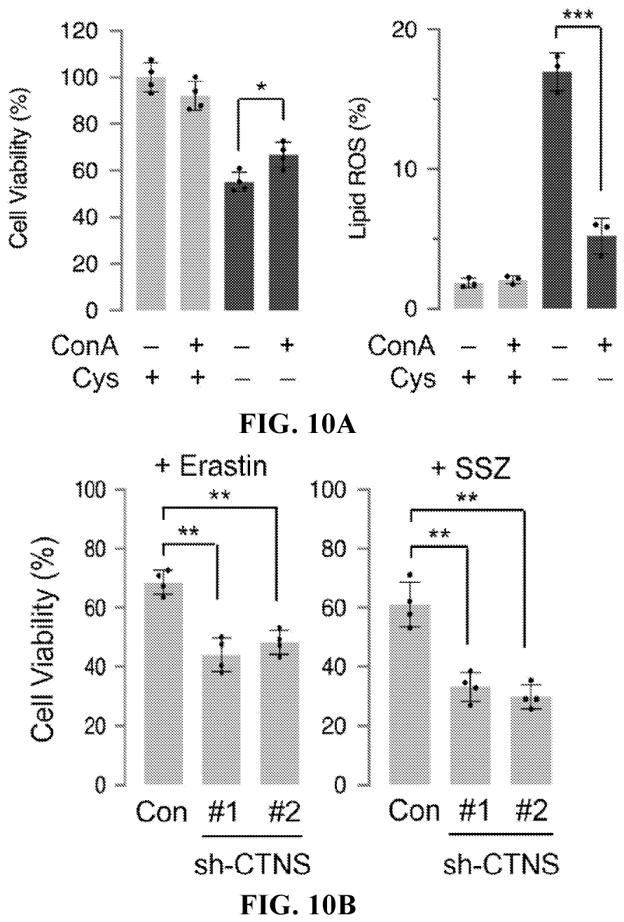

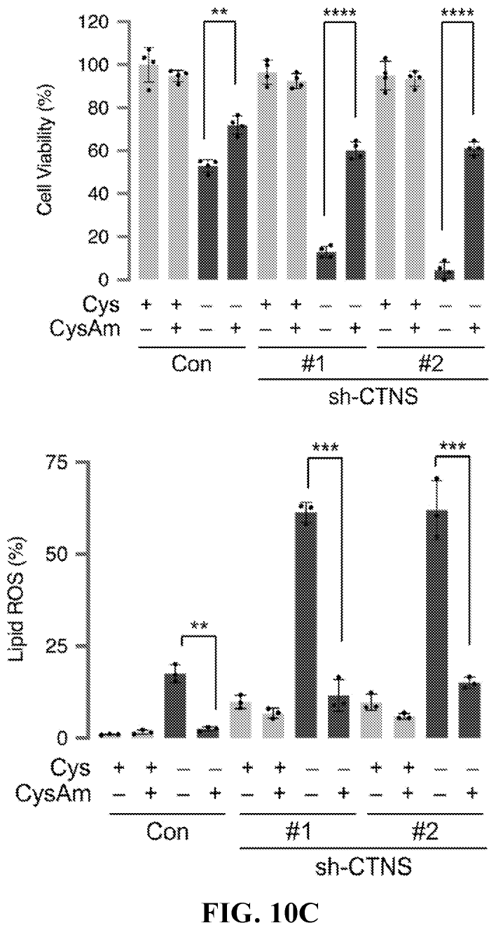

Applicant next investigated the role of lysosomal cystine in ferroptosis. V-ATPase inhibition by BafA1 or ConA prevented ferroptosis in cystine-starved cells and significantly reduced lipid oxidation (FIG. 3A and FIG. 10A). This is likely due to the retention of cysteine in the cytosol as a result of blocked lysosomal influx. Similarly, by mobilizing cystine out of lysosomes, cysteamine treatment also rescued cells from ferroptosis and eliminated lipid oxidation (FIG. 3B). Both treatments mitigate the cysteine scarcity in the cytosol at the expense of lysosomal cystine. Since a shortage of lysosomal cystine induces ATF4 expression via AhR, Applicant attempted to dissect the role of ATF4 in ferroptosis using AhR modulators. Without interfering with the intracellular cyst(e)ine recycling, the AhR inhibitor SR1 promoted ferroptosis of cystine-starved cells.

Strikingly, the AhR activator indirubin completely rescued the cells from ferroptosis with a marked reduction in lipid oxidation (FIG. 3C). Therefore, an increased ATF4 expression effectively counteracts ferroptosis, presumably via the increased X_{c}⁻ and enhanced antioxidant response.

Applicant reasoned that a blockade of lysosomal cystine efflux would maximize ferroptosis because a reduction of cytosolic cysteine is accompanied by accumulation of lysosomal cystine. While the former induces ferroptosis, the latter suppresses the adaptive ATF4 response. Indeed, cells lacking cystinosin became extremely sensitive to cystine withdrawal as evidenced by a drop in cell viability to ˜10% (FIG. 3D). Consistently, there was a surge in lipid oxidation in those cells (˜60%) in comparison to wild type cells (15%). The lack of cystinosin also rendered cells highly susceptible to X_{c}⁻ inhibitors erastin and SSZ (FIG. 10B). This was mainly due to the lysosomal cystine accumulation because cysteamine treatment rescued the ferroptotic cell death (FIG. 10C). Together, these experiments demonstrate that ferroptosis is influenced not only by the cytosolic cysteine levels, but also by lysosomal cystine via the adaptive ATF4 response.

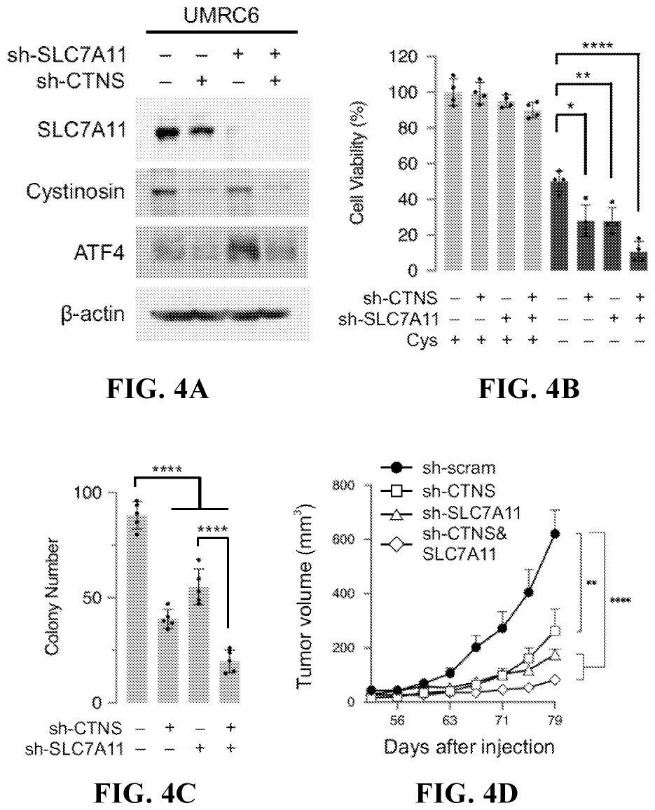

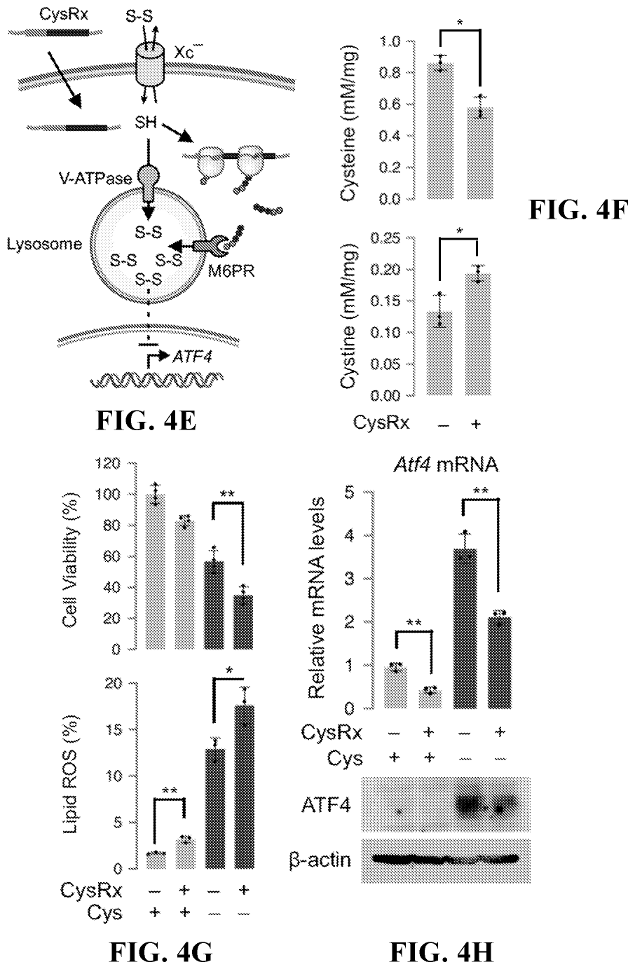

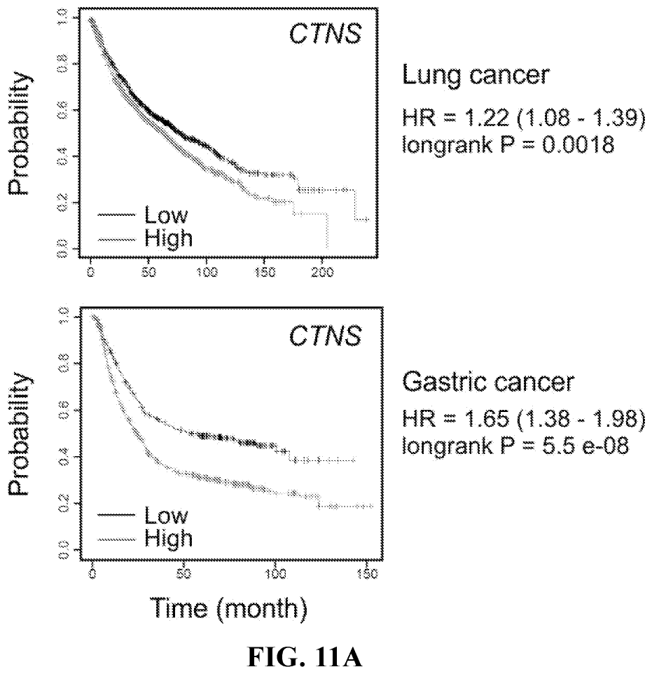

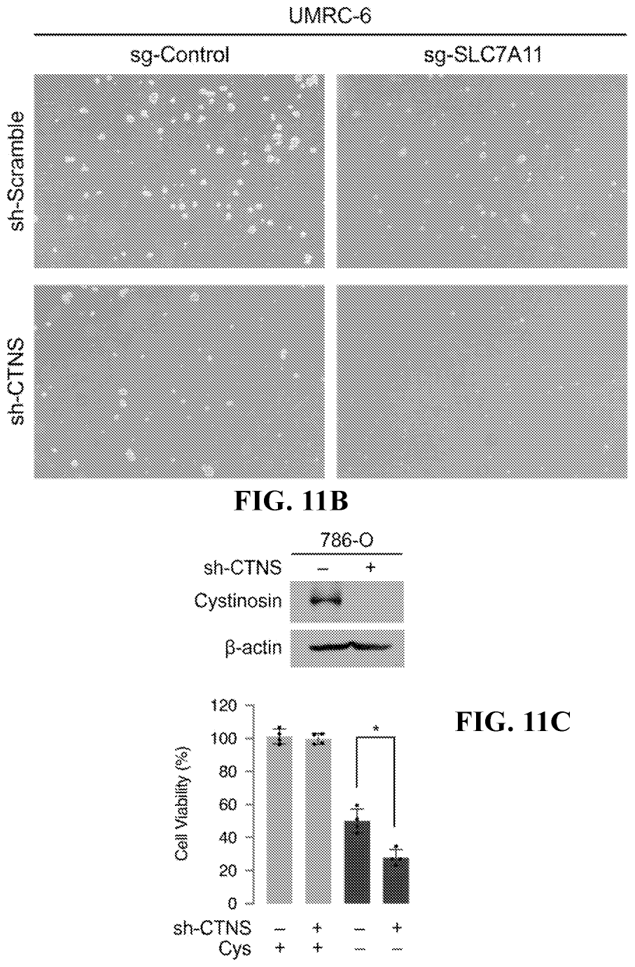

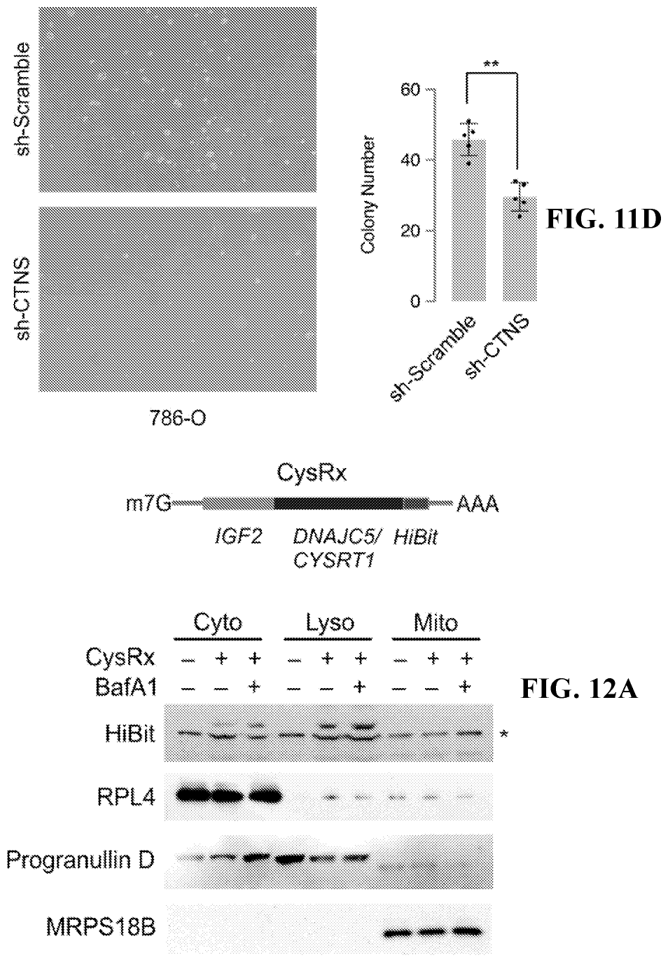

Recent studies of inducible ferroptosis in cancer cells have boosted a perspective for its application in cancer therapeutics (21). The commonly used cyst(e)ine depletion approaches by targeting X_{c}⁻ or using enzymes like cyst(e)inase have shown promising results by inducing tumor-selective ferroptosis (22, 23). However, the efficacy remains unsatisfactory partly due to the rapid adaption of cancer cells to cysteine limitation via induced ATF4 expression. Since a blockade of lysosomal cystine efflux potentiates ferroptosis, Applicant hypothesized that knocking down cystinosin would maximize ferroptoic death of cancer cells. Notably, Kaplan-Meier plotter reveal that high expression of CTNS correlates with decreased overall survival of lung and gastric cancer patients (FIG. 11A), suggesting an oncogenic role for cystinosin. Using a renal carcinoma (RC) cell line UMRC6 that has high levels of SLC7A11, Applicant found that silencing either SLC7A11 or CTNS potentiated ferroptosis upon cystine withdrawal (FIGS. 4A and 4B).

Remarkably, knocking down both SLC7A11 and CTNS caused severe cell death of UMRC6 cells, which was further manifested by deficient colony formation in soft agar (FIG. 4C and FIG. 11B). The tumor suppressive effect of cystinosin knockdown also holds true in another RC cell line 786-O despite the low basal levels of SLC7A11 (FIGS. 11C and 11D). To examine the role of cystinosin in tumorigenesis in vivo, Applicant conducted xenograft experiments using immuno-compromised SCID-Beige mice. While silencing SLC7A11 or CTNS suppressed UMRC6 tumor growth, knocking down both genes resulted in a nearly complete inhibition of tumorigenesis (FIG. 4D).

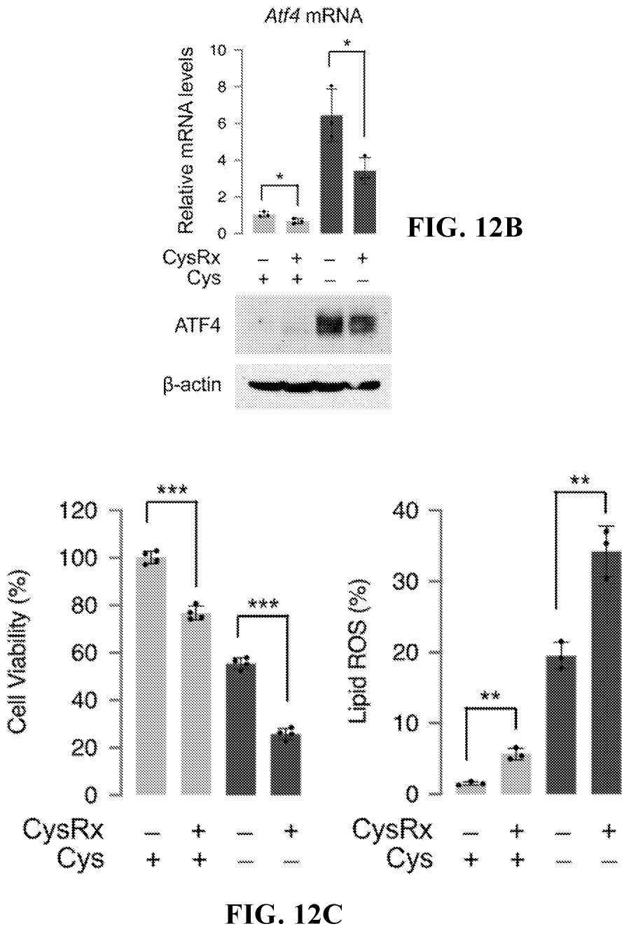

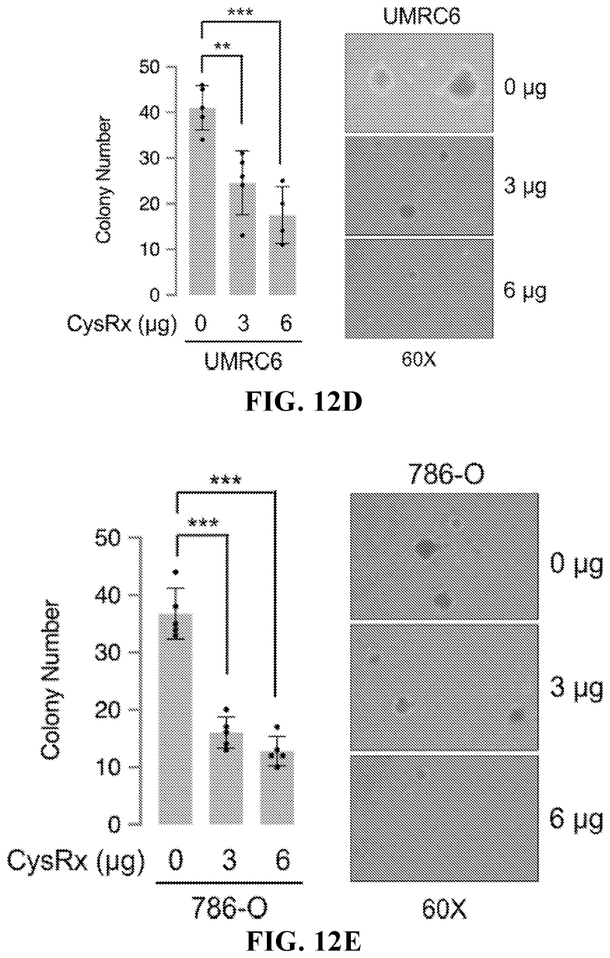

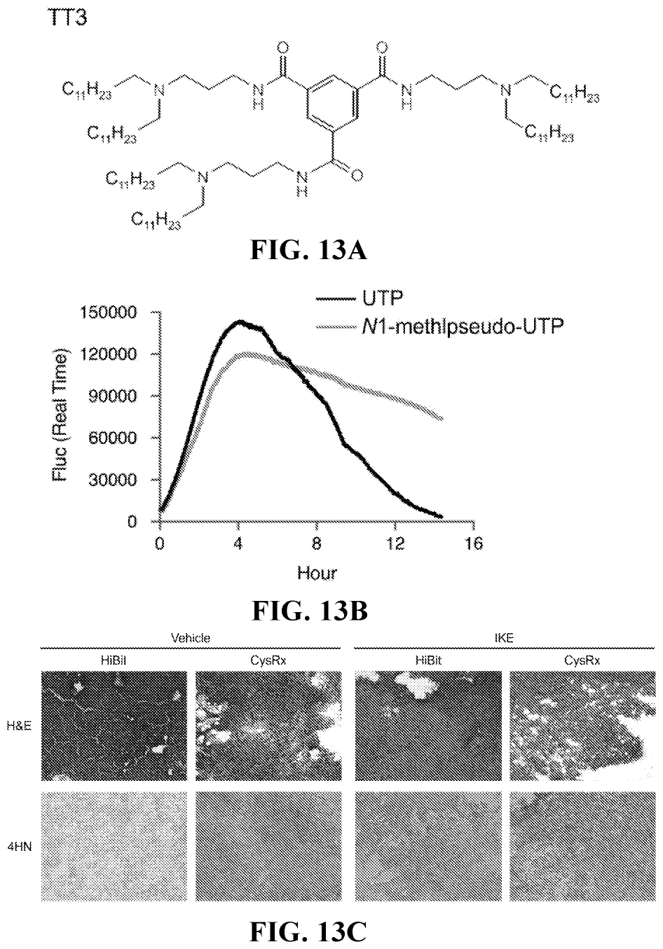

Despite the promising effect of CTNS silencing in promoting cancer cell ferroptosis, the encoded cystinosin is crucial in maintaining lysosomal homeostasis (24). Mutations in CTNS have been associated with cystinosis, a systemic disease with multiple clinical manifestations (16). It is thus highly desirable to create an alternative way to block lysosomal cystine efflux without genetic perturbation. From the therapeutic perspective, non-genetic approaches are more feasible in cost-effective manufacturing and safe administration. Given the recent success of mRNA vaccines in combating COVID-19 (25), therapeutic mRNAs are being developed for a broad range of human diseases. By coupling translation of a cysteine-rich polypeptide and co-translational lysosome targeting, Applicant designed an artificial mRNA construct to direct cytosolic cysteine to the lysosome (FIG. 4E). A survey of human coding sequences revealed many cysteine-rich (CR) domains in a large group of proteins. For instance, a cysteine-string protein (CSP) encoded by DNAJC5 contains 15 cysteines within a stretch of 25 amino acids. Cysteine rich tail 1 (CYSRT1) comprises a similar CR motif with a stretch of 20 amino acids containing 12 cysteines. Applicant fused both CR motifs to insulin-like growth factor 2 (IGF2) to enable lysosomal targeting via the mannose-6-phosphate receptor (26) (FIG. 12A). Applicant named this fusion construct as a cyst(e)ine reverse exchanger (CysRx) because it converts cytosolic cysteine into lysosomal cystine. Cell fractionation analysis confirmed lysosomal localization of CysRx (FIG. 12A). Supporting lysosomal degradation of CysRx, BafA treatment caused CysRx accumulation. Remarkably, CysRx-transfected cells showed a significant decrease of cytosolic cysteine and a corresponding increase of lysosomal cystine (FIG. 4F). Mimicking cystinosin knockdown, CysRx overexpression attenuated ATF4 expression in response to cystine withdrawal (FIG. 12B). As a result, cystine-starved cells became more susceptible to ferroptosis (FIG. 12C).

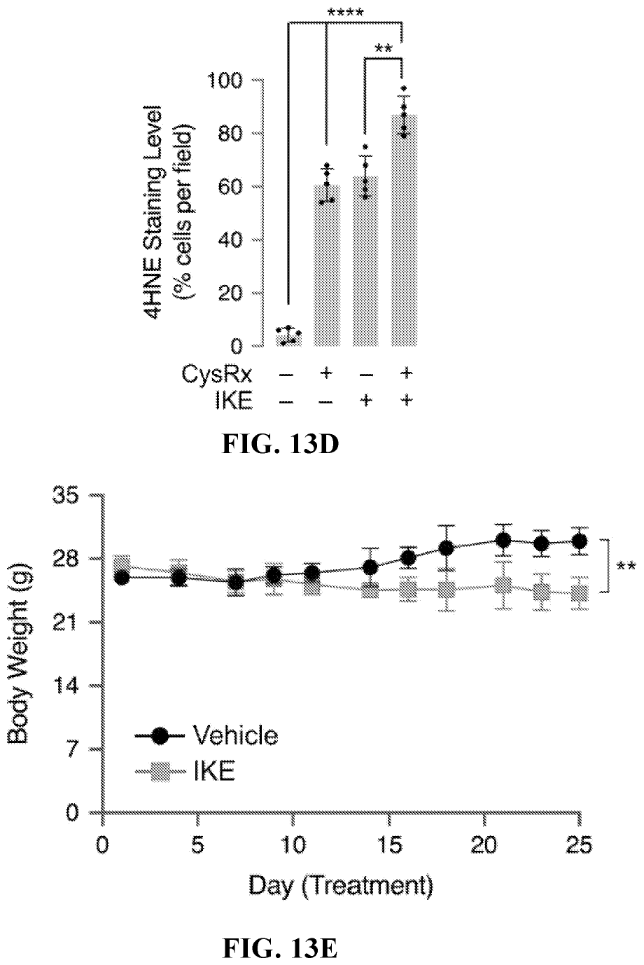

Applicant also transfected UMRC6 cells with CysRx mRNA, which sensitized cells to ferroptosis with significantly increased lipid oxidation (FIG. 4G). By potently suppressing the adaptive ATF4 expression (FIG. 4H), CysRx inhibited colony formation of UMRC6 in a dose-dependent manner (FIG. 12D). The inhibitory role of CysRx in tumorigenesis was also observed in 786-O cells (FIG. 12E), suggesting that the efficacy of CysRx is independent of SLC7A11. To assess the therapeutic potential of CysRx in vivo, Applicant formulated lipid nanoparticles (LNPs) using an ionizable lipid N¹,N³,N⁵-tris(2-aminoethyl)benzene-1,3,5-tricarboxamide derivatives (TT3) that enables efficient nucleic acid encapsulation, cellular delivery, and endosomal release (27) (FIG. 13A). To enhance mRNA stability and minimize immunogenicity, CysRx mRNA was incorporated with N1-methylpseudouridine. The modified mRNAs exhibited prolonged HiBit signals compared to the non-modified version in transfected UMRC6 cells (FIG. 13B). Applicant next evaluated the effect of CysRx-TT3 in the xenografted UMRC6 tumors via intratumor administration. Compared to the HiBit-TT3 control, weekly injection of CysRx-TT3 induced a significant delay in tumor progression (FIG. 4I, upper panel). Supporting enhanced ferroptotic cell death, tumors treated with CysRx-TT3 displayed significant accumulation of 4-hydroxynonenal (4HN), a by-product of lipid peroxidation (FIG. 13C). Applicant further tested the therapeutic potential of CysRx-TT3 in combination with the system X_{c}⁻ inhibitor imidazole ketone erastin (IKE). Similar to prior studies (28), intraperitoneal administration of IKE (10 mg/kg) suppressed tumor growth. Intratumoral administration of CysRx-TT3 further reduced tumor growth with increased 4HN accumulation (FIG. 4I, bottom panel and FIG. 13D), suggesting a synergistic effect in promoting ferroptotic cell death in vivo. Notably, systemic administration of IKE resulted in weight loss (FIG. 13E), which was not evident in mice treated with CysRx-TT3. Together, Applicant's results demonstrate the therapeutic potential of CysRx-TT3 in cancer treatment without altering the function of endogenous genes.

Amino acids are essential nutrients to the survival of all cell types. Although cancer cells often experience reprogrammed metabolism (29), direct targeting of amino acid metabolism for therapeutic intervention is challenging. Additionally, cancer cells can adapt to nutrient stress by upregulating ATF4 through ISR, thereby enabling tumor progression under adverse conditions. How to disable the adaptive ATF4 response within cancer cells has been a formidable task until now. Applicant found that ATF4 is subjected to transcriptional and translational regulation, with the former highly sensitive to lysosomal cystine. Importantly, reducing the cysteine/cystine ratio by blocking the lysosomal cystine efflux sensitizes cells to ferroptosis. By altering intracellular cyst(e)ine homeostasis, it is thus possible to achieve tumor-selective ferroptosis without gross nutrient perturbation. The development of CysRx provides a platform of mRNA engineering to starve cancer cells of specific amino acids without systemic intervention. Serving as a proof-of-principle, CysRx administration in the form of LNP effectively induced tumor ferroptosis in vivo and can be readily applied to other types of cancers. Coupled with targeted delivery, CysRx offers a promising therapeutic approach to many other diseases via nutrient reprogramming.

### Materials and Methods

### Cell Lines and Reagents

MEF cells, HEK293, UMRC6, and 786-O cells were maintained in Dulbecco's Modified Eagle's Medium (DMEM) with 10% fetal bovine serum (FBS). eIF2α(S/S) & eIF2α(A/A) MEFs were additionally supplemented with 5% non-essential amino acids (Invitrogen: 11140-050). The following reagents were used at their indicated experimental concentrations and time points; cycloheximide (Sigma Aldrich: C7698-5G), puromycin (Sigma Aldrich: P7255-250 MG), bafilomycinA (Sigma Aldrich: B1793), concanamycinA (Sigma Aldrich: C9705), cysteamine (Sigma Aldrich: M9768), SR1 (Sigma Aldrich: 182706), indirubin (Sigma Aldrich: SML0280), erastin (Sigma Aldrich: E7781), sulfasalazine (Sigma Aldrich: S0883), Z-VAD-fmk (Invivogen: tlrl-vad), necrostatin-1 (Santa-Cruz Biotechnology: sc-200142), ferrostatin-1 (Sigma Aldrich: SML0583), L-buthionine-sulfoximine (Sigma Aldrich: B2515). Antibodies are listed below: ATF4 (Cell Signaling: 11815S), P-eIF2α (Cell Signaling: 3398S), eIF2α (Cell Signaling: 5324S), β-Actin (Sigma: A5441), Slc7a11 (Abcam: ab37185), Nrf2 (Santa Cruz: sc-365949), Cystinosin (Aviva Systems Biology: ARP44766_P050), GCN2 (Cell Signaling: 3302S), HiBit (Promega: N2410), Rpl4 (Proteintech: 11302-1), Progranulin D (R&D Systems: AF2557), Mrps18b (Proteintech: 16139-1), HSP90 (Cell Signaling: 8165S).

### Amino Acid Starvation

For cystine and methionine deprivation, the experiment was carried out by incubating cells in DMEM, high glucose, no glutamine, no methionine, no cystine (Thermo Fisher: 21013024) with 10% dialyzed FBS (Sigma Aldrich: F0392). For leucine, histidine, and arginine deprivation, the experiment was carried out by incubating cells in DMEM, high glucose, no arginine, no histidine, no leucine, respectively, (custom prepared by Gibco/Invitrogen) with 10% dialyzed FBS (Sigma Aldrich: F0392). For full amino acid starvation, the experiment was carried out by incubating cells in HBSS buffer (Lonza) with 10% dialyzed FBS (Sigma Aldrich: F0392). Samples were collected at indicated experimental time points.

### Real-Time Quantitative PCR

Following experimental conditions, total RNA was isolated by TRIzol reagent (Invitrogen) and reverse transcription was performed using High-Capacity cDNA Reverse Transcription Kit (Invitrogen). Real-time PCR analysis was conducted using Power SYBR Green PCR Master Mix (Applied Biosystems) and data was generated using a LightCycler 480 Real-Time PCR System (Roche Applied Science). qPCR oligo sequences are listed in Table 4.

TABLE 4
qPCR oligo sequences

| | |
|---|---|
| Atf4 | 5′ - CTTGATGTCCCCCTTCGACC - 3′ |
| (mouse) F | (SEQ ID NO: 60) |
| Atf4 | 5′ - CTTGTCGCTGGAGAACCCAT - 3′ |
| (mouse) R | (SEQ ID NO: 61) |
| Nrf2 | 5′ - CCTCGCTGGAAAAAGAAGTG - 3′ |
| (mouse) F | (SEQ ID NO: 62) |
| Nrf2 | 5′ - GGAGAGGATGCTGCTGAAAG - 3′ |
| (mouse) R | (SEQ ID NO: 63) |
| Slc7a11 | 5′ - GGCACCGTCATCGGATCAG - 3′ |
| (mouse) F | (SEQ ID NO: 64) |
| Slc7a11 | 5′ - CTCCACAGGCAGACCAGAAAA - 3′ |
| (mouse) R | (SEQ ID NO: 65) |
| Gapdh | 5′ - CAAGGAGTAAGAAACCCTGGAC - 3′ |
| (mouse) F | (SEQ ID NO: 66) |
| Gapdh | 5′ - GGATGGAAATTGTGAGGGAGAT - 3′ |
| (mouse) R | (SEQ ID NO: 67) |
| Atf4 | 5′ - TCAGTCCCTCCAACAACAGC - 3′ |
| (human) F | (SEQ ID NO: 68) |
| Atf4 | 5′ - TCTGGCATGGTTTCCAGGTC - 3′ |
| (human) R | (SEQ ID NO: 69) |
| Actin | 5′ - AGATGTGGATCAGCAAGC - 3′ |
| (human) F | (SEQ ID NO: 70) |
| Actin | 5′ - TCATCTTGTTTTCTGCGC - 3′ |
| (human) R | (SEQ ID NO: 71) |

Lentiviral shRNAs

All shRNA targeting sequences were cloned into DECIPHER pRSI9-U6-(sh)-UbiC-TagRFP-2A-Puro (Cellecta, CA). shRNA targeting sequences listed below were based on RNAi consortium at the Broad Institute (https://www.broad.mit.edu/rnai/trc). Lentiviral particles were packaged using Lenti-X 293T cells (Clontech) grown in DMEM media. Virus-containing supernatants were collected at 48 hrs post transfection and filtered with Millex-HA Syringe Filter Unit, 0.45 μm (Millipore) to eliminate any debris. Cells were infected with the lentivirus for 48 hrs before selection by 2 mg/mL puromycin. shRNA oligos are listed in Table 5.

TABLE 5
List of shRNA Primers

| | |
|---|---|
| CTNS | ACCGGGGAGGAATTGGCTGCTTATTTCTCGAGAAATAAG |
| #1 | CAGCCAATTCCTCCTTTTTTG (SEQ ID NO: 72) |
| (mouse): | |
| CTNS | CGAACAAAAAAGGAGGAATTGGCTGCTTATTTCTCGAGA |
| #1 | AATAAGCAGCCAATTCCTCCC (SEQ ID NO: 73) |
| (mouse): | |
| CTNS | ACCGGCCCTTGGGCATCCACTAAATTCTCGAGAATTTAG |
| #2 | TGGATGCCCAAGGGTTTTTTG (SEQ ID NO: 74) |
| (mouse): | |
| CTNS | CGAACAAAAAACCCTTGGGCATCCACTAAATTCTCGAGA |
| #2 | ATTTAGTGGATGCCCAAGGGC (SEQ ID NO: 75) |
| (mouse): | |
| CTNS | ACCGGCAGTCACGCTGGTCAAGTATTCTCGAGAATACTT |
| #1 | GACCAGCGTGACTGTTTTTTG (SEQ ID NO: 76) |
| (human): | |
| CTNS | CGAACAAAAAACAGTCACGCTGGTCAAGTATTCTCGAGA |
| #1 | ATACTTGACCAGCGTGACTGC (SEQ ID NO: 77) |
| (human): | |
| Slc7a11 | ACCGGGCAGGCGGTACCGAATCAGCCTCGAGGCTGATTC |
| #1 | GGTACCGCCTGCTTTTTTG (SEQ ID NO: 78) |
| (mouse): | |
| Slc7a11 | CGAACAAAAAAGCAGGCGGTACCGAATCAGCCTCGAGGC |
| #1 | TGATTCGGTACCGCCTGCC (SEQ ID NO: 79) |
| (mouse): | |
| Slc7a11 | ACCGGGAGGGAAACTAAAAATGGAGCTCGAGCTCCATTT |
| #2 | TTAGTTTCCCTCTTTTTTG (SEQ ID NO: 80) |
| (mouse): | |
| Slc7a11 | CGAACAAAAAAGAGGGAAACTAAAAATGGAGCTCGAGCT |
| #2 | CCATTTTTAGTTTCCCTCC (SEQ ID NO: 81) |
| (mouse): | |
| Slc7a11 | ACCGGGGTGTTTCTGAGTAGTAATTACTCGAGTAATTAC |
| #1 | TACTCAGAAACACCTTTTTTG (SEQ ID NO: 82) |
| (human): | |
| Slc7a11 | CGAACAAAAAAGGTGTTTCTGAGTAGTAATTACTCGAGT |
| #1 | AATTACTACTCAGAAACACCC (SEQ ID NO: 83) |
| (human): | |

### SiRNA Transfection

siRNAs targeting mouse Nrf2 (Santa Cruz: sc-37049) or scramble control (Santa Cruz:
sc-37007) were transfected into MEF cells using Lipofectamine 2000 (Invitrogen) according to the manufacturer's instruction. Knockdown efficiency was examined 48 hrs after siRNA administration.

### Immunoblotting

Cells were washed with PBS (Gibco) and then lysed on ice using cell lysis buffer (50 mM Tris [pH7.5], 150 mM NaCl, 1 mM EDTA, 1% Triton X-100, 2 U/ml DNase and protease inhibitor cocktail tablet). The lysates were incubated on ice for 30 min and spun down at 10,000 rpm for 3 mins to collect supernatant. Collected supernatant was measured by protein assay (Bio-Rad: 500-0112) to quantify the protein concentration. Equal amounts of proteins across samples were mixed with SDS-PAGE sample buffer (50 mM Tris [pH6.8], 100 mM dithiothreitol, 2% SDS, 0.1% bromophenol blue, 10% glycerol) and heated for 9 mins at 95° C. Denatured proteins were separated on SDS-PAGE and transferred to PVDF membranes (Fisher). Membranes were blocked in TBS containing 5% non-fat milk and 0.1% Tween-20 for 1 hr. Phospho-proteins were blocked in TBS containing 5% BSA and 0.1% Tween-20 for 1 hr. Blocking was followed by incubation with primary antibodies overnight at 4° C. Membranes were washed using TBST followed by subsequent incubation using horseradish peroxidase-coupled secondary antibodies at room temperature for 1 hr. Immunoblots were washed again using TBST and visualized using enhanced chemiluminescence (ECL-Plus, GE Healthcare).

### Cysteine, Cystine, and Glutathione Measurement

Intracellular cysteine and glutathione levels were measured by methods described previously (30) with optimization. Cells were grown on 100 mm dishes until 80-90% confluent. Cells were lysed on ice in lysis buffer (50 mM Tris [pH7.5], 150 mM NaCl, 1 mM EDTA, 1% Triton X-100, 2 U/ml DNase and protease inhibitor cocktail tablet), followed by centrifugation at 10,000 rpm for 8 min at 4° C. The supernatant was collected and 100 μL of sample was mixed with 10 μL TCEP (50 mM in borate buffer [pH 7.4]) (Sigma Aldrich) in a vial and incubated at 25° C. for 10 min.

90 μL of a solution containing 1% TCA and 1 mM EDTA was then added. The total solution was centrifuged for 10 min at 10,000 g at 4° C. 100 μL of the obtained supernatant, 20 μL of 10 mM CNBF solution (4-chloro-3,5-dinitrobenzotrifluoride [Sigma Aldrich: 197017]), 20 μL of methanol, and 50 μL of borate buffer (0.2 M [pH 8.0])) were mixed and incubated at 25° C. for 20 min. Derivatization was terminated with 10 μL of 2M HCl followed by HPLC analysis (see below).

Intracellular cystine was measured as described previously (31) with optimization. Cells were grown on 100 mm dishes until 80-90% confluent. Cells were lysed on ice in lysis buffer (50 mM Tris [pH7.5], 150 mM NaCl, 1 mM EDTA, 1% Triton X-100, 2 U/ml DNase and protease inhibitor cocktail tablet). 100 μL of sample was mixed with 100 μL of 6%5-Sulfosalicylic Acid Dyhydrate solution (Sigma: S3147), followed by centrifugation at 3,000 g for 15 min at 4° C. The supernatant was collected and 25 μL of freshly made derivatizing solution was added. Samples sat at room temperature for 20 mins, followed by rotary evaporation. Dried material was dissolved in 150 μL of phase A solution followed by HPLC analysis (see below).

All resulting solutions for cysteine, glutathione, and cystine were filtered through a 0.22 μm filter membrane (Millipore Corporation, Belfast, MA, USA) and injected into a chromatographic system. High performance liquid chromatography (HPLC) was performed using a LC-20AT pump with a SPD-20AV UV-vis detector monitored at 270 and 220 nm (Shimadzu, Japan) equip with an Ultra Aqueous C18 column (100 Å, 5 μm, 250 mm×4.6 mm; Restek, USA) at a flow rate of 1 mL/min with a mobile phase containing 0.1% trifluoroacetic acid (TFA) in H2O or acetonitrile. Values were normalized to protein concentration by Bradford assay.

### Polysome Profiling

15% and 45% sucrose solutions were freshly prepared using polysome buffer (10 mM HEPES [pH 7.4], 100 mM KCl, 5 mM MgCl₂, 100 mg/ml cycloheximide, 2% Triton X-100) and loaded into SW41 ultracentrifuge tubes (Beckman). A 15%-45% density gradient was made using a Gradient Master (BioComp Instruments). Following experimental conditions, cells were washed using ice-cold PBS three times and then lysed in polysome lysis buffer (polysome buffer, 100 mg/ml cycloheximide, 10% Triton X-100). Cell debris were removed by centrifugation at 14,000 rpm for 10 min at 4° C. 500 μL of supernatant was loaded onto the sucrose gradient followed by ultra-centrifugation for 2 hr 30 min at 35,000 rpm at 4° C. in a SW41 rotor. Separated samples were fractionated at 0.75 ml/min through an automated fractionation system (Isco) that continually monitors OD254 values.

### Puromycin Labeling

Puromycin labeling was performed as previously described (32) with some modifications. Cells were grown to 70-80% confluence and treated with puromycin (10 μg/ml) for 10 min. Cells were washed twice with ice-cold PBS, and then lysed on ice using cell lysis buffer (50 mM Tris [pH7.5], 150 mM NaCl, 1 mM EDTA, 1% Triton X-100, 2 U/ml DNase and protease inhibitor cocktail tablet). The lysates were incubated on ice for 30 min and spun down at 10,000 rpm for 3 mins to collect supernatant. Collected supernatant was followed by protein assay to measure protein concentration. Equal amounts of proteins across samples were mixed with SDS-PAGE sample buffer (50 mM Tris [pH6.8], 100 mM dithiothreitol, 2% SDS, 0.1% bromophenol blue, 10% glycerol) and heated for 9 mins at 95° C. Denatured proteins were separated on a 10% SDS-PAGE gel and transferred to Immobilon-P membranes. Membranes were blocked for 1 hr in TBS containing 5% nonfat milk and 0.1% Tween 20, followed by incubation with puromycin antibodies (1:100 dilution) overnight at 4° C. The membrane was then washed with TBST and incubated with HRP-conjugated anti-mouse immunoglobulin G (IgG) (1:5000 dilution) for 1 hr at room temperature, followed by TBST wash and visualization using enhanced chemiluminescence.

### Luciferase Reporter Assay

Firefly luciferase reporters were co-transfected with a Renilla reporter plasmid into MEF cells or CTNS knockdown cells for 4 hrs. Transfected cells were treated with amino acid starvation and/or compound treatment at indicated timepoints. Firefly and Renilla luciferase activities were measured using Dual-Luciferase Reporter Assay System (Promega). Relative values of firefly luciferase activities were normalized to Renilla luciferase control. Oligo sequences used for construction of truncated Atf4 promoter regions are listed in Table 6.

TABLE 6
List of pAtf4 Truncated Primers

| | |
|---|---|
| pAtf4 | ATTGACTAGTACGCCTGGGCCAATCAGCTCGAC |
| (−1200 bp) F: | (SEQ ID NO: 84) |
| pAtf4 | ATTGACTAGTTCTCATGGGGCCTTTAGGACGAT |
| (−1000 bp) F: | (SEQ ID NO: 85) |
| pAtf4 | ATTGACTAGTCATTTCTGCTTGCTGTCTGCCGG |
| (−800 bp) F: | (SEQ ID NO: 86) |
| pAtf4 | ATTGACTAGTGCGTTGCCTGCGACGCCGGCGCT |
| (−500 bp) F: | (SEQ ID NO: 87) |
| pAtf4 | ATTGACTAGTGCTCACCGGGGTCCCCGTGTCAT |
| (−400 bp) F: | (SEQ ID NO: 88) |
| pAtf4 | ATTGACTAGTCGTATTAGGACGCGAGGACAAGC |
| (−300 bp) F: | (SEQ ID NO: 89) |
| pAtf4 | ATTGACTAGTCACAATGGCCTTGGGCCCGCGTG |
| (−200 bp) F: | (SEQ ID NO: 90) |
| pAtf4 | ATTGACTAGTCCATCCAGGCTCTTCACGAAATC |
| (−100 bp) F: | (SEQ ID NO: 91) |
| pAtf4 | ATGCGGATCCCCCGAGATGATTAAGCTAAGACA |
| (−400 bp) R: | (SEQ ID NO: 92) |
| pAtf4 | ATGCGGATCCGGCGGCGGCACGCCCTAAACCCG |
| (−800 bp) R: | (SEQ ID NO: 93) |
| pAtf4 | ATGCGGATCCAGAAAAGTGCACTACTTTATAGG |
| (−1000 bp) R: | (SEQ ID NO: 94) |
| pAtf4 | ATGCGGATCCGTTGTGGGGCTTTGCTGGATTCG |
| (0 bp) R: | AG (SEQ ID NO: 95) |

### Cell Viability Assay

Cells were seeded into 96-well plates at 3,000 cells per well. After 16 hr, cells were subject to conditions of the indicated experiments. Cells were incubated for 24 hr in their experimental conditions and cell viability was measured using the CellTiter-Blue viability assay (Promega) following the manufacturer's instructions. Relative cell viability in the presence of starvation and/or compounds was normalized to the vehicle-treated controls after background subtraction.

### Lipid Peroxidation Measurement

Cells were plated in 6-well dishes and followed by indicated treatments. After treatment, cells were incubated with fresh medium containing 2 μM BODIPY 581/591 C11 dye (Invitrogen: D3861) for 15 min. Cells were next collected and washed twice with ice-cold PBS followed by fluorescence-activated cell sorting (FACS) analysis using Thermo Fisher Attune NxT. Fluorescence captured during analysis was gated and plotted using FCS Express 7.

### In Vitro Transcription

Plasmid containing the sequence of eGFP was used as a template for PCR reactions to generate the desired CysRx and control sequences. Transcription reactions were performed at 37° C. for 2 hours using the mMESSAGE mMACHINE T7 Transcription Kit (Invitrogen 1344). Buffer conditions for the reaction contained 50 mM Tris [pH 7.8], 1 mM MgCl2, 5 mM KCl, and 0.8 mM DTT. Triphosphate-derivatives of N1-methylpseudouridine (Ψ) (APExBIO: B8049) were used in place of UTP to generate modified nucleoside-containing RNA. The synthesized RNAs were capped by adding 6 mmol/L purified Vaccina capping enzyme, 0.5 mM GTP, and 0.1 mM SAM to the reaction. The reaction occurred at 37° C. for an additional 2 hours. Following transcription, the template plasmids were digested with Turbo DNase. RNAs were then poly(A) tailed following the manufactures instructions (Invitrogen: AM1350). Reactions were terminated using 2.5 M lithium chloride, and RNAs were ethanol precipitated overnight at −20° C. RNAs were pelleted by centrifugation, washed with 75% ethanol and then reconstituted in nuclease-free water. The concentration of RNA was determined by measuring the optical density at 260 nm.

### Cell Fractionation

MEF cells were grown in four 15 cm dishes until 80% confluent (˜3×10⁸ cells) followed by washing twice with ice-cold PBS. Lysosomes were isolated with lysosome isolation kit (Thermo Fisher—89839) according to the instructions with the following optiprep gradients (8%, 12%, 16%, 19%, 23%, 27%). Lysosomes were enriched in fraction #2 (12%-16%), and mitochondria were enriched in fraction #4 (23%-27%).

### Colony Formation on Soft Agar

A solid base layer was formed by coating a 6-well plate with 2 mL of 0.6% agarose in DMEM growth media. After 30 minutes at 24° C., 1000 cells/mL were mixed with 0.5 mL of 0.3% low melting point agarose and 4.5 mL of DMEM growth media. One milliliter of the mixture was seeded onto the 6-well plate coated with base agar. Cells were allowed to grow for 21 days. Colonies were photographed and counted. For CysRx treatment, cells were treated with either 3 ug or 5 ug of CysRx on day 1, which was placed directly into the top layer mixture.

Formulation of mRNA-Loaded TT3 Lipid Nanoparticles

TT3 lipid nanoparticles were formulated as previously described (33). Briefly, TT3, 2-dioleoyl-sn-glycero-3-phosphoethanolamine (DOPE), cholesterol, 1,2-dimyristoyl-snglycerol, methoxypolyethylene glycol (DMG-PEG2000) were mixed at a molar ratio of 20/30/40/0.75 at the ethanol phase. CysRx or Control mRNA (TT3:mRNA=10:1, mass ratio) was diluted in citrate buffer as the aqueous phase. TT3 lipid nanoparticles were prepared by mixing 1 volume of the ethanol phase with 3 volume of the aqueous phase using a microfluidic device (Precision NanoSystems, Vancouver, BC, Canada).

### Mouse Strains and Husbandry

NOD.Cg-Prkc^{scid}Il2^{tm1Wj1}/SzJ, NSG, mice catalog number 005557 were sourced from The Jackson Laboratory and bred in house (Cornell University, USA) with the supervision of the Center for Animal Resources and Education (CARE) breeding program. All animals used in this study were handled in accordance with federal and institutional guidelines, under a protocol approved by the Cornell University Institutional Animal Care and Use Committee, protocol 2017-0035. Mice were housed under specific pathogen-free conditions in an Association for the Assessment and Accreditation of Laboratory Animal Care International-accredited facility and cared for in compliance with the Guide for the Care and Use of Laboratory Animals.

### Xenotransplantation of UMRC6 Cells

One million UMRC6 (shScramble, shCTNS, and/or shSlc7a11) cells suspended in 100 μL 1×PBS and 100 μL Matrigel were injected subcutaneously, bilaterally on the flanks of NOD.Cg-PrkdcscidIl2rgtm1Wjl/SzJ (NSG) mice. Mice were monitored for tumor growth three times per week using digital calipers. Health and body condition were also monitored concurrently.

Animals were sacrificed by CO₂ euthanasia (3.5 L/min) when tumors reached humane endpoint of 2000 mm³ or body condition started to deteriorate. Tumors were excised from the flank using a surgical 10 blade, weighed and flash frozen or fixed in 4% PFA.

### CysRx Treatment of UMRC6 Tumors

Previously established UMRC6 tumors were immediately excised from CO₂ euthanized mice and minced using a surgical 10 blade. Approximately 2⁻³ mm³ portions of tumor were bilaterally implanted into 6-8-week-old NSG mice. Animals were monitored for tumor growth and randomized into treatment groups when tumors reached 150-200 mm³ in size. IKE/vehicle treated animals received intraperitoneal injections every other day (10 mg/kg). CysRx-TT3 treatment was conducted via weekly intratumoral (IT) injections (100 μL) for three weeks. Tumors for IT injections were demarcated into four quadrants and an equal volume (25 μL) of CysRx-TT3 was injected into each quadrant. HiBit-TT3 was injected into the other flank as control. Mice were monitored for tumor growth three times per week using digital calipers. Health and body condition were also monitored concurrently. Animals were sacrificed by CO₂ euthanasia (3.5 L/min) one week after final administration or when the body condition started to deteriorate. Tumors were excised from the flank using a surgical 10 blade, weighed and either flash frozen or fixed in 4% PFA.

### Magnetic Resonance Imaging of Mice

Magnetic resonance imaging (MRI) of mice was conducted using a 1T M3 compact MRI from Aspect Imaging Ltd. Mice were anesthetized using 2.5% isoflurane and placed onto the specimen arm, coil was then placed around the subject and fixed in place. Mice were scanned using a T2 weighted scan without contrast agent, slice thickness was set to 1 mm, with an inter-slice gap of 0 mm, A total of 20 slices were obtained per animal. Raw image DICOMS were exported from MR system and imported into VivoQuant image analysis software by Invicro, a Konica Minolta Company. A tumor region of interest layer was created using automatic thresholding settings and a thickness of 5; the tumor was followed and highlighted throughout the image stack. Minor, manual modifications were made to the automatic tracing of the tumor when extraneous anatomy was included in the tumor region of interest. Once the tracing was complete a 3-dimensional render was created and outputted into an animated GIF format.

### Immunohistochemistry

Tumors were excised from mice and flash frozen in liquid nitrogen, followed by embedding in O.C.T. (Tissue-Teck: 4583). Tissue sections (15 μm thickness) were created using a Leica Cryostat (CM1950). Slides were dehydrated for 20 mins at room temperature, followed by fixing using 4% PFA for 7 mins. Slides were then rehydrated in graded alcohols and stained with hematoxylin and eosin (H&E) or by immunohistochemistry. For immunohistochemistry, antigen retrieval was completed by microwaving slides at high power in citrate buffer [pH 6.0] for 21 mins. Slides were immersed in 25% hydrogen peroxide in methanol for 10 mins to inhibit endogenous peroxidase activity. Slides were blocked in BSA to prevent non-specific antibody binding for 45 mins, and then incubated with 4HNE (Abcam: ab46545) antibody overnight at 4° C. The next day, slides were incubated in biotinylated secondary antibody, followed by streptavidin HPR conjugate (Invitrogen Histostain) at room temperature. Immunoreactivity was visualized using DAB (Invitrogen), counterstained with hematoxylin (Fisher CS401-1D), dehydrated and mounted. Slides were scanned using a Leica DMi8 microscope and analyzed using Image J. A minimum of five focal planes (20×) per tumor were analyzed and averaged to quantify 4HNE positive cells.

### REFERENCES FOR EXAMPLE 1

1. R. C. Wek, H. Y. Jiang, T. G. Anthony, Coping with stress: eIF2 kinases and translational control. Biochem Soc Trans 34, 7-11 (2006).
2. H. P. Harding, Y. Zhang, H. Zeng, I. Novoa, P. D. Lu, M. Calfon, N. Sadri, C. Yun, B. Popko, R. Paules, D. F. Stojdl, J. C. Bell, T. Hettmann, J. M. Leiden, D. Ron, An integrated stress response regulates amino acid metabolism and resistance to oxidative stress. Mol Cell 11, 619-633 (2003).
3. N. Sonenberg, A. G. Hinnebusch, New modes of translational control in development, behavior, and disease. Mol Cell 28, 721-729 (2007).
4. X. E. Shu, R. V. Swanda, S. B. Qian, Nutrient Control of mRNA Translation. Annu Rev Nutr 40, 51-75 (2020).
5. K. Ameri, A. L. Harris, Activating transcription factor 4. Int J Biochem Cell Biol 40, 14-21 (2008).
6. M. S. Kilberg, J. Shan, N. Su, ATF4-dependent transcription mediates signaling of amino acid limitation. Trends Endocrinol Metab 20, 436-443 (2009).
7. A. G. Hinnebusch, Translational regulation of GCN4 and the general amino acid control of yeast. Annu Rev Microbiol 59, 407-450 (2005).
8. K. M. Vattem, R. C. Wek, Reinitiation involving upstream ORFs regulates ATF4 mRNA translation in mammalian cells. Proc Natl Acad Sci USA 101, 11269-11274 (2004).
9. D. Scheuner, B. Song, E. McEwen, C. Liu, R. Laybutt, P. Gillespie, T. Saunders, S. Bonner-Weir, R. J. Kaufman, Translational control is required for the unfolded protein response and in vivo glucose homeostasis. Mol Cell 7, 1165-1176 (2001).
10. M. Conrad, H. Sato, The oxidative stress-inducible cystine/glutamate antiporter, system x (c) (−): cystine supplier and beyond. Amino Acids 42, 231-246 (2012).
11. S. J. Dixon, K. M. Lemberg, M. R. Lamprecht, R. Skouta, E. M. Zaitsev, C. E. Gleason, D. N. Patel, A. J. Bauer, A. M. Cantley, W. S. Yang, B. Morrison, 3rd, B. R. Stockwell, Ferroptosis: an iron-dependent form of nonapoptotic cell death. Cell 149, 1060-1072 (2012).
12. M. H. Stipanuk, J. E. Dominy, Jr., J. I. Lee, R. M. Coloso, Mammalian cysteine metabolism: new insights into regulation of cysteine metabolism. J Nutr 136, 1652S-1659S (2006).
13. T. Nguyen, P. J. Sherratt, P. Nioi, C. S. Yang, C. B. Pickett, Nrf2 controls constitutive and inducible expression of ARE-driven genes through a dynamic pathway involving nucleocytoplasmic shuttling by Keap1. J Biol Chem 280, 32485-32492 (2005).
14. P. S. Lange, J. C. Chavez, J. T. Pinto, G. Coppola, C. W. Sun, T. M. Townes, D. H. Geschwind, R. R. Ratan, ATF4 is an oxidative stress-inducible, prodeath transcription factor in neurons in vitro and in vivo. J Exp Med 205, 1227-1242 (2008).
15. M. Abu-Remaileh, G. A. Wyant, C. Kim, N. N. Laqtom, M. Abbasi, S. H. Chan, E. Freinkman, D. M. Sabatini, Lysosomal metabolomics reveals V-ATPase- and mTOR-dependent regulation of amino acid efflux from lysosomes. Science 358, 807-813 (2017).
16. W. A. Gahl, J. G. Thoene, J. A. Schneider, Cystinosis. N Engl J Med 347, 111-121 (2002).
17. A. Jezegou, E. Llinares, C. Anne, S. Kieffer-Jaquinod, S. O'Regan, J. Aupetit, A. Chabli, C. Sagne, C. Debacker, B. Chadefaux-Vekemans, A. Journet, B. Andre, B. Gasnier, Heptahelical protein PQLC2 is a lysosomal cationic amino acid exporter underlying the action of cysteamine in cystinosis therapy. Proc Natl Acad Sci USA 109, E3434-3443 (2012).
18. J. V. Schmidt, C. A. Bradfield, Ah receptor signaling pathways. Annu Rev Cell Dev Biol 12, 55-89 (1996).
19. M. S. Denison, S. R. Nagy, Activation of the aryl hydrocarbon receptor by structurally diverse exogenous and endogenous chemicals. Annu Rev Pharmacol Toxicol 43, 309-334 (2003).
20. B. R. Stockwell, J. P. Friedmann Angeli, H. Bayir, A. I. Bush, M. Conrad, S. J. Dixon, S. Fulda, S. Gascon, S. K. Hatzios, V. E. Kagan, K. Noel, X. Jiang, A. Linkermann, M. E. Murphy, M. Overholtzer, A. Oyagi, G. C. Pagnussat, J. Park, Q. Ran, C. S. Rosenfeld, K. Salnikow, D. Tang, F. M. Torti, S. V. Torti, S. Toyokuni, K. A. Woerpel, D. D. Zhang, Ferroptosis: A Regulated Cell Death Nexus Linking Metabolism, Redox Biology, and Disease. Cell 171, 273-285 (2017).
21. X. Chen, R. Kang, G. Kroemer, D. Tang, Broadening horizons: the role of ferroptosis in cancer. Nat Rev Clin Oncol 18, 280-296 (2021).
22. J. K. M. Lim, A. Delaidelli, S. W. Minaker, H. F. Zhang, M. Colovic, H. Yang, G. L. Negri, S. von Karstedt, W. W. Lockwood, P. Schaffer, G. Leprivier, P. H. Sorensen, Cystine/glutamate antiporter xCT (SLC7A11) facilitates oncogenic RAS transformation by preserving intracellular redox balance. Proc Natl Acad Sci USA 116, 9433-9442 (2019).
23. M. A. Badgley, D. M. Kremer, H. C. Maurer, K. E. DelGiorno, H. J. Lee, V. Purohit, I. R. Sagalovskiy, A. Ma, J. Kapilian, C. E. M. Firl, A. R. Decker, S. A. Sastra, C. F. Palermo, L. R. Andrade, P. Sajjakulnukit, L. Zhang, Z. P. Tolstyka, T. Hirschhorn, C. Lamb, T. Liu, W. Gu, E. S. Seeley, E. Stone, G. Georgiou, U. Manor, A. Iuga, G. M. Wahl, B. R. Stockwell, C. A. Lyssiotis, K. P. Olive, Cysteine depletion induces pancreatic tumor ferroptosis in mice. Science 368, 85-89 (2020).
24. V. Kalatzis, S. Cherqui, C. Antignac, B. Gasnier, Cystinosin, the protein defective in cystinosis, is a H(+)-driven lysosomal cystine transporter. EMBO J 20, 5940-5949 (2001).
25. U. Sahin, A. Muik, E. Derhovanessian, I. Vogler, L. M. Kranz, M. Vormehr, A. Baum, K. Pascal, J. Quandt, D. Maurus, S. Brachtendorf, V. Lorks, J. Sikorski, R. Hilker, D. Becker, A. K. Eller, J. Grutzner, C. Boesler, C. Rosenbaum, M. C. Kuhnle, U. Luxemburger, A. Kemmer-Bruck, D. Langer, M. Bexon, S. Bolte, K. Kariko, T. Palanche, B. Fischer, A. Schultz, P. Y. Shi, C. Fontes-Garfias, J. L. Perez, K. A. Swanson, J. Loschko, I. L. Scully, M. Cutler, W. Kalina, C. A. Kyratsous, D. Cooper, P. R. Dormitzer, K. U. Jansen, O. Tureci, COVID-19 vaccine BNT162b1 elicits human antibody and THI T cell responses. Nature 586, 594-599 (2020).
26. A. J. Bochel, C. Williams, A. J. McCoy, H. J. Hoppe, A. J. Winter, R. D. Nicholls, K. Harlos, E. Y. Jones, I. Berger, A. B. Hassan, M. P. Crump, Structure of the Human Cation-Independent Mannose 6-Phosphate/IGF2 Receptor Domains 7-11 Uncovers the Mannose 6-Phosphate Binding Site of Domain 9. Structure 28, 1300-1312 e1305 (2020).
27. B. Li, X. Luo, B. Deng, J. Wang, D. W. McComb, Y. Shi, K. M. Gaensler, X. Tan, A. L. Dunn, B. A. Kerlin, Y. Dong, An Orthogonal Array Optimization of Lipid-like Nanoparticles for mRNA Delivery in Vivo. Nano Lett 15, 8099-8107 (2015).
28. Y. Zhang, H. Tan, J. D. Daniels, F. Zandkarimi, H. Liu, L. M. Brown, K. Uchida, O. A. O'Connor, B. R. Stockwell, Imidazole Ketone Erastin Induces Ferroptosis and Slows Tumor Growth in a Mouse Lymphoma Model. Cell Chem Biol 26, 623-633 e629 (2019).
29. U. E. Martinez-Outschoorn, M. Peiris-Pages, R. G. Pestell, F. Sotgia, M. P. Lisanti, Cancer metabolism: a therapeutic perspective. Nat Rev Clin Oncol 14, 113 (2017).
30. W. Zhang, P. Li, Q. Geng, Y. Duan, M. Guo, Y. Cao, Simultaneous determination of glutathione, cysteine, homocysteine, and cysteinylglycine in biological fluids by ion-pairing high-performance liquid chromatography coupled with precolumn derivatization. J Agric Food Chem 62, 5845-5852 (2014).
31. L. Campanella, G. Crescentini, P. Avino, Simultaneous determination of cysteine, cystine and 18 other amino acids in various matrices by high-performance liquid chromatography. J Chromatogr A 833, 137-145 (1999).
32. E. K. Schmidt, G. Clavarino, M. Ceppi, P. Pierre, SUnSET, a nonradioactive method to monitor protein synthesis. Nat Methods 6, 275-277 (2009).
33. B. Li, X. Luo, B. Deng, J. Wang, D. W. McComb, Y. Shi, K. M. Gaensler, X. Tan, A. L. Dunn, B. A. Kerlin, Y. Dong, An Orthogonal Array Optimization of Lipid-like Nanoparticles for mRNA Delivery in Vivo. Nano Lett 15, 8099-8107 (2015).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:
1. An engineered biomolecule comprising:
   a. a lysosomal targeting moiety;
   b. one or more cysteine-rich motifs, wherein each of the one or more cysteine-rich motifs is coupled to the lysosomal targeting moiety.
2. The engineered biomolecule of aspect 1, wherein the engineered biomolecule is DNA or RNA.
3. The engineered biomolecule of aspect 1, wherein the engineered biomolecule is a polypeptide.
4. The engineered biomolecule of any one of aspects 1-3, wherein the engineered biomolecule comprises at least two cysteine-rich motifs.
5. The engineered biomolecule of any one of aspects 1-4, wherein the lysosomal targeting moiety is selected from: IGF2 or M6PR binding domain thereof, any polypeptide set forth in Table 1, a LIMP-2 ligand, a sortilin ligand, and any combination thereof.
6. The engineered biomolecule of any one of any one of aspects 1-5, wherein the one or more cysteine-rich motifs are independently selected from DNAJC5, CYSRT1, a native cysteine-rich domain of a protein set forth in Table 2, or a protein set forth in Table 2.
7. The engineered biomolecule of any one of any one of aspects 1-6, wherein one or more nucleotides or amino acids of the engineered biomolecule are modified, wherein the modification reduces biomolecule immunogenicity, increases biomolecule stability, or both.
8. The engineered biomolecule of aspect 7, wherein the modification at each modified nucleotide is independently selected from methylpseudouridine, a phosphorothioate linkage, a locked nucleic acid (LNA) nucleotides comprising a methylene bridge between the 2′ and 4′ carbons of the ribose ring, or bridged nucleic acids (BNA), 2′-O-methyl analogs, 2′-deoxy analogs, or 2′-fluoro analogs, 2-aminopurine, 5-bromo-uridine, pseudouridine, (Ψ), N1-methylpseudouridine (me1Ψ), 5-methoxyuridine (5moU), inosine, 7-methylguanosine, inosine, 7-methylguanosine. Examples of guide RNA chemical modifications include, without limitation, incorporation of 2′-O-methyl (M), 2′-O-methyl 3′phosphorothioate (MS), S-constrained ethyl(cEt), or 2′-O-methyl 3′thioPACE (MSP) at one or more terminal nucleotides.
9. The engineered biomolecule of aspect 7, wherein the modification at each modified amino acid is independently selected from phosphorylation, acetylation, ubiquitylation, methylation, glycosylation, SUMOylation, palmitoylation, myristoylation, prenylation, sulfation, a reversible post-translational modification, an irreversible post-translational modification, a protein backbone post-translational modification; Nε-lysine acetylation, a non-histone protein acetylation, any one or more post-translational modifications set forth in one or more of post-translational modification databases: dbPTM, BioGRID. Phosphosite Plus, PTMCodev2, qPTM, PLMD, CPLM, YAAM, HPRD, PHOSIDA, PTM-SD, WERAM, EPSD, PhosphoNET, RegPhos, Phospho.ELM, Phospho3D, dbPSP, pTestis, LymPHOS. P3 DB, UniPep, GlycoFly, GlycoFish, mUbiSiDa, SwissPalm, dbSNO, or any combination thereof.
10. The engineered biomolecule of any one any one of aspects 1-9, wherein the engineered biomolecule is effective to inhibit ATF4 expression induction, reduce cytosolic cysteine, increase lysosomal cysteine, inhibit a cyst(e)ine stress response, or any combination thereof in a cell.
11. The engineered biomolecule of any one of any one of aspects 1-10, wherein the engineered biomolecule is effective to induce and/or potentiate ferroptosis.
12. A vector comprising:
   a. an engineered biomolecule of any one of any one of aspects 1-11, wherein the engineered biomolecule is an engineered polynucleotide; and
   b. optionally, a regulatory element, wherein the engineered polynucleotide is operably coupled to the regulatory element.
13. A delivery vehicle comprising:
   a. an engineered biomolecule of any one of any one of aspects 1-11;
   b. a vector as in aspect 12; or
   c. both.
14. The delivery vehicle of aspect 13, wherein the delivery vehicle comprises a micelle, nanoparticle, a lipid particles, a polymer or polymer-based particle, streptolysin-O, an exosome, an extracellular vesicle, dendrimers, a nanoclew, cell penetrating peptides, a multifunctional envelope-type nanodevice, a virus, a virus like particle, a vector, a vector system, a naked polynucleotide, or any combination thereof.
15. A pharmaceutical formulation comprising:
   a. an engineered biomolecule of any one of any one of aspects 1-11;
   b. a vector as in aspect 12;
   c. a delivery particle as in any one of aspects 13-14; or
   d. any combination of (a)-(c); and
   a pharmaceutically acceptable carrier.
16. The pharmaceutical formulation of aspect 15, further comprising an additional active agent.
17. The pharmaceutical formulation of aspect 16, wherein the additional active agent is effective to induce ferroptosis in a cell.
18. The pharmaceutical formulation of any one of aspects 16-17, wherein the additional active agent inhibits the X_{c}⁻ antiporter.
19. The pharmaceutical formulation of any one of aspects 16-18, wherein the additional active agent is selected from erastin, Ras-selective lethal small molecule 3, sulfasalazine or analogue thereof, lanperisone, sorafenib, fenugreek (trigonelline), acetaminophen, cisplatin, artesunate, siramesine, lapatinib, a combination of siramesine and lapatinib, ferumoxytol, salinomycin (ironomycin), dihydroartemisnin, gemcitabine, paclitaxel, temozolomide, buthionine sulfoximine (BSO), solasonine, siramesine, laptinib, BAY 87-2243,

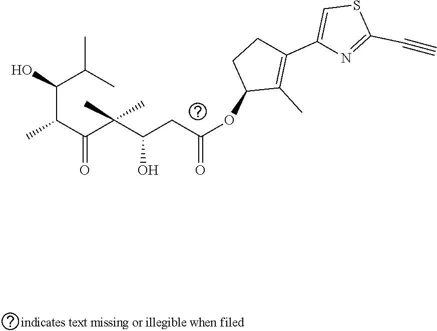

or a derivative or metabolite thereof, optionally

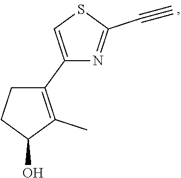

BLZ945, Dyclonine, Oxyfedrine, ML210, SSZ, RSL3, ML162, FIN56, Anti-PD1, gefitnib, erlotinib, TMZ, docetaxel, DAT, vemurafenib, lovastatin, atorvastatin, simvastatin, prominin2, artemisinin or a derivative thereof, vitamin E, baicalein, beta-elemene, gallic acid, buthionine sulfoximine, DP17, FIN56, FINO₂, a statin, deferoxamine mesylate, or any combination thereof.
20. A kit comprising:
   a. an engineered biomolecule of any one of aspects 1-11;
   b. a vector of aspect 12;
   c. a delivery vehicle of any one of aspects 13-14;
   d. a pharmaceutical formulation as in any one of aspects 15-19; or
   e. any combination thereof.
21. The kit of aspect 20, further comprising an additional active agent.
22. The kit of any one of aspects 20-21, wherein the additional active agent is effective to induce ferroptosis in a cell.
23. The kit of any one of aspects 20-22, wherein the additional active agent inhibits the X_{c}⁻ antiporter.
24. The kit of any one of aspects 20-23, wherein the additional active agent is selected from erastin, Ras-selective lethal small molecule 3, sulfasalazine or analogue thereof, lanperisone, sorafenib, fenugreek (trigonelline), acetaminophen, cisplatin, artesunate, siramesine, lapatinib, a combination of siramesine and lapatinib, ferumoxytol, salinomycin (ironomycin), dihydroartemisnin, gemcitabine, paclitaxel, temozolomide, buthionine sulfoximine (BSO), solasonine, siramesine, laptinib, BAY 87-2243,

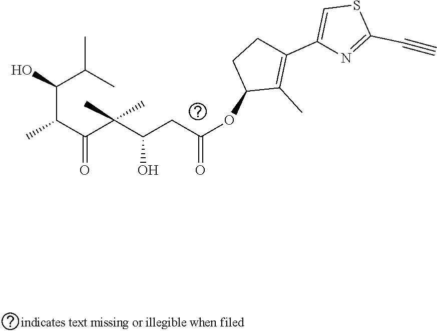

or a derivative or metabolite thereof, optionally

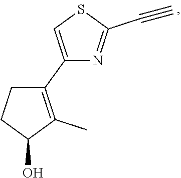

BLZ945, Dyclonine, Oxyfedrine, ML210, SSZ, RSL3, ML162, FIN56, Anti-PD1, gefitnib, erlotinib, TMZ, docetaxel, DAT, vemurafenib, lovastatin, atorvastatin, simvastatin, prominin2, artemisinin or a derivative thereof, vitamin E, baicalein, beta-elemene, gallic acid, buthionine sulfoximine, DP17, FIN56, FINO₂, a statin, deferoxamine mesylate, or any combination thereof.
25. A method comprising:
   delivering to a cell or cell population
      a. an engineered biomolecule of any one of aspects 1-11;
      b. a vector of aspect 12;
      c. a delivery vehicle of any one of aspects 13-14;
      d. a pharmaceutical formulation as in any one of aspects 16-19; or
      e. any combination thereof.
26. The method of aspect 25, wherein ferroptosis is induced and/or potentiated in the cell or cell population.
27. The method of any one of aspects 25-26, wherein cytosolic cysteine is decreased, lysosomal cysteine is increased, or both.
28. The method of any one of aspects 25-27, wherein ATF4 expression is decreased and/or ATF4 expression induction is decreased.
29. The method of any one of aspects 25-28, further comprising delivering to the cell an additional active agent.
30. The method of aspect 29, wherein the additional active agent is effective to induce ferroptosis in the cell or cell population.
31. The method of any one of aspects 29-30, wherein the additional active agent is effective to inhibit the X_{c}⁻ antiporter.
32. The method of any one of aspects 29-31, wherein the additional active agent is selected from erastin, Ras-selective lethal small molecule 3, sulfasalazine or analogue thereof, lanperisone, sorafenib, fenugreek (trigonelline), acetaminophen, cisplatin, artesunate, siramesine, lapatinib, a combination of siramesine and lapatinib, ferumoxytol, salinomycin (ironomycin), dihydroartemisnin, gemcitabine, paclitaxel, temozolomide, buthionine sulfoximine (BSO), solasonine, siramesine, laptinib, BAY 87-2243,

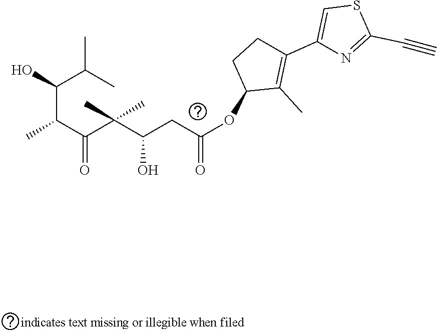

or a derivative or metabolite thereof, optionally

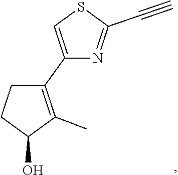

BLZ945, Dyclonine, Oxyfedrine, RSL3, ML210, SSZ, RSL3, ML162, FIN56, Anti-PD1, gefitnib, erlotinib, TMZ, docetaxel, DAT, vemurafenib, lovastatin, atorvastatin, simvastatin, prominin2, artemisinin or a derivative thereof, vitamin E, baicalein, beta-elemene, gallic acid, buthionine sulfoximine, DP17, FIN56, FINO₂, a statin, deferoxamine mesylate, or any combination thereof.
33. The method of any one of aspects 25-32, wherein the cell is a cancer cell.
34. A method of treating a proliferative disease in a subject in need thereof, the method comprising:
   administering to the subject
      a. an engineered biomolecule of any one of aspects 1-11;
      b. a vector of aspect 12;
      c. a delivery vehicle of any one of aspects 13-14;
      d. a pharmaceutical formulation as in any one of aspects 15-19; or
      e. any combination thereof.
35. The method of treating a proliferative disease as in aspect 34, wherein ferroptosis is induced and/or potentiated in a cell or cell population in the subject.
36. The method of treating a proliferative disease in any one aspects 34-35, wherein cytosolic cysteine is decreased in and/or lysosomal cysteine is increased, in a cell or cell population in the subject.
37. The method of treating a proliferative disease as in any one of aspects 34-36, wherein ATF4 expression is decreased and/or ATF4 expression induction is decreased in a cell or cell population in the subject.
38. The method of treating a proliferative disease in any one of aspects 34-37, wherein the cell or cell population is a cancer cell or cancer cell population.
39. The method of treating a proliferative disease in any one of aspects 34-38, further comprising administering an additional active agent to the subject.
40. The method of treating a proliferative disease in any one of claims 34-39, wherein the additional active agent is administered simultaneously, contemporaneously, or serially with (a)-(e).
41. The method of treating a proliferative disease in any one of aspects 34-40, wherein the additional active agent is effective to induce ferroptosis in a cell or cell population in the subject.
42. The method of treating a proliferative disease in any one of aspects 34-41 wherein the additional active agent is effective to inhibit the X_{c}⁻ antiporter in a cell or cell population in the subject.
43. The method of treating a proliferative disease in any one of aspects 34-42, wherein the additional active agent is selected from erastin, Ras-selective lethal small molecule 3, sulfasalazine or analogue thereof, lanperisone, sorafenib, fenugreek (trigonelline), acetaminophen, cisplatin, artesunate, siramesine, lapatinib, a combination of siramesine and lapatinib, ferumoxytol, salinomycin (ironomycin), dihydroartemisnin, gemcitabine, paclitaxel, temozolomide, buthionine sulfoximine (BSO), solasonine, siramesine, laptinib, BAY 87-2243,

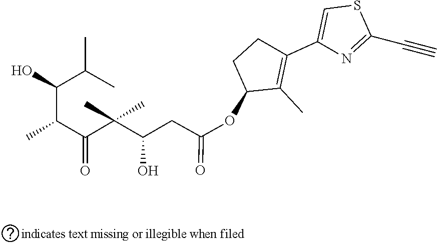

or a derivative or metabolite thereof, optionally

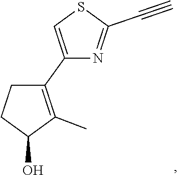

BLZ945, Dyclonine, Oxyfedrine, RSL3, ML210, SSZ, RSL3, ML162, FIN56, Anti-PD1, gefitnib, erlotinib, TMZ, docetaxel, DAT, vemurafenib, lovastatin, atorvastatin, simvastatin, prominin2, artemisinin or a derivative thereof, vitamin E, baicalein, beta-elemene, gallic acid, buthionine sulfoximine, DP17, FIN56, FINO₂, a statin, deferoxamine mesylate, or any combination thereof.
44. The method of treating a proliferative disease in any one of aspects 34-43 wherein cancer cell growth, cancer tumor growth, or both is inhibited, slowed, and/or stopped.
45. A method of inhibiting a cysteine stress response in a cell or cell population, the method comprising:
   delivering to the cell or cell population
      a. an engineered biomolecule of any one of aspects 1-11;
      b. a vector of aspect 12;
      c. a delivery vehicle of any one of aspects 13-14;
      d. a pharmaceutical formulation as in any one of aspects 15-19; or
      e. any combination thereof.
46. The method of aspect 45, wherein ferroptosis is induced and/or potentiated in a cell or cell population.
47. The method of inhibiting a cysteine stress response in a cell or cell population of any one of aspects 45-46, wherein cytosolic cysteine is decreased in and/or lysosomal cysteine is increased in the cell or cell population.
48. The method of inhibiting a cysteine stress response in a cell or cell population of any one of aspects 45-47, wherein ATF4 expression is decreased and/or ATF4 expression induction is decreased in the cell or cell population.
49. The method of inhibiting a cysteine stress response in a cell or cell population of any one of aspects 45-48, wherein the cell or cell population is a cancer cell or cancer cell population.
50. The method of inhibiting a cysteine stress response in a cell or cell population of any one of aspects 45-49, further comprising delivering an additional active agent cell or cell population.
51. The method of inhibiting a cysteine stress response in a cell or cell population of aspect 50, wherein the additional active agent is effective to induce ferroptosis in the cell or cell population.
52. The method of inhibiting a cysteine stress response in a cell or cell population of any one of aspects 50-51, wherein the additional active agent is effective to inhibit the X_{c}⁻ antiporter in the cell or cell population.
53. The method of inhibiting a cysteine stress response in a cell or cell population of any one of aspects 50-52, wherein the additional active agent is delivered simultaneously, contemporaneously, or serially with (a)-(e).
54. The method of inhibiting a cysteine stress response in a cell or cell population of any one of aspects 50-53, wherein the additional active agent is selected from erastin, Ras-selective lethal small molecule 3, sulfasalazine or analogue thereof, lanperisone, sorafenib, fenugreek (trigonelline), acetaminophen, cisplatin, artesunate, siramesine, lapatinib, a combination of siramesine and lapatinib, ferumoxytol, salinomycin (ironomycin), dihydroartemisnin, gemcitabine, paclitaxel, temozolomide, buthionine sulfoximine (BSO), solasonine, siramesine, laptinib, BAY 87-2243,

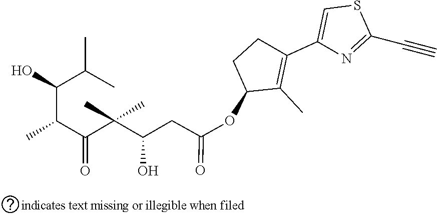

or a derivative or metabolite thereof, optionally

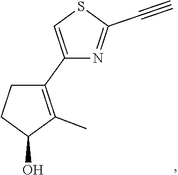

BLZ945, Dyclonine, Oxyfedrine, RSL3, ML210, SSZ, RSL3, ML162, FIN56, Anti-PD1, gefitnib, erlotinib, TMZ, docetaxel, DAT, vemurafenib, lovastatin, atorvastatin, simvastatin, prominin2, artemisinin or a derivative thereof, vitamin E, baicalein, beta-elemene, gallic acid, buthionine sulfoximine, DP17, FIN56, FINO₂, a statin, deferoxamine mesylate, or any combination thereof.

What is claimed is:

1. An engineered biomolecule comprising:
a. a lysosomal targeting moiety;
b. one or more cysteine-rich motifs, wherein each of the one or more cysteine-rich motifs is coupled to the lysosomal targeting moiety.
c.

2. The engineered biomolecule of claim 1, wherein the engineered biomolecule is DNA or RNA.

3. The engineered biomolecule of claim 1, wherein the engineered biomolecule is a polypeptide.

4. The engineered biomolecule of claim 1, wherein the engineered biomolecule comprises at least two cysteine-rich motifs.

5. The engineered biomolecule of claim 1, wherein the lysosomal targeting moiety is selected from: IGF2 or M6PR binding domain thereof, any polypeptide set forth in Table 1, a LIMP-2 ligand, a sortilin ligand, and any combination thereof.

6. The engineered biomolecule of claim 1, wherein the one or more cysteine-rich motifs are independently selected from DNAJC5, CYSRT1, a native cysteine-rich domain of a protein set forth in Table 2, or a protein set forth in Table 2.

7. The engineered biomolecule of claim 1, wherein one or more nucleotides or amino acids of the engineered biomolecule are modified, wherein the modification reduces biomolecule immunogenicity, increases biomolecule stability, or both.

8. The engineered biomolecule of claim 7, wherein the modification at each modified nucleotide is independently selected from methylpseudouridine, a phosphorothioate linkage, a locked nucleic acid (LNA) nucleotides comprising a methylene bridge between the 2′ and 4′ carbons of the ribose ring, or bridged nucleic acids (BNA), 2′-O-methyl analogs, 2′-deoxy analogs, or 2′-fluoro analogs, 2-aminopurine, 5-bromo-uridine, pseudouridine, (Ψ), N1-methylpseudouridine (me1Ψ), 5-methoxyuridine (5moU), inosine, 7-methylguanosine, inosine, 7-methylguanosine. Examples of guide RNA chemical modifications include, without limitation, incorporation of 2′-O-methyl (M), 2′-O-methyl 3′phosphorothioate (MS), S-constrained ethyl (cEt), or 2′-O-methyl 3′thioPACE (MSP) at one or more terminal nucleotides.

9. The engineered biomolecule of claim 7, wherein the modification at each modified amino acid is independently selected from phosphorylation, acetylation, ubiquitylation, methylation, glycosylation, SUMOylation, palmitoylation, myristoylation, prenylation, sulfation, a reversible post-translational modification, an irreversible post-translational modification, a protein backbone post-translational modification; Nε-lysine acetylation, a non-histone protein acetylation, any one or more post-translational modifications set forth in one or more of post-translational modification databases: dbPTM, BioGRID. Phosphosite Plus, PTMCodev2, qPTM, PLMD, CPLM, YAAM, HPRD, PHOSIDA, PTM-SD, WERAM, EPSD, PhosphoNET, RegPhos, Phospho.ELM, Phospho3D, dbPSP, pTestis, LymPHOS. P3 DB, UniPep, GlycoFly, GlycoFish, mUbiSiDa, SwissPalm, dbSNO, or any combination thereof.

10. The engineered biomolecule of claim 1, wherein the engineered biomolecule is effective to inhibit ATF4 expression induction, reduce cytosolic cysteine, increase lysosomal cysteine, inhibit a cyst(e)ine stress response, or any combination thereof in a cell.

11. The engineered biomolecule of claim 1, wherein the engineered biomolecule is effective to induce and/or potentiate ferroptosis.

12. A vector comprising:
a. an engineered biomolecule of any one of claim 1, wherein the engineered biomolecule is an engineered polynucleotide; and
b. optionally, a regulatory element, wherein the engineered polynucleotide is operably coupled to the regulatory element.

13. A delivery vehicle comprising:
a. an engineered biomolecule of claim 1;
b. a vector as in claim 12; or
c. both.

14. The delivery vehicle of claim 13, wherein the delivery vehicle comprises a micelle, nanoparticle, a lipid particles, a polymer or polymer-based particle, streptolysin-O, an exosome, an extracellular vesicle, dendrimers, a nanoclew, cell penetrating peptides, a multifunctional envelope-type nanodevice, a virus, a virus like particle, a vector, a vector system, a naked polynucleotide, or any combination thereof.

15. A pharmaceutical formulation comprising:
a. an engineered biomolecule of claim 1;
b. a vector as in claim 12;
c. a delivery particle as in claim 13; or
d. any combination of (a)-(c); and
a pharmaceutically acceptable carrier.

16. The pharmaceutical formulation of claim 15, further comprising an additional active agent.

17. The pharmaceutical formulation of claim 16, wherein the additional active agent is effective to induce ferroptosis in a cell.

18. The pharmaceutical formulation of any one of claims 16-17, wherein the additional active agent inhibits the X_{c}⁻ antiporter.

19. The pharmaceutical formulation of any one of claims 16-18, wherein the additional active agent is selected from erastin, Ras-selective lethal small molecule 3, sulfasalazine or analogue thereof, lanperisone, sorafenib, fenugreek (trigonelline), acetaminophen, cisplatin, artesunate, siramesine, lapatinib, a combination of siramesine and lapatinib, ferumoxytol, salinomycin (ironomycin), dihydroartemisnin, gemcitabine, paclitaxel, temozolomide, buthionine sulfoximine (BSO), solasonine, siramesine, laptinib, BAY 87-2243, 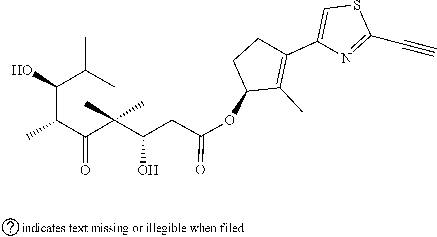
or a derivative or metabolite thereof, optionally 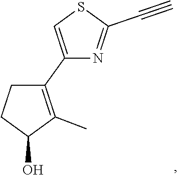
BLZ945, Dyclonine, Oxyfedrine, ML210, SSZ, RSL3, ML162, FIN56, Anti-PD1, gefitnib, erlotinib, TMZ, docetaxel, DAT, vemurafenib, lovastatin, atorvastatin, simvastatin, prominin2, artemisinin or a derivative thereof, vitamin E, baicalein, beta-elemene, gallic acid, buthionine sulfoximine, DP17, FIN56, FINO₂, a statin, deferoxamine mesylate, or any combination thereof.

20. A kit comprising:
a. an engineered biomolecule of claim 1;
b. a vector of claim 12;
c. a delivery vehicle of claim 13;
d. a pharmaceutical formulation as in any one of the preceding claims; or
e. any combination thereof.

21. The kit of claim 20, further comprising an additional active agent.

22. The kit of any one of claims 20-21 wherein the additional active agent is effective to induce ferroptosis in a cell.

23. The kit of any one of claims 20-22, wherein the additional active agent inhibits the X_{c}⁻ antiporter.

24. The kit of any one of claims 20-23, wherein the additional active agent is selected from erastin, Ras-selective lethal small molecule 3, sulfasalazine or analogue thereof, lanperisone, sorafenib, fenugreek (trigonelline), acetaminophen, cisplatin, artesunate, siramesine, lapatinib, a combination of siramesine and lapatinib, ferumoxytol, salinomycin (ironomycin), dihydroartemisnin, gemcitabine, paclitaxel, temozolomide, buthionine sulfoximine (BSO), solasonine, siramesine, laptinib, BAY 87-2243, 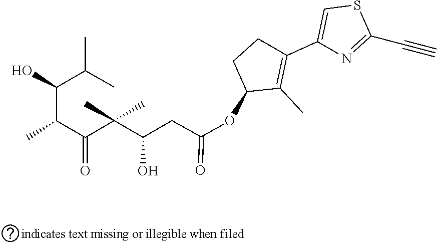
or a derivative or metabolite thereof, optionally 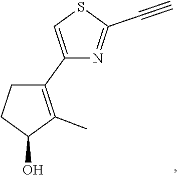
BLZ945, Dyclonine, Oxyfedrine, ML210, SSZ, RSL3, ML162, FIN56, Anti-PD1, gefitnib, erlotinib, TMZ, docetaxel, DAT, vemurafenib, lovastatin, atorvastatin, simvastatin, prominin2, artemisinin or a derivative thereof, vitamin E, baicalein, beta-elemene, gallic acid, buthionine sulfoximine, DP17, FIN56, FINO₂, a statin, deferoxamine mesylate, or any combination thereof.

25. A method comprising:
delivering to a cell or cell population
a. an engineered biomolecule of claim 1;
b. a vector of claim 12;
c. a delivery vehicle of claim 13;
d. a pharmaceutical formulation as in claim 15; or
e. any combination thereof.

26. The method of claim 25, wherein ferroptosis is induced and/or potentiated in the cell or cell population.

27. The method of any one of claims 25-26, wherein cytosolic cysteine is decreased, lysosomal cysteine is increased, or both.

28. The method of any one of claims 25-27, wherein ATF4 expression is decreased and/or ATF4 expression induction is decreased.

29. The method of any one of claims 25-28, further comprising delivering to the cell an additional active agent.

30. The method of claim 29, wherein the additional active agent is effective to induce ferroptosis in the cell or cell population.

31. The method of any one of claims 29-30, wherein the additional active agent is effective to inhibit the X_{c}⁻ antiporter.

32. The method of any one of claims 29-31, wherein the additional active agent is selected from erastin, Ras-selective lethal small molecule 3, sulfasalazine or analogue thereof, lanperisone, sorafenib, fenugreek (trigonelline), acetaminophen, cisplatin, artesunate, siramesine, lapatinib, a combination of siramesine and lapatinib, ferumoxytol, salinomycin (ironomycin), dihydroartemisnin, gemcitabine, paclitaxel, temozolomide, buthionine sulfoximine (BSO), solasonine, siramesine, laptinib, BAY 87-2243, 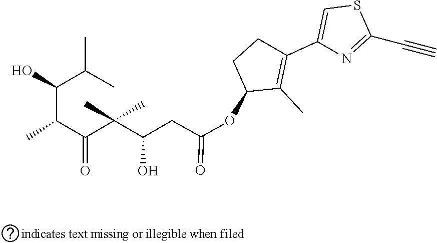
or a derivative or metabolite thereof, optionally 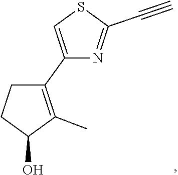
BLZ945, Dyclonine, Oxyfedrine, RSL3, ML210, SSZ, RSL3, ML162, FIN56, Anti-PD1, gefitnib, erlotinib, TMZ, docetaxel, DAT, vemurafenib, lovastatin, atorvastatin, simvastatin, prominin2, artemisinin or a derivative thereof, vitamin E, baicalein, beta-elemene, gallic acid, buthionine sulfoximine, DP17, FIN56, FINO₂, a statin, deferoxamine mesylate, or any combination thereof.

33. The method of any one of claims 29-32, wherein the cell is a cancer cell.

34. A method of treating a proliferative disease in a subject in need thereof, the method comprising:
administering to the subject
a. an engineered biomolecule of claim 1;
b. a vector of claim 12;
c. a delivery vehicle of claim 13;
d. a pharmaceutical formulation as in claim 15; or
e. any combination thereof.

35. The method of treating a proliferative disease as in claim 34, wherein ferroptosis is induced and/or potentiated in a cell or cell population in the subject.

36. The method of treating a proliferative disease as in claim 34, wherein cytosolic cysteine is decreased in and/or lysosomal cysteine is increased, in a cell or cell population in the subject.

37. The method of treating a proliferative disease as in claim 34, wherein ATF4 expression is decreased and/or ATF4 expression induction is decreased in a cell or cell population in the subject.

38. The method of treating a proliferative disease as in claim 34, wherein the cell or cell population is a cancer cell or cancer cell population.

39. The method of treating a proliferative disease as in claim 34, further comprising administering an additional active agent to the subject.

40. The method of treating a proliferative disease as in claim 34, wherein the additional active agent is administered simultaneously, contemporaneously, or serially with (a)-(e).

41. The method of treating a proliferative disease as in claim 34, wherein the additional active agent is effective to induce ferroptosis in a cell or cell population in the subject.

42. The method of treating a proliferative disease as in claim 34, wherein the additional active agent is effective to inhibit the X_{c}⁻ antiporter in a cell or cell population in the subject.

43. The method of treating a proliferative disease as in claim 34, wherein the additional active agent is selected from erastin, Ras-selective lethal small molecule 3, sulfasalazine or analogue thereof, lanperisone, sorafenib, fenugreek (trigonelline), acetaminophen, cisplatin, artesunate, siramesine, lapatinib, a combination of siramesine and lapatinib, ferumoxytol, salinomycin (ironomycin), dihydroartemisnin, gemcitabine, paclitaxel, temozolomide, buthionine sulfoximine (BSO), solasonine, siramesine, laptinib, BAY 87-2243, 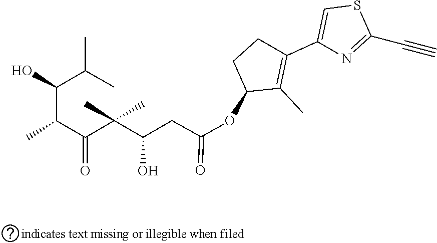
or a derivative or metabolite thereof, optionally 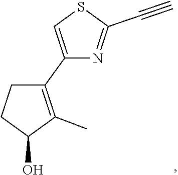
BLZ945, Dyclonine, Oxyfedrine, RSL3, ML210, SSZ, RSL3, ML162, FIN56, Anti-PD1, gefitnib, erlotinib, TMZ, docetaxel, DAT, vemurafenib, lovastatin, atorvastatin, simvastatin, prominin2, artemisinin or a derivative thereof, vitamin E, baicalein, beta-elemene, gallic acid, buthionine sulfoximine, DP17, FIN56, FINO₂, a statin, deferoxamine mesylate, or any combination thereof.

44. The method of treating a proliferative disease as in claim 34, wherein cancer cell growth, cancer tumor growth, or both is inhibited, slowed, and/or stopped.

45. A method of inhibiting a cysteine stress response in a cell or cell population, the method comprising:
delivering to the cell or cell population
a. an engineered biomolecule of any one of the preceding claims;
b. a vector of any one of the preceding claims;
c. a delivery vehicle of any one of the preceding claims;
d. a pharmaceutical formulation as in any one of the preceding claims; or
e. any combination thereof.

46. The method of claim 45, wherein ferroptosis is induced and/or potentiated in a cell or cell population.

47. The method of inhibiting a cysteine stress response in a cell or cell population of claim 45, wherein cytosolic cysteine is decreased in and/or lysosomal cysteine is increased in the cell or cell population.

48. The method of inhibiting a cysteine stress response in a cell or cell population of claim 45, wherein ATF4 expression is decreased and/or ATF4 expression induction is decreased in the cell or cell population.

49. The method of inhibiting a cysteine stress response in a cell or cell population of claim 45, wherein the cell or cell population is a cancer cell or cancer cell population.

50. The method of inhibiting a cysteine stress response in a cell or cell population of claim 45, further comprising delivering an additional active agent cell or cell population.

51. The method of inhibiting a cysteine stress response in a cell or cell population of claim 50, wherein the additional active agent is effective to induce ferroptosis in the cell or cell population.

52. The method of inhibiting a cysteine stress response in a cell or cell population of claim 50, wherein the additional active agent is effective to inhibit the X_{c}⁻ antiporter in the cell or cell population.

53. The method of inhibiting a cysteine stress response in a cell or cell population of claim 50, wherein the additional active agent is delivered simultaneously, contemporaneously, or serially with (a)-(e).

54. The method of inhibiting a cysteine stress response in a cell or cell population of claim 50, wherein the additional active agent is selected from erastin, Ras-selective lethal small molecule 3, sulfasalazine or analogue thereof, lanperisone, sorafenib, fenugreek (trigonelline), acetaminophen, cisplatin, artesunate, siramesine, lapatinib, a combination of siramesine and lapatinib, ferumoxytol, salinomycin (ironomycin), dihydroartemisnin, gemcitabine, paclitaxel, temozolomide, buthionine sulfoximine (BSO), solasonine, siramesine, laptinib, BAY 87-2243, 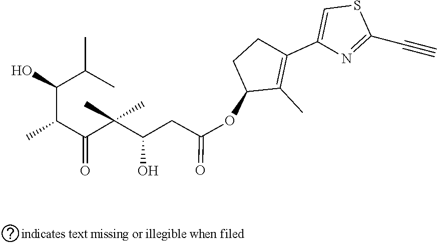
or a derivative or metabolite thereof, optionally 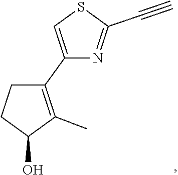
BLZ945, Dyclonine, Oxyfedrine, RSL3, ML210, SSZ, RSL3, ML162, FIN56, Anti-PD1, gefitnib, erlotinib, TMZ, docetaxel, DAT, vemurafenib, lovastatin, atorvastatin, simvastatin, prominin2, artemisinin or a derivative thereof, vitamin E, baicalein, beta-elemene, gallic acid, buthionine sulfoximine, DP17, FIN56, FINO₂, a statin, deferoxamine mesylate, or any combination thereof.
